SEQUENCE LISTING (69 sequences)

SEQ ID NO: 1 - DNA, 20 bp, synthetic construct
  misc_feature 1..20: note = Oligonucleotide
  source 1..20: mol_type = other DNA
cacgctgccttagaagatgg

SEQ ID NO: 2 - DNA, 20 bp, synthetic construct
  misc_feature 1..20: note = Oligonucleotide
  source 1..20: mol_type = other DNA
gttctgcagctgagatgagg

SEQ ID NO: 3 - DNA, 22 bp, synthetic construct
  misc_feature 1..22: note = Oligonucleotide
  source 1..22: mol_type = other DNA
gtttaatccgggctggttccat

SEQ ID NO: 4 - DNA, 22 bp, synthetic construct
  misc_feature 1..22: note = Oligonucleotide
  source 1..22: mol_type = other DNA
tagacccagtggacagatagcg

SEQ ID NO: 5 - DNA, 1323 bp, Saccharomyces cerevisiae
  source 1..1323: mol_type = unassigned DNA
atgcttgctgtcagaagattaacaagatacacattccttaagcgaacgcatccggtgttatatactcgtcgtgcatataaaattttgccttcaagatctactttcctaagaagatcattattacaaacacaactgcactcaaagatgactgctcatactaatatcaaacagcacaaacactgtcatgaggaccatcctatcagaagatcggactctgccgtgtcaattgtacatttgaaacgtgcgcccttcaaggttacagtgattggttctggtaactgggggaccaccatcgccaaagtcattgcggaaaacacagaattgcattcccatatcttcgagccagaggtgagaatgtgggtttttgatgaaaagatcggcgacgaaaatctgacggatatcataaatacaagacaccagaacgttaaatatctacccaatattgacctgccccataatctagtggccgatcctgatcttttacactccatcaagggtgctgacatccttgttttcaacatccctcatcaatttttaccaaacatagtcaaacaattgcaaggccacgtggcccctcatgtaagggccatctcgtgtctaaaagggttcgagttgggctccaagggtgtgcaattgctatcctcctatgttactgatgagttaggaatccaatgtggcgcactatctggtgcaaacttggcaccggaagtggccaaggagcattggtccgaaaccaccgtggcttaccaactaccaaaggattatcaaggtgatggcaaggatgtagatcataagattttgaaattgctgttccacagaccttacttccacgtcaatgtcatcgatgatgttgctggtatatccattgccggtgccttgaagaacgtcgtggcacttgcatgtggtttcgtagaaggtatgggatggggtaacaatgcctccgcagccattcaaaggctgggtttaggtgaaattatcaagttcggtagaatgtttttcccagaatccaaagtcgagacctactatcaagaatccgctggtgttgcagatctgatcaccacctgctcaggcggtagaaacgtcaaggttgccacatacatggccaagaccggtaagtcagccttggaagcagaaaaggaattgcttaacggtcaatccgcccaagggataatcacatgcagagaagttcacgagtggctacaaacatgtgagttgacccaagaattcccattattcgaggcagtctaccagatagtctacaacaacgtccgcatggaagacctaccggagatgattgaagagctagacatcgatgacgaatag

SEQ ID NO: 6 - protein, 440 aa, Saccharomyces cerevisiae
MLAVRRLTRYTFLKRTHPVLYTRRAYKILPSRSTFLRRSLLQTQLHSKMTAHTNIKQHKHCHEDHPIRRSDSAVSIVHLKRAPFKVTVIGSGNWGTTIAKVIAENTELHSHIFEPEVRMWVFDEKIGDENLTDIINTRHQNVKYLPNIDLPHNLVADPDLLHSIKGADILVFNIPHQFLPNIVKQLQGHVAPHVRAISCLKGFELGSKGVQLLSSYVTDELGIQCGALSGANLAPEVAKEHWSETTVAYQLPKDYQGDGKDVDHKILKLLFHRPYFHVNVIDDVAGISIAGALKNVVALACGFVEGMGWGNNASAAIQRLGLGEIIKFGRMFFPESKVETYYQESAGVADLITTCSGGRNVKVATYMAKTGKSALEAEKELLNGQSAQGIITCREVHEWLQTCELTQEFPLFEAVYQIVYNNVRMEDLPEMIEELDIDDE

SEQ ID NO: 7 - DNA, 1710 bp, Saccharomyces cerevisiae
  source 1..1710: mol_type = unassigned DNA
atgaaggatttaaaattatcgaatttcaaaggcaaatttataagcagaaccagtcactggggacttacgggtaagaagttgcggtatttcatcactatcgcatctatgacgggcttctccctgtttggatacgaccaagggttgatggcaagtctaattactggtaaacagttcaactatgaatttccagcaaccaaagaaaatggcgatcatgacagacacgcaactgtagtgcagggcgctacaacctcctgttatgaattaggttgtttcgcaggttctctattcgttatgttctgcggtgaaagaattggtagaaaaccattaatcctgatgggttccgtaataaccatcattggtgccgttatttctacatgcgcatttcgtggttactgggcattaggccagtttatcatcggaagagtcgtcaccggtgttggaacagggttgaatacatctactattcccgtttggcaatcagaaatgtcaaaagctgaaaatagagggttgctggtcaatttagaaggttccacaattgcttttggtactatgattgcttattggattgattttgggttgtcttataccaacagttctgttcagtggagattccccgtgtcaatgcaaatcgtttttgctctcttcctgcttgctttcatgattaaactacctgaatcgccacgttggctgatttctcaaagtcgaacagaagaagctcgctacttggtaggaacactagacgacgcggatccaaatgatgaggaagttataacagaagttgctatgcttcacgatgctgttaacaggaccaaacacgagaaacattcactgtcaagtttgttctccagaggcaggtcccaaaatcttcagagggctttgattgcagcttcaacgcaatttttccagcaatttactggttgtaacgctgccatatactactctactgtattattcaacaaaacaattaaattagactatagattatcaatgatcataggtggggtcttcgcaacaatctacgccttatctactattggttcattttttctaattgaaaagctaggtagacgtaagctgtttttattaggtgccacaggtcaagcagtttcattcacaattacatttgcatgcttggtcaaagaaaataaagaaaacgcaagaggtgctgccgtcggcttatttttgttcattacattctttggtttgtctttgctatcattaccatggatatacccaccagaaattgcatcaatgaaagttcgtgcatcaacaaacgctttctccacatgtactaattggttgtgtaactttgcggttgtcatgttcaccccaatatttattggacagtccggttggggttgctacttattttttgctgttatgaattatttatacattccagttatcttctttttctaccctgaaaccgccggaagaagtttggaggaaatcgacatcatctttgctaaagcatacgaggatggcactcaaccatggagagttgctaaccatttgcccaagttatccctacaagaagtcgaagatcatgccaatgcattgggctcttatgacgacgaaatggaaaaagaggactttggtgaagatagagtagaagacacctataaccaaattaacggcgataattcgtctagttcttcaaacatcaaaaatgaagatacagtgaacgataaagcaaattttgagggttga

SEQ ID NO: 8 - protein, 569 aa, Saccharomyces cerevisiae
MKDLKLSNFKGKFISRTSHWGLTGKKLRYFITIASMTGFSLFGYDQGLMASLITGKQFNYEFPATKENGDHDRHATVVQGATTSCYELGCFAGSLFVMFCGERIGRKPLILMGSVITIIGAVISTCAFRGYWALGQFIIGRVVTGVGTGLNTSTIPVWQSEMSKAENRGLLVNLEGSTIAFGTMIAYWIDFGLSYTNSSVQWRFPVSMQIVFALFLLAFMIKLPESPRWLISQSRTEEARYLVGTLDDADPNDEEVITEVAMLHDAVNRTKHEKHSLSSLFSRGRSQNLQRALIAASTQFFQQFTGCNAAIYYSTVLFNKTIKLDYRLSMIIGGVFATIYALSTIGSFFLIEKLGRRKLFLLGATGQAVSFTITFACLVKENKENARGAAVGLFLFITFFGLSLLSLPWIYPPEIASMKVRASTNAFSTCTNWLCNFAVVMFTPIFIGQSGWGCYLFFAVMNYLYIPVIFFFYPETAGRSLEEIDIIFAKAYEDGTQPWRVANHLPKLSLQEVEDHANALGSYDDEMEKEDFGEDRVEDTYNQINGDNSSSSSNIKNEDTVNDKANFEG

SEQ ID NO: 9 - DNA, 2313 bp, Saccharomyces cerevisiae
  source 1..2313: mol_type = unassigned DNA
atggataacgttgtagatccgtggtatataaatccctcaggcttcgcgaaagacactcaagatgaggagtatgttcaacatcatgataatgtcaatcctaccatacccccacccgacaattatattttgaataatgaaaacgatgatggcctcgataacttgttaggtatggactactataacatcgatgacctgttgactcaagagttaagagatctggatattcctttagtgccttctcctaagacgggcgatggttcttctgataaaaagaatattgatagaacttggaaccttggtgatgaaaacaacaaagtctcccactatagcaaaaaatcaatgtcctcacacaagagaggtctaagtggcacagcgatatttggatttctcggccataataagacattgagtatttccagtttacagcaatccattctaaatatgtctaaagatccgcaacccatggaactcataaatgaattgggtaatcataatacggtaaaaaataacaatgatgactttgaccatataagggaaaatgatggtgaaaatagctatttgagccaagttttgttgaaacagcaggaggagttaagaattgctcttgaaaaacaaaaggaagtgaacgaaaaattggagaagcagttgagagacaatcaaatacagcaagaaaagttgcgtaaagtattagaagagcaagaagaggtggcgcagaagttggtttctggggctacaaattctaattccaaacctggatctccagtaatactaaagacacctgccatgcaaaacggtagaatgaaagataatgctataatcgtcacaacgaactctgcaaatggcggatatcaatttcctcctccgacgttaatatcgcctcggatgtcaaatacttcaataaatggttcaccatccaggaaataccataggcaacgatatccaaataaaagcccagaaagtaatggattgaaccttttttcctctaacagtggttatttgagagattctgaactgctttcattttctccacaaaattataatttaaacttggacggcttgacttataatgaccataataacaccagtgataaaaacaataatgataaaaaaaatagtactggtgataacatattccgtctgttcgaaaagacttccccgggtgggctaagtatctctccaaggataaatggaaatagtttgagatcgcccttcctcgtcggcacagataaaagcagggatgatcgatatgctgctggcacgttcacgcctagaacacagttgtcacctatccacaagaaaagggaatccgtagtttccacggtctcgacaatatcacaactgcaggatgacactgaacccatccacatgcgaaatacccagaacccaacattaagaaatgcaaacgctttagcgtcatcaagtgtactacctcctattcctggttccagcaataacactccaattaagaattctttgccacaaaaacatgtatttcaacatactcccgtcaaagctccaccaaagaacggaagtaacctagctccgcttctaaatgcaccggatttaacagatcatcagttagaaattaagacacccatacgaaataacagtcactgtgaagtggaaagctatccgcaagtaccacctgtcacacatgatattcacaaaagccccactttgcatagtacgtctcctttaccagatgaaataatacctaggactacgccaatgaaaataaccaagaaaccaactactctgcctccgggtaccattgaccagtacgtcaaggaactacccgacaaactattcgagtgcttataccctaactgtaacaaagtattcaagcgtagatacaacataaggtcgcatattcagacacatttgcaagatagaccgtattcatgcgactttcccggttgcaccaaggcgtttgttcgcaatcatgatttaataagacacaaaatctcccataatgccaagaaatacatctgcccatgcggaaagagatttaatagggaggatgctctaatggtgcatagaagtcggatgatttgcaccggcggtaagaaattagaacattcgatcaacaagaaacttacatctcccaaaaaaagcctgcttgacagcccgcatgacacaagtcccgtaaaagaaactatcgcccgggataaagatgggagcgtcctaatgaaaatggaggaacagctgcgagatgatatgcgcaaacatggattactggatccacccccatccacagcagcgcacgagcaaaactcgaaccgcaccctttcaaacgaaactgatgctctctga

SEQ ID NO: 10 - protein, 770 aa, Saccharomyces cerevisiae
MDNVVDPWYINPSGFAKDTQDEEYVQHHDNVNPTIPPPDNYILNNENDDGLDNLLGMDYYNIDDLLTQELRDLDIPLVPSPKTGDGSSDKKNIDRTWNLGDENNKVSHYSKKSMSSHKRGLSGTAIFGFLGHNKTLSISSLQQSILNMSKDPQPMELINELGNHNTVKNNNDDFDHIRENDGENSYLSQVLLKQQEELRIALEKQKEVNEKLEKQLRDNQIQQEKLRKVLEEQEEVAQKLVSGATNSNSKPGSPVILKTPAMQNGRMKDNAIIVTTNSANGGYQFPPPTLISPRMSNTSINGSPSRKYHRQRYPNKSPESNGLNLFSSNSGYLRDSELLSFSPQNYNLNLDGLTYNDHNNTSDKNNNDKKNSTGDNIFRLFEKTSPGGLSISPRINGNSLRSPFLVGTDKSRDDRYAAGTFTPRTQLSPIHKKRESVVSTVSTISQLQDDTEPIHMRNTQNPTLRNANALASSSVLPPIPGSSNNTPIKNSLPQKHVFQHTPVKAPPKNGSNLAPLLNAPDLTDHQLEIKTPIRNNSHCEVESYPQVPPVTHDIHKSPTLHSTSPLPDEIIPRTTPMKITKKPTTLPPGTIDQYVKELPDKLFECLYPNCNKVFKRRYNIRSHIQTHLQDRPYSCDFPGCTKAFVRNHDLIRHKISHNAKKYICPCGKRFNREDALMVHRSRMICTGGKKLEHSINKKLTSPKKSLLDSPHDTSPVKETIARDKDGSVLMKMEEQLRDDMRKHGLLDPPPSTAAHEQNSNRTLSNETDAL

SEQ ID NO: 11 - DNA, 2312 bp, Saccharomyces cerevisiae
  source 1..2312: mol_type = unassigned DNA
atggataacgttgtagatccgtggtatataaatccctcaggcttcgcgaaagacactcaagatgaggagtatgttcaacatcatgataatgtcaatcctaccatacccccacccgacaattatattttgaataatgaaaacgatgatggcctcgataacttgttaggtatggactactataacatcgatgacctgttgactcaagagttaagagatctggatattcctttagtgccttctcctaagacgggcgatggttcttctgataaaaagaatattgatagaacttggaaccttggtgatgaaaacaacaaagtctcccactatagcaaaaaatcaatgtcctcacacaagagaggtctaagtggcacagcgatatttggatttctcggccataataagacattgagtatttccagtttacagcaatccattctaaatatgtctaaagatccgcaacccatggaactcataaatgaattgggtaatcataatacggtaaaaaataacaatgatgactttgaccatataagggaaaatgatggtgaaaatagctatttgagccaagttttgttgaaacagcaggaggagttaagaattgctcttgaaaaacaaaaggaagtgaacgaaaaattggagaagcagttgagagacaatcaaatacagcaagaaaagttgcgtaaagtattagaagagcaagaagaggtggcgcagaagttggtttctggggctacaaattctaattccaaacctggatctccagtaatactaaagacacctgccatgcaaaacggtagaatgaaagataatgctataatcgtcacaacgaactctgcaaatggcggatatcaatttcctcctccgacgttaatatcgcctcggatgtcaaatacttcaataaatggttcaccatccaggaaataccataggcaacgatatccaaataaaagcccagaaagtaatggattgaaccttttttcctctaacagtggttatttgagagattctgaactgctttcattttctccacaaaattataatttaaacttggacggcttgacttataatgaccataataacaccagtgataaaaacaataatgataaaaaaatagtactggtgataacatattccgtctgttcgaaaagacttccccgggtgggctaagtatctctccaaggataaatggaaatagtttgagatcgcccttcctcgtcggcacagataaaagcagggatgatcgatatgctgctggcacgttcacgcctagaacacagttgtcacctatccacaagaaaagggaatccgtagtttccacggtctcgacaatatcacaactgcaggatgacactgaacccatccacatgcgaaatacccagaacccaacattaagaaatgcaaacgctttagcgtcatcaagtgtactacctcctattcctggttccagcaataacactccaattaagaattctttgccacaaaaacatgtatttcaacatactcccgtcaaagctccaccaaagaacggaagtaacctagctccgcttctaaatgcaccggatttaacagatcatcagttagaaattaagacacccatacgaaataacagtcactgtgaagtggaaagctatccgcaagtaccacctgtcacacatgatattcacaaaagccccactttgcatagtacgtctcctttaccagatgaaataatacctaggactacgccaatgaaaataaccaagaaaccaactactctgcctccgggtaccattgaccagtacgtcaaggaactacccgacaaactattcgagtgcttataccctaactgtaacaaagtattcaagcgtagatacaacataaggtcgcatattcagacacatttgcaagatagaccgtattcatgcgactttcccggttgcaccaaggcgtttgttcgcaatcatgatttaataagacacaaaatctcccataatgccaagaaatacatctgcccatgcggaaagagatttaatagggaggatgctctaatggtgcatagaagtcggatgatttgcaccggcggtaagaaattagaacattcgatcaacaagaaacttacatctcccaaaaaaagcctgcttgacagcccgcatgacacaagtcccgtaaaagaaactatcgcccgggataaagatgggagcgtcctaatgaaaatggaggaacagctgcgagatgatatgcgcaaacatggattactggatccacccccatccacagcagcgcacgagcaaaactcgaaccgcaccctttcaaacgaaactgatgctctctga

SEQ ID NO: 12 - protein, 387 aa, Saccharomyces cerevisiae
MDNVVDPWYINPSGFAKDTQDEEYVQHHDNVNPTIPPPDNYILNNENDDGLDNLLGMDYYNIDDLLTQELRDLDIPLVPSPKTGDGSSDKKNIDRTWNLGDENNKVSHYSKKSMSSHKRGLSGTAIFGFLGHNKTLSISSLQQSILNMSKDPQPMELINELGNHNTVKNNNDDFDHIRENDGENSYLSQVLLKQQEELRIALEKQKEVNEKLEKQLRDNQIQQEKLRKVLEEQEEVAQKLVSGATNSNSKPGSPVILKTPAMQNGRMKDNAIIVTTNSANGGYQFPPPTLISPRMSNTSINGSPSRKYHRQRYPNKSPESNGLNLFSSNSGYLRDSELLSFSPQNYNLNLDGLTYNDHNNTSDKNNNDKKIVLVITYSVCSKRLPRV

SEQ ID NO: 13 - protein, 169 aa, Saccharomyces cerevisiae
MDNVVDPWYINPSGFAKDTQDEEYVQHHDNVNPTIPPPDNYILNNENDDGLDNLLGMDYYNIDDLLTQELRDLDIPLVPSPKTGDGSSDKKNIDRTWNLGDENNKVSHYSKKSMSSHKRGLSGTAIFGFLGHNKTLSISSLQQSILNMSKDPQPMELINELGNHNTVKK

SEQ ID NO: 14 - protein, 186 aa, Saccharomyces cerevisiae
MDNVVDPWYINPSGFAKDTQDEEYVQHHDNVNPTIPPPDNYILNNENDDGLDNLLGMDYYNIDDLLTQELRDLDIPLVPSPKTGDGSSDKKNIDRTWNLGDENNKVSHYSKKSMSSHKRGLSGTAIFGFLGHNKTLSISSLQQSILNMSKDPQPMELINELGNHNTVKNNNDDFDHIRENDGEIAI

SEQ ID NO: 15 - protein, 191 aa, Saccharomyces cerevisiae
MDNVVDPWYINPSGFAKDTQDEEYVQHHDNVNPTIPPPDNYILNNENDDGLDNLLGMDYYNIDDLLTQELRDLDIPLVPSPKTGDGSSDKKNIDRTWNLGDENNKVSHYSKKSMSSHKRGLSGTAIFGFLGHNKTLSISSLQQSILNMSKDPQPMELINELGNHNTVKNNNDDFDHIRENDGENSYLSQVC

SEQ ID NO: 16 - protein, 388 aa, Saccharomyces cerevisiae
MDNVVDPWYINPSGFAKDTQDEEYVQHHDNVNPTIPPPDNYILNNENDDGLDNLLGMDYYNIDDLLTQELRDLDIPLVPSPKTGDGSSDKKNIDRTWNLGDENNKVSHYSKKSMSSHKRGLSGTAIFGFLGHNKTLSISSLQQSILNMSKDPQPMELINELGNHNTVKNNNDDFDHIRENDGENSYLSQVLLKQQEELRIALEKQKEVNEKLEKQLRDNQIQQEKLRKVLEEQEEVAQKLVSGATNSNSKPGSPVILKTPAMQNGRMKDNAIIVTTNSANGGYQFPPPTLISPRMSNTSINGSPSRKYHRQRYPNKSPESNGLNLFSSNSGYLRDSELLSFSPQNYNLNLDGLTYNDHNNTSDKNNNDKKIVLVITYSVCSKRLPRVG

SEQ ID NO: 17 - protein, 322 aa, Saccharomyces cerevisiae
MPLNKSNIREYKLVVVGGGGVGKSALTIQLTQSHFVDEYDPTIEDSYRKQVVIDDEVSILDILDTTGQEEYSAMREQYMRNGEGFLLVYSITSKSSLDELMTYYQQILRVKDTDYVPIVVVGNKSDLENEKQVSYQDGLNMAKQMNAPFLETSAKQAINVEEAFYTLARLVRDEGGKYNKTLTENDNSKQTSQDTKGSGANSVPRNSGGHRKMSNAANGKNVNSSTTVVNARNASIESKTGLAGNQATNGKTQTDRTNIDNSTGQAGQANAQSANTVNNRVNNNSKAGQVSNAKQARKQQAAPGGNTSEASKSGSGGCCIIS

SEQ ID NO: 18 - DNA, 969 bp, Saccharomyces cerevisiae
  misc_feature 198: note = n is a, c, g, or t
  source 1..969: mol_type = unassigned DNA
atgcctttgaacaagtcgaacataagagagtacaagctagtcgtcgttggtggtggtggtgttggtaaatctgctttgaccatacaattgacccaatcgcactttgtagatgaatacgatcccacaattgaggattcatacaggaagcaagtggtgattgatgatgaagtgtctatattggacattttggatactacngggcaggaagaatactctgctatgagggaacaatacatgcgcaacggcgaaggattcctattggtttactctataacatccaagtcgtctcttgatgagcttatgacttactatcaacagatattgagagtcaaagataccgactatgttccaattgtggttgttggtaacaaatctgatttagaaaacgaaaaacaggtctcttaccaggacgggttgaacatggcaaagcaaatgaacgctcctttcttggagacatctgctaagcaagcaatcaacgtggaagaggcgttttacactctagcacgtttagttagagacgaaggcggcaagtacaacaagactttgacggaaaatgacaactccaagcaaacttctcaagatacaaaagggagcggtgccaactctgtgcctagaaatagcggtggccacaggaagatgagcaatgctgccaacggtaaaaatgtgaacagtagcacaactgtcgtgaatgccaggaatgcaagcatagagagtaagacagggttggcaggcaaccaggcgacaaatggtaagacacaaactgatcgcaccaatatagacaattccacgggccaagctggtcaggccaacgctcaaagcgctaatacggttaataatcgtgtaaataataatagtaaggccggtcaagtttcaaatgctaaacaggctaggaagcagcaagctgcacccggcggtaacaccagtgaagcctccaagagcggatcgggtggctgttgtattataagttaa

SEQ ID NO: 19 - protein, 322 aa, Saccharomyces cerevisiae
MPLNKSNIREYKLVVVGGGGVGKSALTIQLTQSHFVDEYDPTIEDSYRKQVVIDDEVSILDILDTAGQEEYSAMREQYMRNGEGFLLVYSITSKSSLDELMTYYQQILRVKDTDYVPIVVVGNKSDLENEKQVSYQDGLNMAKQMNAPFLETSAKQAINVEEAFYTLARLVRDEGGKYNKTLTENDNSKQTSQDTKGSGANSVPRNSGGHRKMSNAANGKNVNSSTTVVNARNASIESKTGLAGNQATNGKTQTDRTNIDNSTGQAGQANAQSANTVNNRVNNNSKAGQVSNAKQARKQQAAPGGNTSEASKSGSGGCCIIS

SEQ ID NO: 20 - protein, 3079 aa, Saccharomyces cerevisiae
MSQPTKNKKKEHGTDSKSSRMTRTLVNHILFERILPILPVESNLSTYSEVEEYSSFISCRSVLINVTVSRDANAMVEGTLELIESLLQGHEIISDKGSSDVIESILIILRLLSDALEYNWQNQESLHYNDISTHVEHDQEQKYRPKLHNILPDYSSTHSNGNKHFFHQSKPQALIPELASKLLESCAKLKFNTRTLQILQNMISHVHGNIVTTLSSSILPRHKSYLTRHNHPSHCKMIDSTLGHILRFVAASNPSEYFEFIRKSVQVPVTQTHTHSHSHSHSLPSSVYNSIVPHFDLFSFIHLSKDNFKKYLELIKNLSVTLRKTIYHCLLLHYSAKAIMFWIMTRPAEYYELFNLLKDNNNEHSKSLNTLNHTLFEEIHSTFNVNSMITTNQNAHQGSSSPSSSSPSSPPSSSSSDNNNQNIIAKSLSRQLSHHQSYIQQQSERKLHSSWTTNSQSSTSLSSSTSDSTTTDFSTHTQPGEYDPSLPDTPTMSNITISASSLLSQTPTPTTQLQQRLNSAAAAAAAAASPSNSTPTGYTAEQQSRASYDAHKTGHTGKDYDEHFLSITRLDNVLELYTHFDDTEVLPHTSVLKFLTTLTMFDIDLFNELNATSFKYIPDCTMHRPKERTSSFNNTAHETGSEKTSGIKHITQGLKKLTSLPSSTKKTVKFMKMLLRNLIGNQAVSDVALLDTMRALLSFFTMTSAVFLVDRNLPSVLFAKRLIPIMGTNLSVGQDWNSKINNSLMVCLKKNSTTFVQLQLIFFSSAIQFDHELLLARLSIDTMANNLNMQKLCLYTEGFRIFFDIPSKKELRKAIAVKISKFFKTLFSIIADILLQEFPYFDEQITDIVASILDGTIINEYGTKKHFKGSSPSLCSTTRSRSGSTSQSSMTPVSPLGLDTDICPMNTLSLVGSSTSRNSDNVNSLNSSPKNLSSDPYLSHLVAPRARHALGGPSSIIRNKIPTTLTSPPGTEKSSPVQRPQTESISATPMAITNSTPLSSAAFGIRSPLQKIRTRRYSDESLGKFMKSTNNYIQEHLIPKDLNEATLQDARRIMINIFSIFKRPNSYFIIPHNINSNLQWVSQDFRNIMKPIFVAIVSPDVDLQNTAQSFMDTLLSNVITYGESDENISIEGYHLLCSYTVTLFAMGLFDLKINNEKRQILLDITVKFMKVRSHLAGIAEASHHMEYISDSEKLTFPLIMGTVGRALFVSLYSSQQKIEKTLKIAYTEYLSAINFHERNIDDADKTWVHNIEFVEAMCHDNYTTSGSIAFQRRTRNNILRFATIPNAILLDSMRMIYKKWHTYTHSKSLEKQERNDFRNFAGILASLSGILFINKKILQEMYPYLLDTVSELKKNIDSFISKQCQWLNYPDLLTRENSRDILSVELHPLSFNLLFNNLRLKLKELACSDLSIPENESSYVLLEQIIKMLRTILGRDDDNYVMMLFSTEIVDLIDLLTDEIKKIPAYCPKYLKAIIQMTKMFSALQHSEVNLGVKNHFHVKNKWLRQVTDWFQVSIAREYDFENLSKPLKEMDLVKRDMDILYIDTAIEASTAIAYLTRHTFLEIPPAASDPELSRSRSVIFGFYFNILMKGLEKSSDRDNYPVFLRHKMSVLNDNVILSLTNLSNTNVDASLQFTLPMGYSGNRNIRNAFLEVFINIVTNYRTYTAKTDLGKLEAADKFLRYTIEHPQLSSFGAAVCPASDIDAYAAGLINAFETRNATHIVVSQLIKNEIENSSRPTDILRRNSCATRSLSMLARSKGSEYLIRTLQPLLKKIIQNRDFFEIEKLKPEDSDAERQIELFVKYMNELLESISNSVSYFPPPLFYICQNIYKVACEKFPDHAIIAAGSFVFLRFFCPALVSPDSENIIDISHLSEKRTFISLAKVIQNIANGSENFSRWPALCSQKDFLKECSDRIFRFLAELCRTDRTIDIQVRTDPTPIAFDYQFLHSFVYLYGLEVRRNVLNEAKHDDGDIDGDDFYKTTFLLIDDVLGQLGQPKMEFSNEIPIYIREHMDDYPELYEFMNRHAFRNIETSTAYSPSVHESTSSEGIPIITLTMSNFSDRHVDIDTVAYKFLQIYARIWTTKHCLIIDCTEFDEGGLDMRKFISLVMGLLPEVAPKNCIGCYYFNVNETFMDNYGKCLDKDNVYVSSKIPHYFINSNSDEGLMKSVGITGQGLKVLQDIRVSLHDITLYDEKRNRFTPVSLKIGDIYFQVLHETPRQYKIRDMGTLFDVKFNDVYEISRIFEVHVSSITGVAAEFTVTFQDERRLIFSSPKYLEIVKMFYYAQIRLESEYEMDNNSSTSSPNSNNKDKQQKERTKLLCHLLLVSLIGLFDESKKMKNSSYNLIAATEASFGLNFGSHFHRSPEVYVPEDTTTFLGVIGKSLAESNPELTAYMFIYVLEALKNNVIPHVYIPHTICGLSYWIPNLYQHVYLADDEEGPKNISHIFRILIRLSVRETDFKAVYMQYVWLLLLDDGRLTDIIVDEVINHALERDSENRDWKKTISLLTVLPTTEVANNIIQKILAKIRSFLPSLKLEAMTQSWSELTILVKISIHVFFETSLLVQMYLPEILFIVSLLIDVGPRELRSSLHQLLMNVCHSLAINSALPQDHRNNLDEISDIFAHQKVKFMFGFSEDKGRILQIFSASSFASKFNILDFFINNILLLMEYSSTYEANVWKTRYKKYVLESVFTSNSFLSARSIMIVGIMGKSYITEGLCKAMLIETMKVIAEPKITDEHLFLAISHIFTYSKIVEGLDPNLDLMKHLFWFSTLFLESRHPIIFEGALLFVSNCIRRLYMAQFENESETSLISTLLKGRKFAHTFLSKIENLSGIVWNEDNFTHILIFIINKGLSNPFIKSTAFDFLKMMFRNSYFEHQINQKSDHYLCYMFLLYFVLNCNQFEELLGDVDFEGEMVNIENKNTIPKILLEWLSSDNENANITLYQGAILFKCSVTDEPSRFRFALIIRHLLTKKPICALRFYSVIRNEIRKISAFEQTSDCVPLAFDILNLLVTHSESNSLEKLHEESIERLTKRGLSIVTSSGIFAKNSDMMIPLDVKPEDIYERKRIMTMILSRMSCSA

SEQ ID NO: 21 - DNA, 9240 bp, Saccharomyces cerevisiae
  source 1..9240: mol_type = unassigned DNA
atgtcccagcccactaagaataagaagaaagaacacgggaccgattccaagtcatcccgcatgactcggacgttggttaaccatattctttttgaaagaattctcccgatccttccggtggagtctaatctaagtacctattcagaagtggaagagtattcctcattcatttcatgcagatctgtgctcattaacgttaccgtttcccgagatgcaaacgctatggtggaaggcactttggagttgatagaatcgcttcttcaagggcacgaaatcatttcagataagggtagcagtgacgttattgaatcaatactgattatactaagattgttaagtgatgcgctagagtataattggcaaaatcaagaaagccttcattacaacgacatttcgactcacgtagaacatgaccaagaacagaagtacagaccaaagcttcacaatattcttcccgactactcgtcgactcattccaatggcaacaaacactttttccaccagagcaaacctcaggcactgataccggaactggcatcgaaattgcttgagagttgcgcgaagttgaagttcaatacaagaactttgcaaattttacaaaatatgatcagtcatgttcatggaaacattgtaacgactttgagttcctcgattcttccccgccacaaatcctatttgacaaggcacaaccatccttctcattgtaaaatgattgactctactctaggccatattctccgatttgtagcggcttccaatccgtccgagtattttgaatttatcagaaagagtgtgcaagtgcccgtaacacagacacacacgcattcacactcccattcacactctttgccatcttccgtttataacagcatagtgccccactttgatcttttcagcttcatccatttaagtaaggataattttaagaaatacttggaactcatcaaaaacttatcggtgacgttaaggaaaacgatttatcattgcctacttttgcattacagcgccaaagcaataatgttttggataatgactaggcctgcggaatattatgaactcttcaacttattaaaagataataacaatgaacactcgaaatccttaaacacgttaaaccatacacttttcgaggagatccattcgacttttaatgtgaatagcatgataaccaccaatcaaaatgctcatcaaggctcatcttccccttcgtcctcctcgccatcgtcaccacctagctcatcatcatcggataacaacaatcaaaacataatagcaaaatccttaagtcgtcagctttctcaccaccagtcatacattcaacagcagtctgaaagaaaactacattcttcatggactacaaactctcaatcctctacttcactgtcatcttcaacgtctgattcaacaacaactgatttctctactcacactcaaccaggagaatacgacccttccttaccagatactcccacgatgtctaacatcactattagtgcatcttcattattatcccaaactccaactccaacaacacaattgcaacagcggttgaactcagcagctgcagccgccgccgcagctgcttcaccatcgaattccaccccaactggatacacagcagagcaacaaagtcgagcttcatacgatgcacacaaaactggccatactggtaaggattatgatgaacattttttgtctatcactcgtttggataatgttttggagttatacacgcactttgatgatactgaggtattaccacacacatccgtactgaagtttttaactactttgacaatgttcgatattgacctttttaatgaattaaacgctacatcattcaaatatattcctgattgtactatgcatcgtccaaaagaaagaacgagttctttcaataatactgcacacgagacaggttccgaaaagacttcgggtataaaacatattacgcaaggcttaaagaaattaacttctttaccttcctcaaccaaaaaaactgtaaaatttatgaagatgttgctaagaaatttaattgggaaccaagctgtatcagacgttgccctcttagatacaatgagggccttactatcattcttcacaatgacttctgcggtctttctcgtggataggaacttaccctcagtactttttgccaagagactcatccccataatggggacaaatttaagcgtcggtcaagactggaattcaaaaataaataacagtttgatggtttgtttgaaaaaaaactccaccacgtttgttcaattacaattaatattcttctcttcagctattcaattcgatcatgaattattgctggcacgtctgagcatcgatacaatggccaacaatttaaacatgcagaagctatgcctttatactgaaggattcaggatattcttcgacataccaagtaagaaggaattgcggaaggcaattgcggttaaaatttctaaatttttcaaaacattattctccattatagcagatattcttttacaagaatttccgtattttgatgagcaaatcaccgacatagttgcttccattcttgacggtacaattatcaatgagtatggtacgaagaaacatttcaaggggagctcaccctctttatgttcgacaacccggtcaagatcaggatctacatctcaaagttcaatgacaccagtttctccgctgggactggatactgatatatgtccaatgaacaccctgtctttagttggttcaagtacttcaagaaattctgacaacgttaattcattaaacagttcaccaaagaacttgtcttctgatccatacttgtcacatcttgtggccccaagagcacgtcatgctttaggtgggccatctagtattataaggaataaaataccgactacattgacttcacctccaggaacggaaaaatcttcaccagtacaacgtccgcaaacggaaagcatcagtgccacaccaatggccataacaaattctactccattatcgtcggcagcattcggaattcgatcccctttgcagaaaataagaacgaggcgttattccgatgaaagtttaggaaaattcatgaaatcaacaaataattacattcaagaacatttgataccaaaagatttgaatgaggcaactcttcaagatgctagaagaataatgattaatattttcagtatttttaagagaccgaatagttacttcatcattcctcacaatataaactcgaatttacaatgggtttcgcaggatttcagaaatattatgaaaccgattttcgtcgccatcgtaagtccggatgtagatttacagaatactgctcaatcattcatggataccttattatcgaatgttattacttatggtgaatcagatgagaatatcagtattgaaggttatcatcttctttgcagttacactgtaacattatttgcaatgggccttttcgatttgaaaattaataatgaaaagcgtcaaattctcttggatataactgtcaagtttatgaaggttagatcacatttagcagggatcgcggaggcctcacaccacatggaatacataagtgattctgaaaaactcacctttccgctgattatggggaccgttggtagggccctatttgtttcattatactctagtcaacaaaaaattgaaaagactttaaagattgcttacacagagtatctttctgcaatcaattttcatgagaggaatattgatgatgctgataaaacttgggttcataatattgagtttgtagaagcgatgtgtcatgacaactacacaacttctggttcaattgctttccaaaggaggacaagaaataatattttacgatttgctactattcctaacgctatcttacttgattctatgaggatgatctataagaagtggcatacttacacacacagtaaaagtttagaaaaacaagaacggaacgacttcagaaatttcgcgggtattttagcctctttgtcgggtatcctattcatcaataaaaagatattgcaagaaatgtatccatacctactcgacaccgtttcagaattgaaaaaaaatatagactcttttatctcaaaacaatgccaatggttaaactatccggatttattaacgagagaaaattcaagagatattctaagtgtagaactgcatcctttgtcttttaacttactttttaataatttgaggctcaagttaaaagaacttgcttgttcagacttatcaataccagaaaatgaaagttcctatgttttattagaacaaataatcaaaatgctgcggacaatcctaggtcgtgatgatgacaattatgtaatgatgcttttttccacagagattgtagatcttattgatttattgacagatgaaataaaaaaaataccagcctattgtccaaaatatctcaaggcaattattcaaatgaccaaaatgtttagtgccttgcagcactcagaggttaatttaggtgtcaaaaatcattttcacgttaaaaataaatggttgagacaagtcactgattggtttcaagtgagtattgcgagagagtacgatttcgaaaacttgtcaaaacctctaaaagaaatggatttggtaaaaagagacatggatattctatacatagatacggcaatcgaagcttcaaccgctattgcgtacctcacgagacatactttcttagagattccacctgccgcgtcagatcccgaactatctcgatctaggtctgtgatatttggcttttatttcaacatcttaatgaaaggccttgaaaaaagtagtgatcgtgacaattacccagtattcttgaggcacaaaatgagtgtcctcaacgacaatgtaatactttcattaacaaatctttctaacaccaatgttgatgcgagtttgcagttcaccttaccgatgggctattccggaaatcgaaacattaggaatgcatttttggaggtcttcattaatatcgttacgaactatcggacatacacggctaaaactgaccttggaaaattagaagcagcagacaaatttttgcgatatacgattgaacatccccagctatcgtcctttggagcagcggtttgtcccgctagcgatattgatgcttatgctgctggcttaataaatgcatttgaaacgagaaatgccacccacattgtagtgtcacagttgattaaaaatgaaattgaaaattcttccagacctacggatatccttagaaggaatagctgtgctacgagatcattatctatgctagccaggtccaagggtagcgaatatttgattcgcactttgcaaccattactaaaaaaaattatccagaacagagatttttttgaaattgagaaattaaaaccggaagattcagatgctgaacgtcaaatagagctttttgttaaatacatgaatgaattattggaatccatatccaactccgtatcttattttccccctcctttattttatatttgtcaaaacatttataaagttgcgtgtgaaaaatttccggatcacgcaattatcgccgctgggtctttcgtatttttacggtttttttgtcctgctttagtcagccctgattctgaaaatatcatagatatttctcacttgagcgaaaagcgtaccttcatcagcttggctaaagttatccaaaatattgccaatggctcagaaaatttctccagatggccagctttgtgttcccaaaaggattttcttaaggaatgtagcgatagaattttcagattcctagctgaactttgtagaacagatcgcacgatagacatccaagtgagaacagacccaacgccaattgcatttgactatcaattccttcattcctttgtttacctttacggtcttgaagtgagaaggaatgtgctaaatgaagcaaaacatgatgatggtgacattgatggtgacgatttctataagaccacgtttttacttattgatgatgttcttggccaattaggccaacctaaaatggaattttccaatgaaataccaatatacataagagaacatatggacgactatccggaactgtatgagttcatgaataggcacgcgttcagaaacattgagacttcaacagcgtacagcccaagcgttcacgagtccacctcaagtgaaggcattccaattattacgttaacaatgtcaaatttctcagacagacatgtggacattgatacagttgcttacaagttcttgcaaatttatgctcgaatctggaccaccaaacactgtttaataatcgactgtacagaatttgacgagggagggcttgatatgaggaaatttatttctttggttatgggactattaccagaagttgcacccaaaaattgtataggctgttactactttaacgtaaacgagacatttatggataattatggaaaatgtttggacaaggacaacgtatatgtttcctcgaaaattcctcattatttcattaatagtaactctgatgaaggacttatgaaatctgtgggtataactggacaagggttgaaggttctgcaagatattcgtgtctctctgcatgatatcacgctttatgacgaaaaaagaaatagatttacgccggtatcgttgaaaataggcgatatttactttcaagtcttgcatgaaactcctaggcaatataaaataagagacatgggtactttattcgacgtaaaattcaatgatgtctacgaaattagccgaatattcgaagtacatgtttcgtcaataactggagtggcagctgaatttacagtaacttttcaggacgagagaaggttgatttttagtagtccgaaataccttgaaattgtgaagatgttctattacgcacagatccggttagaaagtgaatatgaaatggataataattcgagtacctcctccccaaattcaaacaacaaggacaaacagcagaaagagagaacaaaattattgtgccacctactgttagtatctcttattggtctgtttgatgagagtaaaaaaatgaaaaacagttcgtataacctaatagctgccactgaggcgtcatttggtttgaactttggctcccattttcatcgctctcccgaggtgtacgtccccgaagatactacaacatttttaggtgttattggaaagtctcttgcagagtctaatccagaactcacagcctatatgtttatctatgttttggaggcattgaagaacaacgtgattcctcatgtttacatccctcataccatttgcggtttgtcttattggatccctaatttataccaacatgtgtatttggctgatgatgaagaaggccccaaaaacatatctcacattttccgaattcttatcaggctctctgtgagagagactgactttaaagccgtatacatgcaatatgtttggctgctacttttagatgatggccgcttaactgacattatcgttgatgaagttattaatcatgcgttagaaagagactccgaaaaccgcgattggaagaaaacaatatcgttactaactgtcctacccactactgaggttgctaataatattattcaaaaaatattggcaaaaattagatcatttttaccgtcattgaagttagaagctatgacccaaagttggtctgaactaacaatattagttaagataagcatccatgttttttttgaaacttctttgctggtacagatgtacttaccagagatcctgtttatcgtatccttattaattgatgttggtccaagggaactcagatcatcactacaccagctattaatgaatgtatgccattccttggctattaactcagctttaccacaagatcatagaaataatctagatgaaataagtgatatatttgcacatcaaaaggtgaagtttatgtttgggttcagcgaggacaaaggacgaattttacagatttttagcgcttcttcttttgcaagcaagtttaatatcctggatttcttcatcaataatatattattgctgatggaatattcttcaacgtacgaagcaaacgtgtggaagacaagatacaagaaatatgtcttggaatctgtgtttacaagtaattcttttctttcggcacgttcaatcatgattgttggtataatgggtaaatcttacataactgaagggttatgcaaggctatgttaattgaaaccatgaaagttatcgccgaaccaaagattactgacgagcatcttttcttagccatatctcatatttttacttattccaaaattgttgaaggtttggatcccaaccttgacttaatgaagcacttattttggttttcaacactcttccttgaatcacgtcacccgataatttttgagggtgcccttctctttgtgtcaaactgtataaggcgcctatacatggcccagtttgaaaatgaaagtgaaacatcattgataagtactttacttaaggggagaaagtttgctcatacctttttaagcaagatagagaatcttagtggtattgtttggaatgaagataattttacacacattctgattttcatcattaataaaggactatccaatcctttcattaagagtacggcttttgatttcttgaagatgatgtttagaaactcctactttgagcatcaaatcaatcagaaatctgatcattatttgtgctatatgttcctattgtattttgttttaaactgtaatcaatttgaggaacttttaggtgacgttgattttgaaggagaaatggttaacattgaaaacaagaacaccattcctaaaattttgttggagtggttgagttcggataacgaaaatgcaaacattaccctctatcaaggtgcgatactgttcaaatgttcagttacggatgaaccaagtagatttaggtttgcgttgattattaggcatctattgacaaagaaacccatttgtgcattgcgtttttacagtgttattcgtaacgaaataagaaaaatatcagcatttgagcaaacttcggattgtgttccacttgctttcgatattttaaacttattagtgacgcattcagagtctaattccttagaaaaacttcacgaagaatccattgaacgtctgaccaaaagaggtttatcaattgtgacttcttctggtatatttgcgaagaattccgacatgatgataccgttagatgtaaaacctgaagatatctatgaacgtaagagaataatgacaatgattttatcaaggatgtcatgttctgcttag

SEQ ID NO: 22 - protein, 3079 aa, Saccharomyces cerevisiae
MSQPTKNKKKEHGTDSKSSRMTRTLVNHILFERILPILPVESNLSTYSEVEEYSSFISCRSVLINVTVSRDANAMVEGTLELIESLLQGHEIISDKGSSDVIESILIILRLLSDALEYNWQNQESLHYNDISTHVEHDQEQKYRPKLHNILPDYSSTHSNGNKHFFHQSKPQALIPELASKLLESCAKLKFNTRTLQILQSMISHVHGNILTTLSSSILPRHKSYLTRHNHPSHCKMIDSTLGHILRFVAASNPSEYFEFIRKSVQVPVTQTHTHSHSHSHSLPSSVYNSIVPHFDLFSFIHLSKDNFKKYLELIKNLSVTLRKTIYHCLLLHYSAKAIMFWIMTRPAEYYELFNLLKDNNNEHSKSLNTLNHTLFEEIHSTFNVNSMITTNQNVHQGSSSPSSSSPSSPPSSSSSDNNNQNIIAKSLSRQHSHHQSYIQQQSERKLHSSWTTNSQSSTSLSSSTSNSTTTDFSTHTQPGEYDPSLPDTPTMSNITISASSLLSQTPTPTTQLQQRLNSAAAAAAAAASPSNSTPTGYTAEQQSRASYDAHKTGHTGKDYDEHFLSITRLDNVLELYTHFDDTEVLPHTSVLKFLTTLTMFDIDLFNELNATSFKYIPDCTMHRPKERTSSFNNTAHETGSEKTSGIKHITQGLKKLTSLPSSTKKTVKFMKMLLRNLIGNQAVSDVALLDTMRALLSFFTMTSAVFLVDRNLPSVLFAKRLIPIMGTNLSVGQDWNSKINNSLMVCLKKNSTTFVQLQLIFFSSAIQFDHELLLARLSIDTMANNLNMQKLCLYTEGFRIFFDIPSKKELRKAIAVKISKFFKTLFSIIADILLQEFPYFDEQITDIVASILDGTIINEYGTKKHFKGSSPSLCSTTRSRSGSTSQSSMTPVSPLGLDTDICPMNTLSLVGSSTSRNSDNVNSLNSSPKNLSSDPYLSHLVAPRARHALGGPSSIIRNKIPTTLTSPPGTEKSSPVQRPQTESISATPMAITNSTPLSSAAFGIRSPLQKIRTRRYSDESLGKFMKSTNNYIQEHLIPKDLNEATLQDARRIMINIFSIFKRPNSYFIIPHNINSNLQWVSQDFRNIMKPIFVAIVSPDVDLQNTAQSFMDTLLSNVITYGESDENISIEGYHLLCSYTVTLFAMGLFDLKINNEKRQILLDITVKFMKVRSHLAGIAEASHHMEYISDSEKLTFPLIMGTVGRALFVSLYSSQQKIEKTLKIAYTEYLSAINFHERNIDDADKTWVHNIEFVEAMCHDNYTTSGSIAFQRRTRNNILRFATIPNAILLDSMRMIYKKWHTYTHSKSLEKQERNDFRNFAGILASLSGILFINKKILQEMYPYLLDTVSELKKNIDSFISKQCQWLNYPDLLTRENSRDILSVELHPLSFNLLFNNLRLKLKELACSDLSIPENESSYVLLEQIIKMLRTILGRDDDNYVMMLFSTEIVDLIDLLTDEIKKIPAYCPKYLKAIIQMTKMFSALQHSEVNLGVKNHFHVKNKWLRQVTDWFQVSIAREYDFENLSKPLKEMDLVKRDMDILYIDTAIEASTAIAYLTRHTFLEIPPAASDPELSRSRSVIFGFYFNILMKGLEKSSDRDNYPVFLRHKMSVLNDNVILSLTNLSNTNVDASLQFTLPMGYSGNRNIRNAFLEVFINIVTNYRTYTAKTDLGKLEAADKFLRYTIEHPQLSSFGAAVCPASDIDAYAAGLINAFETRNATHIVVSQLIKNEIENSSRPTDILRRNSCATRSLSMLARSKGSEYLIRTLQPLLKKIIQNRDFFEIEKLKPEDSDAERQIELFVKYMNELLESISNSVSYFPPPLFYICQNIYKVACEKFPDHAIIAAGSFVFLRFFCPALVSPDSENIIDISHLSEKRTFISLAKVIQNIANGSENFSRWPALCSQKDFLKECSDRIFRFLAELCRTDRTIDIQVRTDPTPIAFDYQFLHSFVYLYGLEVRRNVLNEAKHDDGDIDGDDFYKTTFLLIDDVLGQLGQPKMEFSNEIPIYIREHMDDYPELYEFMNRHAFRNIETSTAYSPSVHESTSSEGIPIITLTMSNFSDRHVDIDTVAYKFLQIYARIWTTKHCLIIDCTEFDEGGLDMRKFISLVMGLLPEVAPKNCIGCYYFNVNETFMDNYGKCLDKDNVYVSSKIPHYFINSNSDEGLMKSVGITGQGLKVLQDIRVSLHDITLYDEKRNRFTPVSLKIGDIYFQVLHETPRQYKIRDMGTLFDVKFNDVYEISRIFEVHVSSITGVAAEFTVTFQDERRLIFSSPKYLEIVKMFYYAQIRLESEYEMDNNSSTSSPNSNNKDKQQKERTKLLCHLLLVSLIGLFDESKKMKNSSYNLIAATEASFGLNFGSHFHRSPEVYVPEDTTTFLGVIGKSLAESNPELTAYMFIYVLEALKNNVIPHVYIPHTICGLSYWIPNLYQHVYLADDEEGPENISHIFRILIRLSVRETDFKAVYMQYVWLLLLDDGRLTDIIVDEVINHALERDSENRDWKKTISLLTVLPTTEVANNIIQKILAKIRSFLPSLKLEAMTQSWSELTILVKISIHVFFETSLLVQMYLPEILFIVSLLIDVGPRELRSSLHQLLMNVCHSLAINSALPQDHRNNLDEISDIFAHQKVKFMFGFSEDKGRILQIFSASSFASKFNILDFFINNILLLMEYSSTYEANVWKTRYKKYVLESVFTSNSFLSARSIMIVGIMGKSYITEGLCKAMLIETMKVIAEPKITDEHLFLAISHIFTYSKIVEGLDPNLDLMKHLFWFSTLFLESRHPIIFEGALLFVSNCIRRLYMAQFENESETSLISTLLKGRKFAHTFLSKIENLSGIVWNEDNFTHILIFIINKGLSNPFIKSTAFDFLKMMFRNSYFEHQINQKSDHYLCYMFLLYFVLNCNQFEELLGDVDFEGEMVNIENKNTIPKILLEWLSSDNENANITLYQGAILFKCSVTDEPSRFRFALIIRHLLTKKPICALRFYSVIRNEIRKISAFEQTSDCVPLAFDILNLLVTHSESNSLEKLHEESIERLTKRGLSIVTSSGIFAKNSDMMIPLDVKPEDIYERKRIMTMILSRMSCSA

SEQ ID NO: 23 - protein, 3079 aa, synthetic construct
  REGION 1..3079: note = Consensus IRA2 sequence
MSQPTKNKKKEHGTDSKSSRMTRTLVNHILFERILPILPVESNLSTYSEVEEYSSFISCRSVLINVTVSRDANAMVEGTLELIESLLQGHEIISDKGSSDVIESILIILRLLSDALEYNWQNQESLHYNDISTHVEHDQEQKYRPKLNSILPDYSSTHSNGNKHFFHQSKPQALIPELASKLLESCAKLKFNTRTLQILQNMISHVHGNILTTLSSSILPRHKSYLTRHNHPSHCKMIDSTLGHILRFVAASNPSEYFEFIRKSVQVPVTQTHTHSHSHSHSLPSSVYNSIVPHFDLFSFIYLSKHNFKKYLELIKNLSVTLRKTIYHCLLLHYSAKAIMFWIMARPAEYYELFNLLKDNNNEHSKSLNTLNHTLFEEIHSTFNVNSMITTNQNAHQGSSSPSSSSPSSPPSSSSSDNNNQNIIAKSLSRQLSHHQSYIQQQSERKLHSSWTTNSQSSTSLSSSTSNSTTTDFSTHTQPGEYDPSLPDTPTMSNITISASSLLSQTPTPTTQLQQRLNSAAAAAAAAASPSNSTPTGYTAEQQSRASYDAHKTGHTGKDYDEHFLSVTRLDNVLELYTHFDDTEVLPHTSVLKFLTTLTMFDIDLFNELNATSFKYIPDCTMHRPKERTSSFNNTAHETGSEKTSGIKHITQGLKKLTSLPSSTKKTVKFVKMLLRNLNGNQAVSDVALLDTMRALLSFFTMTSAVFLVDRNLPSVLFAKRLIPIMGTNLSVGQDWNSKINNSLMVCLKKNSTTFVQLQLIFFSSAIQFDHELLLARLSIDTMANNLNMQKLCLYTEGFRIFFDIPSKKELRKAIAVKISKFFKTLFSIIADILLQEFPYFDEQITDIVASILDGTIINEYGTKKHFKGSSPSLCSTTRSRSGSTSQSSMTPVSPLGLDTDICPMNTLSLVGSSTSRNSDNVNSLNSSPKNLSSDPYLSHLVAPRARHALGGPSSIIRNKIPTTLTSPPGTEKSSPVQRPQTESISATPMAITNSTPLSSAAFGIRSPLQKIRTRRYSDESLGKFMKSTNNYIQEHLIPKDLNEATLQDARRIMINIFSIFKRPNSYFIIPHNINSNLQWVSQDFRNIMKPIFVAIVSPDVDLQNTAQSFMDTLLSNVITYGESDENISIEGYHLLCSYTVTLFAMGLFDLKINNEKRQILLDITVKFMKVRSHLAGIAEASHHMEYISDSEKLTFPLIMGTVGRALFVSLYSSQQKIEKTLKIAYTEYLSAINFHERNIDDADKTWVHNIEFVEAMCHDNYTTSGSIAFQRRTRNNILRFATIPNAILLDSMRMIYKKWHTYTHSKSLEKQERNDFRNFAGILASLSGILFINKKILQEMYPYLLDTVSELKKNIDSFISKQCQWLNYPDLLTRENSRDILSVELHPLSFNLLFNNLRLKLKELACSDLSIPENESSYVLLEQIIKMLRTILGRDDDNYVMMLFSTEIVDLIDLLTDEIKKIPAYCPKYLKAIIQMTKMFSALQHSEVNLGVKNHFHVKNKWLRQITDWFQVSIAREYDFENLSKPLKEMDLVKRDMDILYIDTAIEASTAIAYLTRHTFLEIPPAASDPELSRSRSVIFGFYFNILMKGLEKSSDRDNYPVFLRHKMSVLNDNVILSLTNLSNTNVDASLQFTLPMGYSGNRNIRNAFLEVFINIVTNYRTYTAKTDLGKLEAADKFLRYTIEHPQLSSFGAAVCPASDIDAYAAGLINAFETRNATHIVVAQLIKNEIEKSSRPTDILRRNSCATRSLSMLARSKGNEYLIRTLQPLLKKIIQNRDFFEIEKLKPEDSDAERQIELFVKYMNELLESISNSVSYFPPPLFYICQNIYKVACEKFPDHAIIAAGSFVFLRFFCPALVSPDSENIIDISHLSEKRTFISLAKVIQNIANGSENFSRWPALCSQKDFLKECSDRIFRFLAELCRTDRTIDIQVRTDPTPIAFDYQFLHSFVYLYGLEVRRNVLNEAKHDDGDIDGDDFYKTTFLLIDDVLGQLGQPKMEFSNEIPIYIREHMDDYPELYEFMNRHAFRNIETSTAYSPSVHESTSSEGIPIITLTMSNFSDRHVDIDTVAYKFLQIYARIWTTKHCLIIDCTEFDEGGLDMRKFISLVMGLLPEVAPKNCIGCYYFNVNETFMDNYGKCLDKDNVYVSSKIPHYFINSNSDEGLMKSVGITGQGLKVLQDIRVSLHDITLYDEKRNRFTPVSLKIGDIYFQVLHETPRQYKIRDMGTLFDVKFNDVYEISRIFEVHVSSITGVAAEFTVTFQDERRLIFSSPKYLEIVKMFYYAQIRLESEYEMDNNSSTSSPNSNNKDKQQKERTKLLCHLLLVSLIGLFDESKKMKNSSYNLIAATEASFGLNFGSHFHRSPEVYVPEDTTTFLGVIGKSLAESNPELTAYMFIYVLEALKNNVIPHVYIPHTICGLSYWIPNLYQHVYLADDEEGPKNISHIFRILIRLSVRETDFKAVYMQYVWLLLLDDGRLTDIIVDEVINHALERDSENRDWKKTISLLTVLPTTEVANNIIQKILAKIRSFLPSLKLEAMTQSWSELTILVKISIHVFFETSLLVQMYLPEILFIVSLLIDVGPRELRSSLHQLLMNVCHSLAINSALPQDHRNNLDEISDIFAHQKVKFMFGFSEDKGRILQIFSASSFASKFNILDFFINNILLLMEYSSTYEANVWKTRYKKYVLESVFTSNSFLSARSIMIVGIMGKSYITEGLCKAMLIETMKVIAEPKITDEHLFLAISHIFTYSKIVEGLDPNLDLMKHLFWFSTLFLESRHPIIFEGALLFVSNCIRRLYMAQFENESETSLISTLLKGRKFAHTFLSKIENLSGIVWNEDNFTHILIFIINKGLSNPFIKSTAFDFLKMMFRNSYFEHQINQKSDHYLCYMFLLYFVLNCNQFEELLGDVDFEGEMVNIENKNTIPKILLEWLSSDNENANITLYQGAILFKCSVTDEPSRFRFALIIRHLLTKKPICALRFYSVIRNEIRKISAFEQNSDCVPLAFDILNLLVTHSESNSLEKLHEESIERLTKRGLSIVTSSGIFAKNSDMMIPLDVKPEDIYERKRIMTMILSRMSCSA

SEQ ID NO: 24 - protein, 293 aa, Saccharomyces cerevisiae
MSQPTKNKKKEHGTDSKSSRMTRTLVNHILFERILPILPVESNLSTYSEVEEYSSFISCRSVLINVTVSQDANAMVEGTLELIESLLQGHEIISDKCSSDVIESILIILRLLSDALEYNWQNQESLHYNDISTHVEHDQEQKYRPKLNSILPDYSSTHSNGNKHFFHQSKPQALIPELASKLLESCAKLKFNTRTLQILQNMISHVHGNILTTLSSSILPRHKSYLTRHNHPSHCKMIDSTLGHILRFVAASNPSEYFEFIRKSVQVPVTQTHTHTRIHTPIHTLCHLPFITA

SEQ ID NO: 25 - protein, 3081 aa, Saccharomyces cerevisiae
MSQPTKNKKKEHGTDSKSSRMTRTLVNHILFERILPILPVESNLSTYSEVEEYSSFISCRSVLINVTVSQDANAMVEGTLELIESLLQGHEIISDKCSSDVIESILIILRLLSDALEYNWQNQESLHYNDISTHVEHDQEQKYRPKLNSILPDYSSTHSNGNKHFFHQSKPQALIPELASKLLESCAKLKFNTRTLQILQNMISHVHGNILTTLSSSILPRHKSYLTRHNHPSHCKMIDSTLGHILRFVAASNPSEYFEFIRKSVQVPVTQTHTHTHSHSHSHSLPSSVYNSIVPHFDLFSFIYLSKHNFKKYLELIKNLSVTLRKTIYHCLLLHYSAKAIMFWIMARPAEYYELFNLLKDNNNEHSKSLNTLNHTLFEEIHSTFNVNSMITTNQNAHQGSSSPSSSSPSSPPSSSSSDNNNQNIIAKSLSRQLSHHQSYIQQQSERKLHSSWTTNSQSSTSLSSSTSNSTTTDFSTHTQPGEYDPSLPDTPTMSNITISASSLLSQTPTPTTQLQQRLNSAAAAAAAAASPSNSTPTGYTAEQQSRASYDAHKTGHTGKDYDEHFLSVTRLDNVLELYTHFDDTEVLPHTSVLKFLTTLTMFDIDLFNELNATSFKYIPDCTMHRPKERTSSFNNTAHETGSEKTSGIKHITQGLKKLTSLPSSTKKTVKFMKMLLRNLNGNQAVSDVALLDTMRALLSFFTMTSAVFLVDRNLPSVLFAKRLIPIMGTNLSVGQDWNSKINNSLMVCLKKNSTTFVQLQLIFFSSAIQFDHELLLARLSIDTMANNLNMQKLCLYTEGFRIFFDIPSKKELRKAIAVKISKFFKTLFSIIADILLQEFPYFDEQITDIVASILDGTIINEYGTKKHFKGSSPSLCSTTRSRSGSTSQSSMTPVSPLGLDTDIRPMNTLSLVGSSTSRNSDNVNSLNSSPKNLSSDPYLSHLVAPRARHALGGPSSIIRNKIPTTLTSPPGTEKSSPVQRPQTESISATPMAITNSTPLSSAAFGIRSPLQKIRTRRYSDESLGKFMKSTNNYIQEHLIPKDLNEATLQDARRIMINIFSIFKRPNSYFIIPHNINSNLQWVSQDFRNIMKPIFVAIVSPDVDLQNTAQSFMDTLLSNVITYGESDENISIEGYHLLCSYTVTLFAMGLFDLKINNEKRQILLDITVKFMKVRSHLAGIAEASHHMEYISDSEKLTFPLIMGTVGRALFVSLYSSQQKIEKTLNIAYTEYLSAINFHERNIDDADKTWVHNIEFVEAMCHDNYTTSGSIAFQRRTRNNILRFATIPNAILLDSMRMIYKKWHTYTHSKSLEKQERNDFRNFAGILASLSGILFINKKILQEMYPYLLDTVSELKKNIDSFISKQCQWLNYPDLLTRENSRDILSVELHPLSFNLLFNNLRLKLKELACSDLSIPENESSYVLLEQIIKMLRTILGRDDDNYVMMLFSTEIVDLIDLLTDEIKKIPAYCPKYLKAIIQMTKMFSALQHSEVNLGVKNHFHVKNKWLRQITDWFQVSIAREYDFENLSKPLKEMDLVKRDMDILYIDTAIEASTAIAYLTRHTFLEIPPAASDPELSRSRSVIFGFYFNILMKGLEKSSDRDNYPVFLRHKMSVLNDNVILSLTNLSNTNVDASLQFTLPMGYSGNRNIRNAFLEVFINIVTNYRTYTAKTDLGKLEAADKFLRYTIEHPQLSSFGAAVCPASDIDAYAAGLINAFETRNATHIVVAQLIKNEIEKSSRPTDILRRNSCATRSLSMLARSKGNEYLIRTLQPLLKKIIQNRDFFEIEKLKPEDSDAERQIELFVKYMNELLESISNSVSYFPPPLFYICQNIYKVACEKFPDHAIIAAGSFVFLRFFCPALVSPDSENIIDISHLSEKRTFISLAKVIQNIANGSENFSRWPALCSQKDFLKECSDRIFRFLAELCRTDRTIDIQVRTDPTPIAFDYQFLHSFVYLYGLEVRRNVLNEAKHDDGDIDGDDFYKTTFLLIDDVLGQLGQPKMEFSNEIPIYIREHMDDYPELYEFMNRHAFRNIETSTAYSPSVHESTSSEGIPIITLTMSNFSDRHVDIDTVAYKFLQIYARIWTTKHCLIIDCTEFDEGGLDMRKFISLVMGLLPEVAPKNCIGCYYFNVNETFMDNYGKCLDKDNVYVSSKIPHYFINSNSDEGLMKSVGITGQGLKVLQDIRVSLHDITLYDEKRNRFTPVSLKIGDIYFQVLHETPRQYKIRDMGTLFDVKFNDVYEISRIFEVHVSSITGVAAEFTVTFQDERRLIFSSPKYLEIVKMFYYAQIRLESEYEMDNNSSTSSPNSNNKDKQQKERTKLLCHLLLVSLIGLFDESKKMKNSSYNLIAATEASFGLNFGSHFHRSPEVYVPEYTTTFLGVIGKSLAESNPELTAYMFIYVLEALKNNVIPHVYIPHTICGLSYWIPNLYQHVYLADDEEGPENISHIFRILIRLSVRETDFKAVYMQYVWLLLLDDGRLTDIIVDEVINHALERDSENRDWKKTISLLTVLPTTEVANNIIQKILAKIRSFLPSLKLEAMTQSWSELTILVKISIHVFFETSLLVQMYLPEILFIVSLLIDVGPRELRSSLHQLLMNVCHSLAIDSALSQDHRNNLDEISDIFAHQKVKFMFGFSEDKGRILQIFSASSFASKFNILDFFINNILLLMEYSSTYEANVWKTRYKKYVLESVFTSNSFLSARSIMIVGIMGKSYITEGLCKAMLIETMKVIAEPKITDEHLFLAISHIFTYSKIVEGLDPNLDLMKHLFWFSTLFLESRHPIIFEGALLFVSNCIRRLYMAQFENESETSLISTLLKGRKFAHTFLSKIENLSGIVWNEDNFTHILIFIINKGLSNPFIKSTAFDFLKMMFRNSYFEHQINQKSDHYLCYMFLLYFVLNCNQFEELLGDVDFEGEMVNIENKNTIPKILLEWLSSDNENANITLYQGAILFKCSVTDEPSKFRFALIIRHLLTKKPICALRFYSVIRNEIRKISAFEQNSDCVPLAFDILNLLVTHSESNSLEKLHEESIERLTKRGLSIVTSSGIFAKNSDMMIPLDVKPEDIYERKRIMTMILSRMSCSA

SEQ ID NO: 26 - protein, 309 aa, Saccharomyces cerevisiae
MQGNKSTIREYKIVVVGGGGVGKSALTIQFIQSYFVDEYDPTIEDSYRKQVVIDDKVSILDILDTAGQEEYSAMREQYMRTGEGFLLVYSVTSRNSFDELLSYYQQIQRVKDSDYIPVVVVGNKLDLENERQVSYEDGLRLAKQLNAPFLETSAKQAINVDEAFYSLIRLVRDDGGKYNSMNRQLDNTNEIRDSELTSSATADREKKNNGSYVLDNSLTNAGTGSSSKSAVNHNGETTKRTDEKNYVNQNNNNEGNTKYSSNGNGNRSDISRGNQNNALNSRSKQSAEPQKNSSANARKESSGGCCIIC

SEQ ID NO: 27 - protein, 309 aa, Saccharomyces cerevisiae
MQGNKSTIREYKIVVVGGGGVGKSALTIQFIQSYFVDEYDPTIEDSYRKQVVIDDKVSILDILDTTGQEEYSAMREQYMRTGEGFLLVYSVTSRNSFDELLSYYQQIQRVKDSDYIPVVVVGNKLDLENERQVSYEDGLRLAKQLNAPFLETSAKQAINVDEAFYSLIRLVRDDGGKYNSMNRQLDNTNEIRDSELTSSATADREKKNNGSYVLDNSLTNAGTGSSSKSAVNHNGETTKRTDEKNYVNQNNNNEGNTKYSSNGNGNRSDISRGNQNNALNSRSKQSAEPQKNSSANARKESSGGCCIIC

SEQ ID NO: 28 - DNA, 9240 bp, Saccharomyces cerevisiae
  source 1..9240: mol_type = unassigned DNA
atgtcccagcccactaagaataagaagaaagaacacgggaccgattccaagtcatcccgcatgactcggacgttggttaaccatattctttttgaaagaattctcccgatccttccggtggagtctaatctaagtacctattcagaagtggaagagtattcctcattcatttcatgcagatctgtgctcattaacgttaccgtttcccgagatgcaaacgctatggtggaaggcactttggagttgatagaatcgcttcttcaagggcacgaaatcatttcagataagggtagcagtgacgttattgaatcaatactgattatactaagattgttaagtgatgcgctagagtataattggcaaaatcaagaaagccttcattacaacgacatttcgactcacgtagaacatgaccaagaacagaagtacagaccaaagcttcacaatattcttcccgactactcgtcgactcattccaatggcaacaaacactttttccaccagagcaaacctcaggcactgataccggaactggcatcgaaattgcttgagagttgcgcgaagttgaagttcaatacaagaactttgcaaattttacaaagtatgatcagtcatgttcatggaaacattctaacgactttgagttcctcgattcttccccgccacaaatcctatttgacaaggcacaaccatccttctcattgtaaaatgattgactctactctaggccatattctccgatttgtagcggcttccaatccgtccgagtattttgaatttatcagaaagagtgtgcaagtgcccgtaacacagacacacacgcattcacactcccattcacactctttgccatcttccgtttataacagcatagtgccccactttgatcttttcagcttcatccatttaagtaaggataattttaagaaatacttggaactcatcaaaaacttatcggtgacgttaaggaaaacgatttatcattgcctacttttgcattacagcgccaaagcaataatgttttggataatgactaggcctgcggaatattatgaactcttcaacttattaaaagataataacaatgaacactcgaaatccttaaacacgttaaaccatacacttttcgaggagatccattcgacttttaatgtgaatagcatgataaccaccaatcaaaatgttcatcaaggctcatcttccccttcgtcctcctcgccatcgtcaccacctagctcatcatcatcggataacaacaatcaaaacataatagcaaaatccttaagtcgtcagcattctcaccaccagtcatacattcaacagcagtctgaaagaaaactacattcttcatggactacaaactctcaatcctctacttcactgtcatcttcaacgtctaattcaacaacaactgatttctctactcacactcaaccaggagaatacgacccttccttaccagatactcccacgatgtctaacatcactattagtgcatcttcattattatcccaaactccaactccaacaacacaattgcaacagcggttgaactcagcagctgcagccgccgccgcagctgcttcaccatcgaattccaccccaactggatacacagcagagcaacaaagtcgagcttcatacgatgcacacaaaactggccatactggtaaggattatgatgaacattttttgtctatcactcgtttggataatgttttggagttatacacgcactttgatgatacagaggtactaccacacacatccgtactgaagtttttaactactttgacaatgttcgatattgacctttttaatgaattaaacgctacatcattcaaatatattcctgactgtactatgcatcgtccaaaagaaagaacaagttctttcaataatactgcacacgagacaggttccgaaaagacttcgggtataaaacatattacgcaaggcttaaagaaattaacttctttaccttcctcaaccaaaaaaactgtaaaatttatgaagatgttgctaagaaatttaattgggaaccaagctgtatcagacgttgccctcttagatacaatgagggccttactatcattcttcacaatgacttctgcggtctttctcgtggataggaacttaccctcagtactttttgccaagagactcatccccataatggggacaaatttaagcgtcggtcaagactggaattcaaaaataaataacagtttgatggtttgtttgaaaaaaaactccaccacgtttgttcaattacaattaatattcttctcttcagctattcaattcgatcatgaattattgctggcacgtctgagcatcgatacaatggccaacaatttaaacatgcagaagctatgcctttatactgaaggattcaggatattcttcgacataccaagtaagaaggaattgcggaaggcaattgcggttaaaatttctaaatttttcaaaacattattctccattatagcagatattcttttacaagaatttccgtattttgatgagcaaatcaccgacatagttgcttccattcttgacggtacaattatcaatgagtatggtacgaagaaacatttcaaggggagctcaccctctttatgttcgacaacccggtcaagatcaggatctacatctcaaagttcaatgacaccagtttctccgctgggactggatactgatatatgtccaatgaacaccctgtctttagttggttcaagtacttcaagaaattctgacaacgttaattcattaaacagttcaccaaagaacttgtcttctgatccatacttgtcacatcttgtggccccaagagcacgtcatgctttaggtgggccatctagtattataaggaataaaataccgactacattgacttcacctccaggaacggaaaaatcttcaccagtacaacgtccgcaaacggaaagcatcagtgccacaccaatggccataacaaattctactccattatcgtcggcagcattcggaattcgatcccctttgcagaaaataagaacgaggcgttattccgatgaaagtttaggaaaattcatgaaatcaacaaataattacattcaagaacatttgataccaaaagatttgaatgaggcaactcttcaagatgctagaagaataatgattaatattttcagtatttttaagagaccgaatagttacttcatcattcctcacaatataaactcgaatttacaatgggtttcgcaggatttcagaaatattatgaaaccgattttcgtcgccatcgtaagtccggatgtagatttacagaatactgctcaatcattcatggataccttattatcgaatgttattacttatggtgaatcagatgagaatatcagtattgaaggttatcatcttctttgcagttacactgtaacattatttgcaatgggccttttcgatttgaaaattaataatgaaaagcgtcaaattctcttggatataactgtcaagtttatgaaggttagatcacatttagcagggatcgcggaggcctcacaccacatggaatacataagtgattctgaaaaactcacctttccgctgattatggggaccgttggtagggccctatttgtttcattatactctagtcaacaaaaaattgaaaagactttaaagattgcttacacagagtatctttctgcaatcaattttcatgagaggaatattgatgatgctgataaaacttgggttcataatattgagtttgtagaagcgatgtgtcatgacaactacacaacttctggttcaattgctttccaaaggaggacaagaaataatattttacgatttgctactattcctaacgctatcttacttgattctatgaggatgatctataagaagtggcatacttacacacacagtaaaagtttagaaaaacaagaacggaacgacttcagaaatttcgcgggtattttagcctctttgtcgggtatcctattcatcaataaaaagatattgcaagaaatgtatccatacctactcgacaccgtttcagaattgaaaaaaaatatagactcttttatctcaaaacaatgccaatggttaaactatccggatttattaacgagagaaaattcaagagatattctaagtgtagaactgcatcctttgtcttttaacttactttttaataatttgaggctcaagttaaaagaacttgcttgttcagacttatcaataccagaaaatgaaagttcctatgttttattagaacaaataatcaaaatgctgcggacaatcctaggtcgtgatgatgacaattatgtaatgatgcttttttccacagagattgtagatcttattgatttattgacagatgaaataaaaaaaataccagcctattgtccaaaatatctcaaggcaattattcaaatgaccaaaatgtttagtgccttgcagcactcagaggttaatttaggtgtcaaaaatcattttcacgttaaaaataaatggttgagacaagtcactgattggtttcaagtgagtattgcgagagagtacgatttcgaaaacttgtcaaaacctctaaaagaaatggatttggtaaaaagagacatggatattctatacatagatacggcaatcgaagcttcaaccgctattgcgtacctcacgagacatactttcttagagattccacctgccgcgtcagatcccgaactatctcgatctaggtctgtgatatttggcttttatttcaacatcttaatgaaaggccttgaaaaaagtagtgatcgtgacaattacccagtattcttgaggcacaaaatgagtgtcctcaacgacaatgtaatactttcattaacaaatctttctaacaccaatgttgatgcgagtttgcagttcaccttaccgatgggctattccggaaatcgaaacattaggaatgcatttttggaggtcttcattaatatcgttacgaactatcggacatacacggctaaaactgaccttggaaaattagaagcagcagacaaatttttgcgatatacgattgaacatccccagctatcgtcctttggagcagcggtttgtcccgctagcgatattgatgcttatgctgctggcttaataaatgcatttgaaacgagaaatgccacccacattgtagtgtcacagttgattaaaaatgaaattgaaaattcttccagacctacggatatccttagaaggaatagctgtgctacgagatcattatctatgctagccaggtccaagggtagcgaatatttgattcgcactttgcaaccattactaaaaaaaattatccagaacagagatttttttgaaattgagaaattaaaaccggaagattcagatgctgaacgtcaaatagagctttttgttaaatacatgaatgaattattggaatccatatccaactccgtatcttattttccccctcctttattttatatttgtcaaaacatttataaagttgcgtgtgaaaaatttccggatcacgcaattatcgccgctgggtctttcgtatttttacggtttttttgtcctgctttagtcagccctgattctgaaaatatcatagatatttctcacttgagcgaaaagcgtaccttcatcagcttggctaaagttatccaaaatattgccaatggctcagaaaatttctccagatggccagctttgtgttcccaaaaggattttcttaaggaatgtagcgatagaattttcagattcctagctgaactttgtagaacagatcgcacgatagacatccaagtgagaacagacccaacgccaattgcatttgactatcaattccttcattcctttgtttacctttacggtcttgaagtgagaaggaatgtgctaaatgaagcaaaacatgatgatggtgacattgatggtgacgatttctataagaccacgtttttacttattgatgatgttcttggccaattaggccaacctaaaatggaattttccaatgaaataccaatatacataagagaacatatggacgactatccggaactgtatgagttcatgaataggcacgcgttcagaaacattgagacttcaacagcgtacagcccaagcgttcacgagtccacctcaagtgaaggcattccaattattacgttaacaatgtcaaatttctcagacagacatgtggacattgatacagttgcttacaagttcttgcaaatttatgctcgaatctggaccaccaaacactgtttaataatcgactgtacagaatttgacgagggagggcttgatatgaggaaatttatttctttggttatgggactattaccagaagttgcacccaaaaattgtataggctgttactactttaacgtaaacgagacatttatggataattatggaaaatgtttggacaaggacaacgtatatgtttcctcgaaaattcctcattatttcattaatagtaactctgatgaaggacttatgaaatctgtgggtataactggacaagggttgaaggttctgcaagatattcgtgtctctctgcatgatatcacgctttatgacgaaaaaagaaatagatttacgccggtatcgttgaaaataggcgatatttactttcaagtcttgcatgaaactcctaggcaatataaaataagagacatgggtactttattcgacgtaaaattcaatgatgtctacgaaattagccgaatattcgaagtacatgtttcgtcaataactggagtggcagctgaatttacagtaacttttcaggacgagagaaggttgatttttagtagtccgaaataccttgaaattgtgaagatgttctattacgcacagatccggttagaaagtgaatatgaaatggataataattcgagtacctcctccccaaattcaaacaacaaggacaaacagcagaaagagagaacaaaattattgtgccacctactgttagtatctcttattggtctgtttgatgagagtaaaaaaatgaaaaacagttcgtataacctaatagctgccactgaggcgtcatttggtttgaactttggctcccattttcatcgctctcccgaggtgtacgtccccgaagatactacaacatttttaggtgttattggaaagtctcttgcagagtctaatccagaactcacagcctatatgtttatctatgttttggaggcattgaagaacaacgtgattcctcatgtttacatccctcataccatttgcggtttgtcttattggatccctaatttataccaacatgtgtatttggctgatgatgaagaaggccccgaaaacatatctcacattttccgaattcttatcaggctctctgtgagagagactgactttaaagccgtatacatgcaatatgtttggctgctacttttagatgatggccgcttaactgacattatcgttgatgaagttattaatcatgcgttagaaagagactccgaaaaccgcgattggaagaaaacaatatcgttactaactgtcctacccactactgaggttgctaataatattattcaaaaaatattggcaaaaattagatcatttttaccgtcattgaagttagaagctatgacccaaagttggtctgaactaacaatattagttaagataagcatccatgttttttttgaaacttctttgctggtacagatgtacttaccagagatcctgtttatcgtatccttattaattgatgttggtccaagggaactcagatcatcactacaccagctattaatgaatgtatgccattccttggctattaactcagctttaccacaagatcatagaaataatctagatgaaataagtgatatatttgcacatcaaaaggtgaagtttatgtttgggttcagcgaggacaaaggacgaattttacagatttttagcgcttcttcttttgcaagcaagtttaatatcctggatttcttcatcaataatatattattgctgatggaatattcttcaacgtacgaagcaaacgtgtggaagacaagatacaagaaatatgtcttggaatctgtgtttacaagtaattcttttctttcggcacgttcaatcatgattgttggtataatgggtaaatcttacataactgaagggttatgcaaggctatgttaattgaaaccatgaaagttatcgccgaaccaaagattactgacgagcatcttttcttagccatatctcatatttttacttattccaaaattgttgaaggtttggatcccaaccttgacttaatgaagcacttattttggttttcaacactcttccttgaatcacgtcacccgataatttttgagggtgcccttctctttgtgtcaaactgtataaggcgcctatacatggcccagtttgaaaatgaaagtgaaacatcattgataagtactttacttaaggggagaaagtttgctcatacctttttaagcaagatagagaatcttagtggtattgtttggaatgaagataattttacacacattctgattttcatcattaataaaggactatccaatcctttcattaagagtacggcttttgatttcttgaagatgatgtttagaaactcctactttgagcatcaaatcaatcagaaatctgatcattatttgtgctatatgttcctattgtattttgttttaaactgtaatcaatttgaggaacttttaggtgacgttgattttgaaggagaaatggttaacattgaaaacaagaacaccattcctaaaattttgttggagtggttgagttcggataacgaaaatgcaaacattaccctctatcaaggtgcgatactgttcaaatgttcagttacggatgaaccaagtagatttaggtttgcgttgattattaggcatctattgacaaagaaacccatttgtgcattgcgtttttacagtgttattcgtaacgaaataagaaaaatatcagcatttgagcaaacttcggattgtgttccacttgctttcgatattttaaacttattagtgacgcattcagagtctaattccttagaaaaacttcacgaagaatccattgaacgtctgaccaaaagaggtttatcaattgtgacttcttctggtatatttgcgaagaattccgacatgatgataccgttagatgtaaaacctgaagatatctatgaacgtaagagaataatgacaatgattttatcaaggatgtcatgttctgcttag

SEQ ID NO: 29 - protein, 475 aa, Streptococcus mutans
MTKQYKNYVNGEWKLSENEIKIYEPASGAELGSVPAMSTEEVDYVYASAKKAQPAWRSLSYIERAAYLHKVADILMRDKEKIGAVLSKEVAKGYKSAVSEVVRTAEIINYAAEEGLRMEGEVLEGGSFEAASKKKIAVVRREPVGLVLAISPFNYPVNLAGSKIAPALIAGNVIAFKPPTQGSISGLLLAEAFAEAGLPAGVFNTITGRGSEIGDYIVEHQAVNFINFTGSTGIGERIGKMAGMRPIMLELGGKDSAIVLEDADLELTAKNIIAGAFGYSGQRCTAVKRVLVMESVADELVEKIREKVLALTIGNPEDDADITPLIDTKSADYVEGLINDANDKGAAALTEIKREGNLICPILFDKVTTDMRLAWEEPFGPVLPIIRVTSVEEAIEISNKSEYGLQASIFTNDFPRAFGIAEQLEVGTVHINNKTQRGTDNFPFLGAKKSGAGIQGVKYSIEAMTTVKSVVFDIK

SEQ ID NO: 30 - DNA, 1428 bp, synthetic construct
  misc_feature 1..1428: note = Encoding SEQ ID NO: 29 and codon-optimized for expression in Saccharomyces cerevisiae
  source 1..1428: mol_type = other DNA
atgacaaaacaatataaaaattatgtcaatggcgagtggaagctttcagaaaatgaaattaaaatctacgaaccggccagtggagctgaattgggttcagttccagcaatgagtactgaagaagtagattatgtttatgcttcagccaagaaagctcaaccagcttggcgatcactttcatacatagaacgtgctgcctaccttcataaggtagcagatattttgatgcgtgataaagaaaaaataggtgctgttctttccaaagaggttgctaaaggttataaatcagcagtcagcgaagttgttcgtactgcagaaatcattaattatgcagctgaagaaggccttcgtatggaaggtgaagtccttgaaggcggcagttttgaagcagccagcaagaaaaaaattgccgttgttcgtcgtgaaccagtaggtcttgtattagctatttcaccatttaactaccctgttaacttggcaggttcgaaaattgcaccggctcttattgcgggaaatgttattgcttttaaaccaccgacgcaaggatcaatctcagggctcttacttgctgaagcatttgctgaagctggacttcctgcaggtgtctttaataccattacaggtcgtggttctgaaattggagactatattgtagaacatcaagccgttaactttatcaatttcactggttcaacaggaattggggaacgtattggcaaaatggctggtatgcgtccgattatgcttgaactcggtggaaaagattcagccatcgttcttgaagatgcagaccttgaattgactgctaaaaatattattgcaggtgcttttggttattcaggtcaacgctgtacagcagttaaacgtgttcttgtgatggaaagtgttgctgatgaactggtcgaaaaaatccgtgaaaaagttcttgcattaacaattggtaatccagaagacgatgcagatattacaccgttgattgatacaaaatcagctgattatgtagaaggtcttattaatgatgccaatgataaaggagccgctgcccttactgaaatcaaacgtgaaggtaatcttatctgtccaatcctctttgataaggtaacgacagatatgcgtcttgcttgggaagaaccatttggtcctgttcttccgatcattcgtgtgacatctgtagaagaagccattgaaatttctaacaaatcggaatatggacttcaggcttctatctttacaaatgatttcccacgcgcttttggtattgctgagcagcttgaagttggtacagttcatatcaataataagacacagcgcggtacggacaacttcccattcttaggggctaaaaaatcaggtgcaggtattcaaggggtaaaatattctattgaagctatgacaactgttaaatccgtcgtatttgatatcaaataa

SEQ ID NO: 31 - protein, 535 aa, Alternaria alternata
MDKISKYNVVHRYEKRSLLIAINCIAGLSILFFGYDQGMMGGVNTAYDYYTLMGFGHKGADGGPVVDDTLLQGGIVSVYYLGTLVGCLVGGSIGDRFGRIKTIFVGAAVATVGACLQCSAMNHEWMICARLVNGWGTGILNSIIPVWATETAEHTSRGQFIAIEFTLNILGVVIAYWLEYACSFYGDGTSSFIWRFPIAFQIPMLMILMAAVMFFPESPRWLVKVGREAEGRYVLSRLRGDAGEDRERAETEFQEIVASCELERSNFRKQSYFHMLFGIGSGKLHTGRRVQLVIWLQIMQEWVGIAGVTIYAPTIFGIAGMSPAKRQWISGLNNIFYMFATLICVFTLDRIGRRWTCYWGSAGQGIAMALAGGFSYLGQEATKRGDTSAASSYGNAAVSMVFIFTAIFGATWLTVPWLYPAEIFPLEVRAKGNAWGVVGWSIGNGWLTLLCPIMFEALGEKTLYIFAACNAITIPMVWALYPETNQRTLEEIDLLFASDSIWNWEAEKNFAALQETLPFEATSHAAKNDIERVSL

SEQ ID NO: 32 - protein, 542 aa, Aspergillus fumigatus
MWTTTSGLSGRSLRLSITFAAVVGFSLFGYNQGMMAGLLNGDEFVNSFPILKMPDNPTAGEKHYIDVIRGAVTSCYELGCFFGALFSMFCGNRLGRTRLIFMGASILIVGALLTTVCYTGKWEVGQFVIGRVVSGIGNGMNTATIPVWQSECSGAHNRGFLVCFEGAMIAGGTFIAYWVVFGISHAADSVQWRFPVALQIFFALVVATGALMLPDSPSWFVSRGLDNEACEVLGKIKGTSPDSDQVLHDFNLIKTDMESTKSEQSNWKTVFTFGKTQEFQRLLIGCSGQFFQQFTGCNAAIYYSTLLFQENLHMEKYLSLIMGGVFASVYALATIPSFFMIERVGRRKLYLIGFLGQGLSFVITFACLIKETEENSKGAAVGIFLFITFFAFTLLPLPWIYPPEINPLRTRTVGASASTCTNWMCNFAVVMFTPLFAGQSPWGVYLFFALFNFVGLIFGYFFYVETAGRELEEVDIIYAKAHVEGKMPFRVAHDLPKLSFEEIVQQSRELGLDTNDHVMLEKKELGLSSDSAQETEEVYEKQ

SEQ ID NO: 33 - protein, 539 aa, Aspergillus terreus
MWTTTSGTSGRLLRASITFTAVMGFSLFGYNQGMMSGLIAGDEFTKTFGTLAMPDNPDDATYRHINVIRGAVTACYEIGCFFGALFSMFFGEKIGRTRIIFSGALVLIIGAVISTAAFGPQWGLGQFVVGRVISGLGNGMNTATIPVWQSECSSAHNRGLLVCFEGAMIAVGTFIAYWVVFGLSYVPETVQWRFPVALQVFFALIVAAGAMMFPESPRWFVMRGYHKEACEVIAKLKNSTPDSDEVLTEFNFMKADVEMTSASQASWKTVFTFGKTQEFQRLLVGCSGQFFQQFTGCNAAIYYSTLLFEENLNLEHRLALIMGGVFATVYALATIPSFFMVERVGRRKLYLIGFLGQGLSFVITFACLIKPTTENSKGAIVGIFLFITFFAFTLLPLPWIYPPEINPLRTRTVGAAASTCTNWICNFAVVMFTPLFAASSGWGVYLFFALINLIGVPFGWFFFVETAGRELEEIDIVYAKAHVEKKWPFMVAKDMPKLSLEEITQQSRELGLDLNDSSPPDQNESGLSSDNDQSVAEKQ

SEQ ID NO: 34 - protein, 572 aa, Cordyceps militaris
MVDFKALTEKHNVAHKLYKSSLLNTVCLVAGLSIFFFGYDQGLMGGVNTTRNYAETMGFGHWDEEQGIVVVDKPLLQGGIVAVYYLPGTLAGCLLGGWMGDRYGRITTIGLACIWCIFAAALQSAAQNASWMFCARVLNGIGTGILNAITPVWATETASHTSRGQFVAFEFTLNIFGVVVAYWLEFGTSKYHDPESSFIWRFPVAFQILPLIVLLAVIWFMPESPRWLVKVGREEEARFILGRLRGNEGEEGEAAEAELQDIISIKNLENDTSEQQSYFHMFFGIGSGKLHTGRRVQLVIWLQILQEWIGIAGITIYGPQIFTIAGIGASDRLWVSGVNNITYMFATLICVFTLDRIGRRWTLYWGAVGQGICMFAGGGLARATINASDSNRGQIGGAATFFVFLYTAIFGATWLTVPWLYPAEIFPLQVRAKGNAWGVVGWSIGNGWCVLLLPTIFDKLKEKTLYIFGAVNVVSIVAVWALYPESNQRTLEEMDLVFASDSIWTWDAERNFAKLKAENPDLIKSHKDTRGANDLERGIFASRRASKASATPPSVDKETTVENVENSSRPHP

SEQ ID NO: 35 - protein, 524 aa, Diplodia corticola
MGAKGLFGCTGHAIERLQLALIVAPSFILFGYNQAGLGGLLTEADWVKTFPEIDTVNTEGAEKSHNSTIQGLVVATFVIGALIGSLSCSYTGDKYGRRAVVMAGAICTLIGEVLECSSFGLPQFIVGRIIVGLGIGQLSATVPVWQSETSGAKNRGQHVVLDGLFICVGYVLESWIDLGFFELPEGQVTWRPPIAIAIVFSLILIGSVYLFPESPRWLVRKARNDEARSAIALFRGHEEDAIEVQAELAGIELSLEETSKSGAKLKDMLKMGEDKLLYRFGLCILLQFYQQMSGSNLISVYTSILFQQNLGLSPELSRVLSGGALTWKFLSSFVAFFTIDRFGRRAVFMFSGAGMSLCMIALAITTSMTDSHGAQVAAGCFIYMFNFFVPIGFLGANFLYCTEVAPLRLRVAMSSISTANHWLWNFVVIMVTPVALANIAWRYYIVYAAVAFCIPLSVYFFYPETMGRNLEELDMMFRESPSVMGTVNFAKTRPIAMPQEFAVDHTKGGAEHESDLEAKEKDLV

SEQ ID NO: 36 - protein, 558 aa, Eremothecium gossypii
MIKESFSRTATGGFSGRALRLAVTITAVTGFSLFGYDQGLLSGLINGEQFNHEFPATLEQGDNDRHATVVQGAVTSCYELGCFFGSLYVMVDGERRGRKPLIIMGSLLTILGTVVSVAAFREHWGLGQFVIGRVVTGLGTGLNTSTIPVWQSEMSKPKNRGLLVNLEGSVIAVGTMIAYWIDFGLSYVDSSVQWRFPVAMQIVFAVLLLVGIVQLPESPRWLMAHGRTAESKYVLGKLDNLDPSDDSVLAEAAAIEDAVNRFKHEKRSLKDALTGGRGQNLQRTLVACSTQFFQQFTGCNAAIYYSTVLFHKTINLEYRLSLILGGVFSTVYTLATIPSFFLIETVGRRKLFLIGALGQGFSFTITFACLINDTKNNAKGAAVGLFLFIIFFGMTILSLPWIYPPEISSMKVRSITNAMSTCTNWLCNFAVVMFTPIFIHEAGWGCYLFFAVMNFLYVPIIFFFYPETAGRSLEEIDIIYAKSFVDGTQAWRVAANLPKLSLKEVEEYGIQLGLYNNEHDFKDEMKVITEAEGQVPRSSISDSNMESSSTGNGIMKKP

SEQ ID NO: 37 - protein, 557 aa, Eremothecium sinecaudum
MESTVRKWFSRTATASLSGRALRLAITITAVTGFSLFGYDQGLLSGLINGDKFNHEFPATLEKHDNDTHATVVQGAVTSCYELGCFFGSLFVVMKGEKMGRKPLIICGALLTIVGTIISTLAFREHWGLGQFVVGRVITGLGTGFNTSTIPVWQSEMSKPENRGLLVNLEGSVIAVGTMIAYWIDFGLSYVDSSAQWRFPVAMQIVFAVFLLIGCTQLPESPRWLMAHGRGEEARYILSQLDGVPIDDPTVLAEAAAIEEVVDKFKNQRWSLKDAFTGGRGQNLQRTLVACSTQFFQQFTGCNAAIYYSTVLFHKTIKLEYRMSLILGGVFATVYTLSTIPSHFLIETVGRRKLFLVGALGQGVAFTITFACLIHDTKQNAKGAAFGLFLFIVFFGMTILSLPWIYPPEISSLRVRSLTNSLSTCTNWLSNFAVVMFTPIFIQKSSWGCYMFFAIVNFSYLPIIFFFYPETSGRSLEEIDIIYAKSNKDGIASWRVAAHLPKLSLKEIEQYAIEYDLYDGPVSEASTESDNERNQSELDLKQDSPQNLADKSKDGSL

SEQ ID NO: 38 - protein, 525 aa, Gaeumannomyces tritici
MTVATTGVSKSYFGGLTGHRLAVLQTALITAPSFILFGYNQAGLGGLIGLKDWERTFPEIDTTHHKESSVSTLKGFVVATFVIGALFGSLLCSYTGDKFGRRHVIFFAGLCTLVGEVLECAAFGLAQLIVGRVIVGFGIGQLSSIVPVWQSETSGAKNRGRHVVLDGVFICLGFVLESWINLGFFQFGDSPITWRPPIAIAILFSLILSGSVYLFPESPRWLMAKNRKSDAVAVLSVLRGLPQDSIEVQAEISGIELSLEDVTGKDAKLSEMLSPKNEDKLLYRFGLCILLQFFQQMSGTNLVSVYATILFQDNLGMSPQLARVLTGGALTWKFLSSFLAFFCIDRFGRRVCFMISGGGMAACMVALAVATSFPEDNRGAQISAACFLYLFNTFVPIGFLGANFLYCTEVAPVRLRMVMTSISTANHWLWNFVIVMITPVAISTIGSRYYIMYAVIAACIPLSVYFLFPETMGRNLEEINLMFRDSPSVWATVKYARNRPIGMPQEFDRKQKTDHIEGNGDESSK

SEQ ID NO: 39 - protein, 550 aa, Isaria fumosorosea
MTRKTYMGMSGSNLNWAISTIAGCDFLLFGYDQGVMGGILTLPIFLDQFPQINDLADARNMAQLPGTRSQRSLNQGIAIASYNLGCFLGAVITIFIGNPLGRRRMIFLGTAIMTVGAALQASAFTIEHFIIGRIITGIGNGGNTSTVPMWQSETCSSHKRGKLVMIEGALITFGIMVSYWIDLGLSFTDSSVSWRFPLAFQLVFCIFILAFVLGLPESPRWLILKGQEDEARAVIAAIADKEIEDPFVANEFRAIKDTAVKSAGGYGEVFHMDENRTLHRTILGYVNQMFQQISGINLITYYAAKIYADLGMSPFLARLLAALNGTEYFLASWPAVFLVERVGRRKLMLFGAVGQACTMAILAGVNSHKTDASRIAGVVFLFVFNSFFAVGWLGMTWLYPAEITPLRTRAPANALSTSANWIFNFMVVMITPVSFTNIDYHTYTIFAVINAIMVPSVYFFFPETAYRSLEEMDSIFRKVTGLRGALDVVKVAREMPHRYGKNGELLIAFDESTEKVQAEHASGSVSDGSGNNNNTMFTKTDEETSRPREK

SEQ ID NO: 40 - protein, 498 aa, Metarhizium majus
MAVPELEGRALLLTVTTLTSLGFMLIGYDNGLMGGLVNSPAFGSSFNYPGSKMIGVIVAIFEVGCFLGSILSAIFGERLGRRSTIGHGCWIMIVGAVLQASSYGRAQLIVGRIVSGLGLGIINSTVPVLQAEFSPKATRGVYVCAQLSTLNFGIFLVYWIDYAFSSHTSSYAWRVPVILQCVCILPMMGILMLIPETPRWLASHDRPDDCLRVLARMKSSSINDPEVQRQHHNILQVVAFEASIGTGSWKDLLSNDRVKSQTRLLIACSIQAFQQLGGINAVIYYTNTLFSKSIGFDERMSALMSGFLQTWFFVASFIPWFLIDRIGRKPLFVSMISLMAAAMAVQAGLIYQVQNETNISHSAGIGAAVMLFVFQGAFTIGFQATVWVYPSEILPLRLRQKGSSISTAANWIFNYMVVQITPIAIDNIGWKTYIIFAILNATWVPIIFFFFPETKGLELEDVDRLFAGDDVIESVGEKSMALATVEHVGRTDGTNERV

SEQ ID NO: 41 - protein, 498 aa, Metarhizium robertsii
MAVPELEGRALLLTVTALTSLGFMLIGYDNGLMGGLVNSPAFGSSFDYPDSKMIGVIVAIFEVGCFLGSILSAIFGERLGRRSTIGHGCWIMIVGAVLQASSYGRAQLIVGRIVSGLGLGIINSTVPVLQAEFSPKATRGVYVCAQLSTLNFGIFLVYWIDYAFSSHTSSYAWRVPVILQCVCILPMMGILMLIPETPRWLASHDRPDDCLRVLARMKSSSINDPEVQRQHHNILQVVAFEASIGTGSWKDLLSNDRVKSQTRLLIACSIQAFQQLGGINAVIYYTNTLFSKSIGFDERMSALMSGFLQTWFFVASFIPWFLIDRIGRKPLFVSMISLMAAAMAVQAGLIYQVQNETNISHSAGIGAAVMLFVFQGAFTIGFQATVWVYPSEILPLRLRQKGSSISTAANWIFNYMVVQITPIAIENIGWKTYIIFAILNATWVPIIFFFFPETKGLELEDVDRLFAGDDVIESVGEKSMALATVEHVGRTDGTNERV

SEQ ID NO: 42 - protein, 551 aa, Nannizzia gypsea
MGVGRQYFGLRGTKLNIAIGIIAGLDFLGVMGGLLTLPSFEKVFPEIATSKEAVVGLTQAEKNHRSTIQGISVASYNLGCFVGAIACIWVGDRLGRRKTIWLGAAVMVVGAALQASAFGLPHFIVGRLVTGFGNGLNTSTVPTWQSECSKSHRRGQLVMVEGALITGGICISYWLDFGFSFLEPSSVTWRFPIAFQIVFALIIMLVVMGLPESPRWLVLKGQEDEAMNVLAALSDLDREDRFVHAEFSAIKDTVIEMQKGGFRDLFTMDKDRHLHRVILAYVNQMFQQISGINLITYYAATIYEGSIGLSPFLSRVLAACNGTEYFIASWIAVFVVEKIGRRILMLFGAVGMSLSMAVLAIATSFKGQTEAGIVAAVFLFVFNTFFAIGWLGMTWLYPAEIVPLRIRAPANALATSGNWIFNFMVVMITPVSFSSIEYKTYIIFAVINAFIVPVVYFFYPETAYRSLEEMDSIFRKTKSIFTVVKIAHETPRRYGKNGEVLIDYDETDEHRARAGITQEETTTSFPEKSHANPDHDAETGNSNSPSNQSTA

SEQ ID NO: 43 - protein, 634 aa, Pachysolen tannophilus
MDSNIDDTAIPPSGYLVGKPLLYFTSVFVSLGVFLFGYDQGVMSGIITGPYFKYYFNDPSSTTIGTMVAILEIGALISSLLVGRMGDIIGRRRTIRYGAFIFVVGGSIQTFATDMHHLILGRIVSGVAVGLLSATVPVYQSEISQPHNRGQLSCVQFTGNIFGYATSVWTDYGCSFFESNLSWRFPLFVQCVIGLLLFLGTFVIVETPRWLLNNDHDAEGIVVLADLYSNGDVHDIRARNEFRNIKEDVLMSRLEDTGTSYSYMWKRYKTRILIAMSSQMFAQFNGINVISYYAPLVFEQAGWFGRDAILMTGINSIIYFLSSIPPWYLVDRWGRKPILIIGGIIMAISLFSISFVLFINVPKTPVYVVILVIIYNALFGFSWGPIPWLLPPEILPLSIRSKGASLSTATNWFCNFLVGELTPVLQETIKWRLYLIHGTSCVLSFLVVHYIYPETKGLTLEDMDSVFDDRSSTFSFQSSNSVTGLNQQQQQQHPGAGTGGFGTNYGSITNEDGLPVQYQSPAVLARHPIVAAQQLQNLKSSTPSLRSTSNYMNNVSPLIQPHELEPPNIEEIRAYKLSDNNSIKGNIRRSSENIGSMFHKVFNNNSLKRSDSTSEFTNDSEDEEANLTSNSGRH

SEQ ID NO: 44 - protein, 484 aa, Paracoccidioides lutzii
MSQKIRPALALWGTRMAFMSYGWDAGVLGGVLETAPFQDAMKHPSTTTMSMIVAAFLLASWLGCCIVASPWSDRVGRRMWVISGAAIQIIGTIISTASYSSGQMIAGRTIIGIGNGIVVASAPVYIAEITPTTSMRGPLIGILMGFACTGTTLAYWVDFAFTHARGQVVWRVPVGLQIIWSLLTIILTLPNMDSPRWYYLRNRDHEGLNVLQQLHPDQEVALRVQGEILKELREEKEEKLQLSNLIFDKSPTQAMRRIRDGVVLVGVAYLMGINMIFYYMTTIFHVYIGLPAKTSSCLSGGATTLLAIGVFVGSYFCEKSGRRKWLLWGSATQSVFIIAFTGLLAAGKKTTSSAAAAMLFGWILVFSPTWAPLPYIYVSETMPLRHRHTGVGLSMSSQWLMAFLTVYAGPIAIAKVGWKAWIWFAVFNVAAFPFVYFFIRETRGRSLENMNNLFGDEHLIDGNSSSGDTSDDVKEVKATPVSKA

SEQ ID NO: 45 - protein, 523 aa, Paraphaeosphaeria sporulosa
MARKYLGGSGERLTIWISIAASTVLIFYGYDQGVFGNVIINEHFLTTFGHPSANMQGVMTSIYNIGCFIGAMSTIWTGDILGRPRQIILGSTVIGIGAIIQTASYGVPQMMVGRIVAGLGTGMNTATAGVWQAETSKMSSRGKLVIIQMANCITGFSISNWLTLAFSFAPGDVAWRFPLAFQLFFTFCIYALCPFLPDSPRLLIRKGKPDEALEVLAALEGHGATPESASVRTQYNIIKDILDREHMNTYTWWQLLSGKGPSGVLRRMILGAWMQAMNQISGINVTSYYMSYIFINALGISELLSRILAAAGSVDYLVFACLAFFVIERYGRRKVMMVSAAACSTCWIVIAIALGLSANGGDSYKLGIVAVSFFFVFFASFGMGVLGVPWLYPTEINALEMRTKGASLAMSTNWIMNYAVVQVTLPGIQNIGWKFWIIWAVICFSFIPITYFFYPETANRTLEDIDRFFETNPGLFVHRNKLAIQLHRPVEFIEADERIATAQAEEEKNLGEKTDFVEIKEAV

SEQ ID NO: 46 - protein, 198 aa, Penicillium digitatum
MEKYLSLIMVGAFATVYTFATIPSIFLIERENEDNAKGAAVVFPFITFFAFTLLPLLWIYPPEINPLSTRTLIASTCANWICNFALVLFTPLVADHSPRSVDLFFALFKFIGLIFGVFFYVETAGRQLGEVDRIYAKAHIEGKMAWRVAQDMPKLNFEEIVQQFRGLGLDTNELAAHEKIELGLNSNSGQELEEVREK

SEQ ID NO: 47 - protein, 533 aa, Phialophora attinorum
MGRRYLGGSGQRLTVWISIASSTVLIFYGYDQGVFGNVLVSEDFLRTVGYPSVTAQGTMTSVYNLGCFAGALSTLYTGDKLGRPRTLILGSCTIAVGAIVQAACMNAAMQYAGRVIAGMGTGMNTATAGVWQSETSKMRSRGKLIIIQMANCITGFAISNWLTLGFSFAPGSVSWRFPLAFQMFFTILICLMCPFLPDSPRLLIRKGKYDEAYEVLAALEGNGATVNSPVVRTQFAIIKQVLDEEYAVKYTWWQILTGKGPSGVLRRMVLGAWMQASNQISGINVTSYYMTYVFINAINFSQLTARILAAAGAMDYLFFSFMAYFVIERFGRRSVMMTSAAACSICWTVIAISLGLSETGRADSYTMGAVAVSFFFLFFASFAMGVLGVPWLYPTEVNALAFRAKGASLAMSTNWIMNYMVAQITPPGIDNLGYKFWIIWAVICAAFVPITYLFYPETANRSLEDIDRFFHSNHGILVFNNKVATQLKRPEIYEEADRRVAAAHEKVGAGADQADEGKEAGATLVEENGAARA

SEQ ID NO: 48 - protein, 213 aa, Pochonia chlamydosporia
MGLIGRPLNWAITATAGAGFLLFGYDQGVMSGLLAGDAFTRTFPEMDTTESGHGSASLQGTVVAIYEIGCFFGALIAFVFAERLGRRRTIMLGCVILSIGGALQACASTIPHMIAGRIVAGLGNGLNTSTIPVCHSELMVASKRGKGLCIELSITVFGVMIAYWVDCGMSYVPNDAQFRFPLALQCLFAIITVIGILFLPESPRWLVAHDRHD

SEQ ID NO: 49 - protein, 498 aa, Pyrenophora tritici-repentis
MPELRGRALMLTISVLTSLGFMLIGYDNGLMGGLVGAPAFNKTFDHPSSDMIGTIVAIFEIGCFFGAMATAVIGEKLGRRKSVAIGAVISILGALLQATAYGRAHLIVGRIVSGVGLGIINSTVPVMQAEFSPKASRGIYVCAQLSTLNFGIFLVYWIDYAFVSHTSDYAWRIPTILQCIIVLAILGLLTVIPETPRWLAAHDRPDECLRVLARVADVPETDPEVQRLHTVITETVAFEQSRQAGWKDIVRSDPIKSRRRFLIACGIQMFQQLGGINAIIYYSGTLFQKSIGFDTHMSALMSGFLQTWFFVASFIPWFLIDRVGRRPLLLSMISLMAAVMAVQSGLIYQVQYKTASAKGAGIAAAAMLFIFQGAFTIGFQATVWVYPSEILPLRLRQRGSAISTAANWICNYIIVQITPRAISNIGWKTYIIFAVLNGLWVPIIFFFFPETKGLELEDVDRLFSGEASRTDLLDKDLDDERVESVVVAKTVECAGHVS

SEQ ID NO: 50 - protein, 533 aa, Pyricularia oryzae
MWLTDKFYGLRGKSLNYAIVCIAGVDFLLFGYDQGVMGGILTLPVFLSQFPTINPEADGLTSIESAQRATNQGIAVASYNLGCFVGAVIAIWISNPLGRKRMIILGTSIMVVGTIIKITAFSLVHLVIGRIIMGLGNGMNTSTVPTWQSETSSSHKRGKMVMIEGALITCGIMISYWIDLGLSFAPGSVAWRFPIAFQLVFCFFILAFVWNLPESPRWLILKGHHEEAKLVIAAIADLEVEDNFVQNEFLAIKETVEEMSKGSFRDLFATNENRNLHRTFLAYLNQVFQQISGINLITYYAAVIYSGLGMSDFMSRLLAALNGTEYFIASWPAVWLVERVGRRKLMLFGAAGQALTMAASAGVTSRSEEGFQIAGVVLLFIFNTFFAIGWLGMTWLYPAEIVPLRIRAPANALSTSANWIFNFMVVMITPVAFNTIKHHTYTIFAIINAIMVPSVYFLFPETAYRSLEEMDTIFQKVHGWKGLFTVVRQAEIEPRRYGKNGELLLDVDAVRAEKGDSSSAGRENEHREVRTEP

SEQ ID NO: 51 - protein, 540 aa, Scedosporium apiospermum
MSVQENGVCIPTFWGTSGRKLQMLVTAVATADFLLFGYDQGVMSGIISADAFTEDFPEVVTGGSAYEGFVTSIYAVGCFLGAVFILLFGDHLGRRMSIYLGATTMIVGVIIQVSCVPVSGGTTAQFIIGRCITGVGNGINTSTIPTYQAECSHSHNRGKLICIEGGNVAIGTLIAYWIDYGAIYGPHDFTWRFPIAFQCVFAITVLILNTRLPESPRWLLTKDKHEEAAMVLAALAGKPTDDYEVRSQMTAIVESIKASGHSGGVTPMSALFTNGKTQHFRRMILGFSSQMMQQLSGCNAVIYYFPILFQTSIGVSHNMALLLGGVNMIVYSIFATTSWFAVERIGRRKLFLIGTVGQCLSMVLAFGALIPGTEAAARGAAVGLFTYIAFFGATWLPLPWLYPAEINPLKTRTKANAVSTVSNWLWNFFIVMITPVLVDNIGWGTYLFFAVLNAIFFPIIYFFYPETSQRSLEEIDHIFAKGYTENMSYVRAAKELPRLSGEINAQTVGRDDVDVEKSGEVSDEMDSRSVENTEKLSHLD

SEQ ID NO: 52 - protein, 512 aa, Sphaerulina musiva
MAFELRGSKLIAVILLASGLDFLLFGYDQGLFGGILGGKRFKAMLGEPGPTMTGFVTGVYDIGCAIGAVAAFFFGEAIGRKKSIIYANVIVIIGATIQTACYSYAQMAVARVIAGVGVGLSTVAVPILQSETLPSHNRGALLVVQSALIIIGVAIASWLCFATLFANSSLQWRFPIACQILFSLLVLCCCPFLPETPRWLCKHNRIDEARYTISRLLDKPEDDAEVKGQLHEILANIEAENEDGEPSWSEVFSNATKARNLQRVLLGMGPYMMNQWSGINALCYYLAYILETYLDFSQNMSLILASVAFTQYAIFSWPPYFYIDKIGRRWTVMLSSIGCAICMAVIAGCLIKQSYSSAAAAVAFMFLYLDCFTLGILPVSWSYSAEIQPLRVRNKATAVGVFSHWTSNFVVVMVTPIGLNHIGGNYFWIWAIVCASFVPLTYFFGVETSGRTLEQIDESFFENPRILMGLDKKNTVVIKASKQDEESRFRALAKEEEKHPERVSVEQVEEKS

SEQ ID NO: 53 - protein, 534 aa, Talaromyces atroroseus
MWTTTSGLRGKKLHLAITFTSVIGFSLFGYDQGLMSGLISGDQFVKEFPVLDGDSLHVSVLQGAVTSCYEIGCFFGAIFTLLFGQRIGRTPLLVGGGALMVVGTVISTASFGPHWGLGQFVIGRVISGVGNGMDTATIPVWQSECSRAHNRGFLVCFEGAIIAVGTFIAYWIDFGLSYVESSVQWRFPVAFQILFALLVVIGALMLPESPRWFIMSGKTQEALHVLAQLNDSSEDADDVLRDFNLMQADLKSLENAEASSWKTLFTFGKTQEFQRMMIGCSGQFFQQFTGCNAAIYYSTLLFKNNLHMTGKLPLVLGGVFATVYALATIPSFFMVEKVGRRNLFLIGFLGQGLSFIITMGCLVDDTTQNAKGAAVGIFLFITFFAFTTLPLPWIYPPEINPLRTRTMAASASTCVNWICNFAVVMFTPVFSNKSAWGIYLFFALVNFIAIPFAWFFYVETAGRDLEEVDIIFAKAHVENKWPFQIAQQMPKLTHEEISQQLFDLGLSVSDHSASSESEKVEIAATNNEHRTASD

SEQ ID NO: 54 - protein, 488 aa, Trichophyton benhamiae
MAGIISAMPFNTVFPETKDNPTNQGFVTAIYEIGCLLGAVSIIWGGDMLGRRKSIVTGAIIMAIGAIIQVTSFVGHQPYAQFIIGRIITGVGNGINTSTIPTYQAECSHASNRGLLICIEGATIAFGTLIAYWIDYGASYGPDSFSWRFPIAFQIAFSIVMVTGMIWLPESPRWLCMRDRSDEGERVIAALHGVPITDPLVQAEKNAVMESIRASGEVGKPTPLSVVFTGGKTQHRRRMFLGVFGQFAQQLSGCNAIIYFFPVLFEKSIGVDHNMATLLGGVNMIVYSIFATTSWFLIERAGRRKLFLYGAAGQAISMTITFACLIPNTPATAKGAAVGLFTYIASFGATWLPLPWLYAAEISPIKTRAKANALSTCSNWLFNFFIVMITPVMLAGIGWGTYLFFAIINVCFLPIIYFFYPETAKRSLEEIDIIFAKGYCENKSYVQAARELPYLTEEEISRMDAEYGHGKPSETASPVNEKESDSEQ

SEQ ID NO: 55 - protein, 472 aa, Verticillium alfalfae
MSEKSDRADTINNHQGTVDSTPKSGFSRFCSKMGDLPQWKVNGKLLRGAALNWGIGVIASCGFLMFGYDQGVLSGLLTLDDFQKNQALMTPLDASNPLCWNDDGSRDERYCHGDANTQAAGVAMYQIGCFLGAVLILFYGESWGRRSSTFWGSLIMIIGGIMQAASLEYGLFVSGRVIGGRFKRLLTNRPSQKLRRTLLGIAAQFFQQICGITLITYYATFVFENSLGFGPQLSRLLAALNGTEYFLASLVALPLIERVGRRKLMLFGAFGMMGSMAILAGTTSTGTTNEDGAPQLSTAYGVTAVVFLFAFNSFFAVGWLGMTWLYPAEVTGLNIRIQANALSTCSNWISNFLIVMITPPAFANLQWKTYVMFAVFNAALIPCVYLYFPETSKRSLEEIDLYFARAWSEGVSPVKMAKTMPRYTATELDNELAKYFSAADIEQRRGSMLQSRRPSAMTQAPDEPSVNKNATQ

SEQ ID NO: 56 - protein, 533 aa, Verticillium dahliae
MGYTTWWKRLSPRQLNIAIQTFSVISIFFEGYDQGVMGGVNASPRYVEEVGIGLPDGTVTDTLHQGGIVSVYYLGCIAGCFAGGWLADRIGRINGLFIGCIFAIIGGALQAAAQSSNFIIVARVITGIGTGALTGITPVMVSETSTAEHRGGFLGYVFIANYLGISIAYWLSFGLAFIDGGYSDIRWRFQLAFQCLPALLLFLGIKILPDTPRFLASVGRYDEAREVIEHVRGNFGPLVEREFLEIRTVAEESTKSSPIEFIKILFGRGPKPGYNLGQRAWLCLFLQIMASWTGITAVTAYSPILLSQAGYTELTQNGLAGGLNTVGIVGTIISAQIVDRLGRRTCLMGGALALSAVNLIAGALYEGSRAHPDRASQFAPVAVAMLFLFNLSYAATWGTVAFLIPTEIWSSDLRAQGNGFGITGWAVGVGMTTLVNPIMFGVLKNWTYFLFAGLNLLWVPVVFLFYPETSGRSLESIDALFAANSIFNTKMERSYMAHGDVLAERGNHDNQVLSASDSGSKPGPPGSVEKLQV

SEQ ID NO: 57 - protein, 573 aa, Torulaspora delbrueckii
MIEKKSLKSRFFSRTSHFGLTGKTLRYVITLCAMTGFSLFGYDQGLMASLITGTQFNYEFPATKSKSDNDTHASTVQGAVTSCYEIGCFFGSLFVMFYGEKIGRKPLIVIGSVITIVGAVISTTAFRDYWALGQFVVGRVITGVGTGLNTSTIPVWQSEMSDPSIRGILVNLEGSTIAIGTMLAYWIDFGFSFIDSSVQWRFPVSMQILFALILCFMIVNLPESPRWLISQSRTEEARYLLGQLDDVDPNDDRIVAEVAMIHDAVNRSKQEKNSMSVLFSGGKSQNMQRALVAASTQFFQQFTGCNAAIYYSTVLFHETIQLSPRLSMILGAVFSTVYALSTIPSFFLIERLGRRKLFLLGATGQAISFTITFACLVRQTEENAKGAAVGLFLFIVFFGCSMLSLPWIYPPEIASMKVRASTNAFSTCTNWLCNFAVVMFTPIFINKSGWGCYLFFACINYLYIPVIFFFYPETAGRSLEEIDIIYAKSHEEGTQAWRVAAHLPKLSLQEVDDHANALGLYEDDLEKEDFAAEEEGKEAGAHGYALFARNTSTSNNEDGSSESEKDQNATPRA

SEQ ID NO: 58 - protein, 532 aa, Penicillium rubens
MGYTTLWKRLSPRQLNVAIQIFSLISIFFEGYDQGVMGGVNNSPRYVEEVGIGKPDGTVTDTTHQGGIVSIYYLGAIFGCFAGGWLADRVGRINGLLAGSLFALVGGALQAGAQNSDFMLCARVITGIGTGALTGITPVLVSETSSANHRGGFLGYVFIANYLGISVAYWISFGLAFVDNGYSDVRWRFLLAFQCFPALLLAAFIKMLPDSPRFLASVGRNDEARDLLNRIRKDRASQDDIDREYLEIIVTAKGSKFSSPIEFVKILFGKGGRPGMNLGRRAWLCVWLQIMASWTGITAVTAYSPVLLAQAGYSDIKQNGLAGGINTIGIIGTIISAIIIDRLGRRVCLMGGAAVLFAVNLIAGAVYEGSLHNPEKASQYAPGAVTMLFLFNLGYAATWGTVAFLVPTEIFPSDLRAQGNGFGITGWAIGVGMTTLVNPIMFDVMTSRTYFLFAGLNLIWIPIVYLFYPETRNRSLESIDALFSTPSPFHWKMEQAYKLHGDVLAEHGVNRNEALGDGKSELTTSPTELGTV

SEQ ID NO: 59 - protein, 237 aa, Trichophyton rubrum
MAKKYLGGSGDKLTIWISIAASTVLIFYGYDQGVFGNVLIGEDFLQTMGYPSTNLQGTMTSVYNIGCFVGAMSTVWTGDYFGRPRQIIVGSTIIAIGGIIQASAYGVPQMMVGRVVAGLGTGMNTSTAGVWQSETSKMSSRGKLVIIQMVFFTLCIYAMCPFLPDSPRLLIRKGEYSEALEVLAALEGNGATSNSHSVKTQFNVIKDVLDRENLNSYTWFKLLMGKGESSRFPSVYT

SEQ ID NO: 60 - protein, 568 aa, Lachancea thermotolerans
MPSRLSVNRTSTLGLNGRSLRLAITITSVIGFSLFGYDQGLMSGLITGKEFNSEFPATGGDDRRTKLVQGAVTACYEIGCFFGSLFVMFRGEQIGRKPLVILGACLTIVGTVISTAAFGPHWGLGQFVVGRVCTGVGTGLNTSTIPVWQSEMSKPENRGILVNLEGSMIAVGTMIAYWIDFGLSYVDSSVQWRFPVAMQIFFALLLLAGIWELPDSPRWLMSRGRRDDALHVLAKLDNLPEDDDAIIAEATVIQDAVNRFRHEKRSVKDLFTGGKTQNLQRALVASSTQFFQQFTGCNAAIYYSTVLFQESIGLTGKLPLILGGVFATIYACFTIPSFFLVETLGRRKLFMLGAAGQAISFTITFGCLTKDDTEVAKGAAVGLFLFICFFGMSMLSLPWIYPPEIASMRVRSATNALSTCTNWLCNFAVVMFTPIFIMDTGYGCYLFFAVMNYLYLPVIFFFYPETAGRSLEEIDIIFAKAHVDGTMPWRVAANLPKLSFSEIEDQANALGLYEDDNEKQDLDLDEAADREDAIRQNSGVQGYTLFEKRENGAESGQPDGSSTSQKSD

SEQ ID NO: 61 - protein, 542 aa, Aspergillus oryzae
MWTTTSGLSGRSLRLSITFAAVVGFSLFGYNQGMMAGLLNGDEFVDSFPILKMPDNPTAGEKHYIDVIRGAVTSCYELGCFFGALFSMFLGDKLGRTRLIFMGASILIIGALLTTVCFTGHWEVGQFVIGRVVSGIGNGMNTATIPVWQSECSGAHNRGFLVCFEGAMIAGGTFIAYWVVFGMSHAADSVQWRFPVALQIFFALVVAAGAMMLPDSPSWFVMRGLDKEACEVLGKLKGTSPDSDQVLHDFNFLKQDMESSKNTQSNWKTVFTFGKTQEFQRLLIGCSGQFFQQFTGCNAAIYYSTLLFQENLGMEKYLSLIMGGVFATVYVLATIPSFFMIEKVGRRNLYLVGFLGQGLSFVITFACLIKETEENSKGAAVGIFLFITFFAFTLLPLPWIYPPEINPLRTRTVGASASTCTNWICNFAVVMFTPLFAGQSPWGVYLFFALFNFLGLIFGFFFYVETAGRELEEVDIIYAKAHVEGKMAWRVANTMPKLSFEEITQQSRELGLDTNDHGVHEKTELGLSSDSGQETEEVHEKH

SEQ ID NO: 62 - protein, 488 aa, Trichophyton verrucosum
MAGIISAMPFNTVFPETKDNPTNQGFVTAIYEIGCLLGAVSIIWSGDMLGRRKSIVTGAIIMAIGAIIQVTSFVGHQPYAQFIIGRIITGVGNGINTSTIPTYQAECSHASNRGLLICIEGATIAFGTLIAYWIDYGASYGADSFSWRFPIAFQIAFSIVMVTGMIWLPESPRWLCMRDRSDEGERVIAALHGVPVTDPLVQAEKNAVMESIRASGEVGKPTPLSVVFTGGKTQHRRRMFLGVFGQFAQQLSGCNAIIYFFPVLFEKSIGVDHNMATLLGGVNMIVYSIFATTSWFLIERAGRRKLFLYGAAGQAISMTITFACLIPNTPATAKGAAVGLFTYIASFGATWLPLPWLYAAEISPIKTRAKANALSTCSNWLFNFFIVMITPVMLAGIGWGTYLFFAIINVCFLPIIYFFYPETAKRSLEEIDIIFAKGYCENKSYVQAARELPYLTEEEISRMDAEYGHGKPSETASPVNEKESDSDQ

SEQ ID NO: 63 - protein, 488 aa, Neofusicoccum parvum
MSGLLTGSAFTKVFPEIDTQNGGSSSLQGTVVAIYEIGCFAGALITFAFGEQLGRRKCIMAGCTILTIGATIQCASYGIPQLIVGRIVAGIGNGLNTSTIPVWHAELMQAHDRGKGLAIEFILNIFGVALAYWVDYAFSFVDNESQFRFPIAFQIAFALVTLASIIFLPESPRWLLNHDREAEARNILWRLQPNAKEIAEDSDVVNNEMAIIQHALYEEKEVAGGTTFKAIFKDGPQRFRYRTLLGIGGQFMQQLSGINLITYYAAVIFETSIGMSHNTALLVAGANGIAYFLSTFPVVWVLDRLGRRKLMLFAVIGQSCCMAILAGTVSNGGKSAGIVAAVMLFLFNFFFAIGLLAIPWLLPAEYAPLAIRTKAASLATASNWIFTFLVVEIVPVSINNIAWRTYIYFAVFNAFFVPIIYFFYPETKNLSLEEIDMLFTGDKVLMHLPDSMRIPANDNAAVASAIRGEKDNVAVVEDVNNGNNSEKS

SEQ ID NO: 64 - protein, 516 aa, Eutypa lata
MSSPTTFLGFQGSSLTLAQLFLVVCPAFVLFGYNQSGLGGLVGLQDWSQTFPRIDTLNTEGAQKDNNATIQGLVVATFTLGALPGCLSCAYTADRFGRRTVIFVGALLTLIGEVLEASAFHLAQMIVGRVILGAGVGMLSGVVPTWQSECSNSKNRGKHVVLEGLFISMGYVLQAWINLGFYQFETGPVTWRPPIAIPIFFSLVLMSFIYLMPESPRWLIRQGRVSEARAAMSALKGLADDAQEIHAEVAAVELSLEETGQKKAALADLLRMDEDKLLYRFGICILLQFFQQMSGGNLISVYSTIIFQRGLNLEAETSRILSGGTLTWKFLSCFVSFFTIDRFGRRVALMVSGTGMAVCMMSLAIATSFPTSNLAAQIVSVLFVFLFNFFIPIGFLGANFLYCTEVAPLRLRVAMSSISTANHWLWNFVVTMITPVAIESIGYKYYIVYTVVGFCIPLTVYFLYPETMGMRLEDIDLVFRESPSVLATVKYARSRSQRSNEEVLADKKKVEYAEKI

SEQ ID NO: 65 - protein, 517 aa, Phaeoacremonium minimum
MGFKTAFGLTGHALSILQIALIVAPSFVLFGYNQAGIGGLLSEEDWVKTFPEIDTVHATGTTKSSKSTLQGFVVATFVIGALIGALSCSYTGDIFGRRNVIFAGAVFTLVGEVLEASSFSLAQFIVGRVLIGAGVGQLSSIVPVWQSETSGAKNRGRSVVVTGLFICLGYVLESWIDLGFFEFKTGPLTWRPPIAIAVAFSLVLMASVYVFPESPRWLLMKNRVQEARESLSVLRGHAEDSLEVQAELAGIELSLEETSGNAAKLGDMLKMGEEKLLYRFFLCMLLQFYQQMSGSNLVSVYATTLFQTNLGLSSELSRVLTGGALTWKFLSSFIAFVTIDRFGRRAVFILSGIGMSCCMIALAVSTSFGKENRAAQIAAGCFIYLYNTFVPIGFLGANFLYCTEVAPIRLRMAMSSISTANHWLWNFVVVMVTPVAIETIGWQFYIVFAVIAACVPVSVYFLFPETMGRNLEEIDMVFRESPSVWATVRFARSRPALTAVEYAEKHDNVDHLEKTAE

SEQ ID NO: 66 - protein, 517 aa, Aureobasidium namibiae
MVGYLDGIQGKTLYKIMSAACGSAFMLYGWDAGVLGGIQETKEFRAAIGDPQGAFIIPIIAAIYNLAAGVMSLCVSFYGMQIGRKGTILLGCLLICIGALLQASTYSVGQIIVGRIVTGAGIGNIAAAVPTYMAEMSLEAKERGPEVSYQLALLITGVALAYWIDLGFVQGLDRHPWLWRIPLALQSCFAIFSAVLLFMLPDTPRWYYARGKEAKGDRVLARLHGLPVEHQNVQAVKADIMASMEEEDETGKISIVSLFWDNTELQFGRRLRTSFLINWAQQFLGINMLVYFSTQIFSNLNYSPLLSGILAGVLNTAFAIASYPPIWYIEKVGRRAMMIWSALGCGVCMLIYVVLTTLPAHMQSAGTNWGAVAIIILYEIVFAFGWLGTCWIYGPEIAPLKYRHVAGSLGAAGEWFSTFVMVFGGGTGINAVGPKIFIWPLLCCFLAAAYVYFLCPETTGKTLEEIDALFARSPEVRERLERDIAARRAGVLPGNEKSMSRDSSDMSKMEVSNIEKI

SEQ ID NO: 67 - protein, 677 aa, Kluyveromyces lactis
MSDSTDKDRHNKNSSRGIVNTDIEDDNSPSPLDTTDKKGAPELQKPGNPSLFKESALKNSDQLQFRNSFNIPNAVGGTNGIVIPPNVQPLDQQRIPPSYTDHLQVKDTYLTGRTLLYFTSIFVSLGVFLFGYDQGVMSGIITGPYFKTYFNNPTAATIGTMVSILEIGALVSSLLVSNIGEKFGRRFTIKYGSLIFILGGLVQTFSWEMGHMIFGRIISGIGVGLLSTIVPIYQSEISPPHNRGKLACIEFTGNIVGYASSVWVDYACSYIESDTSWRLPLFIQCVMGLLLFLGSFVIVETPRWLLNHDHDIEGLVVIADLHSDGDVLHSKAHEEYKLIKETVLISRLEGEKKSLRFAFKRYRTRMLIAMSSQMFAQLNGINVISYYAPLVFEQAGWVGREALLMTGINSIIYILSTILPWKLVDKWGRKPILLSGALVMGTSLLAIAMSLWANVAATPRLVVVFVIIFNAFFGYSWGPIPWLYPVEIAPAMARSAMASASTATNWLFNWLVGIMTPILQEKIHWRMYLIHTVSCYLSFWCVLKVYPETAGLRLEDMDSVFDDRSSTFSFQSGTSAEIEQQSHLVSGGGEVAPSTRSRKSVYSNAQSMFNKDEIQPPTLTQVLQWKEERTQTKPLKKFIRRGSETVCLIYNKVRNLRSTNDTNQIEYGAVSNNQPPN

SEQ ID NO: 68 - protein, 163 aa, Rasamsonia emersonii
MTSIGGPKCGIVAATFLFVFNTFFALGWLSIPWLYPAELVPLEIRAQANALSTSANWIFNFMVVMITPVAFSSIGWRTYIIFAVFNAASIPILYWCYPETAYRSLEEMDIIFAKATGVVDAVRVARTEPRHFGKHGEVLREMVPDVKDTTGIGSEKGGVEHVQ

SEQ ID NO: 69 - protein, 575 aa, Pachysolen tannophilus
MFKKIDKIDKSDYVASSSKKYLGMRGAPLHKAIATIAGLGFLLFGYDQGVMGSLLTLDSFLETFPQINDSVDTSKSTLKGFVIAVYELGCMTGAFFTMWKGDIFGRRKMIFYGSIIMTIGGILQCTSYSVAQLAVARVVSGVGNGFITSTIPTLQSECAKPHRRGALIMMSGALISFGICFSYWVDFGLYFATGDVQWRFPIAFQIVFSLLLTSLIFELPESPRWLVKIHEIERARETFAALDDVSVDDPLIDDEIKDIQAVLKRDLDLGADKFSFSVVFKFDEKKTFHRTMLAYFVQVMQQISGINLITYYAGTIYETYIGMNALDSRILAACNGTEYFLASLIPFYTVERFGRRSLFLFGTAGQAITMAILTGVQWASEYKGDQGAAIACAVFLFVFNTFFAIGMLGMTWLLPPELVTLESRASVTGLSTSANWLFNFVVVMITPVCFTHIGPYTYTIFAVVNAIMVPCIFFFYPETKGRSLEEMDRIFEQSNPKTPWDVVRIAREMPFENRDIDNEDEEDKINLDRSSETSSVSNEKGSASFTLDSVNDTGFFVKNEENKNEQETSQPEKKE

(19) United States
(12) Patent Application Publication
(10) Pub. No.: US 2026/0146256 A1
(43) Pub. Date: May 28, 2026

(54) YEAST STRAINS EXHIBITING PROLONGED PERSISTENCE DURING A PLURALITY OF FERMENTATION CYCLES

(71) Applicant: Danstar Ferment AG, Zug (CH)

(72) Inventors: Emily Agnes Stonehouse, Lebanon, NH (US); John McBride, Lebanon, NH (US); Justin Van Rooyen, Lebanon, NH (US); Zach Losordo, Lebanon, NH (US); Petra Deane, Lebanon, NH (US)

(21) Appl. No.: 19/270,989

(22) Filed: Jul. 16, 2025

Related U.S. Application Data
(63) Continuation of application No. 17/843,741, filed on Jun. 17, 2022, now Pat. No. 12,385,052.
(60) Provisional application No. 63/211,831, filed on Jun. 17, 2021.

(51) Int. Cl. C12N 15/81; C12N 1/165

(52) U.S. Cl. CPC C12N 15/81; C12N 1/165; C12N 2510/02; C12N 2511/00; C12Y 101/01177

(57) ABSTRACT

The present disclosure provides yeasts, which can be recombinant yeast host cells, exhibiting prolonged persistence when submitted to a plurality of fermentation cycles. The yeasts exhibit at least one of the following phenotypic trait: a fast settling phenotype, a rugose phenotype, an improved invertase activity, triploidy, increased signaling in a RAS/cAMP/PKA pathway or combinations thereof.

### CROSS-REFERENCE TO RELATED APPLICATION AND DOCUMENTS

This application is a continuation of U.S. application Ser. No. 17/843,741, filed Jun. 17, 2022, which claims priority from U.S. provisional application 63/211,831 filed on Jun. 17, 2021, both of which are herewith incorporated in their entirety.

### REFERENCE TO AN ELECTRONIC SEQUENCE LISTING

The contents of the electronic sequence listing (580127-436C1-SL.xml; Size: 128,879 bytes; and Date of Creation: Jul. 15, 2025) is herein incorporated by reference in its entirety.

### TECHNOLOGICAL FIELD

The present disclosure concerns yeast host strains exhibiting prolonged persistence during a plurality of fermentation cycles in which the fermenting population is recycled.

### BACKGROUND

Multiple fermentations cycles using a recycled biomass are susceptible to contaminations by wild yeasts. For example, in Brazilian fuel ethanol fermentations, the yeasts are pitched at the beginning of the sugarcane crushing season and are continually recycled for more than 200 days. The yeasts are recycled using continuous centrifugation and acid washing to improve productivity. Wild yeast contaminants are continually entering the fermentation since the fermentation substrates (e.g., sugarcane juice and molasses) are not sterilized. In addition, the predominate Saccharomyces cerevisiae yeast strains used in the Brazilian fuel ethanol industry are highly heterozygous and are known to have genomic rearrangements which creates challenges to the traditional molecular identification methods used to monitor yeast populations (such as, for example, microsatellite and inter-delta sequence amplification, random amplified polymorphic DNA (RAPD) or karyotyping by pulse-field get electrophoresis (PFGE)).

Limiting contamination is important in continuous fermentations from both an economic and a processing perspective. Contaminating yeast have been associated with decreased ethanol yields, flocculation, and foaming. Greater than 95% of the contaminating yeasts are reported to be other Saccharomyces strains many of which have unfavorable fermentation characteristics and can lead to large productivity losses if allowed to proliferate. Less than 5% are non-Saccharomyces such as Dekkera bruxellensis, Candida krusei and Schizosaccharomyces pombe, but these strains can cause issues if left unchecked.

There is a need for limiting wild yeast contamination over numerous fermentation cycles using a recycled biomass or continuous fermentations so as to prolong the persistence of fermenting recombinant yeast host cells.

### BRIEF SUMMARY

The present disclosure provides a yeast exhibiting prolonged persistence when submitted to a plurality of fermentation cycles. The yeast exhibits at least one of the following phenotypic trait: a fast settling phenotype, a rugose phenotype, an improved invertase activity, triploidy, increased signaling in a RAS/cAMP/PKA pathway or combinations thereof.

In a first aspect, the present disclosure provides a recombinant yeast host cell capable of modulating the activity or the expression of a first polypeptide and/or a second polypeptide for increasing, when compared to a parental cell, the conversion of a biomass into a fermentation product and/or for reducing the conversion of the biomass into a fermentation by-product. The recombinant yeast host cell comprises of at least one of phenotypic trait providing persistence of the recombinant yeast host cell in a plurality of fermentation cycles. The at least one phenotypic trait is: a fast settling phenotype, a rugose phenotype, an improved invertase activity, triploidy, increased signaling in a RAS/cAMP/PKA pathway or combinations thereof. In an embodiment, the recombinant yeast host cell, after a total of 40 fermentation cycles, is present in a proportion to at least 99% in a fermenting population. In an embodiment, the fermentation product is an alcohol, such as, for example, ethanol. In another embodiment, the fermentation by-product is glycerol.

In another embodiment, the recombinant yeast host cell is capable of increasing the expression of the first polypeptide. In yet another embodiment, the first polypeptide is a heterologous polypeptide capable of being expressed (and in some embodiments is expressed) in the recombinant yeast host cell and the recombinant yeast host cell comprises a first heterologous nucleic acid encoding the first polypeptide. In yet a further embodiments, the first polypeptide is a sugar transporter-like protein (STL1). In still another embodiment, STL1 has the amino acid sequence of SEQ ID NO: 8, is a variant of the amino acid sequence of SEQ ID NO: 8 having glycerol transport activity or is a fragment of the amino acid sequence of SEQ ID NO: 8 having glycerol transport activity and/or wherein the first heterologous nucleic acid molecule comprises the nucleic acid sequence of SEQ ID NO: 7 or is a degenerate sequence of SEQ ID NO: 7 encoding SEQ ID NO: 8. In still a further embodiment, the recombinant yeast host cell is capable of decreasing the expression of the second polypeptide, wherein the second polypeptide is a native polypeptide. In an embodiment, the native gene encoding the second polypeptide is inactivated. In some embodiments, the second polypeptide has NAD-dependent glycerol-3-phosphate dehydrogenase activity, such as, for example, NAD-dependent glycerol-3-phosphate dehydrogenase is glycerol-3-phosphate 1 (GPD1) and/or NAD-dependent glycerol-3-phosphate is glycerol-3-phosphate 2 (GPD2).

In some embodiments, the recombinant yeast host cell exhibits the fast settling phenotype. In some further embodiments, at least 5% of a population consisting essentially of the recombinant yeast host cells is able to sediment by gravity after 5 minutes. In some additional embodiment, a population consisting essentially of the recombinant yeast host cells is able to sediment by gravity after 5 minutes in a proportion equal to or higher than 15% than a control population consisting essentially of control yeast cells lacking the fast settling phenotypic trait. In an embodiment, the control yeast cells are from a Saccharomyces cerevisiae PE-2 strain.

In some embodiments, the recombinant yeast host cell exhibits the rugose phenotype. In some further embodiments, at least 90% of a population consisting essentially of the recombinant yeast host cells, after exponential growth in a medium inoculated at low recombinant yeast host cell density, has at least two daughter cells attached. In some additional embodiments, the recombinant yeast host cell is capable of reducing the transcription factor activity of a Activator of CUP1 Expression (ACE2) polypeptide. In some further embodiments, the recombinant yeast host cell is capable of expressing (and in some embodiments expresses) a mutated ACE2 polypeptide, wherein the mutated ACE2 polypeptide has decreased activity when compared to a wild type ACE2 polypeptide. In yet some additional embodiments, the mutated ACE2 polypeptide is a variant or a fragment of the amino acid sequence of SEQ ID NO: 10.

In some embodiments, the recombinant yeast host cell exhibits the improved invertase activity phenotypic trait. In some further embodiments, a population consisting essentially of the recombinant yeast host cells is able to consume hydrolyze more than 0.05 gram of sucrose per gram of dry cell weight per minute and/or exhibits more than 1.0 time more invertase activity than a control population consisting essentially of control yeast cells lacking the improved invertase activity phenotypic trait. The invertase activity can be measured after exponential growth of the population diluted to a concentration of 9 mg/mL on a wet cell weight in a buffer and wherein the buffer comprises 40 g/L of sucrose, is at of pH 5 and at a temperature of 35° C. In an embodiment, the control yeast cells are from a Saccharomyces cerevisiae PE-2 strain. In an embodiment, the recombinant yeast host cell is capable of increasing the enzymatic activity of at least one polypeptide having invertase activity. In still another embodiment, the at least one polypeptide having invertase activity comprises SUC1, SUC2, SUC3, SUC4, SUC5, SUC6, SUC7, SUC8 or SUC9.

In some embodiments, the recombinant yeast host cell is a triploid cell.

In some embodiments, the recombinant yeast host cell exhibits the increased signaling in the RAS/cAMP/PKA pathway phenotypic trait. In some additional embodiments, a population consisting essentially of the recombinant yeast host cells is able to exhibit a fold increase in the production of cAMP of equal to or less than 1.7 and/or a fold increase in the production of cAMP of less than 70% when compared a control population consisting essentially of control yeast cells lacking the increased signaling in the RAS/cAMP/PKA pathway. In some embodiments, the production of cAMP is measured in the population having been glucose depleted and 5 minutes after a glucose spike. In an embodiment, the control yeast cells are from a Saccharomyces cerevisiae PE-2 strain. In another embodiment, the recombinant yeast host cell is capable of expressing (and in some embodiments, expresses) a mutated polypeptide involved in the RAS/cAMP/PKA pathway. In some further embodiments, the mutated polypeptide involved in the RAS/cAMP/PKA pathway comprises a mutated RAS2 polypeptide having increased activity when compared to a wild-type RAS2 polypeptide. In yet additional embodiments, the mutated RAS2 polypeptide is a variant or a fragment of the amino acid sequence of SED ID NO: 19. In some further embodiments, the mutated polypeptide involved in the RAS/cAMP/PKA pathway comprises a mutated IRA2 polypeptide having a reduced inhibitory activity towards a wild-type RAS1 and/or a wild-type RAS2 polypeptide when compared to a wild-type IRA2 polypeptide. In still yet another embodiment, the mutated IRA2 polypeptide is a variant and/or a fragment of the amino acid sequence of SEQ ID NO: 22.

In some embodiments, the recombinant yeast host cell is from the genus Saccharomyces sp. or from the species Saccharomyces cerevisiae.

According to a second aspect, the present disclosure provides a process for prolonging the persistence of a yeast in a fermenting population in a plurality of fermentation cycles. The plurality of fermentation cycles comprises an initial fermentation cycle and at least one further fermentation cycle. The initial fermentation cycle comprises: (i) contacting an initial fermentation medium comprising a fermentable carbohydrate with an initial fermenting population to obtain a fermented medium comprising a fermentation product and a fermenting population and (ii) substantially isolating the fermenting population from the fermented medium. Each further fermentation cycle comprises: (iii) contacting the fermented population obtained from a previous fermentation cycle with a further fermentation medium comprising the fermentable carbohydrate to obtain a further fermented medium and (iv) substantially isolating the fermenting population from the further fermented medium. In the process, the initial fermenting population consists essentially of persistent yeast cells having at least one of the phenotypic trait as defined herein.

In some embodiments, the persistent yeast cell exhibits the fast settling phenotype. In some further embodiments, at least 5% of a population consisting essentially of the persistent yeast cells is able to sediment by gravity after 5 minutes. In some additional embodiment, a population consisting essentially of the persistent yeast cells is able to sediment by gravity after 5 minutes in a proportion equal to or higher than 15% than a control population consisting essentially of control yeast cells lacking the fast settling phenotypic trait. In an embodiment, the control yeast cells are from a Saccharomyces cerevisiae PE-2 strain.

In some embodiments, the persistent yeast cell exhibits the rugose phenotype. In some further embodiments, at least 90% of a population consisting essentially of the persistent yeast cells, after exponential growth in a medium inoculated at low persistent yeast cell density, has at least two daughter cells attached. In some additional embodiments, the persistent yeast cell is capable of reducing the transcription factor activity of a Activator of CUP1 Expression (ACE2) polypeptide. In some further embodiments, the persistent yeast cell is capable of expressing (and in some embodiments expresses) a mutated ACE2 polypeptide, wherein the mutated ACE2 polypeptide has decreased activity when compared to a wild type ACE2 polypeptide. In yet some additional embodiments, the mutated ACE2 polypeptide is a variant or a fragment of the amino acid sequence of SEQ ID NO: 10.

In some embodiments, the persistent yeast cell exhibits the improved invertase activity phenotypic trait. In some further embodiments, a population consisting essentially of persistent yeast cells is able to consume hydrolyze more than 0.05 gram of sucrose per gram of dry cell weight per minute and/or exhibits more than 1.0 time more invertase activity than a control population consisting essentially of control yeast cells lacking the improved invertase activity phenotypic trait. The invertase activity can be measured after exponential growth of the population diluted to a concentration of 9 mg/mL on a wet cell weight in a buffer and wherein the buffer comprises 40 g/L of sucrose, is at of pH 5 and at a temperature of 35° C. In an embodiment, the control yeast cells are from a Saccharomyces cerevisiae PE-2 strain. In an embodiment, the persistent yeast cell is capable of increasing the enzymatic activity of at least one polypeptide having invertase activity. In still another embodiment, the at least one polypeptide having invertase activity comprises SUC1, SUC2, SUC3, SUC4, SUC5, SUC6, SUC7, SUC8 or SUC9.

In some embodiments, the persistent yeast cell is a triploid cell.

In some embodiments, the persistent yeast cell exhibits the increased signaling in the RAS/cAMP/PKA pathway phenotypic trait. In some additional embodiments, a population consisting essentially of the persistent yeast cells is able to exhibit a fold increase in the production of cAMP of equal to or less than 1.7 and/or a fold increase in the production of cAMP of less than 70% when compared a control population consisting essentially of a control yeast cell lacking the increased signaling in the RAS/cAMP/PKA pathway. In some embodiments, the production of cAMP is measured in the population having been glucose depleted and 5 minutes after a glucose spike. In an embodiment, the control yeast cells are from a Saccharomyces cerevisiae PE-2 strain. In another embodiment, the persistent yeast cell is capable of expressing (and in some embodiments, expresses) a mutated polypeptide involved in the RAS/cAMP/PKA pathway. In some further embodiments, the mutated polypeptide involved in the RAS/cAMP/PKA pathway comprises a mutated RAS2 polypeptide having increased activity when compared to a wild-type RAS2 polypeptide. In yet additional embodiments, the mutated RAS2 polypeptide is a variant or a fragment of the amino acid sequence of SED ID NO: 19. In some further embodiments, the mutated polypeptide involved in the RAS/cAMP/PKA pathway comprises a mutated IRA2 polypeptide having a reduced inhibitory activity towards a wild-type RAS1 and/or a wild-type RAS2 polypeptide when compared to a wild-type IRA2 polypeptide. In still yet another embodiment, the mutated IRA2 polypeptide is a variant and/or a fragment of the amino acid sequence of SEQ ID NO: 22.

In some embodiments, the persistent yeast cell is from the genus Saccharomyces sp. or from the species Saccharomyces cerevisiae.

In some embodiments, the persistent yeast cells can be a recombinant yeast host cell as defined herein. In additional embodiments of the process, the persistent yeast cells, after a total of 40 fermentation cycles, are present in a proportion to at least 99% in the substantially isolated fermenting population. In an embodiment, the fermentation product is an alcohol, such as, for example, ethanol. In yet another embodiment, the fermentation by-product is glycerol. In still further embodiments, the initial and/or the further fermentation medium comprises sugarcane, a sugarcane derivative, molasses, a molasses derivative or a mixture thereof. In yet additional embodiments, the plurality of fermentation cycles comprises at least one continuous fermentation and/or at least one batch fermentation. In further embodiments, the substantially isolating step comprises centrifuging the fermented medium and/or the further fermented medium to substantially isolate the fermenting population. In yet some additional embodiments, each fermentation cycle further comprises acid washing the substantially isolated fermenting population prior to a further fermentation cycle. In some embodiments, the process comprises at least two or more fermentation cycles. In some embodiments, the process further comprises recuperating the fermentation product from the fermented medium and/or the further fermented medium.

According to a third aspect, the present disclosure provides a fermentation medium comprising the persistent yeast cell having the at least one phenotype trait defined herein. In some embodiments, the persistent yeast cell can be the recombinant yeast host cell described herein.

According to a fourth aspect, the present disclosure provides a fermenting population comprising a proportion of at least 99% of persistent yeast cells having the at least one phenotype trait defined herein. In some embodiments, the persistent yeast cells can be the recombinant yeast host cells described herein. In some embodiments, the persistent yeast cells have been submitted a substantially isolating step and/or an acid-washing step.

According to a fifth aspect, the present disclosure provides a process for making a yeast composition. The process comprises propagating a persistent yeast cell having the at least one phenotypic trait defined herein in a propagation medium to obtain a propagated medium, propagated persistent yeast cells. In some embodiments, the process can also comprise substantially isolating the propagated persistent yeast cells or the propagated recombinant yeast host cells from the propagated medium to obtain the yeast composition. In an embodiment, the persistent yeast cells can be the recombinant yeast host cells described herein.

According to a sixth aspect, the present disclosure provides a yeast composition comprising propagated persistent yeast cells having the at least one phenotypic trait defined herein. In an embodiment, the persistent yeast cells are propagated recombinant yeast host cells described herein.

According to a seventh aspect, the present disclosure provides a process for making a persistent yeast cell. The process comprises submitting an initial fermenting population consisting essentially of an initial cell to a plurality of fermentation cycles as defined herein and substantially isolating at least one yeast cell from the fermenting population to obtain the persistent yeast cell. In an embodiment, the process further comprises introducing at least genetic modification for modulating the activity or the expression of a polypeptide for increasing, when compared to a parental cell, the conversion of a biomass into a fermentation product and/or for reducing the conversion of the biomass into a fermentation by-product.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 is an embodiment of the process of the present disclosure.

FIGS. 2A to 2D show the sampling of inputs and outputs of the centrifugation process. Microscopic and plating results from sampling of inputs and outputs from the centrifugation process. (FIG. 2A) provides the percentage of recombinant yeasts host cells (Y1 strain) (with respect to the total number of yeasts recuperated) in the beer (prior to centrifugation), the wine (the supernatant proceeding to distillation also referred to a “de-yeasted wine”) and the cream (the pellet intended to be recycled in the process) as measured by qPCR. Microscopic (top panel) and macroscopic (bottom panel) of (FIG. 2B) the beer, (FIG. 2C) the wine, and (FIG. 2D) the cream.

FIG. 3 provides the results of the settling assay data of a non-aggregated control, a sample of the beer, of the wine and of the cream. Results are shown as the % of sedimentation in 3.5 min in function of the sample tested.

FIGS. 4A to 4C compares the characteristics of yeasts of the rugose and of the smooth phenotype. (FIG. 4A) Example of rugose (multicellular) and smooth (monocellular or budding) occurring on a sample of yeast taken from a commercial ethanol mill. (FIG. 4B) Light (top panel) and electron scanning electron microscopy (middle (scale bar 10 μM) and lower panel (scale bar 3 μM)) images of a smooth yeast strain. (FIG. 4C) Light (top panel) and electron scanning microscopy (middle (scale bar 10 μM) and lower panel (scale bar 3 μM)) of a rugose yeast strain.

FIG. 5 provides the calculated exponential washout rates at eight commercial ethanol facilities. Strain(s) used to inoculate various facilities (each bar represents a different facility) is/are provided below each bar (Y1 alone, or a combination of Y1 and Y2).

FIG. 6 shows the alignment of the functional A8 and nonfunctional A7 alleles at ACE2 for Y1, indicating the translation frameshift and early stop codon introduced at amino acid residue 389 (identified with an arrow).

FIG. 7 provides the sedimentation of various isolates from commercial mills. Yeasts were isolated from five different mills and tested for their sedimentation in a liquid assay measuring the change in OD₆₀₀ in 5 minutes of settling. Results are shown as the percent of the wild yeast settling faster than strain Y2.

FIG. 8 provides the percentage of the A7 ACE2 mutation in three different commercial wild yeast populations (e.g., Mill 7, Mille 4 and Mill 5). Results are shown as the percentage of the A7 allele in the wild yeast population.

FIG. 9 shows the increase of the concentration of strain Y2 during a commercial implementation. Results are shown as the percentage of strain Y2 (estimated from the determination of allele frequency) in function of the number of fermentation cycle based on the estimation of the CDA1-2 allele (♦) or the ACE2-A7 allele (▪).

FIGS. 10A and 10B show the results of strains fermented in a cell recycle process with acid wash at 33.5° C. for 10 hours with a 7 hour feed of industrially sourced must. (FIG. 10A) Sucrose consumption (measured by HPLC at time 0 (black bars), 1 (white bars) and 2 hrs (grey bars) after the start of feed in function of the yeast strains tested. (FIG. 10B) Fermentation kinetics monitored by CO₂ off gas over the course of the fermentation in function of the yeast strains tested (Y0=dark gray line; Y3=light grey line, Y5=stapled line).

FIGS. 11A to 11C shows the phenotypic characterization of wild yeast isolates (Y9, Y10, Y11 and Y12) compared to their parental strains Y0 and Y1. The strains or isolates were spot plated on (FIG. 11A) YPD medium, (FIG. 11B) YPS medium supplemented with 2-deoxyglucose and (FIG. 11C) YPD supplemented with rapamycin.

FIG. 12 compares the response of yeast produced cAMP in basal conditions (dark grey bars) and in response to a 100 mM glucose spike (light gray bars) for yeast strains Y1, Y3 and Y13. Results are shown as the cAMP produced (pmol/mL) in function of the experimental conditions and the strain tested.

FIG. 13 shows the population monitoring of co-cultures in a lab scale cell recycling process. Results are shown as the percentage of competing yeasts (as measure by qPCR) for two different yeast strain populations (Y1/Y0 and Y1/Y3) prior to (dark grey bars) and after three recycling cycles (light gray bars).

FIGS. 14A to 14C show the strain ploidy of wild yeast isolates from commercial cane ethanol facilities. Results are shown as the ratio of the alternate allele depth/total read region in function of frequency. Boxes highlight the triploid strains. Diploids are not boxed. Results are shown for the following strains (left to right, top to bottom). (FIG. 14A): Y14 (triploid), Y15 (triploid), Y16 (triploid), Y17 (triploid), Y18 (triploid), Y19 (triploid), Y20 (triploid), Y21 (triploid), Y22 (triploid), Y23 (triploid), Y24 (triploid), Y25 (triploid); (FIG. 14B): Y26 (triploid), Y27 (triploid), Y28 (triploid), Y29 (triploid), Y30 (triploid), Y31 (triploid), Y32 (triploid), Y33 (diploid), Y34 (triploid); (FIG. 14C): Y35 (triploid), Y36 (triploid), Y3 (triploid), Y37 (diploid), Y38 (triploid), Y39 (triploid), Y40 (triploid), and Y10 (diploid).

FIG. 15 compares the sedimentation rate of various yeast strains. Results are shown as the percentage of sedimentation in function of the yeast strain tested (Y0, Y1, Y2, Y4 or Y6).

FIGS. 16A and 16B provide the yield increase and kinetics of engineered yeasts with persistence traits. Y0, Y2, Y4 and Y6 were recycled for 13 cycles of acid treatment and fermentation on commercial must. (FIG. 16A) The percentage in yield increase (dark gray bars, left axis) and percentage glycerol reduction (light gray squares, right axis) are shown relative to the control strain Y0. (FIG. 16B) The fermentation kinetics were monitored by measuring CO₂ production during the 10 hour fermentation. Results are shown as CO₂ production (ml/min) in function of time (hours) and strain used (Y0: solid black line; Y2: dashed line; Y4: dotted line; Y6: solid light gray line).

FIGS. 17A and 17B provide the population tracking of yeast strains Y2 compared with (FIG. 17A) Y4 or (FIG. 17B) Y6 over 13 cycles of acid treatment and fermentation. Results are shown as the percentage of the yeast strain in function of the number of cycles.

FIGS. 18A and 18B provide the fermentation yields obtained with strains Y0, Y2, Y4 and Y0. (FIG. 18A) shows the % change in ethanol yield (top panel, grey bars) and in glycerol production (lower panel, black bars) in function of Y0. (FIG. 18B) shows the K value of each strain in function of contaminating strain Y7.

FIG. 19 provides the amount of reducing sugar per gram of dry cell weight (gDCW) per minute for yeast strains Y0, Y2, Y3, Y4, Y7 or Y8 on commercial must. The horizontal line within the box represents the median sample value. The ends of the box represent the 25^{th} and 75^{th} quantiles, also expressed as the 1^{st} and 3^{rd} quartile, respectively. The difference between the 1^{st} and 3^{rd} quartiles is referred to as the interquartile range. The whiskers extend from the ends of the box to the outermost data point that falls within the distances computed as follows: 1^{st} quartile −1.5*(interquartile range) and 3^{rd} quartile+1.5*(interquartile range). If the data points do not reach the computed ranges, then the whiskers are determined by the upper and lower data point values (not including outliers).

FIGS. 20A and 20B compare the performances of yeast strains Y2, Y4 and Y61 during multiple rounds of cocultures. (FIG. 20A) provides the calculated exponential washout rates of each tested strains. The origin of different commercial substrates is identified with different symbols. (FIG. 20B) provides the percentage in ethanol change (top panel) and in glycerol change (bottom panel) for each of the tested strains when compared to yeast strain Y0.

### DETAILED DESCRIPTION

It was sought to obtain yeasts having a prolonged presence (e.g., to persist) over a plurality of fermentation cycles. In order to do so, Applicant has identified phenotypic traits which allowed a recombinant yeast host cell to persist during a the plurality of fermentation cycles. Recombinant yeast host cells lacking such phenotypic traits were rapidly selected out from the fermenting population.

The persistent yeast cells of the present disclosure can be submitted to a plurality of fermentation cycles and persist in the fermenting population that is being used throughout the fermentation cycles. The plurality of fermentation cycles in which the persistent yeast cells are submitted comprises at least two distinct fermentation cycles: an initial fermentation cycle and one or more further fermentation cycles. In the initial fermentation cycle, a fermenting population consisting essentially of persistent yeast cells exhibiting the at least one phenotypic traits disclosed herein is contacted with a fermentation medium under conditions so as to obtain a fermentation product (and concurrently a fermented medium). As used in the present disclosure, “a fermenting population consisting essentially of the yeasts” refers to a population of cells which contains the yeasts having the at least one phenotypic traits and is substantially free of contaminating (wild) yeasts. In some embodiments, when the yeasts are recombinant yeast host cells, “a fermenting population consisting essentially of the recombinant yeast host” refers to a population of cells which contains the recombinant yeast host cells as described herein and is substantially free of contaminating (wild, non-genetically modified) yeasts. The fermenting population obtained at the end of this initial fermentation cycle is recycled for a further fermentation cycle (e.g., substantially isolated and used to inoculate a further fermentation medium). In this further fermentation cycle, no additional persistent yeast cells are added to the fermenting population. As such, the fermenting population used to inoculate the further fermentation medium consists essentially in the fermenting population substantially isolated in the initial fermentation cycle. It is recognized that the fermenting population used to inoculate the further fermentation medium can include contaminating wild yeasts which may have been introduced in the fermentation medium of the initial fermentation cycle. The inoculated further fermentation medium is then placed under conditions so as to obtain the fermented product and subsequently substantially isolate a (further) fermenting population (from a further fermented medium). The substantially isolated further fermenting population can be recycled and used to conduct one or more further fermentation cycle. It is understood that, in yet a further fermentation cycle, no additional persistent yeast cells are added to the further fermenting population. As such, the further fermenting population used to inoculate the yet further fermentation medium consists essentially in the fermenting population substantially isolated in the further fermentation cycle. It is recognized that the fermenting population used to inoculate the yet further fermentation medium can include contaminating wild yeasts which may have been introduced in the further fermentation medium of the further fermentation cycle. In an embodiment, a fermenting population obtained by using an initial fermenting population consisting essentially of the persistent yeast cells as described herein submitted to at least 40 fermentation cycles (total) comprises at least 90%, 99%, 99.9% or more persistent yeast cells having the at least one phenotype traits as described herein. In another embodiment, a fermenting population obtained by using an initial fermenting population consisting essentially of the recombinant persistent yeast host cells as described herein submitted to at least 40 fermentation cycles (total) comprises at least 90%, 99%, 99.9% or more of the recombinant persistent yeast host cells described herein.

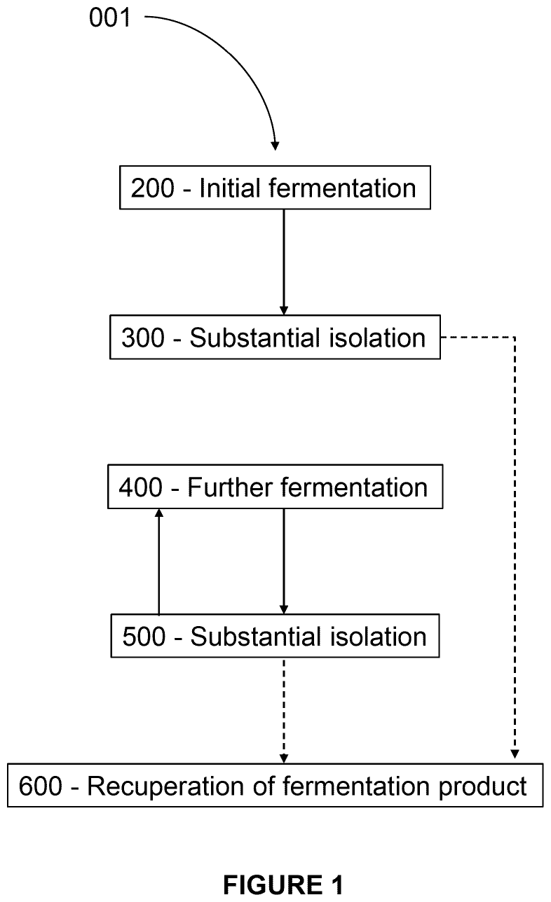

An embodiment of a fermentation process 001 using a plurality of fermentation cycles is shown as FIG. 1. In the embodiments shown on FIG. 1, steps 200 and 300 refer to the initial fermentation cycle and steps 400 and 500 (which can be repeated) refer to the further fermentation cycle(s). In process 001, at step 200, an initial fermenting population is inoculated in a fermentation medium which is then submitted to an initial fermentation. The initial fermenting population that is added to the fermentation medium consists essentially of the persistent yeast cells (which include, in some embodiments, the recombinant yeast host cells exhibiting the one or more phenotypic trait as described herein). It is understood that the fermentation medium of step 200 can initially include contaminating wild yeasts or can be contaminated during fermentation with wild yeasts. Once the initial fermentation has been completed (e.g., a fermentation product and a fermenting population have accumulated in the fermentation medium to provide a fermented fermentation medium), the resulting fermenting population, at step 300, is substantially isolated from the fermented fermentation medium. As it will be explained below, the isolating step can include, without limitation, centrifuging the fermented fermentation medium and/or acid washing the substantially isolated fermenting population (not shown on FIG. 1). Once the initial fermentation cycle has been completed (at the conclusion of step 300), the substantially isolated fermenting population is placed, at step 400, into contact (e.g., used to inoculate) a further fermentation medium and allowed to perform a further fermentation. Once the further fermentation has been completed (e.g., a fermentation product and a further fermenting population have accumulated in the further fermentation medium to provide a further fermented fermentation medium), the resulting fermenting population, at step 500, is substantially isolated from the fermented fermentation medium. As it will be explained below, the isolating step can include, without limitation, centrifuging the further fermented fermentation medium and/or acid washing the substantially isolated fermenting population (not shown on FIG. 1). In some embodiments, no additional persistent yeast cell exhibiting the one or more phenotypic trait, including the recombinant yeast host cell described herein, is added to the fermentation medium after step 200, including during the one or more further fermentation cycles. However, in some embodiments, especially in the presence of contaminating microbes (such as bacteria and/or yeasts), it may be possible to add further persistent yeasts cells at the beginning of step 400 to perform the further fermentation. The substantially isolated fermenting population obtained at step 500 can be submitted to yet a further fermentation cycle at step 400. In some embodiments, the process can also include, after steps 300 or 500, recuperating, at step 600, the fermentation product from the fermented fermentation medium or the further fermented fermentation medium. This can be used, for example, by distilling the fermented fermentation medium or the further fermented fermentation medium (not shown on FIG. 1).

In some embodiments, the persistent yeast cells of the present disclosure are recombinant yeast host cells capable of modulating the expression of one or more polypeptide for increasing, when compared to a non-genetically modified parental cell, the conversion of a biomass into a fermentation product during a fermentation and/or for reducing the production of a fermentation by-product during the fermentation. In one embodiment, the recombinant yeast host cells of the present disclosure include at least one genetic modification to increase or decrease the activity (and in some embodiments the expression) of one or more polypeptide involved in the conversation of a biomass into a fermentation product and optionally the reduction of a fermentation by-product. In some optional embodiments, the recombinant yeast host cells of the present disclosure can also include one or more further genetic modification for providing the at least one phenotypic traits disclosed herein.

When the genetic modification is aimed at increasing the activity of a specific targeted polypeptide (which may native or heterologous) or the expression of a specific targeted gene (which may native or heterologous), the genetic modification can be made in one or multiple genetic locations. When the genetic modification is aimed at reducing or inhibiting the activity of a specific targeted polypeptide (which is native) or the expression of a specific targeted gene (which is native), the genetic modifications can be made in one or all copies of the targeted gene(s).

In the context of the present disclosure, the one or more genetic modifications are aimed at increasing, when compared to a parental cell, the conversion of a biomass into a fermentation product and/or at reducing the conversion of the biomass into a fermentation by-product in the recombinant yeast host cell. In an embodiment, when the one or more genetic modifications are aimed at increasing the conversion of a biomass (e.g., sugarcane or a biomass derived therefrom) into a fermentation product (e.g., an alcohol, for example, ethanol), an increase of at least 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5% or more of ethanol change in the recombinant yeast host cell when compared to a parental cell can be observed. In an embodiment, the parental cell is the Saccharomyces cerevisiae PE-2 strain. In another embodiment, when the one or more genetic modifications are aimed at reducing the conversion of the biomass (e.g., sugarcane or a biomass derived therefrom) into a fermentation by-product (e.g., glycerol), a decrease of at least 10, 11, 12, 13, 14, 15, 16, 17, 18, 19, 20, 21, 22, 23, 24, 25, 26, 27, 28, 29, 30% or more of glycerol change in in the recombinant yeast host cell when compared to a parental cell can be observed. In an embodiment, the parental cell is the Saccharomyces cerevisiae PE-2 strain.

In the context of the present disclosure, the recombinant yeast host cells are qualified as being “genetically engineered”, e.g., they have been manipulated to either add at least one or more heterologous or exogenous nucleic acid residue and/or remove at least one endogenous (or native) nucleic acid residue. In some embodiments, the one or more nucleic acid residues that are added can be derived from a heterologous cell or the recombinant cell itself. In the latter scenario, the nucleic acid residue(s) is (are) added at a genomic location which is different than the native genomic location. The genetic manipulations did not occur in nature and are the results of in vitro manipulations of the native yeast.

In some embodiments, the genetic modification can be encoded on one or more heterologous molecules. In some embodiments, the heterologous nucleic acid molecule can encode one or more polypeptide (which may be additional copies of a native gene). In other embodiments, the heterologous nucleic acid molecules can encode a promoter or other regulatory sequence for upregulating or downregulating the expression of a native gene encoding a native polypeptide. In some embodiments, the heterologous nucleic acid molecules of the present disclosure can include a signal sequence to favor the secretion of the heterologous polypeptide or the native polypeptide.

The term “heterologous” when used in reference to a nucleic acid molecule (such as a promoter, a terminator or a coding sequence) or a protein/polypeptide refers to a nucleic acid molecule or a protein/polypeptide that is not natively found in the recombinant host cell. “Heterologous” also includes a native coding region/promoter/terminator, or portion thereof, that was removed from the source organism and subsequently reintroduced into the source organism in a form that is different from the corresponding native gene, e.g., not in its natural location in the organism's genome. The heterologous nucleic acid molecule is purposively introduced into the recombinant yeast host cell. For example, a heterologous element could be derived from a different strain of host cell, or from an organism of a different taxonomic group (e.g., different kingdom, phylum, class, order, family genus, or species, or any subgroup within one of these classifications). As used herein, the term “native” when used in inference to a gene, polypeptide, enzymatic activity, or pathway refers to an unmodified gene, polypeptide, enzymatic activity, or pathway originally found in the recombinant host cell. In some embodiments, heterologous polypeptides derived from a different strain of host cell, or from an organism of a different taxonomic group (e.g., different kingdom, phylum, class, order, family genus, or species, or any subgroup within one of these classifications) can be used in the context of the present disclosure.

The heterologous nucleic acid molecules of the present disclosure can comprise a coding region for the heterologous polypeptide. A DNA or RNA “coding region” is a DNA or RNA molecule (preferably a DNA molecule) which is transcribed and/or translated into a heterologous polypeptide in a cell in vitro or in vivo when placed under the control of appropriate regulatory sequences. “Suitable regulatory regions” refer to nucleic acid regions located upstream (5′ non-coding sequences), within, or downstream (3′ non-coding sequences) of a coding region, and which influence the transcription, RNA processing or stability, or translation of the associated coding region. Regulatory regions may include promoters, transcription terminators, translation leader sequences, RNA processing site, effector binding site and stem-loop structure. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell (such as the recombinant yeast host cell of the present disclosure), a polyadenylation signal and transcription termination sequence will usually be located 3′ to the coding region. In an embodiment, the coding region can be referred to as an open reading frame. “Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

The heterologous nucleic acid molecules described herein can comprise transcriptional and/or translational control regions. “Transcriptional and translational control regions” are DNA regulatory regions, such as promoters, enhancers, terminators, and the like, that provide for the expression of a coding region in a recombinant host cell. In eukaryotic cells, polyadenylation signals are considered control regions.

In some embodiments, the heterologous nucleic acid molecules of the present disclosure include a coding sequence for a heterologous polypeptide, optionally in combination with a promoter and/or a terminator. In some embodiments, the heterologous nucleic acid molecules of the present disclosure include a nucleic acid sequence encoding a promoter for overexpressing a native gene encoding a native polypeptide. In the heterologous nucleic acid molecules of the present disclosure, the promoter and the terminator (when present) are operatively linked to the nucleic acid coding sequence of the heterologous or native polypeptide, e.g., they control the expression and the termination of expression of the nucleic acid sequence of the heterologous or the native polypeptide. The heterologous nucleic acid molecules of the present disclosure can also include a nucleic acid sequence coding for a signal sequence, e.g., a short peptide sequence for exporting the heterologous polypeptide outside the host cell. When present, the nucleic acid sequence coding for the signal sequence is directly located upstream and in frame of the nucleic acid sequence coding for the heterologous polypeptide.

In the persistent yeast cells described herein, the nucleic acid molecule coding for the promoter and the nucleic acid molecule coding for the heterologous or the native polypeptide are operatively linked to one another. In the context of the present disclosure, the expressions “operatively linked” or “operatively associated” refers to fact that the promoter is physically associated to the nucleotide acid molecule coding for the heterologous or the native polypeptide in a manner that allows, under certain conditions, for expression of the heterologous polypeptide from the nucleic acid molecule. In an embodiment, the promoter can be located upstream (5′) of the nucleic acid sequence coding for the heterologous polypeptide. In still another embodiment, the promoter can be located downstream (3′) of the nucleic acid sequence coding for the heterologous polypeptide. In the context of the present disclosure, one or more than one promoter can be included in the heterologous nucleic acid molecule. When more than one promoter is included in the heterologous nucleic acid molecule, each of the promoters is operatively linked to the nucleic acid sequence coding for the heterologous or native polypeptide. The promoters can be located, in view of the nucleic acid molecule coding for the heterologous or native polypeptide, upstream, downstream as well as both upstream and downstream.

The term “promoter” refers to a DNA fragment capable of controlling the expression of a coding sequence or functional RNA. The term “expression,” as used herein, refers to the transcription and stable accumulation of sense mRNA from the heterologous nucleic acid molecule or the native gene described herein. Expression may also refer to translation of mRNA into a polypeptide. Promoters may be derived in their entirety from the promoter of a native gene, or be composed of different elements derived from different promoters found in nature, or even comprise synthetic DNA segments. It is understood by those skilled in the art that different promoters may direct the expression at different stages of development, or in response to different environmental or physiological conditions. Promoters which cause a gene to be expressed in most cells at most times at a substantial similar level are commonly referred to as “constitutive promoters”. Promoters which cause a gene to be expressed during the propagation phase of a yeast cell are herein referred to as “propagation promoters”. Propagation promoters include both constitutive and inducible promoters, such as, for example, glucose-regulated, molasses-regulated, stress-response promoters (including osmotic stress response promoters) and aerobic-regulated promoters. Promoters which cause a gene to be expressed during the fermentation phase of a yeast cell are herein referred to as “fermentation promoters”. Fermentation promoters include both constitutive and inducible promoters such as, for example, anaerobic promoters. In the context of the present disclosure, a “glycolytic promoter” is a promoter (or a combination of promoters) allowing the expression (or, in some embodiments, the overexpression) of a gene operatively associated thereto when the recombinant microbial cell is in placed in glycolytic conditions. The glycolytic promoter can be a constitutive promoter or a glucose-inducible promoter. Glycolytic promoters exclude glucose-repressible promoters. It is further recognized that since in most cases the exact boundaries of regulatory sequences have not been completely defined, DNA fragments of different lengths may have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as protein binding domains (consensus sequences) responsible for the binding of the polymerase.

The promoter can be native or heterologous to the nucleic acid molecule encoding the native or the heterologous polypeptide. The promoter can be heterologous to the native gene encoding the native polypeptide to be overexpressed. The promoter can be heterologous or derived from a strain being from the same genus or species as the recombinant host cell. In an embodiment, the promoter is derived from the same genus or species of the yeast host cell and the heterologous polypeptide is derived from a different genus than the host cell. The promoter can be a single promotor or a combination of different promoters.

In the context of the present disclosure, the promoter controlling the expression of the heterologous polypeptide or the native polypeptide can be a constitutive promoter (such as, for example, tef2p (e.g., the promoter of the tef2 gene), cwp2p (e.g., the promoter of the cwp2 gene), ssa1p (e.g., the promoter of the ssa1 gene), eno1p (e.g., the promoter of the eno1 gene), hxk1 (e.g., the promoter of the hxk1 gene) and pgk1p (e.g., the promoter of the pgk1 gene). In some embodiment, the promoter is tef2p (e.g., the promoter of the tef2 gene). In some embodiment, the promoter is adh1p (e.g., the promoter of the adh1 gene). However, in some embodiments, it is preferable to limit the expression of the polypeptide. As such, the promoter controlling the expression of the heterologous polypeptide or the native polypeptide can be an inducible or modulated promoters such as, for example, a glucose-regulated promoter (e.g., the promoter of the hxt7 gene (referred to as hxt7p)) or a sulfite-regulated promoter (e.g., the promoter of the gpd2 gene (referred to as gpd2p or the promoter of the fzf1 gene (referred to as the fzf1p)), the promoter of the ssu1 gene (referred to as ssu1p), the promoter of the ssu1-r gene (referred to as ssur1-rp). In an embodiment, the promoter is an anaerobic-regulated promoters, such as, for example tdh1p (e.g., the promoter of the tdh1 gene), pau5p (e.g., the promoter of the pau5 gene), hor7p (e.g., the promoter of the hor7 gene), adh1p (e.g., the promoter of the adh1 gene), tdh2p (e.g., the promoter of the tdh2 gene), tdh3p (e.g., the promoter of the tdh3 gene), gpd1p (e.g., the promoter of the gdp1 gene), cdc19p (e.g., the promoter of the cdc19 gene), eno2p (e.g., the promoter of the eno2 gene), pdc1p (e.g., the promoter of the pdc1 gene), hxt3p (e.g., the promoter of the hxt3 gene), dan1 (e.g., the promoter of the dan1 gene) and tpi1p (e.g., the promoter of the tpi1 gene). One or more promoters can be used to allow the expression of each heterologous polypeptides in the recombinant yeast host cell.

Still in the context of the present disclosure, the promoter controlling the expression of the heterologous polypeptide or the native polypeptide can be a glycolytic promoter. For example, the glycolytic promoter can be a promoter (or a combination of promoters) from an alcohol dehydrogenase gene, a glucose-6-phosphate isomerase gene, a phosphofructokinase gene, an aldolase gene, a triosephosphate isomerase gene, a glyceraldehyde-3-phosphate dehydrogenase gene, a 3-phosphoglycerate kinase gene, a phosphoglycerate mutase, an enolase and/or a pyruvate kinase gene.

One or more promoters can be used to allow the expression of each heterologous/native polypeptides in the persistent yeast cell. In the context of the present disclosure, the expression “functional fragment of a promoter” when used in combination to a promoter refers to a shorter nucleic acid sequence than the native promoter which retain the ability to control the expression of the nucleic acid sequence encoding the heterologous polypeptide. Usually, functional fragments are either 5′ and/or 3′ truncation of one or more nucleic acid residue from the native promoter nucleic acid sequence.

The heterologous nucleic acid molecule of the present disclosure can be integrated in the chromosome(s) of the recombinant yeast host cell. The term “integrated” as used herein refers to genetic elements that are placed, through molecular biology techniques, into the chromosome of a host cell. In some embodiments, the heterologous nucleic acid molecule(s) is/are integrated at one or more neutral integration site. For example, genetic elements can be placed into the chromosomes of the host cell as opposed to in a vector such as a plasmid carried by the host cell. Methods for integrating genetic elements into the chromosome of a host cell are well known in the art and include homologous recombination. The heterologous nucleic acid molecule can be present in one or more copies in the recombinant yeast host cell's chromosome. Alternatively, the heterologous nucleic acid molecule can be independently replicating from the yeast's chromosome. In such embodiment, the nucleic acid molecule can be stable and self-replicating. The heterologous nucleic acid molecules can be present in one or more copies in the recombinant yeast host cell. For example, each heterologous nucleic acid molecules can be present in 1, 2, 3, 4, 5, 6, 7, 8, 9, 10, 11, 12 copies or more per chromosome.

In some embodiments, the heterologous nucleic acid molecules which can be introduced into the recombinant host cells are codon-optimized with respect to the intended recipient recombinant yeast host cell. As used herein the term “codon-optimized coding region” means a nucleic acid coding region that has been adapted for expression in the cells of a given organism by replacing at least one, or more than one, codons with one or more codons to optimize expression levels. In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism.

The heterologous nucleic acid molecules can be introduced in the yeast host cell using a vector. A “vector,” e.g., a “plasmid”, “cosmid” or “artificial chromosome” (such as, for example, a yeast artificial chromosome) refers to an extra chromosomal element and is usually in the form of a circular double-stranded DNA molecule. Such vectors may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

### Phenotypic Traits

The persistent yeast cells of the present disclosure exhibits one or more phenotypic trait which allows them to be present over more fermentation cycles (e.g., persist during a plurality of fermentation cycles) than a corresponding control cell which lacks the phenotypic trait(s). The persistent yeast cells can be selected for the presence of one or more phenotypic trait or could be genetically engineered to provide one or more phenotypic trait. In some embodiments, the persistent yeast cells can be selected for the presence of at least one phenotypic trait and can be genetically engineered for the another phenotypic trait.

As it will be explained into more details below, the recombinant yeast host cells of the present disclosure necessarily includes at least one genetic modification (for modulating the activity or the expression of a first and/or a second polypeptide). It is possible to select/engineer a parental yeast host cell which possess the one or more phenotypic traits and modify such parental yeast host cell to include the genetic modification(s) for modulating the activity or the expression of a first and/or a second polypeptide. It is also possible to first introduce in a first yeast host cell the genetic modification(s) for modulating the activity or the expression of a first and/or a second polypeptide and afterwards select/engineer for the at least one phenotypic traits to obtain the recombinant yeast host cells of the present disclosure.

In an embodiment, the persistent yeast cells of the present disclosure exhibit at least one of the following phenotypic trait: a fast settling phenotype, a rugose phenotype, an improved invertase activity, triploidy, or increased signaling in a RAS/cAMP/PKA pathway. In another embodiment, the persistent yeast cells of the present disclosure exhibits at least two of the following phenotypic trait: a fast settling phenotype, a rugose phenotype, an improved invertase activity, triploidy, or increased signaling in a RAS/cAMP/PKA pathway. In a further embodiment, the persistent yeast cells of the present disclosure exhibits at least three of the following phenotypic trait: a fast settling phenotype, a rugose phenotype, an improved invertase activity, triploidy, or increased signaling in a RAS/cAMP/PKA pathway. In yet another embodiment, the persistent yeast cells of the present disclosure exhibits at least four of the following phenotypic trait: a fast settling phenotype, a rugose phenotype, an improved invertase activity, triploidy, or increased signaling in a RAS/cAMP/PKA pathway. In one embodiment, the persistent yeast cells of the present disclosure exhibits the following phenotypic traits: a fast settling phenotype, a rugose phenotype, an improved invertase activity, triploidy, and increased signaling in a RAS/cAMP/PKA pathway.

The persistent yeast cells of the present disclosure can advantageously be used in a plurality of fermentation cycles for converting a biomass into a fermentation product (e.g., an alcohol, such as, for example, ethanol) and in some optional embodiments, for reducing the conversion of the biomass into a fermentation by-product (e.g., distinct from ethanol, such as, for example, glycerol). In some embodiments, when the persistent yeast cells are provided as an initial fermenting population in a plurality of fermentation cycles, after 40, 45, 50, 55, 60, 65, 70, 75, 80, 90 or more fermentation cycles, the persistent yeast cells remain present in a proportion of at least 90, 91, 92, 93, 94, 95, 96, 97, 98, 99.0, 99.1, 99.2, 99.3, 99.4, 99.5, 99.6, 99.7, 99.8, 99.9% or more in the resulting fermenting population (when measured as its contribution to the DNA of the resulting fermenting population).

### Fast Settling Phenotype

In some embodiments, the persistent yeast cells of the present disclosure can exhibit a fast settling phenotype. Yeasts exhibiting the fast settling phenotype are able to be centrifuged more efficiently and are therefore positively selected to be present in the substantially isolated fermenting population (and therefore persist further during subsequent fermentation cycles). As used in the context of the present disclosure, a “fast settling phenotype” refers to the ability of the persistent yeast cell to settle more rapidly (either by gravity or during centrifugation) than a control yeast lacking the fast settling phenotype. The fast settling phenotype can be due, at least in part, with an increased ability of the persistent yeast cell to flocculate when compared to a control yeast lacking the fast settling phenotype. The fast settling phenotype can be due, at least in part, by an increase ability of the persistent yeast cell to form cell clumps when compared to a control yeast lacking the fast settling phenotype. The fast setting phenotype can be due, at least in part, by the presence of the rugose phenotype in the persistent yeast cell.

In one embodiment, the persistent yeast cell of the present disclosure has the ability to settle more rapidly that the a control non-persistent yeast, e.g., the PE-2 strain. The commercially available strain PE-2 (described in Argueso et al., 2009 as well as Basso et al., 2011 and having the JAY291 genome in the Saccharomyces Genome Database (SGD)) lacks the fast settling phenotype. In the Example of the present disclosure, strain PE-2 is referred to as Y0.

In a specific embodiment, when a population consisting essentially of the persistent yeast cells of the present disclosure is provided in a relatively homogeneous distribution in a liquid medium, at least 5, 6, 7, 8, 9, 10, 11, 12, 13, 14, 15, 16, 17, 18, 19, 20% or more of the persistent yeast cells of the population will sediment by gravity after 5 minutes (when measured using optical density). In another specific embodiment, when a population consisting essentially of the persistent yeast cells of the present disclosure is provided in a relatively homogeneous distribution in a liquid medium, at least 5% of the persistent yeast cells of the population will sediment by gravity after 5 minutes (when measured using optical density). In still a further embodiment, a higher proportion (at least 15, 20, 25, 30, 35, 40, 45, 50, 60, 65, 70, 75, 80, 85, 90, 95 or 100%) of a population consisting essentially of persistent yeast cells of the present disclosure, when compared to a control population consisting essentially of control yeast cells, will sediment by gravity after 5 minutes (when measured using optical density). The control yeast cells lack the fast settling phenotype. In an embodiment, the control population consists essentially of the Saccharomyces cerevisiae PE-2 strain which is was shown in the Example to lack the fast settling phenotype. In order to determine the proportion of the populations that is able to sediment by gravity, the populations are provided in a relatively homogeneous distribution in a liquid medium and the proportion can be determined by optical density.

The persistent yeast cell can be selected, from a population, for the fast settling phenotype. This can be done, for example, by submitting a population of yeasts to a plurality of fermentation cycles which includes centrifuging the fermenting population in between cycles and selecting the persistent yeast cells having the ability to settle more rapidly during the process. Alternatively or in combination, the yeasts can be genetically engineered to provide a fast settling phenotype. This can be done, for example, by introducing one or more genetic modification to change the phenotype of a yeast from being smooth to rugose.

In an embodiment, the persistent yeast cell exhibits the fast settling phenotype optionally in combination with at least one of the following additional phenotypic traits: a rugose phenotype, an improved invertase activity, triploidy or increased signaling in a RAS/cAMP/PKA pathway. In another embodiment, the persistent yeast cell exhibits the fast settling phenotype optionally in combination with at least two of the following additional phenotypic traits: a rugose phenotype, an improved invertase activity, triploidy or increased signaling in a RAS/cAMP/PKA pathway. In another embodiment, the persistent yeast cell exhibits the fast settling phenotype optionally in combination with at least three of the following additional phenotypic traits: a rugose phenotype, an improved invertase activity, triploidy or increased signaling in a RAS/cAMP/PKA pathway. In a further embodiment, the persistent yeast cell exhibits the fast settling phenotype in combination with the following additional phenotypic traits: a rugose phenotype, an improved invertase activity, triploidy and increased signaling in a RAS/cAMP/PKA pathway.

### Rugose Phenotype

In some embodiments, the persistent yeast cells of the present disclosure can exhibit the rugose phenotype. The rugose phenotype can be observed in cells after having been exponentially grown in a medium inoculated at low density. In an embodiment, low density refers to a density which would allow for 3 to 10 generations. In some embodiments, the low density refers to a density between about 0.01 and about 1 g/L of dry cell weight. The rugose phenotype is associated with the reduced ability of the persistent yeast cells to sever the septum between daughter cells. Yeasts exhibiting the rugose phenotype are able to form clumps in liquid medium which allows them to settle more rapidly (either by gravity or by centrifugation). Yeasts exhibiting the rugose phenotype are thus preferably selected to be present in the substantially isolated fermenting population (and therefore further persist in subsequence fermentation cycles). Colonies of persistent yeast cells exhibiting the rugose phenotype have irregular edges when cultured on a solid medium.

In a specific embodiment, when a population consisting essentially of the persistent yeast cells having the rugose phenotype is provided after exponential growth in a medium inoculated at low cell density, at least 90, 91, 92, 93, 94, 95, 96, 97, 98, 99% or more of the persistent yeast cells having at least two daughter cells attached. This assessment can be made, for example, by microscopic observation of the population after the exponential growth. In some embodiments, a population consisting essentially of persistent yeast cell exhibiting the rugose phenotype, after exponential growth in a medium inoculated at high cell density, may exhibit a lesser percentage of cells having at least two daughter cells attached. In some embodiments, the expression “high cell density” refers to a cell density of at least about 1, 2, 3, 4, 5, 6, 7, 8, 9, 10, 11, 12, 13, 14, 15, 16, 17, 18, 19, 20, 21, 22, 23, 24, 25, 26, 27, 28 29 pr 30 g/L of dry cell weight. In further embodiments, the expression “high cell density” refers to a cell density between about 1 to about 30 g/L of dry cell weight.

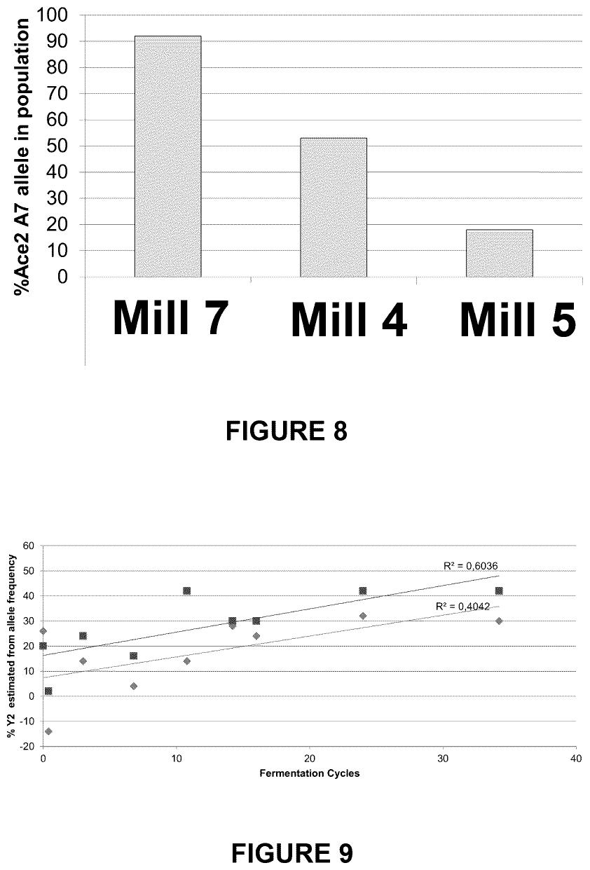

In an embodiment, the persistent yeast cells exhibiting the rugose phenotype have a reduced transcription factor activity of a Activator of CUP1 Expression (ACE2) polypeptide (when compared to a control yeast expressing a wild-type ACE2 polypeptide, such as, for example, the control PE-2 strain). This reduction in activity can be caused, in some embodiments, by the presence of a genetic modification (e.g., insertion, deletion, substitution or indel) in the regulatory region (e.g., promoter) or the coding region of the gene encoding the ACE2 polypeptide. In a further embodiment, the persistent yeast cells of the present disclosure includes at least one genetic modification (when compared to the wild-type nucleic acid sequence of the ACE2 gene provided as SEQ ID NO: 9 or a degenerate sequence encoding the amino acid sequence of SEQ ID NO: 10) in one, two or all alleles encoding the ACE2 gene. Modifications of the ACE2 gene to provide a rugose phenotype are known (Oud et al., 2013) and can introduced in the recombinant yeast host cell of the present disclosure to provide a persistent yeast cell. In a specific example, the persistent yeast cells of the present disclosure can include a deletion of at least one or more nucleic acid residues (which, in some embodiments, may be a deletion at or in the 3′ region of the ACE2 gene) when compared to the to the wild-type nucleic acid sequence of the ACE2 gene provided as SEQ ID NO: 9 or a degenerate sequence encoding SEQ ID NO: 10. In some embodiments, this genetic modification is provided in FIG. 8, Tables 4 or 5. The genetic modification in the ACE2 gene may be present in one, two or all alleles of the ACE2 gene. As such, the persistent yeast cells of the present disclosure may be heterozygous or homozygous with respect to the genetic modification in the ACE2 gene.

In yet another embodiment, the persistent yeast cells of the present disclosure can include at least one amino acid modification (when compared to the wild-type amino acid sequence of the ACE2 polypeptide provided as SEQ ID NO: 10) in a mutated ACE2 polypeptide. In a specific embodiment, the persistent yeast cells exhibiting the rugose phenotype expresses a fragment of the wild-type ACE2 polypeptide, such as, for example, a C-terminal truncation of the wild-type ACE2 polypeptide (when compared to the wild-type amino acid sequence of the ACE2 polypeptide provided as SEQ ID NO: 10). In still further embodiments, the persistent yeast cells of the present disclosure can express a mutated ACE2 polypeptide having the amino acid sequence of SEQ ID NO: 12, 13, 14,15 or 16. In some embodiments, the persistent yeast cells exhibiting the rugose phenotype are capable of expressing a variant of a mutated ACE2 polypeptide having the amino acid sequence of SEQ ID NO: 12, 13, 14, 15 or 16 having reduced transcription factor activity (when compared to the wild-type ACE2 polypeptide). In some further embodiments, the persistent yeast cells exhibiting the rugose phenotype are capable of expressing a fragment of a mutated ACE2 polypeptide having the amino acid sequence of SEQ ID NO: 12, 13, 14, 15 or 16 having reduced transcription factor activity (when compared to the wild-type ACE2 polypeptide).

In a specific embodiment, the persistent yeast cells of the present disclosure can express a mutated ACE2 polypeptide having the amino acid sequence of SEQ ID NO: 12. In such embodiment, the persistent yeast cells of the present disclosure can include one or both mutated ACE2 alleles comprising the nucleic acid sequence of SEQ ID NO: 11 (or a degenerate sequence encoding SEQ ID NO: 12). In some embodiments, the mutated allele for the ACE2 polypeptide can be located on a heterologous nucleic acid molecule introduced in the recombinant yeast host cell.

The persistent yeast cells can be selected, from a population of yeast cells, for the rugose phenotype. This can be done, for example, by submitting a population of yeast cells to a plurality of fermentation cycles which includes centrifuging the fermenting population in between cycles and selecting the yeast cells having the ability to settle more rapidly during the process (which are believed to have the rugose phenotype). This can also be done, for example, by culturing on a solid medium a population of yeast cells, and selecting the colonies exhibiting the rugose phenotype (either by visual observation or by microscopy for example). Alternatively or in combination, the yeast can be genetically engineered to provide a rugose phenotype. This can be done, for example, by introducing one or more genetic modification to change the phenotype of the yeast from being smooth to rugose and in some embodiments, for expressing a mutated ACE2 polypeptide.

In an embodiment, the persistent yeast cell exhibits the rugose phenotypic trait optionally in combination with at least one of the following additional phenotypic traits: a fast settling phenotype, an improved invertase activity, triploidy or increased signaling in a RAS/cAMP/PKA pathway. In an embodiment, the persistent yeast cell exhibits the rugose phenotypic trait optionally in combination with at least two of the following additional phenotypic traits: a fast settling phenotype, an improved invertase activity, triploidy or increased signaling in a RAS/cAMP/PKA pathway. In an embodiment, the persistent yeast cell exhibits the rugose phenotypic trait optionally in combination with at least three of the following additional phenotypic traits: a fast settling phenotype, an improved invertase activity, triploidy or increased signaling in a RAS/cAMP/PKA pathway. In an embodiment, the persistent yeast cell exhibits the rugose phenotypic trait in combination the following additional phenotypic traits: a fast settling phenotype, an improved invertase activity, triploidy and increased signaling in a RAS/cAMP/PKA pathway.

### Improved Invertase Activity

In some embodiments, the persistent yeast cells of the present disclosure can exhibit improved invertase activity when compared to a control yeast cell lacking the phenotype. Yeasts exhibiting the improved invertase activity phenotype are able to hydrolyse more rapidly and/or at lower pH a carbohydrate source (e.g., a disaccharide (such as, for example, sucrose) or a trisaccharide (such as, for example, raffinose or kestose)) that is present in the biomass of the fermentation medium. In some embodiments, the persistent yeast cells of the present disclosure are used in fermentations in which the initial levels of fermentable glucose and fructose are relatively low (e.g., between 1 to 5 g/L) and as such their improved invertase activity can provide a selective advantage by accessing a complementary carbohydrate source (and consequently begin the fermentation process more rapidly).

In one embodiment, the persistent yeast cells of the present disclosure exhibit increased invertase activity when compared to the a control strain (e.g., such as the PE-2 strain) in comparable conditions. A population consisting essentially of the persistent yeast cells of the present disclosure is able to consume more than 1.0, 1.7, 2, 3, 4, 5, 6, 7, 8, 9, 10, 11 times sucrose when compared to a population consisting essentially of control yeasts (e.g., such as the PE-2 strain) in comparable conditions. In order to make such comparison, both populations, after exponential growth, can be diluted, on a wet cell basis, to a concentration of 9 mg/mL in a buffer and sucrose consumption can be measured (by HPLC for example) at a specified time interval. In some embodiments, invertase activity is measured prior to saturation of the assay. In some specific embodiments, invertase activity is measured within 1, 2, 3, 4, 5, 6, 7, 8, 9, 10, 11, 12, 13, 14 or 15 minutes of the beginning of the assay. In some additional embodiments, invertase activity is measured 12 to 15 minutes after the beginning of the assay. Still in such embodiment, the buffer initially comprises 40 g/L of sucrose, is at of pH 5 and at a temperature of 35° C.

In a specific embodiment, a population consisting essentially of the persistent yeast cells of the present disclosure, after exponential growth, diluted to a concentration of 9 mg/mL, on a wet cell basis, in a buffer is able to consume more than 0.05 gram of sucose per gram of dry cell weight per minute. In some additional embodiments, a population consisting essentially of the persistent yeast cells of the present disclosure, after exponential growth, diluted to a concentration of 9 mg/mL, on a wet cell basis, in a buffer is able to consume at least 0.06, 0.07, 0.08, 0.09, 0.10, 0.11, 0.12, 0.13, 0.14, 0.15, 0.16, 0.17, 0.18, 0.19, 0.20 or more gram of sucrose per gram of dry cell weight per minute. As indicate above, the invertase assay can use a buffer initially comprising 40 g/L of sucrose, at of pH 5 and at a temperature of 35° C. In some embodiments, invertase activity is measured prior to saturation of the assay. In some specific embodiments, invertase activity is measured within 1, 2, 3, 4, 5, 6, 7, 8, 9, 10, 11, 12, 13, 14 or 15 minutes of the beginning of the assay. In some additional embodiments, invertase activity is measured 12 to 15 minutes after the beginning of the assay. Furthermore, in additional embodiments, the sugar consumption can be measured, for example, by HPLC.

The persistent yeast cells of the present disclosure can have increased enzymatic activity of at least one polypeptide having invertase activity. This increased enzymatic activity can be observed, in some embodiments, at low pH (for example, at a pH equal to or lower than 5.0, 4.9, 4.8, 4.7, 4.6, 4.5, 4.4, 4.3, 4.2, 4.1, 4.0 or lower). This increased invertase activity can be associated with a genetic modification in the regulatory region (e.g., promoter region) and/or the coding region of a gene encoding an invertase. This increased invertase activity can be associated with additional copies or duplications of a gene encoding an invertase. In yeasts, the following polypeptides are known to exhibit invertase activity: SUC1 (encoded by the SUC1 gene), SUC2 (encoded by the SUC2 gene), SUC3 (encoded by the SUC3 gene), SUC4 (encoded by the SUC4 gene), SUC5 (encoded by the SUC5 gene), SUC6 (encoded by the SUC6 gene), SUC7 (encoded by the SUC7 gene), SUC8 (encoded by the SUC8 gene) and SUC9 (encoded by the SUC9 gene). In some embodiments, the persistent yeast cells of the present disclosure have a genetic modification in one or more genes encoding an invertase. In further embodiments, the genetic modification(s) present in the persistent yeast cells can be associated with a modification in the amino acid sequence of the polypeptide having invertase activity (providing the modified polypeptide with increased invertase activity).

The persistent yeast cells can be selected, from a population of yeast cells, for the improved invertase activity phenotype. This can be done, for example, by submitting a population of yeast cells to a plurality of fermentation cycles which includes limiting the amount of fermentable sugars (like glucose and fructose) and selecting the yeast cells having the ability to digest unfermentable sugars more rapidly (like sucrose). Alternatively or in combination, the yeasts can be genetically engineered to provide an improved invertase activity phenotype. This can be done, for example, by introducing one or more genetic modification to increase the activity or the expression of one or more polypeptide having invertase activity.

In an embodiment, the persistent yeast cell exhibits the improved invertase activity phenotypic trait optionally in combination with at least one of the following additional phenotypic traits: a fast settling phenotype, a rugose, triploidy or increased signaling in a RAS/cAMP/PKA pathway. In an embodiment, the persistent yeast cell exhibits the improved invertase activity phenotypic trait optionally in combination with at least two of the following additional phenotypic traits: a fast settling phenotype, a rugose, triploidy or increased signaling in a RAS/cAMP/PKA pathway. In an embodiment, the persistent yeast cell exhibits the improved invertase activity phenotypic trait optionally in combination with at least three of the following additional phenotypic traits: a fast settling phenotype, a rugose, triploidy or increased signaling in a RAS/cAMP/PKA pathway. In an embodiment, the persistent yeast cell exhibits the improved invertase activity phenotypic trait in combination with the following additional phenotypic traits: a fast settling phenotype, a rugose, triploidy and increased signaling in a RAS/cAMP/PKA pathway.

### Triploidy

In some embodiments, the persistent yeast cells of the present disclosure are triploids. Cell size (volume) is larger in higher ploidy strains and is associated with robustness to toxic compounds as well as increased adaptive fitness.

The persistent yeast cells can be selected, from a population of yeast cells, for a higher ploidy, like triploidy. This can be done, for example, by submitting a population of yeast cells to a plurality of fermentation cycles and selecting the yeast cells being triploids. Alternatively or in combination, the persistent yeast cells can be submitted to a process to provide triploidy. This can be done, for example, by using various mating techniques known in the art.

In an embodiment, the persistent yeast cell is a triploid optionally in combination with at least one of the following additional phenotypic traits: a fast settling phenotype, a rugose phenotype, improved invertase activity or increased signaling in a RAS/cAMP/PKA pathway. In an embodiment, the persistent yeast cell is a triploid optionally in combination with at least two of the following additional phenotypic traits: a fast settling phenotype, a rugose phenotype, improved invertase activity or increased signaling in a RAS/cAMP/PKA pathway. In an embodiment, the persistent yeast cell is a triploid optionally in combination with at least three of the following additional phenotypic traits: a fast settling phenotype, a rugose phenotype, improved invertase activity or increased signaling in a RAS/cAMP/PKA pathway. In an embodiment, the persistent yeast cell is a triploid optionally in combination with the following additional phenotypic traits: a fast settling phenotype, a rugose phenotype, improved invertase activity and increased signaling in a RAS/cAMP/PKA pathway.

### Increased Signaling in a RAS/cAMP/PKA Pathway

In another embodiment, the persistent yeast cells exhibit increased signaling in a RAS/cAMP/PKA pathway when compared to control yeast cell lacking this phenotype (e.g., the PE-2 strain for example). Signaling via the RAS/cAMP/PKA pathway is important for nutrient signaling in yeasts and is associated to responses to glucose, nitrogen and phosphate levels. Increasing signaling in the RAS/cAMP/PKA pathway is associated with glucose de-repression which eventually leads to better glucose and nitrogen uptake even in the presence of low levels of glucose (e.g., between 1 to 5 g/L) or other nutrients.

As used in the context of the present disclosure a persistent yeast cell having “increased signaling activity in the RAS/cAMP/PKA pathway” exhibits an increase in biological activity in one or more protein in the RAS/cAMP/PKA pathway. Persistent yeast cells exhibiting the increased signaling activity in the RAS/cAMP/PKA pathway phenotypic trait can show a decrease in the production of cAMP after a glucose spike, when compared to corresponding control yeasts lacking the increased signaling activity in the RAS/cAMP/PKA pathway phenotype. Alternatively or in combination, persistent yeast cells exhibiting the increased signaling activity in the RAS/cAMP/PKA pathway phenotypic trait, can show an increase in the production of cAMP in a basal medium, when compared to corresponding control yeasts lacking the increased signaling activity in the RAS/cAMP/PKA pathway phenotype. In some embodiments, the the control yeasts lacking the increased signaling activity in the RAS/cAMP/PKA pathway phenotype are from the Saccharomyces cerevisiae PE-2 strain. As it is known in the art, the increase in cAMP caused by this biological pathway causes the dissociation of the PKA protein into the BCY1 protein and the TPK1-3 protein. This increase in RAS/cAMP/PKA signaling is preferably observed during fermentation (e.g., for example, in anaerobic conditions) and, in some embodiments, is not observed during propagation (e.g., for example, in glucose-limited aerobic conditions).

In one embodiment, the persistent yeast cells of the present disclosure exhibit increased signaling in the RAS/cAMP/PKA pathway when compared to a control strain lacking the increased signaling activity in the RAS/cAMP/PKA pathway phenotype (e.g., the PE-2 strain for example) in comparable conditions. In an embodiment, a population consisting essentially of the persistent yeast cells exhibits a fold increase in cAMP production less than 70, 65, 60, 55, 50, 45, 40, 35, 30, 25% or less when compared to the fold increase in cAMP production in a population consisting essentially of control yeast cells lacking the increased signaling activity in the RAS/cAMP/PKA pathway phenotype (e.g., the PE-2 strain) in comparable conditions. In order to make such comparison, both populations can be glucose depleted and submitted to a glucose spike. In addition, the cAMP production can be measured before and after a predetermined time (e.g., 5 min) of a glucose spike.

In a specific embodiment, a population consisting essentially of the persistent yeast cells having been glucose depleted and submitted to a glucose spike exhibits a fold increase in cAMP production equal to or less than 1.7, 1.6, 1.5, 1.4, 1.3 or less. In order to make such determination, the cAMP production can be measured before and after a predetermined time (e.g., 5 min) of a glucose spike.

In order to achieve such increase in RAS/cAMP/PKA signaling, the expression and/or activity of one or more polypeptide of the RAS/cAMP/PKA pathway can be increased (when compared to a corresponding control yeast lacking the phenotype, such as, for example, the PE-2 strain). The one or more polypeptides whose expression or biological activity can be increased include, but are not limited to a CDC25 polypeptide (a membrane bound guanine nucleotide exchange factor capable of activating a RAS1 polypeptide and/or a RAS2 polypeptide), a SDC25 polypeptide (a Ras guanine nucleotide exchange factor capable of activating the RAS1 polypeptide and/or the RAS2 polypeptide), a RAS1 polypeptide (GTPase whose activity increase the activity of the Cyr1 polypeptide) and/or a RAS2 polypeptide (a GTPase whose activity increases the activity of the Cyr1).

In an embodiment, the RAS2 polypeptide expression or its biological activity is increased to cause an increase in the signaling activity of the RAS/cAMP/PKA pathway in the persistent yeast cell. In such embodiment, the persistent yeast cell can include a mutation in the RAS2 polypeptide (herein referred to as a mutated RAS2 polypeptide) which increases its biological activity. For example, the mutated RAS2 polypeptide can be a variant or a fragment of the wild-type RAS2 polypeptide resulting in an increase in the biological activity of the RAS2 polypeptide. The RAS2 polypeptide is a GTPase and as such its biological activity includes binding to GTP and hydrolyzing GTP into GDP. As such, in the context of the present disclosure, a mutated RAS2 polypeptide having increased (biological) activity can exhibit a higher binding affinity for GTP, a higher GTP hydrolyzing activity or both, when compared to the wild-type RAS2 polypeptide. In an embodiment, the mutated RAS2 polypeptide can have one or more amino acid substitutions with respect to the amino acid sequence of the wild-type RAS2 polypeptide.

For example, the mutated RAS2 polypeptide can have an amino acid substitution at a residue corresponding to location 66 of SEQ ID NO: 19 (or a corresponding residue in another wild-type RAS2 polypeptide). In an embodiment, the amino acid substitution of the mutated RAS2 polypeptide is limited to the residue located at position 66 of SEQ ID NO: 19 (or a corresponding residue in another wild-type RAS2 polypeptide). In the wild-type RAS2 polypeptide of S. cerevisiae (SEQ ID NO: 19), the amino acid residue at location 66 is an alanine residue. In an embodiment, the mutated RAS2 polypeptide does not have an alanine residue located at position 66 of SEQ ID NO: 19 (or at a corresponding position in another wild-type RAS2 polypeptide), but instead has an histidine, an isoleucine, an arginine, a leucine, an asparagine, a lysine, an aspartic acid, a methionine, a cysteine, a phenylalanine, a glutamic acid, a threonine, a glutamine, a tryptophan, a glycine, a valine, a proline, a serine or a tyrosine residue. In an embodiment, the mutated RAS2 polypeptide has, at position 66 of SEQ ID NO: 19 (or at a corresponding position in another wild-type RAS2 polypeptide) does not have an aliphatic amino acid residue, such as, for example, a glycine, a valine, a leucine or an isoleucine residue. In still another embodiment, the mutated RAS2 polypeptide has, at position 66 of SEQ ID NO: 19 (or at a corresponding position in another wild-type RAS2 polypeptide) a hydroxyl or sulfur/selenium-containing amino acid, such as, for example, a serine, a cysteine, a threonine or a methionine residue. In yet another embodiment, the mutated RAS2 polypeptide has, at position 66 of SEQ ID NO: 19 (or at a corresponding position in another wild-type RAS2 polypeptide) a threonine residue. In still a further embodiment, the mutated RAS2 polypeptide has the amino acid sequence of SEQ ID NO: 17 and can be encoded by a nucleic acid molecule having a nucleic acid molecule having the sequence of SEQ ID NO: 18 or a degenerate sequence of SEQ ID NO: 18 encoding SEQ ID NO: 17.

In another example, the mutated RAS2 polypeptide can have an amino acid substitution at a residue corresponding to location 19 of SEQ ID NO: 19 (or a corresponding residue in another wild-type RAS2 polypeptide). In the wild-type RAS2 polypeptide of S. cerevisiae (SEQ ID NO: 19), the amino acid residue at location 19 is a glycine residue. In an embodiment, the mutated RAS2 polypeptide does not have a glycine residue located at position 19 of SEQ ID NO: 19 (or at a corresponding position in another wild-type RAS2 polypeptide), but instead has an histidine, an isoleucine, an arginine, a leucine, an asparagine, a lysine, an aspartic acid, a methionine, a cysteine, a phenylalanine, a glutamic acid, a threonine, a glutamine, a tryptophan, an alanine, a valine, a proline, a serine or a tyrosine residue. In yet another embodiment, the mutated RAS2 polypeptide has, at position 19 of SEQ ID NO: 19 (or at a corresponding position in another wild-type RAS2 polypeptide) a valine residue.

In another example, the mutated RAS2 polypeptide can have an amino acid substitution at a residue corresponding to location 77 of SEQ ID NO: 19 (or a corresponding residue in another wild-type RAS2 polypeptide). In the wild-type RAS2 polypeptide of S. cerevisiae (SEQ ID NO: 19), the amino acid residue at location 77 is a glutamine residue. In an embodiment, the mutated RAS2 polypeptide does not have a glutamine residue located at position 77 of SEQ ID NO: 19 (or at a corresponding position in another wild-type RAS2 polypeptide), but instead has an histidine, an isoleucine, an arginine, a leucine, an asparagine, a lysine, an aspartic acid, a methionine, a cysteine, a phenylalanine, a glutamic acid, a threonine, an alanine, a tryptophan, a glycine, a valine, a proline, a serine or a tyrosine residue. In yet another embodiment, the mutated RAS2 polypeptide has, at position 77 of SEQ ID NO: 19 (or at a corresponding position in another wild-type RAS2 polypeptide) a lysine residue.

In another example, the mutated RAS2 polypeptide can have an amino acid substitution at a residue corresponding to location 112 of SEQ ID NO: 19 (or a corresponding residue in another wild-type RAS2 polypeptide). In the wild-type RAS2 polypeptide of S. cerevisiae (SEQ ID NO: 19), the amino acid residue at location 112 is an aspartic acid residue. In an embodiment, the mutated RAS2 polypeptide does not have an aspartic acid residue located at position 112 of SEQ ID NO: 19 (or at a corresponding position in another wild-type RAS2 polypeptide), but instead has an histidine, an isoleucine, an arginine, a leucine, an asparagine, a lysine, a glutamine, a methionine, a cysteine, a phenylalanine, a glutamic acid, a threonine, an alanine, a tryptophan, a glycine, a valine, a proline, a serine or a tyrosine residue. In yet another embodiment, the mutated RAS2 polypeptide has, at position 112 of SEQ ID NO: 19 (or at a corresponding position in another wild-type RAS2 polypeptide) a tyrosine residue.

In an embodiment, the RAS1 polypeptide expression or biological activity is increased to cause an increase in the signaling activity of the RAS/cAMP/PKA pathway in the persistent yeast cell. In such embodiment, the persistent yeast cell includes a mutation in the RAS1 polypeptide (herein referred to as a mutated RAS1 polypeptide) which increases its biological activity. For example, the mutated RAS1 polypeptide can be a variant or a fragment of the wild-type RAS1 polypeptide resulting in an increase in the biological activity of the RAS1 polypeptide. The RAS1 polypeptide is a GTPase and as such its biological activity include binding to GTP and hydrolyzing GTP into GDP. As such, in the context of the present disclosure, a mutated RAS1 polypeptide having increased (biological) activity can exhibit a higher binding affinity for GTP, a higher GTP hydrolyzing activity or both, when compared to the wild-type RAS1 polypeptide. In an embodiment, the mutated RAS1 polypeptide can have an amino acid substitution. For example, the mutated RAS1 polypeptide can have an amino acid substitution at a residue corresponding to location 66 of SEQ ID NO: 26 (or at a corresponding residue in another wild-type RAS1 polypeptide). In an embodiment, the amino acid substitution of the mutated RAS1 polypeptide is limited to the residue located at position 66 of SEQ ID NO: 26 (or a corresponding residue in another wild-type RAS1 polypeptide). In the wild-type RAS1 polypeptide of S. cerevisiae (SEQ ID NO: 26), the amino acid residue at location 66 is an alanine residue. In an embodiment, the mutated RAS1 polypeptide does not have an alanine residue located at position 66 of SEQ ID NO: 26 (or at a corresponding position in another wild-type RAS1 polypeptide), but instead has an histidine, an isoleucine, an arginine, a leucine, an asparagine, a lysine, an aspartic acid, a methionine, a cysteine, a phenylalanine, a glutamic acid, a threonine, a glutamine, a tryptophan, a glycine, a valine, a proline, a serine or a tyrosine residue. In an embodiment, the mutated RAS1 polypeptide has, at position 66 of SEQ ID NO: 26 (or at a corresponding position in another wild-type RAS1 polypeptide) does not have an aliphatic amino acid residue, such as, for example, a glycine, a valine, a leucine or an isoleucine residue. In still another embodiment, the mutated RAS1 polypeptide has, at position 66 of SEQ ID NO: 26 (or at a corresponding position in another wild-type RAS1 polypeptide) a hydroxyl or sulfur/selenium-containing amino acid, such as, for example, a serine, a cysteine, a threonine or a methionine residue. In yet another embodiment, the mutated RAS1 polypeptide has, at position 66 of SEQ ID NO: 26 (or at a corresponding position in another wild-type RAS1 polypeptide) a threonine residue. In still a further embodiment, the mutated RAS1 polypeptide has the amino acid sequence of SEQ ID NO: 27.

In another example, the expression and/or activity of one or more polypeptide of the RAS/cAMP/PKA pathway can be decreased (when compared to a corresponding control yeast lacking the phenotype, such as the PE-2 strain) to achieve an increase in the signaling activity RAS/cAMP/PKA pathway. The one or more polypeptide whose expression or biological activity can be decreased include, but is not limited to, a IRA1 polypeptide (a GTPase-activating polypeptide whose activity decreases the activity of the wild-type Ras1 polypeptide and/or the wild-type RAS2 polypeptide) and/or an IRA2 polypeptide (a GTPase-activating polypeptide whose activity decreases the activity of the wild-type RAS1 polypeptide and/or the wild-type RAS2 polypeptide).

As indicated above, in an embodiment, the expression and/or activity of the IRA2 polypeptide can be decreased to achieve an increase in the signaling activity in the RAS/cAMP/PKA pathway in the persistent yeast cell. In an embodiment, the persistent yeast cell can include a mutation in the IRA2 polypeptide (herein referred to as a mutated IRA2 polypeptide) which decreases its biological activity. As it is known in the art, the IRA2 polypeptide converts the wild-type RAS1 polypeptide or the wild-type RAS2 polypeptide from their GTP-bound to their GDP-bound inactive form. The biological activity of the IRA2 polypeptide includes binding to the wild-type RAS1 polypeptide and to the wild-type RAS2 polypeptide. As such, in the context of the present disclosure, a mutated IRA2 polypeptide having decreased (biological activity) can exhibit a lower binding affinity for the wild-type RAS1 polypeptide, the wild-type RAS2 polypeptide or both, when compared to the wild-type IRA2. For example, the mutated IRA2 polypeptide can be a variant or a fragment of the wild-type IRA2 polypeptide (which can have, in some embodiments, the amino acid sequence of SEQ ID NO: 22 or be encoded by a nucleic acid molecule having the nucleic acid sequence of SEQ ID NO: 28 or be a degenerate sequence encoding the amino acid sequence of SEQ ID NO: 22) exhibiting in an increase in the biological activity of the wild-type RAS1 polypeptide and/or the wild-type RAS2 polypeptide.

In an embodiment, the consensus sequence of the mutated IRA2 polypeptide can have the amino acid sequence of SEQ ID NO: 23. In yet a further embodiment, the mutated IRA2 polypeptide can have a mutation at the amino acid residue located at position 2440 of the wild-type IRA2 polypeptide of SEQ ID NO 22 (or a corresponding position in another wild-type IRA2 polypeptide). This mutation at position 2440 can cause the substitution of a glutamic acid residue to a lysine residue. In a specific embodiment, the mutated IRA2 polypeptide can have the amino acid sequence of SEQ ID NO: 20 and/or be encoded by a nucleic acid molecule having the nucleic acid sequence of SEQ ID NO: 21 or a degenerate sequence of SEQ ID NO: 21 encoding SEQ ID NO: 20. In a specific embodiment, the mutated IRA2 polypeptide can be a truncated IRA2 polypeptide encoded by a nucleic acid molecule or a gene which includes a frame-shift mutation. The mutated fragment of the IRA2 polypeptide can have the amino acid sequence of SEQ ID NO: 24.

In an embodiment, the expression and/or activity of the IRA1 polypeptide can be decreased to achieve an increase in the signaling activity in the RAS/cAMP/PKA pathway in the persistent yeast cell. In an embodiment, the persistent yeast cell expresses a mutation in the IRA1 polypeptide (herein referred to as a mutated IRA1 polypeptide) which decreases its biological activity. For example, the mutated IRA1 polypeptide can be a variant or a fragment of the wild-type IRA1 polypeptide resulting in an increase in the biological activity of the wild-type RAS1 polypeptide and/or the wild-type RAS2 polypeptide. The IRA1 polypeptide converts the wild-type RAS1 polypeptide or the wild-type RAS2 polypeptide from their GTP-bound to their GDP-bound inactive form. The biological activity of the IRA1 polypeptide includes binding to the wild-type RAS1 polypeptide and/or to the wild-type RAS2 polypeptide. As such, in the context of the present disclosure, a mutated IRA1 polypeptide having decreased (biological activity) can exhibit a lower binding affinity for the wild-type RAS1 polypeptide, the wild-type RAS2 polypeptide or both. In a specific embodiment, the mutated IRA1 polypeptide can be a truncated IRA1 polypeptide encoded by a nucleic acid molecule or a gene which includes a frame-shift mutation.

In yet another example, the expression and/or activity of one or more polypeptide of the RAS/cAMP/PKA pathway can be increased and the expression and/or activity of one or more polypeptide of the RAS/cAMP/PKA pathway can be decreased both in comparison with a corresponding control yeast lacking the phenotype (like the PE-2 strain for example) to achieve an increase in the signaling activity RAS/cAMP/PKA pathway.

In order to achieve such increase in RAS/cAMP/PKA signaling, it is also possible to regulate the activity of one or more polypeptide of the RAS/cAMP/PKA signaling pathway at the post-transcriptional level. For example, it is possible to genetically modify the recombinant yeast host cell to allow for the glucose-induced polypeptide turnover of one or more polypeptides in the RAS/cAMP/PKA signaling pathway (e.g., the IRA1 polypeptide and/or the IRA2 polypeptide for example).

The persistent yeast cell can be selected, from a population of yeast cells, for the increased signaling in the RAS/cAMP/PKA pathway. This can be done, for example, by submitting a population of yeast cells to a plurality of fermentation cycles in limited nutrient availability and selecting the yeast cells exhibiting increased signaling in the RAS/cAMP/PKA pathway. Alternatively or in combination, the persistent yeast cell can be genetically engineered to provide increased signaling in the RAS/cAMP/PKA pathway. This can be done, for example, by introducing one or more genetic modification to introduce one or more genetic modification in the polypeptides involved in the RAS/cAMP/PKA pathway.

In an embodiment, the persistent yeast cell exhibits the increased signaling in a RAS/cAMP/PKA pathway phenotypic trait optionally in combination with at least one of the following additional phenotypic traits: a fast settling phenotype, a rugose phenotype, an improved invertase activity or triploidy. In an embodiment, the persistent yeast cell exhibits the increased signaling in a RAS/cAMP/PKA pathway phenotypic trait optionally in combination with at least two of the following additional phenotypic traits: a fast settling phenotype, a rugose phenotype, an improved invertase activity or triploidy. In an embodiment, the persistent yeast cell exhibits the increased signaling in a RAS/cAMP/PKA pathway phenotypic trait optionally in combination with at least three of the following additional phenotypic traits: a fast settling phenotype, a rugose phenotype, an improved invertase activity or triploidy. In an embodiment, the persistent yeast cell exhibits the increased signaling in a RAS/cAMP/PKA pathway phenotypic trait in combination with the following additional phenotypic traits: a fast settling phenotype, a rugose phenotype, an improved invertase activity and triploidy.

### Recombinant Yeast Host Cells

The recombinant yeast host cells of the present disclosure are capable of modulating the activity or the expression of at least one polypeptide for increasing, with compared to a parental cell, the conversion of a biomass into a fermentation product and optionally for reducing the production of a fermentation by-product. As used in the context of the present application, a “non-genetically modified parental cell” refer to the parental cell that was modified to provide the recombinant yeast host cell. The parental host cell does not include the one or more genetic modification that are present in the recombinant yeast host cell renders or augments the capacity of the latter capable to increase the conversion of a biomass into a fermentation product and optionally reducing the production of a fermentation by-product. The parental host cell can be a non-genetically modified cell. Alternatively, the parental host cell can include one or more genetic modifications that are unrelated to the conversion of a biomass into a fermentation product or in the production of a fermentation by-product. In some embodiments, the parental host cell can be a persistent yeast cell and include the one or more phenotypic traits described herein.

The recombinant yeast host cells of the present disclosure are intended to be used in a plurality of fermentation cycles as described herein. In each fermentation cycles, the recombinant yeast host cells of the present disclosure is involved in fermentation, e.g. the conversion of a biomass into a fermentation product (which can be an alcohol, such as, for example, ethanol). In one embodiment, the polypeptide(s) whose activity or expression is modulated in the recombinant yeast host cells can be directly involved in converting the biomass into the fermentation product. In another embodiment, the polypeptide(s) whose activity or expression is modulated in the recombinant yeast host cells can be indirectly involved in converting the biomass into the fermentation product by reducing or limiting the production of a fermentation by-product (such as, for example, glycerol) and ultimately increasing the fermentation yield (e.g., the yield of the fermentation product).

During yeast metabolism, a major by-product of biomass fermentation is glycerol. Glycerol is produced in microorganisms, such as yeasts, in response to a redox or osmotic stress. The glycerol produced is then exported from the cell where it is considered waste. While the production of glycerol is important to protect microorganisms from various stressors, it also tends to decrease ethanol yields, especially when the microorganisms are growing or encountering osmotic stress.

In yeasts, glycerol is a required metabolic end-product of ethanol fermentation allowing the yeast to balance its redox state and regenerate NAD+ used as a cofactor during glycolysis. During anaerobic growth on carbohydrates, production of ethanol and carbon dioxide is redox neutral, while the reactions that create cell biomass and associated carbon dioxide are more oxidized relative to carbohydrates. The production of glycerol, which is more reduced relative to carbohydrates, functions as an electron sink to off-set cell biomass formation, so that overall redox neutrality is conserved. This is essential from a theoretical consideration of conservation of mass, and in practice strains unable to produce glycerol are unable to grow under anaerobic conditions. As glycerol is a byproduct with low value, it can be an undesirable by-product of fermentation. There is a strong commercial incentive to reduce glycerol as a by-product during the production of fuels and chemicals, as reduction typically results in an increased yield of the desired compound.

In an embodiment, the recombinant yeast host cells of the present disclosure are capable of increasing the activity or the expression of a first polypeptide involved in the conversion of a biomass into a fermentation product and/or the reduction in the production of a fermentation by-product. As such, the recombinant yeast host cells of the present disclosure can include one or more genetic modification for increasing the activity or the expression of a first polypeptide. In an embodiment, the genetic modification can be located in a regulatory region (such as a promoter region) of a native gene encoding a native (first) polypeptide so as to increase the expression (and ultimately the activity) of the native (first polypeptide). Alternatively or in combination, the genetic modification can be the introduction of one or more (first) heterologous nucleic acid molecules encoding a heterologous (first) polypeptide in the recombinant yeast host cells so as to increase or provide the recombinant yeast host cell with increased activity of the first polypeptide. In embodiments in which the genetic modification is intended to cause the expression of a secreted first polypeptide, the first nucleic acid heterologous nucleic acid molecule can also include a portion encoding a signal sequence operatively associated with another portion encoding the secreted polypeptide. The heterologous nucleic acid molecules that may be present in the recombinant yeast host cells can be integrated at the same or different integration sites.

In an embodiment, the first polypeptide is a saccharolytic enzyme (or a polypeptide having saccharolytic activity) and as such the recombinant yeast host cell comprises one or more first genetic modification for overexpressing a native saccharolytic or expressing heterologous saccharolytic enzymes. As used in the context of the present disclosure, a “saccharolytic enzyme” can be any enzyme involved in carbohydrate digestion, metabolism and/or hydrolysis, including amylases, cellulases, hemicellulases, cellulolytic and amylolytic accessory enzymes, inulinases, levanases, and pentose sugar utilizing enzymes. amylolytic enzyme. In an embodiment, the saccharolytic enzyme is an amylolytic enzyme. As used herein, the expression “amylolytic enzyme” refers to a class of enzymes capable of hydrolyzing starch or hydrolyzed starch. Amylolytic enzymes include, but are not limited to alpha-amylases (EC 3.2.1.1, sometimes referred to fungal alpha-amylase, see below), maltogenic amylase (EC 3.2.1.133), glucoamylase (EC 3.2.1.3), glucan 1,4-alpha-maltotetraohydrolase (EC 3.2.1.60), pullulanase (EC 3.2.1.41), iso-amylase (EC 3.2.1.68) and amylomaltase (EC 2.4.1.25).

In an embodiment, the one or more amylolytic enzymes can be, a maltogenic alpha-amylase from Geobacillus stearothermophilus, a glucoamylase from Saccharomycopsis fibuligera, a glucan 1,4-alpha-maltotetraohydrolase from Pseudomonas saccharophila, a pullulanase from Bacillus naganoensis, a pullulanase from Bacillus acidopullulyticus, an iso-amylase from Pseudomonas amyloderamosa, and/or amylomaltase from Thermus thermophilus. Some amylolytic enzymes have been described in US20220127564 and are incorporated herein by reference.

For example, the heterologous alpha-amylase can be from a Rhizomucor sp., such as, for example, from Rhizomucor pusillus. In an embodiment, the heterologous alpha-amylase corresponds to Uniprot M9T189. For example, the heterologous alpha-amylase can be from a Aspergillus sp., such as, for example, from Aspergillus luchuensis. In an embodiment, the heterologous alpha-amylase corresponds to Uniprot A0A146F6W4 or to GenBank Accession Number GAT21778. In an embodiment, the heterologous alpha-amylase corresponds to Uniprot 013296 or to GenBank Accession Number BAA22993. For example, the heterologous alpha-amylase can be from Aspergillus oryzae. In an embodiment, the heterologous alpha-amylase corresponds to Uniprot Q2UIS5 or to GenBank Accession Number XP_001820542. For example, the heterologous alpha-amylase can be from Aspergillus niger. In an embodiment, the heterologous alpha-amylase corresponds to Uniprot A2QTS4 or to GenBank Accession Number XP_001393626. In an embodiment, the heterologous alpha-amylase corresponds to Uniprot A2R6F9 or to GenBank Accession Number XP_001397301. In an embodiment, the heterologous alpha-amylase corresponds to GenBank Accession Number XP_001395328. In an embodiment, the heterologous alpha-amylase corresponds to Uniprot A0A370BQ30 or to GenBank Accession Number RDH15462. For example, the heterologous alpha-amylase can be from Aspergillus fischeri. In an embodiment, the heterologous alpha-amylase corresponds to Uniprot A1CYB1 or to GenBank Accession Number XP_001265628. For example, the heterologous alpha-amylase can be from a Homo sp., such as, for example, from Homo sapiens. In an embodiment, the heterologous alpha-amylase corresponds to GenBank Accession Number 1B2Y_A.

For example, the yeast cell can bear one or more genetic modifications allowing for the production of a heterologous glucoamylase. Many microbes produce an amylase to degrade extracellular starches. In addition to cleaving the last α(1-4) glycosidic linkages at the non-reducing end of amylose and amylopectin, yielding glucose, γ-amylase will cleave a(1-6) glycosidic linkages. The heterologous glucoamylase can be derived from any organism. In an embodiment, the heterologous polypeptide is derived from a γ-amylase, such as, for example, the glucoamylase of Saccharomycopsis filbuligera (e.g., encoded by the glu 0111 gene). Examples of recombinant yeast cells bearing such genetic modifications and expressing saccharolytic enzymes are described in US20130323822 as well as in US20180265853 and are both herewith incorporated in its entirety.

In another embodiment, the first polypeptide can be involved in the transport of a fermentation by-product, like glycerol, in the recombinant yeast cell. In an embodiment, the first polypeptide is a glycerol transporter and can, in some further embodiments, be responsible for the import of glycerol in the recombinant yeast host cells. In a specific embodiment, the first polypeptide is a sugar transporter-like protein (STL1). As such, the recombinant yeast host cells comprise one or more first genetic modification for overexpressing a native STL1 or expressing a heterologous STL1. By increasing the activity or expression of the STL1 polypeptide, it is possible to control, to some extent, glycerol synthesis and ultimately increase the fermentation (ethanol) yield.

The STL1 polypeptide is natively expressed in yeasts and fungi, therefore the heterologous polypeptide functioning to import glycerol can be derived from yeasts and fungi. STL1 genes encoding the STL1 polypeptide include, but are not limited to, Gene ID: 852149 (encoded by SEQ ID NO: 7 and shown in SEQ ID NO: 8), Candida albicans, Kluyveromyces lactis Gene ID: 2896463 (SEQ ID NO: 67), Eremothecium gossypii Gene ID: 4620396 (SEQ ID NO: 36), Eremothecium sinecaudum Gene ID: 28724161 (SEQ ID NO: 37), Torulaspora delbrueckii Gene ID: 11505245 (SEQ ID NO: 57), Lachancea thermotolerans Gene ID: 8290820 (SEQ ID NO: 60), Phialophora attinorum Gene ID: 28742143 (SEQ ID NO: 47), Penicillium digitatum Gene ID: 26229435 (SEQ ID NO: 46), Aspergillus oryzae Gene ID: 5997623 (SEQ ID NO: 61), Aspergillus fumigatus Gene ID: 3504696 (SEQ ID NO: 32), Talaromyces atroroseus Gene ID: 31007540 (SEQ ID NO: 53), Rasamsonia emersonii Gene ID: 25315795 (SEQ ID NO: 68), Aspergillus terreus Gene ID: 4322759 (SEQ ID NO: 33), Penicillium rubens Gene ID: 8310605 (SEQ ID NO: 58), Alternaria alternata Gene ID: 29120952 (SEQ ID NO: 31), Paraphaeosphaeria sporulosa Gene ID: 28767590 (SEQ ID NO: 45), Pyrenophora tritici-repentis Gene ID: 6350281 (SEQ ID NO: 49), Metarhizium robertsii Gene ID: 19259252 (SEQ ID NO: 41), Isaria fumosorosea Gene ID: 30023973 (SEQ ID NO: 39), Cordyceps militaris Gene ID: 18171218 (SEQ ID NO: 34), Pochonia chlamydosporia Gene ID: 28856912 (SEQ ID NO: 48), Metarhizium majus Gene ID: 26274087 (SEQ ID NO: 40), Neofusicoccum parvum Gene ID:19029314 (SEQ ID NO: 63), Diplodia corticola Gene ID: 31017281 (SEQ ID NO: 35), Verticillium dahliae Gene ID: 20711921 (SEQ ID NO: 56), Verticillium alfalfa Gene ID: 9537052 (SEQ ID NO: 55), Paracoccidioides lutzii Gene ID: 9094964 (SEQ ID NO: 44), Trichophyton rubrum Gene ID: 10373998 (SEQ ID NO: 59), Nannizzia gypsea Gene ID: 10032882 (SEQ ID NO: 42), Trichophyton verrucosum Gene ID: 9577427 (SEQ ID NO: 62), Trichophyton benhamiae Gene ID: 9523991 (SEQ ID NO: 54), Pyricularia oryzae Gene ID: 2678012 (SEQ ID NO: 50), Gaeumannomyces tritici Gene ID: 20349750 (SEQ ID NO: 38), Phaeoacremonium minimum Gene ID: 19329524 (SEQ ID NO: 65), Eutypa lata Gene ID: 19232829 (SEQ ID NO: 64), Scedosporium apiospermum Gene ID: 27721841 (SEQ ID NO: 51), Aureobasidium namibiae Gene ID: 25414329 (SEQ ID NO: 66), Sphaerulina musiva Gene ID: 27905328 (SEQ ID NO: 52) as well as Pachysolen tannophilus GenBank Accession Numbers JQ481633 (SEQ ID NO: 69) and JQ481634 (SEQ ID NO: 43).

The first polypeptide can be encoded by STL1 gene as indicated herein or a STL1 gene ortholog or paralog. The heterologous polypeptide functioning to import glycerol can be a STL1 polypeptide as defined herein, a variant of the STL1 polypeptide and/or a fragment of the STL1 polypeptide. In addition, when more than one copy of the first heterologous nucleic acid molecule encoding STL1 is included in the recombinant yeast host cell, the plurality of first heterologous nucleic acid molecules encoding the STL1 polypeptide could be the same or different, integrated at the same or different integration sites.

In a specific embodiment, the recombinant yeast host cells of the present disclosure is capable of expressing a STL1 polypeptide having the amino acid sequence of SEQ ID NO: 8, a variant of the amino acid sequence of SEQ ID NO:8 having glycerol transport activity or a fragment of the amino acid sequence of SEQ ID NO: 8 having glycerol transporter activity. In another specific embodiment, the recombinant yeast host cells of the present disclosure comprises a heterologous nucleic acid molecule comprising the nucleic acid sequence of SEQ ID NO: 7, corresponds to a degenerate sequence of the nucleic acid sequence of SEQ ID NO: 7 (encoding SEQ ID NO:8) or encodes the variant or the fragment of the amino acid sequence of SEQ ID NO: 8. The heterologous nucleic acid molecule encoding the STL1 polypeptide, variant or fragment can be present in one copy in the recombinant yeast host cell. The heterologous nucleic acid molecule encoding the STL1 polypeptide, variant or fragment can be present in two copies in the recombinant yeast host cell. The heterologous nucleic acid molecule encoding the STL1 polypeptide, variant or fragment can be present in three copies in the recombinant yeast host cell. The heterologous nucleic acid molecule encoding the STL1 polypeptide, variant or fragment can be present in four copies in the recombinant yeast host cell. The heterologous nucleic acid molecule encoding the STL1 polypeptide, variant or fragment can be present in five copies in the recombinant yeast host cell. The heterologous nucleic acid molecule encoding the STL1 polypeptide, variant or fragment can be present in six copies in the recombinant yeast host cell. The heterologous nucleic acid molecule encoding the STL1 polypeptide, variant or fragment can be present in seven copies in the recombinant yeast host cell. The heterologous nucleic acid molecule encoding the STL1 polypeptide, variant or fragment can be present in eight copies in the recombinant yeast host cell. The heterologous nucleic acid molecule encoding the STL1 polypeptide, variant or fragment can be present in nine copies in the recombinant yeast host cell. The heterologous nucleic acid molecule encoding the STL1 polypeptide, variant or fragment can be present in ten copies in the recombinant yeast host cell. The heterologous nucleic acid molecule encoding the STL1 polypeptide, variant or fragment can be present in eleven copies in the recombinant yeast host cell. The heterologous nucleic acid molecule encoding the STL1 polypeptide, variant or fragment can be present in twelve copies in the recombinant yeast host cell.

In an embodiment, the first polypeptide is a heterologous glyceraldehyde-3-phosphate dehydrogenase. Glyceraldehyde-3-phosphate dehydrogenases allow the catalysis of the reaction of glyceraldehyde-3-phosphate to 3-phosphoglycerate in glycolysis, using NADP+ as a cofactor. In some embodiments, regeneration of NADPH and/or NADH by way a glycolytic pathway using glyceraldehyde-3-phosphate also improves ethanol production and reduces glycerol production. The glyceraldehyde-3-phosphate dehydrogenase is a non-phosphorylating glyceraldehyde-3-phosphate dehydrogenase, e.g., it is incapable of mediating a phosphorylation reaction. In some embodiments, the glyceraldehyde-3-phosphate dehydrogenase is of enzyme commission (EC) class 1.2.1, however it excludes the enzymes capable of mediating a phosphorylating reaction. The glyceraldehyde-3-phosphate dehydrogenase of the present disclosure specifically exclude enzymes capable of directly using or generating of 3-phospho-D-glyceroyl phosphate, such as enzymes of EC 1.2.1.13. Enzymes of EC 1.2.1.13 catalyze the following reaction:

$$D - glyceraldehyde 3 - phosphate + {NADP}^{+} <= \text{>} 3 - phospho - D - glyceroyl phosphate + NADPH$$

In one embodiment, the glyceraldehyde-3-phosphate dehydrogenase is NADP⁺ dependent (EC1.2.1.9) and allows the conversion of NADP⁺ to NADPH. Enzymes of EC1.2.1.9 can only use NADP⁺ as a cofactor.

In one embodiment, the glyceraldehyde-3-phosphate dehydrogenase is bifunctional NADP⁺/NAD⁺ dependent (EC1.2.1.90) and allows the conversion of NADP⁺ to NADPH and/or NAD⁺ to NAD⁺. Enzymes of EC1.2.1.90 can use NADP⁺ or NAD⁺ as a cofactor. In some embodiments, glyceraldehyde-3-phosphate dehydrogenase uses NADP⁺ and/or NAD⁺ as a cofactor. In one embodiment, the glyceraldehyde-3-phosphate dehydrogenase is encoded by a GAPN gene. In one embodiment, the glyceraldehyde-3-phosphate dehydrogenase is GAPN. In some embodiments, the recombinant yeast host cell includes two first genetic modifications and is capable of expressing STL1 and GAPN.

In some embodiments, the glyceraldehyde-3-phosphate dehydrogenase can be derived from a bacteria, for example, from the genus Streptococcus and, in some instances, from the species Strepotococcus mutans. The glyceraldehyde-3-phosphate dehydrogenase can be encoded by the GAPN gene from Streptococcus mutans, or a GAPN gene ortholog, or a GAPN gene paralog. In an embodiment, the GAPN gene comprises the nucleic acid sequence of SEQ ID NO: 30, is a variant of the nucleic acid sequence of SEQ ID NO: 30 (including but not limited to a degenerate variant of SEQ ID NO: 30 encoding the amino acid sequence of SEQ ID NO: 29) or is a fragment of the nucleic acid sequence of SEQ ID NO: 30. In an embodiment, the GAPN has the amino acid sequence of SEQ ID NO: 29, is a variant of the amino acid of SEQ ID NO: 29 or is a fragment of SEQ ID NO: 29.

In some embodiments, the glyceraldehyde-3-phosphate dehydrogenase can be derived from a bacteria, for example, from the genus Lactobacillus and, in some instances, from the species Lactobacillus delbrueckii. The glyceraldehyde-3-phosphate dehydrogenase can be encoded by the GAPN gene from Lactobacillus delbrueckii, or a GAPN gene ortholog, or a GAPN gene paralog.

In some embodiments, the glyceraldehyde-3-phosphate dehydrogenase can be derived from a bacteria, for example, from the genus Streptococcus and, in some instances, from the species Strepotococcus thermophilus. The glyceraldehyde-3-phosphate dehydrogenase can be encoded by the GAPN gene from Streptococcus thermophilus, or a GAPN gene ortholog, or a GAPN gene paralog.

In some embodiments, the glyceraldehyde-3-phosphate dehydrogenase can be derived from a bacteria, for example, from the genus Streptococcus and, in some instances, from the species Strepotococcus macacae. The glyceraldehyde-3-phosphate dehydrogenase can be encoded by the GAPN gene from Streptococcus macacae, or a GAPN gene ortholog, or a GAPN gene paralog.

In some embodiments, the glyceraldehyde-3-phosphate dehydrogenase can be derived from a bacteria, for example, from the genus Streptococcus and, in some instances, from the species Strepotococcus hyointestinalis. The glyceraldehyde-3-phosphate dehydrogenase can be encoded by the GAPN gene from Streptococcus hyointestinalis, or a GAPN gene ortholog, or a GAPN gene paralog.

In some embodiments, the glyceraldehyde-3-phosphate dehydrogenase can be derived from a bacteria, for example, from the genus Streptococcus and, in some instances, from the species Strepotococcus urinalis. The glyceraldehyde-3-phosphate dehydrogenase can be encoded by the GAPN gene from Streptococcus urinalis, or a GAPN gene ortholog, or a GAPN gene paralog.

In some embodiments, the glyceraldehyde-3-phosphate dehydrogenase can be derived from a bacteria, for example, from the genus Streptococcus and, in some instances, from the species Strepotococcus canis. The glyceraldehyde-3-phosphate dehydrogenase can be encoded by the GAPN gene from Streptococcus canis, or a GAPN gene ortholog, or a GAPN gene paralog.

In some embodiments, the glyceraldehyde-3-phosphate dehydrogenase can be derived from a bacteria, for example, from the genus Streptococcus and, in some instances, from the species Strepotococcus thoraltensis. The glyceraldehyde-3-phosphate dehydrogenase can be encoded by the GAPN gene from Streptococcus thoraltensis, or a GAPN gene ortholog, or a GAPN gene paralog.

In some embodiments, the glyceraldehyde-3-phosphate dehydrogenase can be derived from a bacteria, for example, from the genus Streptococcus and, in some instances, from the species Strepotococcus dysgalactiae. The glyceraldehyde-3-phosphate dehydrogenase can be encoded by the GAPN gene from Streptococcus dysgalactiae, or a GAPN gene ortholog, or a GAPN gene paralog.

In some embodiments, the glyceraldehyde-3-phosphate dehydrogenase can be derived from a bacteria, for example, from the genus Streptococcus and, in some instances, from the species Strepotococcus pyogenes. The glyceraldehyde-3-phosphate dehydrogenase can be encoded by the GAPN gene from Streptococcus pyogenes, or a GAPN gene ortholog, or a GAPN gene paralog.

In some embodiments, the glyceraldehyde-3-phosphate dehydrogenase can be derived from a bacteria, for example, from the genus Streptococcus and, in some instances, from the species Strepotococcus ictaluri. The glyceraldehyde-3-phosphate dehydrogenase can be encoded by the GAPN gene from Streptococcus ictaluri, or a GAPN gene ortholog, or a GAPN gene paralog.

In some embodiments, the glyceraldehyde-3-phosphate dehydrogenase can be derived from a bacteria, for example, from the genus Clostridium and, in some instances, from the species Clostridium perfringens. The glyceraldehyde-3-phosphate dehydrogenase can be encoded by the GAPN gene from Clostridium perfringens, or a GAPN gene ortholog, or a GAPN gene paralog.

In some embodiments, the glyceraldehyde-3-phosphate dehydrogenase can be derived from a bacteria, for example, from the genus Clostridium and, in some instances, from the species Clostridium chromiireducens. The glyceraldehyde-3-phosphate dehydrogenase can be encoded by the GAPN gene from Clostridium chromiireducens, or a GAPN gene ortholog, or a GAPN gene paralog.

In some embodiments, the glyceraldehyde-3-phosphate dehydrogenase can be derived from a bacteria, for example, from the genus Clostridium and, in some instances, from the species Clostridium botulinum. The glyceraldehyde-3-phosphate dehydrogenase can be encoded by the GAPN gene from Clostridium botulinum, or a GAPN gene ortholog, or a GAPN gene paralog.

In some embodiments, the glyceraldehyde-3-phosphate dehydrogenase can be derived from a bacteria, for example, from the genus Bacillus and, in some instances, from the species Bacillus cereus. The glyceraldehyde-3-phosphate dehydrogenase can be encoded by the GAPN gene from Bacillus cereus, or a GAPN gene ortholog, or a GAPN gene paralog.

In some embodiments, the glyceraldehyde-3-phosphate dehydrogenase can be derived from a bacteria, for example, from the genus Bacillus and, in some instances, from the species Bacillus anthracis. The glyceraldehyde-3-phosphate dehydrogenase can be encoded by the GAPN gene from Bacillus anthracis, or a GAPN gene ortholog, or a GAPN gene paralog.

In some embodiments, the glyceraldehyde-3-phosphate dehydrogenase can be derived from a bacteria, for example, from the genus Bacillus and, in some instances, from the species Bacillus thuringiensis. The glyceraldehyde-3-phosphate dehydrogenase can be encoded by the GAPN gene from Bacillus thuringiensis, or a GAPN gene ortholog, or a GAPN gene paralog.

In some embodiments, the glyceraldehyde-3-phosphate dehydrogenase can be derived from a bacteria, for example, from the genus Pyrococcus and, in some instances, from the species Pyrococcus furiosus. The glyceraldehyde-3-phosphate dehydrogenase can be encoded by the GAPN gene from Pyrococcus furiosus, or a GAPN gene ortholog, or a GAPN gene paralog.

Embodiments of glyceraldehyde-3-phosphate dehydrogenase can also be derived, without limitation, from the following (the number in brackets correspond to the Gene ID number): Triticum aestivum (543435); Streptococcus mutans (1028095); Streptococcus agalactiae (1013627); Streptococcus pyogenes (901445); Clostridioides difficile (4913365); Mycoplasma mycoides subsp. mycoides SC str. (2744894); Streptococcus pneumoniae (933338); Streptococcus sanguinis (4807521); Acinetobacter pittii (11638070); Clostridium botulinum A str. (5185508); [Bacillus thuringiensis] serovar konkukian str. (2857794); Bacillus anthracis str. Ames (1088724); Phaeodactylum tricornutum (7199937); Emiliania huxleyi (17251102); Zea mays (542583); Helianthus annuus (110928814); Streptomyces coelicolor (1101118); Burkholderia pseudomallei(3097058, 3095849); variants thereof as well as fragments thereof.

Additional embodiments of glyceraldehyde-3-phosphate dehydrogenase can also be derived, without limitation, from the following (the number in brackets correspond to the Pubmed Accession number): Streptococcus macacae (WP_003081126.1), Streptococcus hyointestinalis (W P_115269374.1), Streptococcus urinalis (W P_006739074.1), Streptococcus canis (WP_003044111.1), Streptococcus pluranimalium (WP_104967491.1), Streptococcus equi (WP_012678132.1), Streptococcus thoraltensis (WP_018380938.1), Streptococcus dysgalactiae (WP_138125971.1), Streptococcus halotolerans (WP_062707672.1), Streptococcus pyogenes (WP_136058687.1), Streptococcus ictaluri (WP_008090774.1), Clostridium perfringens (WP_142691612.1), Clostridium chromiireducens (WP_079442081.1), Clostridium botulinum (WP_012422907.1), Bacillus cereus (WP_000213623.1), Bacillus anthracis (WP_098340670.1), Bacillus thuringiensis (WP_087951472.1), Pyrococcus furiosus (WP_011013013.1) as well as variants thereof and fragments thereof.

Embodiments of glyceraldehyde-3-phosphate dehydrogenase as well as yeasts expressing same are disclosed in US patent application 20210380989, incorporated herewith in its entirety.

In an embodiment, the recombinant yeast host cells of the present disclosure are capable of decreasing the activity or the expression of a second polypeptide involved in the conversion of a biomass into a fermentation product and/or the reduction in the production of a fermentation by-product. In still another embodiment, the recombinant yeast host cells is capable of reducing the production of the fermentation by-product, such as, for example, glycerol. The recombinant yeast host cells of the present disclosure can include one or more genetic modification for decreasing the activity or the expression of a second polypeptide. In an embodiment, the genetic modification can be located in a regulatory region (such as a promoter region) of a native gene encoding a native (second) polypeptide and/or in the coding region of a native gene encoding a native (second) polypeptide. Alternatively or in combination, the genetic modification can be the introduction of one or more (second) heterologous nucleic acid molecules in the recombinant yeast host cells so as to inactivate, at least in part or totally, the expression of the second native polypeptide. In such embodiment, the second heterologous nucleic acid molecule(s) can be placed in the open-reading frame of the native gene to be inactivated. In still another embodiment, the second heterologous nucleic acid molecule(s) can be replace the open-reading frame of the native gene to be inactivated. The second genetic modification can be made in at least one (an in some embodiments in all) allele(s) of a copy of the native gene to be inactivated.

In still a further embodiment, the recombinant yeast host cells of the present disclosure are capable of reducing the activity or the expression of a (first) native NAD-dependent glycerol-3-phosphate dehydrogenase (GPD) polypeptide. Recombinant yeast host cells having decreased GPD activity have been described in US20200224209 which is herewith incorporated in its entirety.

Most mammalian cells express two different glycerol-3-phosphate dehydrogenases (GPDs) which are necessary for glycerol production and they are expressed in response to different cellular signals: the GPD1 and the GPD2 polypeptides. Both polypeptides share 75% amino acid identity and, while they catalyze the same reaction, the differences in their amino acid sequence make them more efficient enzymes under the environmental conditions that induce their expression. GPD2 is known to be unable to fully substitute for GPD1 in the production of osmotically induced glycerol production suggesting that this enzyme has lower activity than GPD1 under osmotic stress.

In an embodiment, the recombinant yeast host cells bear a genetic modification in at least one of the gpd1 gene (encoding the GPD1 polypeptide), the gpd2 gene (encoding the GPD2 polypeptide), the gpp1 gene (encoding the GPP1 polypeptide) or the gpp2gene (encoding the GPP2 polypeptide). In another embodiment, the recombinant yeast host cells bear a genetic modification in at least two of the gpd1 gene (encoding the GPD1 polypeptide), the gpd2 gene (encoding the GPD2 polypeptide), the gpp1 gene (encoding the GPP1 polypeptide) or the gpp2 gene (encoding the GPP2 polypeptide). Examples of recombinant yeast cells bearing such genetic modification(s) leading to the reduction in the production of one or more native enzymes that function to produce glycerol are described in WO 2012/138942. In some embodiments, the recombinant yeast host cells have a genetic modification (such as a genetic deletion or insertion) only in one enzyme that functions to produce glycerol, in the gpd2 gene, which would cause the yeast cell to have a knocked-out gpd2 gene. In some embodiments, the recombinant yeast host cell can have a genetic modification in the gpd1 gene and the gpd2 gene resulting is a recombinant yeast host cell being knock-out for the gpd1 gene and the gpd2 gene. In some specific embodiments, the yeast cell can be a knock-out for the gpd1 gene and have duplicate copies of the gpd2 gene (in some embodiments, under the control of the gpd1 promoter). In yet another embodiment, the recombinant yeast host cells do not bear such genetic modification and includes its native genes coding for the GPP/GDP polypeptides.

The recombinant yeast host cells of the present disclosure can include a genetic modification to inhibit (at least partially or totally) the expression of a first NAD-dependent glycerol-3-phosphate dehydrogenase. This first NAD-dependent glycerol-3-phosphate dehydrogenase can be a NAD-dependent glycerol-3-phosphate 1 (GPD1) polypeptide or a GPD1 gene ortholog or paralog. The second genetic modification can include a deletion, deletion or substitution of one or more of a nucleic acid residue(s) in a gene (or a gene ortholog) encoding the GPD1 polypeptide (particularly in the gene's coding sequence) which would cause a reduction in the activity of the GPD1 polypeptide. In an embodiment, the second genetic modification can include the deletion of all of the coding sequence of a gene (or a gene ortholog) encoding the GPD1 polypeptide. Alternatively or in combination, the recombinant yeast host cell can express a heterologous GPD1 polypeptide variant or fragment having a reduced activity when compared to the native GPD1 polypeptide.

The GPD1 polypeptide is natively expressed in yeasts, fungi, mammalian and plant cells. GPD1 genes encoding the GPD1 polypeptide include, but are not limited to Saccharomyces cerevisiae Gene ID: 851539, Schizosaccharomyces pombe Gene ID: 2540547, Schizosaccharomyces pombe Gene ID: 2540455, Neurospora crassa Gene ID: 3873099, Candida albicans Gene ID: 3643924, Scheffersomyces stipitis Gene ID: 4840320, Spathaspora passalidarum Gene ID: 18874668, Trichoderma reesei Gene ID: 18482691, Nectria haematococca Gene ID: 9668637, Candida dubliniensis Gene ID: 8046432, Chlamydomonas reinhardtii Gene ID: 5716580, Brassica napus Gene ID: 106365675, Chlorella variabilis Gene ID: 17355036, Brassica napus Gene ID: 106352802, Mus musculus Gene ID: 14555, Homo sapiens Gene ID: 2819, Rattus norvegicus Gene ID: 60666, Sus scrofa Gene ID: 100153250, Gallus gallus Gene ID: 426881, Bos taurus Gene ID: 525042, Xenopus tropicalis Gene ID: 448519, Pan troglodytes Gene ID: 741054, Canis lupus familiaris Gene ID: 607942, Callorhinchus milii Gene ID: 103188923, Columba livia Gene ID: 102088900, Macaca fascicularis Gene ID: 101865501, Myotis brandtii Gene ID: 102257341, Heterocephalus glaber Gene ID: 101702723, Nannospalax galili Gene ID: 103746543, Mustela putorius furo Gene ID: 101681348, Callithrix jacchus Gene ID: 100414900, Labrus bergylta Gene ID: 109980872, Monopterus albus Gene ID: 109969143, Castor canadensis Gene ID: 109695417, Paralichthys olivaceus Gene ID: 109635348, Bos indicus Gene ID: 109559120, Hippocampus comes Gene ID: 109507993, Rhinolophus sinicus Gene ID: 109443801, Hipposideros armiger Gene ID: 109393253, Crocodylus porosus Gene ID: 109324424, Gavialis gangeticus Gene ID: 109293349, Panthera pardus Gene ID: 109249099, Cyprinus carpio Gene ID: 109094445, Scleropages formosus Gene ID: 108931403, Nanorana parkeri Gene ID: 108789981, Rhinopithecus bieti Gene ID: 108543924, Lepidothrix coronata Gene ID: 108509436, Pygocentrus nattereri Gene ID: 108444060, Manis javanica Gene ID: 108406536, Cebus capucinus imitator Gene ID: 108316082, Ictalurus punctatus Gene ID: 108255083, Kryptolebias marmoratus Gene ID: 108231479, Miniopterus natalensis Gene ID: 107528262, Rousettus aegyptiacus Gene ID: 107514265, Coturnix japonica Gene ID: 107325705, Protobothrops mucrosquamatus Gene ID: 107302714, Parus major Gene ID: 107215690, Marmota marmota marmota Gene ID: 107148619, Gekko japonicus Gene ID: 107122513, Cyprinodon variegatus Gene ID: 107101128, Acinonyx jubatus Gene ID: 106969233, Poecilia latipinna Gene ID: 106959529, Poecilia mexicana Gene ID: 106929022, Calidris pugnax Gene ID: 106891167, Sturnus vulgaris Gene ID: 106863139, Equus asinus Gene ID: 106845052, Thamnophis sirtalis Gene ID: 106545289, Apteryx australis mantelli Gene ID: 106499434, Anser cygnoides domesticus Gene ID: 106047703, Dipodomys ordii Gene ID: 105987539, Clupea harengus Gene ID: 105897935, Microcebus murinus Gene ID: 105869862, Propithecus coquereli Gene ID: 105818148, Aotus nancymaae Gene ID: 105709449, Cercocebus atys Gene ID: 105580359, Mandrillus leucophaeus Gene ID: 105527974, Colobus angolensis palliatus Gene ID: 105507602, Macaca nemestrina Gene ID: 105492851, Aquila chrysaetos canadensis Gene ID: 105414064, Pteropus vampyrus Gene ID: 105297559, Camelus dromedarius Gene ID: 105097186, Camelus bactrianus Gene ID: 105076223, Esox lucius Gene ID: 105016698, Bison bison bison Gene ID: 105001494, Notothenia coriiceps Gene ID: 104967388, Larimichthys crocea Gene ID: 104928374, Fukomys damarensis Gene ID: 04861981, Haliaeetus leucocephalus Gene ID: 104831135, Corvus cornix cornix Gene ID: 104683744, Rhinopithecus roxellana Gene ID: 104679694, Balearica regulorum gibbericeps Gene ID: 104630128, Tinamus guttatus Gene ID: 104575187, Mesitornis unicolor Gene ID: 104539793, Antrostomus carolinensis Gene ID: 104532747, Buceros rhinoceros silvestris Gene ID: 104501599, Chaetura pelagica Gene ID: 104385595, Leptosomus discolor Gene ID: 104353902, Opisthocomus hoazin Gene ID: 104326607, Charadrius vociferus Gene ID: 104284804, Struthio camelus australis Gene ID: 104144034, Egretta garzetta Gene ID: 104132778, Cuculus canorus Gene ID: 104055090, Nipponia nippon Gene ID: 104011969, Pygoscelis adeliae Gene ID: 103914601, Aptenodytes forsteri Gene ID: 103894920, Serinus canaria Gene ID: 103823858, Manacus vitellinus Gene ID: 103760593, Ursus maritimus Gene ID: 103675473, Corvus brachyrhynchos Gene ID: 103613218, Galeopterus variegatus Gene ID: 103598969, Equus przewalskii Gene ID: 103546083, Calypte anna Gene ID: 103536440, Poecilia reticulata Gene ID: 103464660, Cynoglossus semilaevis Gene ID: 103386748, Stegastes partitus Gene ID: 103355454, Eptesicus fuscus Gene ID: 103285288, Chlorocebus sabaeus Gene ID: 103238296, Orycteropus afer afer Gene ID: 103194426, Poecilia formosa Gene ID: 103134553, Erinaceus europaeus Gene ID: 103118279, Lipotes vexillifer Gene ID: 103087725, Python bivittatus Gene ID: 103049416, Astyanax mexicanus Gene ID: 103021315, Balaenoptera acutorostrata scammoni Gene ID: 103006680, Physeter catodon Gene ID: 102996836, Panthera tigris altaica Gene ID: 102961238, Chelonia mydas Gene ID: 102939076, Peromyscus maniculatus bairdii Gene ID: 102922332, Pteropus alecto Gene ID: 102880604, Elephantulus edwardii Gene ID: 102844587, Chrysochloris asiatica Gene ID: 102825902, Myotis davidii Gene ID: 102754955, Leptonychotes weddellii Gene ID: 102730427, Lepisosteus oculatus Gene ID: 102692130, Alligator mississippiensis Gene ID: 102576126, Vicugna pacos Gene ID: 102542115, Camelus ferus Gene ID: 102507052, Tupaia chinensis Gene ID: 102482961, Pelodiscus sinensis Gene ID: 102446147, Myotis lucifugus Gene ID: 102420239, Bubalus bubalis Gene ID: 102395827, Alligator sinensis Gene ID: 102383307, Latimeria chalumnae Gene ID: 102345318, Pantholops hodgsonii Gene ID: 102326635, Haplochromis burtoni Gene ID: 102295539, Bos mutus Gene ID: 102267392, Xiphophorus maculatus Gene ID: 102228568, Pundamilia nyererei Gene ID: 102192578, Capra hircus Gene ID: 102171407, Pseudopodoces humilis Gene ID: 102106269, Zonotrichia albicollis Gene ID: 102070144, Falco cherrug Gene ID: 102047785, Geospiza fortis Gene ID: 102037409, Chinchilla lanigera Gene ID: 102014610, Microtus ochrogaster Gene ID: 101990242, Ictidomys tridecemlineatus Gene ID: 101955193, Chrysemys picta Gene ID: 101939497, Falco peregrinus Gene ID: 101911770, Mesocricetus auratus Gene ID: 101824509, Ficedula albicollis Gene ID: 101814000, Anas platyrhynchos Gene ID: 101789855, Echinops telfairi Gene ID: 101641551, Condylura cristata Gene ID: 101622847, Jaculus jaculus Gene ID: 101609219, Octodon degus Gene ID: 101563150, Sorex araneus Gene ID: 101556310, Ochotona princeps Gene ID: 101532015, Maylandia zebra Gene ID: 101478751, Dasypus novemcinctus Gene ID: 101446993, Odobenus rosmarus divergens Gene ID: 101385499, Tursiops truncatus Gene ID: 101318662, Orcinus orca Gene ID: 101284095, Oryzias latipes Gene ID: 101154943, Gorilla gorilla Gene ID: 101131184, Ovis aries Gene ID: 101119894, Felis catus Gene ID: 101086577, Takifugu rubripes Gene ID: 101079539, Saimiri boliviensis Gene ID: 101030263, Papio anubis Gene ID: 101004942, Pan paniscus Gene ID: 100981359, Otolemur garnettii Gene ID: 100946205, Sarcophilus harrisii Gene ID: 100928054, Cricetulus griseus Gene ID: 100772179, Cavia porcellus Gene ID: 100720368, Oreochromis niloticus Gene ID: 100712149, Loxodonta africana Gene ID: 100660074, Nomascus leucogenys Gene ID: 100594138, Anolis carolinensis Gene ID: 100552972, Meleagris gallopavo Gene ID: 100542199, Ailuropoda melanoleuca Gene ID: 100473892, Oryctolagus cuniculus Gene ID: 100339469, Taeniopygia guttata Gene ID: 100225600, Pongo abelii Gene ID: 100172201, Ornithorhynchus anatinus Gene ID: 100085954, Equus caballus Gene ID: 100052204, Mus musculus Gene ID: 100198, Xenopus laevis Gene ID: 399227, Danio rerio Gene ID: 325181, Danio rerio Gene ID: 406615, Melopsittacus undulatus Gene ID: 101872435, Ceratotherium simum simum Gene ID: 101408813, Trichechus manatus latirostris Gene ID: 101359849 and Takifugu rubripes Gene ID: 101071719). In the present disclosure, the recombinant yeast cell can reduce or inhibit the expression of a GDP1 gene (or a GPD1 gene ortholog) encoding a GDP1 polypeptide, variant or fragment.

The recombinant yeast host cells of the present disclosure can include a genetic modification to inhibit (at least partially or totally) the expression of a scond NAD-dependent glycerol-3-phosphate dehydrogenase. This second NAD-dependent glycerol-3-phosphate dehydrogenase can be a NAD-dependent glycerol-3-phosphate 2 (GPD2) polypeptide or a GPD2 gene ortholog or paralog. The genetic modification can include a deletion, deletion or substitution of one or more of a nucleic acid residue(s) in a gene (or a gene ortholog) encoding the GPD2 polypeptide (particularly in the gene's coding sequence) which would cause a reduction in the activity of the GPD2 polypeptide. In an embodiment, the second genetic modification can include the deletion of all of the coding sequence of a gene (or a gene ortholog) encoding the GPD2 polypeptide. Alternatively or in combination, the recombinant yeast host cell can express a heterologous GPD2 polypeptide variant or fragment having a reduced activity when compared to the native GPD2 polypeptide.

In some embodiments, the recombinant yeast host cells of the present disclosure, while having reduced activity or expression in a first NAD-dependent glycerol-3-phosphate (e.g., GPD1), can express a second NAD-dependent glycerol-3-phosphate dehydrogenase exhibiting less enzymatic activity than the first NAD-dependent glycerol-3-phosphate (e.g., GPD2). For example, the recombinant yeast host cells of the present disclosure, while having a reduced GPD1 activity or express, is capable of expressing a heterologous NAD-dependent glycerol-3-phosphate dehydrogenase 2 (GPD2) polypeptide (which exhibits less enzymatic activity than GPD1). As such, the second genetic modification can include modifying the recombinant host cells to express a heterologous NAD-dependent glycerol-3-phosphate dehydrogenase 2 (GPD2) polypeptide. This can be done, for example, by expressing a heterologous nucleic acid encoding the heterologous GPD2 polypeptide using an osmotic promoter (such as, for example, the promoter of the GPD1 gene). The second heterologous nucleic acid molecule can, in some additional embodiments, replace the open-reading frame of at least one copy of the native GPD1 gene. The second heterologous nucleic acid molecule can, in some embodiments, replace the open-reading frame of all copes of the native GPD1 gene. In some embodiments, at least a single native copy of the gene (or the gene ortholog) encoding the GPD2 polypeptide be under the control of the native GPD2 promoter.

The GPD2 polypeptide is expressed in bacteria, yeasts, fungi, mammalian and plant cells. GPD2 genes encoding the GPD2 polypeptide include, but are not limited to Mus musculus Gene ID: 14571, Homo sapiens Gene ID: 2820, Saccharomyces cerevisiae Gene ID: 854095, Rattus norvegicus Gene ID: 25062, Schizosaccharomyces pombe Gene ID: 2541502, Mus musculus Gene ID: 14380, Danio rerio Gene ID: 751628, Caenorhabditis elegans Gene ID: 3565504, Mesocricetus auratus Gene ID: 101825992, Xenopus tropicalis Gene ID: 779615, Macaca mulatta Gene ID: 697192, Bos taurus Gene ID: 504948, Canis lupus familiaris Gene ID: 478755, Cavia porcellus Gene ID: 100721200, Gallus gallus Gene ID: 424321, Pan troglodytes Gene ID: 459670, Oryctolagus cuniculus Gene ID: 100101571, Candida albicans Gene ID: 3644563, Xenopus laevis Gene ID: 444438, Macaca fascicularis Gene ID: 102127260, Ailuropoda melanoleuca Gene ID: 100482626, Cricetulus griseus Gene ID: 100766128, Heterocephalus glaber Gene ID: 101715967, Scheffersomyces stipitis Gene ID: 4838862, Ictalurus punctatus Gene ID: 108273160, Mustela putorius furo Gene ID: 101681209, Nannospalax galili Gene ID: 103741048, Callithrix jacchus Gene ID: 100409379, Lates calcarifer Gene ID: 108873068, Nothobranchius furzeri Gene ID: 07384696, Acanthisitta chloris Gene ID: 103808746, Acinonyxjubatus Gene ID: 106978985, Alligatormississippiensis Gene ID: 102562563, Alligator sinensis Gene ID: 102380394, Anas platyrhynchos, Anolis carolinensis Gene ID: 100551888, Anser cygnoides domesticus Gene ID: 106043902, Aotus nancymaae Gene ID: 105719012, Apaloderma vittatum Gene ID: 104281080, Aptenodytes forsteri Gene ID: 103893867, Apteryx australis mantelli Gene ID: 106486554, Aquila chrysaetos canadensis Gene ID: 105412526, Astyanax mexicanus Gene ID: 103029081, Austrofundulus limnaeus Gene ID: 106535816, Balaenoptera acutorostrata scammoni Gene ID: 103019768, Balearica regulorum gibbericeps, Bison bison bison Gene ID: 104988636, Bos indicus Gene ID: 109567519, Bos mutus Gene ID: 102277350, Bubalus bubalis Gene ID: 102404879, Buceros rhinoceros silvestris Gene ID: 104497001, Calidris pugnax Gene ID: 106902763, Callorhinchus milii Gene ID: 103176409, Calypte anna Gene ID: 103535222, Camelus bactrianus Gene ID: 105081921, Camelus dromedarius Gene ID: 105093713, Camelus ferus Gene ID: 102519983, Capra hircus Gene ID: 102176370, Cariama cristata Gene ID: 104154548, Castor canadensis Gene ID: 109700730, Cebus capucinus imitator Gene ID: 108316996, Cercocebus atys Gene ID: 105576003, Chaetura pelagica Gene ID: 104391744, Charadrius vociferus Gene ID: 104286830, Chelonia mydas Gene ID: 102930483, Chinchilla lanigera Gene ID: 102017931, Chlamydotis macqueenii Gene ID: 104476789, Chlorocebus sabaeus Gene ID: 103217126, Chrysemys picta Gene ID: 101939831, Chrysochloris asiatica Gene ID: 102831540, Clupea harengus Gene ID: 105902648, Colius striatus Gene ID: 104549356, Colobus angolensis palliatus Gene ID: 105516852, Columba livia Gene ID: 102090265, Condylura cristata Gene ID: 101619970, Corvus brachyrhynchos, Coturnix japonica Gene ID: 107316969, Crocodylus porosus Gene ID: 109322895, Cuculus canorus Gene ID: 104056187, Cynoglossus semilaevis Gene ID: 103389593, Dasypus novemcinctus Gene ID: 101428842, Dipodomys ordii Gene ID: 105996090, Echinops telfairi Gene ID: 101656272, Egretta garzetta Gene ID: 104135263, Elephantulus edwardii Gene ID: 102858276, Eptesicus fuscus Gene ID: 103283396, Equus asinus Gene ID: 106841969, Equus caballus Gene ID: 100050747, Equus przewalskii Gene ID: 103558835, Erinaceus europaeus Gene ID: 103114599, Eurypyga helias Gene ID: 104502666, Falco cherrug Gene ID: 102054715, Falco peregrinus Gene ID: 101912742, Felis catus Gene ID: 101089953, Ficedula albicollis Gene ID: 101816901, Fukomys damarensis Gene ID: 104850054, Fundulus heteroclitus Gene ID: 105936523, Galeopterus variegatus Gene ID: 103586331, Gavia stellata Gene ID: 104250365, Gavialis gangeticus Gene ID: 109301301, Gekko japonicus Gene ID: 107110762, Geospiza fortis Gene ID: 102042095, Gorilla gorilla Gene ID: 101150526, Haliaeetus albicilla Gene ID: 104323154, Haliaeetus leucocephalus Gene ID: 104829038, Haplochromis burtoni Gene ID: 102309478, Hippocampus comes Gene ID: 109528375, Hipposideros armiger Gene ID: 109379867, Ictidomys tridecemlineatus Gene ID: 101965668, Jaculus jaculus Gene ID: 101616184, Kryptolebias marmoratus Gene ID: 108251075, Labrus bergylta Gene ID: 109984158, Larimichthys crocea Gene ID: 104929094, Latimeria chalumnae Gene ID: 102361446, Lepidothrix coronata Gene ID: 108501660, Lepisosteus oculatus Gene ID: 102691231, Leptonychotes weddellii Gene ID: 102739068, Leptosomus discolor Gene ID: 104340644, Lipotes vexillifer Gene ID: 103074004, Loxodonta africana Gene ID: 100654953, Macaca nemestrina Gene ID: 105493221, Manacus vitellinus Gene ID: 103757091, Mandrillus leucophaeus Gene ID: 105548063, Manis javanica Gene ID: 108392571, Marmota marmota marmota Gene ID: 107136866, Maylandia zebra Gene ID: 101487556, Mesitornis unicolor Gene ID: 104545943, Microcebus murinus Gene ID: 105859136, Microtus ochrogaster Gene ID: 101999389, Miniopterus natalensis Gene ID: 107525674, Monodelphis domestica Gene ID: 100014779, Monopterus albus Gene ID: 109957085, Myotis brandtii Gene ID: 102239648, Myotis davidii Gene ID: 102770109, Myotis lucifugus Gene ID: 102438522, Nanorana parkeri Gene ID: 108784354, Nestor notabilis Gene ID: 104399051, Nipponia nippon Gene ID: 104012349, Nomascus leucogenys Gene ID: 100590527, Notothenia coriiceps Gene ID: 104964156, Ochotona princeps Gene ID: 101530736, Octodon degus Gene ID: 101591628, Odobenus rosmarus divergens Gene ID: 101385453, Oncorhynchus kisutch Gene ID: 109870627, Opisthocomus hoazin Gene ID: 104338567, Orcinus orca Gene ID: 101287409, Oreochromis niloticus Gene ID: 100694147, Ornithorhynchus anatinus Gene ID: 100081433, Orycteropus afer afer Gene ID: 103197834, Oryzias latipes Gene ID: 101167020, Otolemur garnettii Gene ID: 100966064, Ovis aries Gene ID: 443090, Pan paniscus Gene ID: 100970779, Panthera pardus Gene ID: 109271431, Panthera tigris altaica Gene ID: 102957949, Pantholops hodgsonii Gene ID: 102323478, Papio anubis Gene ID: 101002517, Paralichthys olivaceus Gene ID: 109631046, Pelodiscus sinensis Gene ID: 102454304, Peromyscus maniculatus bairdii Gene ID: 102924185, Phaethon lepturus Gene ID: 104624271, Phalacrocorax carbo Gene ID: 104049388, Physeter catodon Gene ID: 102978831, Picoides pubescens Gene ID: 104296936, Poecilialatipinna Gene ID: 106958025, Poecilia mexicana Gene ID: 106920534, Poecilia reticulata Gene ID: 103473778, Pongo abelii Gene ID: 100452414, Propithecus coquereli Gene ID: 105807399, Protobothrops mucrosquamatus Gene ID: 107289584, Pseudopodoces humilis Gene ID: 102109711, Pterocles gutturalis Gene ID: 104461236, Pteropus alecto Gene ID: 102879110, Pteropus vampyrus Gene ID: 105291402, Pundamilia nyererei Gene ID: 102200268, Pygocentrus nattereri Gene ID: 108411786, Pygoscelis adeliae Gene ID: 103925329, Python bivittatus Gene ID: 103059167, Rhincodon typus Gene ID: 109920450, Rhinolophus sinicus Gene ID: 109445137, Rhinopithecus bieti Gene ID: 108538766, Rhinopithecus roxellana Gene ID: 104654108, Rousettus aegyptiacus Gene ID: 107513424, Saimiri boliviensis Gene ID: 101027702, Salmo salar Gene ID: 106581822, Sarcophilus harrisii Gene ID: 100927498, Scleropages formosus Gene ID: 108927961, Serinus canaria Gene ID: 103814246, Sinocyclocheilus grahami Gene ID: 107555436, Sorex araneus Gene ID: 101543025, Stegastes partitus Gene ID: 103360018, Struthio camelus australis Gene ID: 104138752, Sturnus vulgaris Gene ID: 106861926, Sugiyamaella lignohabitans Gene ID: 30033324, Sus scrofa Gene ID: 397348, Taeniopygia guttata Gene ID: 100222867, Takifugu rubripes Gene ID: 101062218, Tarsius syrichta Gene ID: 103254049, Tauraco erythrolophus Gene ID: 104378162, Thamnophis sirtalis Gene ID: 106538827, Tinamus guttatus Gene ID: 104572349, Tupaia chinensis Gene ID: 102471148, Tursiops truncatus Gene ID: 101330605, Ursus maritimus Gene ID: 103659477, Vicugna pacos Gene ID: 102533941, Xiphophorus maculatus Gene ID: 102225536, Zonotrichia albicollis Gene ID: 102073261, Ciona intestinalis Gene ID: 100183886, Meleagris gallopavo Gene ID: 100546408, Trichechus manatus latirostris Gene ID: 101355771, Ceratotherium simum simum Gene ID: 101400784, Melopsittacus undulatus Gene ID: 101871704, Esox lucius Gene ID: 10502249 and Pygocentrus nattereri Gene ID: 108411786. In an embodiment, the GPD2 polypeptide is encoded by Saccharomyces cerevisiae Gene ID: 854095. In some embodiments, the GPD2 polypeptide has the amino acid sequence of SEQ ID NO: 6, is a variant of the amino acid sequence of SEQ ID NO: 6 having NAD-dependent glycerol-3-phosphate activity or is a fragment of the amino acid sequence of SEQ ID NO: 6 having NAD-dependent glycerol-3-phosphate activity. In some embodiments, the second heterologous nucleic acid molecule comprises the nucleic acid sequence of SEQ ID NO: 5, is a degenerate sequence of SEQ ID NO: 5 (encoding SEQ ID NO: 6) or encodes a variant or a fragment of the amino acid sequence of SEQ ID NO: 6.

In some embodiments, this overall reduction in GPD activity can be observed in high osmotic conditions. As used herein, the expression “high osmotic conditions” refers to the presence of a high osmotic pressure, usually caused by an increase in the solute concentration in the environment surrounding the recombinant yeast host cell. In yeasts, “high osmotic conditions” are associated with an upregulation of the HOG pathway, a concentration of sugars higher than about 50 g/L and/or equivalent to at least 1 g/L of a salt (such as NaCl). This decrease in NAD-dependent glycerol-3-phosphate activity can be observed with respect to the same recombinant yeast cell in normal or low osmotic conditions or with respect to a recombinant yeast host cell lacking the one or more second genetic modification. As also used in the present disclosure, the expression “normal or low osmotic conditions” refers to conditions that are not associated with high osmotic pressure.

The second heterologous nucleic acid molecules of the present disclosure can include or be operatively associated with an osmotic promoter. In the context of the present disclosure, an “osmotic promoter” can be a promoter (or a combination of promoters) allowing the expression (or, in some embodiments, the overexpression) of a gene when the recombinant yeast host cell is placed in high osmotic conditions but refraining the expression (or, in some embodiments, the overexpression) of a gene when the recombinant yeast host cell is placed in normal or low osmotic conditions. In this embodiment, the osmotic promoter can be an inducible promoter. Osmotic promoters are usually associated with genes in the HOG1 pathway and promoters controlling the expression of genes which are upregulated in the HOG1 pathway can be used in the recombinant yeast host cell of the present disclosure. Enzymes in the HOG1 pathway whose expression is upregulated in high osmotic conditions include, but are not limited to, a NAD-dependent glycerol-3-phosphate dehydrogenase 1 gene, a dihydroxyacetone kinase gene and a trehalose-phosphatase gene. As such, in the context of the present disclosure, the osmotic promoter can be a promoter (or a combination of promoters) from a NAD-dependent glycerol-3-phosphate dehydrogenase 1 gene, a dihydroxyacetone kinase gene and/or a trehalose-phosphatase gene. In Saccharomyces cerevisiae, enzymes in the HOG1 pathway whose expression is upregulated in the presence of high osmotic conditions include, but are not limited to, a GPD1 gene, a DAK1 gene and a TPS2 gene. As such, in the context of the present disclosure, the osmotic promoter can be a promoter (or a combination of promoters) from a GPD1 gene (referred to as the GPD1 promoter or gpd1p), a DAK1 gene (referred to as the DAK1 promoter or dak1p) and/or a TPS2 gene (referred to as the TPS2 promoter or tps2p).

An “osmotic promoter” can also be a constitutive promoter which allows the expression of coding sequences operatively associated thereto during osmotic conditions. In some embodiments, it is preferred that the constitutive promoter be a “low” constitutive promoter. Exemplary “low” constitutive promoters could be associated with the expression of housekeeping genes, and, for example, can include the promoter of the CYC1 gene. In some embodiment, the osmotic promoter is not a high constitutive promoter.

In yet another embodiment, the recombinant yeast host cells of the present disclosure are capable of reducing the activity or the expression of a second polypeptide capable of exporting glycerol from the recombinant yeast host cell. Exemplary polypeptides capable of functioning to export glycerol include aquaporins as well as glycerol facilitators. The fdpl support (FPS1) polypeptide (encoded by Gene ID 850683 in Saccharomyces cerevisiae) is a glycerol facilitator capable of importing glycerol. As such, the polypeptide capable of functioning to export glycerol can be a FPS1 polypeptide or a polypeptide encoded by a FPS1 gene ortholog. The FPS1 polypeptide can be derived, for example, from Saccharomyces cerevisiae or a corresponding ortholog found in Pachysolen tannophilus, Komagataella pastoris, Yarrowia lipolytica and/or Cyberlindnera jadinii.

In the present disclosure, it is possible to express a variant of a first polypeptide or of a second polypeptide in the recombinant yeast host cells. A variant comprises at least one amino acid difference (substitution or addition) when compared to the amino acid sequence of the wild type polypeptide and still exhibits the biological activity of the wild type polypeptide (e.g., for a STL1 variant, glycerol transport activity; for a glucoamylase, a starch-degrading activity; for a GPD2 variant, a NAD-depending glycerol-3-phosphate dehydrogenase activity, etc.). In an embodiment, the variant polypeptide exhibits at least 10%, 15%, 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% of the activity of the wild-type polypeptide. The variants also have at least 70%, 80%, 81%, 82%, 83%, 84%, 85%, 86%, 87%, 88%, 89%, 90%, 91%, 92%, 93%, 94%, 95%, 96%, 97%, 98% or 99% identity when compared to the wild-type polypeptide over its entire length. The term “percent identity”, as known in the art, is a relationship between two or more polypeptide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant polypeptides described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative amino acid substitutions are known in the art and are included herein. Non-conservative substitutions, such as replacing a basic amino acid with a hydrophobic one, are also well-known in the art.

A variant polypeptide can also be a conservative variant or an allelic variant. As used herein, a conservative variant refers to alterations in the amino acid sequence that do not adversely affect the biological functions of the variant polypeptide. A substitution, insertion or deletion is said to adversely affect the polypeptide when the altered sequence prevents or disrupts a biological function associated with the variant polypeptide. For example, the overall charge, structure or hydrophobic-hydrophilic properties of the polypeptide can be altered without adversely affecting a biological activity. Accordingly, the amino acid sequence can be altered, for example to render the variant polypeptide more hydrophobic or hydrophilic, without adversely affecting its biological activity.

The present disclosure also provide fragments of the first and/or the second polypeptide described herein. A fragment comprises at least one less amino acid residue when compared to the amino acid sequence of the wild type polypeptide or variant and still possess the biological activity of the full-length wild type polypeptide. In an embodiment, the fragment exhibits at least 10%, 15%, 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% activity when compared to the full-length wild type polypeptide or variant. The fragments can also have at least 70%, 80%, 81%, 82%, 83%, 84%, 85%, 86%, 87%, 88%, 89%, 90%, 91%, 92%, 93%, 94%, 95%, 96%, 97%, 98% or 99% identity when compared to the wild type polypeptide or the variant. The fragment can be, for example, a truncation of one or more amino acid residues at the amino-terminus, the carboxy terminus or both termini of the wild type polypeptide or variant. Alternatively or in combination, the fragment can be generated from removing one or more internal amino acid residues.

### Persistent Yeast Compositions and Processes for Propagating Persistent Yeast Cells

The present disclosure provides compositions comprising the persistent yeast cells as well as processes for making such compositions. Broadly, the process for making the yeast composition comprises two steps: a first step of propagating the persistent yeast cells and a second step of formulating the persistent yeast cells into a composition. It is understood that the process for making the fermented product (described herein below in more details) can include some propagation of the persistent yeast cells but mainly concerns converting the biomass into the fermentation product. The propagation step of the process for making the persistent yeast cell composition minimizes the conversion of the biomass into the fermentation product and concerns mainly concerns maximizing cellular division of the persistent yeast cells. The propagation can be performed by sampling the fermentation medium of an initial and/or a further fermentation (obtained, for example, at steps 200 or 400 described in FIG. 1) or the substantially isolated persistent yeast host cells (obtained, for example, at steps 300 and/or 500 of FIG. 1). The sample comprises persistent yeast cells. In some embodiments, the sample of the fermentation medium and/or the substantially isolated persistent yeast host cell can be placed into contact directly with a propagation medium allowing propagation in order to be propagated. In such embodiment, the propagation medium can be a fresh medium and/or also allow fermentation. The propagation medium can include, for example, molasses, cane juice, one or more nutrient and/or one or more antibiotic. In additional embodiments, the sample of the fermentation medium and/or the substantially isolated persistent yeast host cell can be placed directly in a fermentor or, in further embodiments, in a small vessel (such as, for example, a shake flask) to scale up the propagation prior to fermentation. In some embodiments, the sample of the fermentation medium and/or the substantially isolated persistent yeast host cell can be placed into contact with a solid medium (e.g., an agar plate for example) prior to propagation. Prior to being placed in a medium allowing for propagation, the sampled persistent yeast cells can be diluted or washed (with water for example) and/or concentrated (with centrifugation or filtration for example). The sampled persistent yeast cells can, prior to or during propagation, be supplemented with one or more nutrient or one or more antibiotic to maintain or prolong viability. The sampled persistent yeast cells can, prior to propagation, be stored. After having been propagated, the sampled persistent yeast cells can be diluted or washed (with water for example) and/or concentrated (with centrifugation or filtration for example).

The propagation can be conducted according to a traditional baker's yeast production process with the persistent yeast cells as described herein. The propagation step can be a continuous propagation, a batch propagation or a fed-batch propagation. The propagation medium intended to be inoculated with the persistent yeast cells can comprise a carbon source (such as, for example, molasses, sucrose, glucose, dextrose syrup, ethanol, corn, glycerol, corn steep liquor and/or a lignocellulosic biomass), a nitrogen source (such as, for example, ammonia or another inorganic source of nitrogen) and a phosphorous source (such as, for example, phosphoric acid or another inorganic source of phosphorous). The propagation medium can further comprise additional micronutrients such as vitamins and/or minerals to support the propagation of the persistent yeast cells. In some embodiments, the propagation medium can comprise molasses or be derived from molasses.

In the propagation process, the persistent yeast cells are placed in a propagation medium which can, in some embodiments, allow for a specific growth rate of 0.25, 0.24, 0.23, 0.22, 0.21, 0.20, 0.19, 0.18, 0.17, 0.16 or 0.15 h⁻¹ or less. In order to limit the growth rate of the persistent yeast cells, in some embodiments, the process can further comprise controlling the addition of nutriments, such as carbohydrates, during the propagation step. Limiting the growth rate of the persistent yeast cells during propagation can be achieved by maintaining the concentration of carbohydrates below 0.1, 0.01, 0.001 or 0.0001 weight % with respect to the volume of the fermentation medium. Controlling the concentration of carbohydrates of the propagation medium can be done by various means known in the art and can involve sampling the propagation medium at various intervals, determining the carbohydrate concertation, fermentation product (e.g., alcohol) concentration and/or gas (e.g., CO₂) concentration in those samples and adding or refraining from adding, if necessary, additional carbohydrates in the propagation medium to accelerate or decelerate the growth of the persistent yeast cells. In some embodiments, the process provides for adding nitrogen and/or phosphorous to match/support the growth rate of the persistent yeast cells.

The propagation process is preferably conducted under high aeration conditions. For example, in some embodiments, the process can include controlling the aeration of the propagation medium (which is contained in a vessel of a specific volume) to achieve a specific aeration rate, for example, of at least 0.2, 0.3, 0.4, 0.5, 0.6, 0.7, 0.8, 0.9, 1.0, 1.1, 1.2 or 1.3 air volume/vessel volume/minute.

The propagation process can be conducted at a specific pH and/or a specific temperature which may be optimal for the propagation of the persistent yeast cells. As such, in embodiments in which the persistent yeast cell is from the genus Saccharomyces, the process can comprise controlling the pH of the propagation medium to between about 3.0 to about 6.0, about 3.5 to about 5.5 or about 4.0 to about 5.5. In a specific embodiment, the pH is controlled at about 4.5. In another example, in embodiments in which the persistent yeast cell is from the genus Saccharomyces, the process can comprise controlling the temperature of the propagation medium between about about 20° C. to about 40° C., about 25° C. to about 30° C. or about 30° C. to about 35° C. In a specific embodiment, the temperature is controlled at between about about 30° C. to about 35° C. (32° C. for example).

At the end of the propagation step, a propagated medium comprising propagated persistent yeast cells (which can be propagated recombinant yeast host cells) is obtained. In some embodiments, a specific concentration of the propagated persistent yeast cells can be sought or achieved in the propagated medium. In some embodiments, the concentration of the propagated persistent yeast cells is at least about 0.1, 0.15, 0.2, 0.25, 0.3, 0.35, 0.4, 0.45, 0.5, 1.0, 1.5, 2.0, 2.5, 3.0, 3.5, 4.0, 4.5, 5.0 or more weight % with respect to the volume of the propagated medium. In a specific embodiment in which the persistent yeast cells are propagated using a fed-batch process, the concentration of the propagated persistent yeast cells is at least about 0.25 weight % with respect to the volume of the propagated medium.

In the formulating step, the mixture obtained after propagation (comprising the propagated persistent yeast cells) is modified to provide a yeast composition. In an embodiment of the formulating step, at least one component of the mixture obtained after propagation is removed from the propagated medium to provide the yeast composition. This component can be, without limitation, water, amino acids, peptides and proteins, nucleic acid residues and nucleic acid molecules, cellular debris, fermentation products, etc. In an embodiment, the formulating step comprises substantially isolating the propagated persistent yeast cells (e.g., the biomass) from the components of the propagated medium. As used in the context of the of the formulating step, the expression “substantially isolating” refers to the removal of the majority of the components of the propagated medium from the propagated persistent yeast cells. In some embodiments, “substantially isolating” refers to concentrating the propagated persistent yeast cells to at least 5, 10, 15, 20, 25, 30, 35, 45% or more when compared to the concentration of the persistent yeast cells prior to the substantial isolation. In order to provide the yeast composition, the propagated yeasts can be centrifuged (and the resulting cellular pellet comprising the propagated persistent yeast cells can optionally be washed), filtered and/or dried (optionally using a vacuum-drying technique). The formulation step can, in some embodiments, preserve the viability (at least in part) of the recombinant yeast host cells. As such, the yeast composition can be provided in an active or a semi-active form. The yeast composition can be provided in a liquid, semi-solid or dry form. In an embodiment, the composition can be provided in the form of a cream yeast.

### Processes for Prolonging Persistence

The persistent yeast cells of the present disclosure are useful because their presence in a fermenting population over a plurality of fermentation cycles (in which the fermenting population is recycled) is prolonged. This prolonged presence is due, at least in part, by the presence of the one or more phenotypic traits present in the persistent yeast cells. As shown in the FIG. 1 and explained above, the plurality of fermentation cycles comprises at least one initial fermentation cycle and at least one or more further fermentation cycles. In the processes of the present disclosure, the persistent yeast cells are only exogenously added in the initial fermentation cycle and are then recycled in further fermentation cycles. Each fermentation cycle of the process includes contacting a fermentation medium (comprising a fermentable carbohydrate) with a fermenting population under conditions so as to allow the conversion of the fermentable carbohydrate in a fermentation product (e.g., fermentation). At the end of the fermentation, the fermenting population present in the fermented fermentation medium is substantially isolated from the fermented fermentation medium and use to initiate another fermentation cycle. It is understood that initial fermenting population consists essentially in the persistent yeast cells of the present disclosure and that, during the plurality of the fermentation cycles, the recycled fermenting population can include some contaminating wild (non-genetically modified) yeasts. The persistent yeast cells of the present disclosure persist for a longer time in the plurality of the fermentation cycles when compared to a control recombinant yeast host cell (having the ability to modulate the activity or the expression of the first and/or the second polypeptide) and lacking the phenotypic traits of the persistent yeast cell of the present disclosure. The plurality of fermentation cycles can include at least one continuous fermentation. The plurality of fermentation cycles can only include continuous fermentations. The plurality of fermentation cycles can include at least one batch fermentation. The plurality of fermentation cycles can only include batch fermentations. The processes of the present disclosure can include an initial fermentation cycle at least one, two, three, four, five, six, seven, eight, nine, 10, 15, 20, 25, 30, 35, 40,45, 50, 55, 60, 65, 70, 75, 80, 85, 90, 95, 100, 105, 110, 115, 120, 125, 130, 135, 140,145, 150, 155, 160, 165, 170, 175, 180, 185, 190, 195, 200 or more further fermentation cycles. In a specific embodiments, the processes of the present disclosure include an initial fermentation cycle at least 39 further fermentation cycles. In a specific embodiments, the processes of the present disclosure include an initial fermentation cycle at least 49 further fermentation cycles. In a specific embodiments, the processes of the present disclosure include an initial fermentation cycle at least 59 further fermentation cycles. In a specific embodiments, the processes of the present disclosure include an initial fermentation cycle at least 69 further fermentation cycles. In a specific embodiments, the processes of the present disclosure include an initial fermentation cycle at least 79 further fermentation cycles. In a specific embodiments, the processes of the present disclosure include an initial fermentation cycle at least 89 further fermentation cycles. In a specific embodiments, the processes of the present disclosure include an initial fermentation cycle at least 99 further fermentation cycles. In a specific embodiments, the processes of the present disclosure include an initial fermentation cycle at least 109 further fermentation cycles. In a specific embodiments, the processes of the present disclosure include an initial fermentation cycle at least 119 further fermentation cycles. In a specific embodiments, the processes of the present disclosure include an initial fermentation cycle at least 129 further fermentation cycles. In a specific embodiments, the processes of the present disclosure include an initial fermentation cycle at least 139 further fermentation cycles. In a specific embodiments, the processes of the present disclosure include an initial fermentation cycle at least 149 further fermentation cycles. In a specific embodiments, the processes of the present disclosure include an initial fermentation cycle at least 159 further fermentation cycles. In a specific embodiments, the processes of the present disclosure include an initial fermentation cycle at least 169 further fermentation cycles. In a specific embodiments, the processes of the present disclosure include an initial fermentation cycle at least 179 further fermentation cycles. In a specific embodiments, the processes of the present disclosure include an initial fermentation cycle at least 189 further fermentation cycles. In a specific embodiments, the processes of the present disclosure include an initial fermentation cycle at least 199 further fermentation cycles.

In an embodiment, after a total 40 fermentation cycles (comprising both the initial fermentation cycle and 39 further fermentation cycles) according to the present process, the persistent yeast cells of the present disclosure are present in a proportion of at least 90% in the fermenting population (when measured with respect to its DNA contribution of the total fermenting population). In an embodiment, after a total 40 fermentation cycles (comprising both the initial fermentation cycle and 39 further fermentation cycles) according to the present process, the persistent yeast cells of the present disclosure are present in a proportion of at least 91% in the fermenting population (when measured with respect to its DNA contribution of the total fermenting population). In an embodiment, after a total 40 fermentation cycles (comprising both the initial fermentation cycle and 39 further fermentation cycles) according to the present process, the persistent yeast cells of the present disclosure are present in a proportion of at least 92% in the fermenting population (when measured with respect to its DNA contribution of the total fermenting population). In an embodiment, after a total 40 fermentation cycles (comprising both the initial fermentation cycle and 39 further fermentation cycles of the total fermenting population) according to the present process, the persistent yeast cells of the present disclosure are present in a proportion of at least 93% in the fermenting population (when measured with respect to its DNA contribution of the total fermenting population). In an embodiment, after a total 40 fermentation cycles (comprising both the initial fermentation cycle and 39 further fermentation cycles of the total fermenting population) according to the present process, the persistent yeast cells of the present disclosure are present in a proportion of at least 94% in the fermenting population (when measured with respect to its DNA contribution of the total fermenting population). In an embodiment, after a total 40 fermentation cycles (comprising both the initial fermentation cycle and 39 further fermentation cycles of the total fermenting population) according to the present process, the persistent yeast cells of the present disclosure are present in a proportion of at least 95% in the fermenting population (when measured with respect to its DNA contribution of the total fermenting population). In an embodiment, after a total 40 fermentation cycles (comprising both the initial fermentation cycle and 39 further fermentation cycles of the total fermenting population) according to the present process, the persistent yeast cells of the present disclosure are present in a proportion of at least 96% in the fermenting population (when measured with respect to its DNA contribution of the total fermenting population). In an embodiment, after a total 40 fermentation cycles (comprising both the initial fermentation cycle and 39 further fermentation cycles) according to the present process, the persistent yeast cells of the present disclosure are present in a proportion of at least 97% in the fermenting population (when measured with respect to its DNA contribution of the total fermenting population). In an embodiment, after a total 40 fermentation cycles (comprising both the initial fermentation cycle and 39 further fermentation cycles of the total fermenting population) according to the present process, the persistent yeast cells of the present disclosure are present in a proportion of at least 98% in the fermenting population (when measured with respect to its DNA contribution of the total fermenting population). In an embodiment, after a total 40 fermentation cycles (comprising both the initial fermentation cycle and 39 further fermentation cycles) according to the present process, the persistent yeast cells of the present disclosure are present in a proportion of at least 99% in the fermenting population (when measured with respect to its DNA contribution of the total fermenting population). In an embodiment, after a total 40 fermentation cycles (comprising both the initial fermentation cycle and 39 further fermentation cycles) according to the present process, the persistent yeast cells of the present disclosure are present in a proportion of at least 99.1% in the fermenting population (when measured with respect to its DNA contribution of the total fermenting population). In an embodiment, after a total 40 fermentation cycles (comprising both the initial fermentation cycle and 39 further fermentation cycles) according to the present process, the persistent yeast cells of the present disclosure are present in a proportion of at least 99.2% in the fermenting population (when measured with respect to its DNA contribution of the total fermenting population). In an embodiment, after a total 40 fermentation cycles (comprising both the initial fermentation cycle and 39 further fermentation cycles) according to the present process, the persistent yeast cells of the present disclosure are present in a proportion of at least 99.3% in the fermenting population (when measured with respect to its DNA contribution of the total fermenting population). In an embodiment, after a total 40 fermentation cycles (comprising both the initial fermentation cycle and 39 further fermentation cycles) according to the present process, the persistent yeast cells of the present disclosure are present in a proportion of at least 99.4% in the fermenting population (when measured with respect to its DNA contribution of the total fermenting population). In an embodiment, after a total 40 fermentation cycles (comprising both the initial fermentation cycle and 39 further fermentation cycles) according to the present process, the persistent yeast cells of the present disclosure are present in a proportion of at least 99.5% in the fermenting population (when measured with respect to its DNA contribution of the total fermenting population). In an embodiment, after a total 40 fermentation cycles (comprising both the initial fermentation cycle and 39 further fermentation cycles) according to the present process, the persistent yeast cells of the present disclosure are present in a proportion of at least 99.6% in the fermenting population (when measured with respect to its DNA contribution of the total fermenting population). In an embodiment, after a total 40 fermentation cycles (comprising both the initial fermentation cycle and 39 further fermentation cycles) according to the present process, the persistent yeast cells of the present disclosure are present in a proportion of at least 99.7% in the fermenting population (when measured with respect to its DNA contribution of the total fermenting population). In an embodiment, after a total 40 fermentation cycles (comprising both the initial fermentation cycle and 39 further fermentation cycles) according to the present process, the persistent yeast cells of the present disclosure are present in a proportion of at least 99.8% in the fermenting population (when measured with respect to its DNA contribution of the total fermenting population). In an embodiment, after a total 40 fermentation cycles (comprising both the initial fermentation cycle and 39 further fermentation cycles) according to the present process, the persistent yeast cells of the present disclosure are present in a proportion of at least 99.9% in the fermenting population (when measured with respect to its DNA contribution of the total fermenting population).

In an embodiment, after a total 50 fermentation cycles (comprising both the initial fermentation cycle and 49 further fermentation cycles) according to the present process, the persistent yeast cells of the present disclosure are present in a proportion of at least 90% in the fermenting population (when measured with respect to its DNA contribution of the total fermenting population). In an embodiment, after a total 50 fermentation cycles (comprising both the initial fermentation cycle and 49 further fermentation cycles) according to the present process, the persistent yeast cells of the present disclosure are present in a proportion of at least 91% in the fermenting population (when measured with respect to its DNA contribution of the total fermenting population). In an embodiment, after a total 50 fermentation cycles (comprising both the initial fermentation cycle and 49 further fermentation cycles) according to the present process, the persistent yeast cells of the present disclosure are present in a proportion of at least 92% in the fermenting population (when measured with respect to its DNA contribution of the total fermenting population). In an embodiment, after a total 50 fermentation cycles (comprising both the initial fermentation cycle and 49 further fermentation cycles of the total fermenting population) according to the present process, the persistent yeast cells of the present disclosure are present in a proportion of at least 93% in the fermenting population (when measured with respect to its DNA contribution of the total fermenting population). In an embodiment, after a total 50 fermentation cycles (comprising both the initial fermentation cycle and 49 further fermentation cycles of the total fermenting population) according to the present process, the persistent yeast cells of the present disclosure are present in a proportion of at least 94% in the fermenting population (when measured with respect to its DNA contribution of the total fermenting population). In an embodiment, after a total 50 fermentation cycles (comprising both the initial fermentation cycle and 49 further fermentation cycles of the total fermenting population) according to the present process, the persistent yeast cells of the present disclosure are present in a proportion of at least 95% in the fermenting population (when measured with respect to its DNA contribution of the total fermenting population). In an embodiment, after a total 50 fermentation cycles (comprising both the initial fermentation cycle and 49 further fermentation cycles of the total fermenting population) according to the present process, the persistent yeast cells of the present disclosure are present in a proportion of at least 96% in the fermenting population (when measured with respect to its DNA contribution of the total fermenting population). In an embodiment, after a total 50 fermentation cycles (comprising both the initial fermentation cycle and 49 further fermentation cycles) according to the present process, the persistent yeast cells of the present disclosure are present in a proportion of at least 97% in the fermenting population (when measured with respect to its DNA contribution of the total fermenting population). In an embodiment, after a total 50 fermentation cycles (comprising both the initial fermentation cycle and 49 further fermentation cycles of the total fermenting population) according to the present process, the persistent yeast cells of the present disclosure are present in a proportion of at least 98% in the fermenting population (when measured with respect to its DNA contribution of the total fermenting population). In an embodiment, after a total 50 fermentation cycles (comprising both the initial fermentation cycle and 49 further fermentation cycles) according to the present process, the persistent yeast cells of the present disclosure are present in a proportion of at least 99% in the fermenting population (when measured with respect to its DNA contribution of the total fermenting population). In an embodiment, after a total 50 fermentation cycles (comprising both the initial fermentation cycle and 49 further fermentation cycles) according to the present process, the persistent yeast cells of the present disclosure are present in a proportion of at least 99.1% in the fermenting population (when measured with respect to its DNA contribution of the total fermenting population). In an embodiment, after a total 50 fermentation cycles (comprising both the initial fermentation cycle and 49 further fermentation cycles) according to the present process, the persistent yeast cells of the present disclosure are present in a proportion of at least 99.2% in the fermenting population (when measured with respect to its DNA contribution of the total fermenting population). In an embodiment, after a total 50 fermentation cycles (comprising both the initial fermentation cycle and 49 further fermentation cycles) according to the present process, the persistent yeast cells of the present disclosure are present in a proportion of at least 99.3% in the fermenting population (when measured with respect to its DNA contribution of the total fermenting population). In an embodiment, after a total 50 fermentation cycles (comprising both the initial fermentation cycle and 49 further fermentation cycles) according to the present process, the persistent yeast cells of the present disclosure are present in a proportion of at least 99.4% in the fermenting population (when measured with respect to its DNA contribution of the total fermenting population). In an embodiment, after a total 50 fermentation cycles (comprising both the initial fermentation cycle and 49 further fermentation cycles) according to the present process, the persistent yeast cells of the present disclosure are present in a proportion of at least 99.5% in the fermenting population (when measured with respect to its DNA contribution of the total fermenting population). In an embodiment, after a total 50 fermentation cycles (comprising both the initial fermentation cycle and 49 further fermentation cycles) according to the present process, the persistent yeast cells of the present disclosure are present in a proportion of at least 99.6% in the fermenting population (when measured with respect to its DNA contribution of the total fermenting population). In an embodiment, after a total 50 fermentation cycles (comprising both the initial fermentation cycle and 49 further fermentation cycles) according to the present process, the persistent yeast cells of the present disclosure are present in a proportion of at least 99.7% in the fermenting population (when measured with respect to its DNA contribution of the total fermenting population). In an embodiment, after a total 50 fermentation cycles (comprising both the initial fermentation cycle and 49 further fermentation cycles) according to the present process, the persistent yeast cells of the present disclosure are present in a proportion of at least 99.8% in the fermenting population (when measured with respect to its DNA contribution of the total fermenting population). In an embodiment, after a total 50 fermentation cycles (comprising both the initial fermentation cycle and 49 further fermentation cycles) according to the present process, the persistent yeast cells of the present disclosure are present in a proportion of at least 99.9% in the fermenting population (when measured with respect to its DNA contribution of the total fermenting population).

In an embodiment, after a total 60 fermentation cycles (comprising both the initial fermentation cycle and 59 further fermentation cycles) according to the present process, the persistent yeast cells of the present disclosure are present in a proportion of at least 90% in the fermenting population (when measured with respect to its DNA contribution of the total fermenting population). In an embodiment, after a total 60 fermentation cycles (comprising both the initial fermentation cycle and 59 further fermentation cycles) according to the present process, the persistent yeast cells of the present disclosure are present in a proportion of at least 91% in the fermenting population (when measured with respect to its DNA contribution of the total fermenting population). In an embodiment, after a total 60 fermentation cycles (comprising both the initial fermentation cycle and 59 further fermentation cycles) according to the present process, the persistent yeast cells of the present disclosure are present in a proportion of at least 92% in the fermenting population (when measured with respect to its DNA contribution of the total fermenting population). In an embodiment, after a total 60 fermentation cycles (comprising both the initial fermentation cycle and 59 further fermentation cycles of the total fermenting population) according to the present process, the persistent yeast cells of the present disclosure are present in a proportion of at least 93% in the fermenting population (when measured with respect to its DNA contribution of the total fermenting population). In an embodiment, after a total 60 fermentation cycles (comprising both the initial fermentation cycle and 59 further fermentation cycles of the total fermenting population) according to the present process, the persistent yeast cells of the present disclosure are present in a proportion of at least 94% in the fermenting population (when measured with respect to its DNA contribution of the total fermenting population). In an embodiment, after a total 60 fermentation cycles (comprising both the initial fermentation cycle and 59 further fermentation cycles of the total fermenting population) according to the present process, the persistent yeast cells of the present disclosure are present in a proportion of at least 95% in the fermenting population (when measured with respect to its DNA contribution of the total fermenting population). In an embodiment, after a total 60 fermentation cycles (comprising both the initial fermentation cycle and 59 further fermentation cycles of the total fermenting population) according to the present process, the persistent yeast cells of the present disclosure are present in a proportion of at least 96% in the fermenting population (when measured with respect to its DNA contribution of the total fermenting population). In an embodiment, after a total 60 fermentation cycles (comprising both the initial fermentation cycle and 59 further fermentation cycles) according to the present process, the persistent yeast cells of the present disclosure are present in a proportion of at least 97% in the fermenting population (when measured with respect to its DNA contribution of the total fermenting population). In an embodiment, after a total 60 fermentation cycles (comprising both the initial fermentation cycle and 59 further fermentation cycles of the total fermenting population) according to the present process, the persistent yeast cells of the present disclosure are present in a proportion of at least 98% in the fermenting population (when measured with respect to its DNA contribution of the total fermenting population). In an embodiment, after a total 60 fermentation cycles (comprising both the initial fermentation cycle and 59 further fermentation cycles) according to the present process, the persistent yeast cells of the present disclosure are present in a proportion of at least 99% in the fermenting population (when measured with respect to its DNA contribution of the total fermenting population). In an embodiment, after a total 60 fermentation cycles (comprising both the initial fermentation cycle and 59 further fermentation cycles) according to the present process, the persistent yeast cells of the present disclosure are present in a proportion of at least 99.1% in the fermenting population (when measured with respect to its DNA contribution of the total fermenting population). In an embodiment, after a total 60 fermentation cycles (comprising both the initial fermentation cycle and 59 further fermentation cycles) according to the present process, the persistent yeast cells of the present disclosure are present in a proportion of at least 99.2% in the fermenting population (when measured with respect to its DNA contribution of the total fermenting population). In an embodiment, after a total 60 fermentation cycles (comprising both the initial fermentation cycle and 59 further fermentation cycles) according to the present process, the persistent yeast cells of the present disclosure are present in a proportion of at least 99.3% in the fermenting population (when measured with respect to its DNA contribution of the total fermenting population). In an embodiment, after a total 60 fermentation cycles (comprising both the initial fermentation cycle and 59 further fermentation cycles) according to the present process, the persistent yeast cells of the present disclosure are present in a proportion of at least 99.4% in the fermenting population (when measured with respect to its DNA contribution of the total fermenting population). In an embodiment, after a total 60 fermentation cycles (comprising both the initial fermentation cycle and 59 further fermentation cycles) according to the present process, the persistent yeast cells of the present disclosure are present in a proportion of at least 99.5% in the fermenting population (when measured with respect to its DNA contribution of the total fermenting population). In an embodiment, after a total 60 fermentation cycles (comprising both the initial fermentation cycle and 59 further fermentation cycles) according to the present process, the persistent yeast cells of the present disclosure are present in a proportion of at least 99.6% in the fermenting population (when measured with respect to its DNA contribution of the total fermenting population). In an embodiment, after a total 60 fermentation cycles (comprising both the initial fermentation cycle and 59 further fermentation cycles) according to the present process, the persistent yeast cells of the present disclosure are present in a proportion of at least 99.7% in the fermenting population (when measured with respect to its DNA contribution of the total fermenting population). In an embodiment, after a total 60 fermentation cycles (comprising both the initial fermentation cycle and 59 further fermentation cycles) according to the present process, the persistent yeast cells of the present disclosure are present in a proportion of at least 99.8% in the fermenting population (when measured with respect to its DNA contribution of the total fermenting population). In an embodiment, after a total 60 fermentation cycles (comprising both the initial fermentation cycle and 59 further fermentation cycles) according to the present process, the persistent yeast cells of the present disclosure are present in a proportion of at least 99.9% in the fermenting population (when measured with respect to its DNA contribution of the total fermenting population).

In an embodiment, after a total 70 fermentation cycles (comprising both the initial fermentation cycle and 69 further fermentation cycles) according to the present process, the persistent yeast cells of the present disclosure are present in a proportion of at least 90% in the fermenting population (when measured with respect to its DNA contribution of the total fermenting population). In an embodiment, after a total 70 fermentation cycles (comprising both the initial fermentation cycle and 69 further fermentation cycles) according to the present process, the persistent yeast cells of the present disclosure are present in a proportion of at least 91% in the fermenting population (when measured with respect to its DNA contribution of the total fermenting population). In an embodiment, after a total 70 fermentation cycles (comprising both the initial fermentation cycle and 69 further fermentation cycles) according to the present process, the persistent yeast cells of the present disclosure are present in a proportion of at least 92% in the fermenting population (when measured with respect to its DNA contribution of the total fermenting population). In an embodiment, after a total 70 fermentation cycles (comprising both the initial fermentation cycle and 69 further fermentation cycles of the total fermenting population) according to the present process, the persistent yeast cells of the present disclosure are present in a proportion of at least 93% in the fermenting population (when measured with respect to its DNA contribution of the total fermenting population). In an embodiment, after a total 70 fermentation cycles (comprising both the initial fermentation cycle and 69 further fermentation cycles of the total fermenting population) according to the present process, the persistent yeast cells of the present disclosure are present in a proportion of at least 94% in the fermenting population (when measured with respect to its DNA contribution of the total fermenting population). In an embodiment, after a total 70 fermentation cycles (comprising both the initial fermentation cycle and 69 further fermentation cycles of the total fermenting population) according to the present process, the persistent yeast cells of the present disclosure are present in a proportion of at least 95% in the fermenting population (when measured with respect to its DNA contribution of the total fermenting population). In an embodiment, after a total 70 fermentation cycles (comprising both the initial fermentation cycle and 69 further fermentation cycles of the total fermenting population) according to the present process, the persistent yeast cells of the present disclosure are present in a proportion of at least 96% in the fermenting population (when measured with respect to its DNA contribution of the total fermenting population). In an embodiment, after a total 70 fermentation cycles (comprising both the initial fermentation cycle and 69 further fermentation cycles) according to the present process, the persistent yeast cells of the present disclosure are present in a proportion of at least 97% in the fermenting population (when measured with respect to its DNA contribution of the total fermenting population). In an embodiment, after a total 70 fermentation cycles (comprising both the initial fermentation cycle and 69 further fermentation cycles of the total fermenting population) according to the present process, the persistent yeast cells of the present disclosure are present in a proportion of at least 98% in the fermenting population (when measured with respect to its DNA contribution of the total fermenting population). In an embodiment, after a total 70 fermentation cycles (comprising both the initial fermentation cycle and 69 further fermentation cycles) according to the present process, the persistent yeast cells of the present disclosure are present in a proportion of at least 99% in the fermenting population (when measured with respect to its DNA contribution of the total fermenting population). In an embodiment, after a total 70 fermentation cycles (comprising both the initial fermentation cycle and 69 further fermentation cycles) according to the present process, the persistent yeast cells of the present disclosure are present in a proportion of at least 99.1% in the fermenting population (when measured with respect to its DNA contribution of the total fermenting population). In an embodiment, after a total 70 fermentation cycles (comprising both the initial fermentation cycle and 69 further fermentation cycles) according to the present process, the persistent yeast cells of the present disclosure are present in a proportion of at least 99.2% in the fermenting population (when measured with respect to its DNA contribution of the total fermenting population). In an embodiment, after a total 70 fermentation cycles (comprising both the initial fermentation cycle and 69 further fermentation cycles) according to the present process, the persistent yeast cells of the present disclosure are present in a proportion of at least 99.3% in the fermenting population (when measured with respect to its DNA contribution of the total fermenting population). In an embodiment, after a total 70 fermentation cycles (comprising both the initial fermentation cycle and 69 further fermentation cycles) according to the present process, the persistent yeast cells of the present disclosure are present in a proportion of at least 99.4% in the fermenting population (when measured with respect to its DNA contribution of the total fermenting population). In an embodiment, after a total 70 fermentation cycles (comprising both the initial fermentation cycle and 69 further fermentation cycles) according to the present process, the persistent yeast cells of the present disclosure are present in a proportion of at least 99.5% in the fermenting population (when measured with respect to its DNA contribution of the total fermenting population). In an embodiment, after a total 70 fermentation cycles (comprising both the initial fermentation cycle and 69 further fermentation cycles) according to the present process, the persistent yeast cells of the present disclosure are present in a proportion of at least 99.6% in the fermenting population (when measured with respect to its DNA contribution of the total fermenting population). In an embodiment, after a total 70 fermentation cycles (comprising both the initial fermentation cycle and 69 further fermentation cycles) according to the present process, the persistent yeast cells of the present disclosure are present in a proportion of at least 99.7% in the fermenting population (when measured with respect to its DNA contribution of the total fermenting population). In an embodiment, after a total 70 fermentation cycles (comprising both the initial fermentation cycle and 69 further fermentation cycles) according to the present process, the persistent yeast cells of the present disclosure are present in a proportion of at least 99.8% in the fermenting population (when measured with respect to its DNA contribution of the total fermenting population). In an embodiment, after a total 70 fermentation cycles (comprising both the initial fermentation cycle and 69 further fermentation cycles) according to the present process, the persistent yeast cells of the present disclosure are present in a proportion of at least 99.9% in the fermenting population (when measured with respect to its DNA contribution of the total fermenting population).

In an embodiment, after a total 80 fermentation cycles (comprising both the initial fermentation cycle and 79 further fermentation cycles) according to the present process, the persistent yeast cells of the present disclosure are present in a proportion of at least 90% in the fermenting population (when measured with respect to its DNA contribution of the total fermenting population). In an embodiment, after a total 80 fermentation cycles (comprising both the initial fermentation cycle and 79 further fermentation cycles) according to the present process, the persistent yeast cells of the present disclosure are present in a proportion of at least 91% in the fermenting population (when measured with respect to its DNA contribution of the total fermenting population). In an embodiment, after a total 80 fermentation cycles (comprising both the initial fermentation cycle and 79 further fermentation cycles) according to the present process, the persistent yeast cells of the present disclosure are present in a proportion of at least 92% in the fermenting population (when measured with respect to its DNA contribution of the total fermenting population). In an embodiment, after a total 80 fermentation cycles (comprising both the initial fermentation cycle and 79 further fermentation cycles of the total fermenting population) according to the present process, the persistent yeast cells of the present disclosure are present in a proportion of at least 93% in the fermenting population (when measured with respect to its DNA contribution of the total fermenting population). In an embodiment, after a total 80 fermentation cycles (comprising both the initial fermentation cycle and 79 further fermentation cycles of the total fermenting population) according to the present process, the persistent yeast cells of the present disclosure are present in a proportion of at least 94% in the fermenting population (when measured with respect to its DNA contribution of the total fermenting population). In an embodiment, after a total 80 fermentation cycles (comprising both the initial fermentation cycle and 79 further fermentation cycles of the total fermenting population) according to the present process, the persistent yeast cells of the present disclosure are present in a proportion of at least 95% in the fermenting population (when measured with respect to its DNA contribution of the total fermenting population). In an embodiment, after a total 80 fermentation cycles (comprising both the initial fermentation cycle and 79 further fermentation cycles of the total fermenting population) according to the present process, the persistent yeast cells of the present disclosure are present in a proportion of at least 96% in the fermenting population (when measured with respect to its DNA contribution of the total fermenting population). In an embodiment, after a total 80 fermentation cycles (comprising both the initial fermentation cycle and 79 further fermentation cycles) according to the present process, the persistent yeast cells of the present disclosure are present in a proportion of at least 97% in the fermenting population (when measured with respect to its DNA contribution of the total fermenting population). In an embodiment, after a total 80 fermentation cycles (comprising both the initial fermentation cycle and 79 further fermentation cycles of the total fermenting population) according to the present process, the persistent yeast cells of the present disclosure are present in a proportion of at least 98% in the fermenting population (when measured with respect to its DNA contribution of the total fermenting population). In an embodiment, after a total 80 fermentation cycles (comprising both the initial fermentation cycle and 79 further fermentation cycles) according to the present process, the persistent yeast cells of the present disclosure are present in a proportion of at least 99% in the fermenting population (when measured with respect to its DNA contribution of the total fermenting population). In an embodiment, after a total 80 fermentation cycles (comprising both the initial fermentation cycle and 79 further fermentation cycles) according to the present process, the persistent yeast cells of the present disclosure are present in a proportion of at least 99.1% in the fermenting population (when measured with respect to its DNA contribution of the total fermenting population). In an embodiment, after a total 80 fermentation cycles (comprising both the initial fermentation cycle and 79 further fermentation cycles) according to the present process, the persistent yeast cells of the present disclosure are present in a proportion of at least 99.2% in the fermenting population (when measured with respect to its DNA contribution of the total fermenting population). In an embodiment, after a total 80 fermentation cycles (comprising both the initial fermentation cycle and 79 further fermentation cycles) according to the present process, the persistent yeast cells of the present disclosure are present in a proportion of at least 99.3% in the fermenting population (when measured with respect to its DNA contribution of the total fermenting population). In an embodiment, after a total 80 fermentation cycles (comprising both the initial fermentation cycle and 79 further fermentation cycles) according to the present process, the persistent yeast cells of the present disclosure are present in a proportion of at least 99.4% in the fermenting population (when measured with respect to its DNA contribution of the total fermenting population). In an embodiment, after a total 80 fermentation cycles (comprising both the initial fermentation cycle and 79 further fermentation cycles) according to the present process, the persistent yeast cells of the present disclosure are present in a proportion of at least 99.5% in the fermenting population (when measured with respect to its DNA contribution of the total fermenting population). In an embodiment, after a total 80 fermentation cycles (comprising both the initial fermentation cycle and 79 further fermentation cycles) according to the present process, the persistent yeast cells of the present disclosure are present in a proportion of at least 99.6% in the fermenting population (when measured with respect to its DNA contribution of the total fermenting population). In an embodiment, after a total 80 fermentation cycles (comprising both the initial fermentation cycle and 79 further fermentation cycles) according to the present process, the persistent yeast cells of the present disclosure are present in a proportion of at least 99.7% in the fermenting population (when measured with respect to its DNA contribution of the total fermenting population). In an embodiment, after a total 80 fermentation cycles (comprising both the initial fermentation cycle and 79 further fermentation cycles) according to the present process, the persistent yeast cells of the present disclosure are present in a proportion of at least 99.8% in the fermenting population (when measured with respect to its DNA contribution of the total fermenting population). In an embodiment, after a total 80 fermentation cycles (comprising both the initial fermentation cycle and 79 further fermentation cycles) according to the present process, the persistent yeast cells of the present disclosure are present in a proportion of at least 99.9% in the fermenting population (when measured with respect to its DNA contribution of the total fermenting population).

In an embodiment, after a total 90 fermentation cycles (comprising both the initial fermentation cycle and 89 further fermentation cycles) according to the present process, the persistent yeast cells of the present disclosure are present in a proportion of at least 90% in the fermenting population (when measured with respect to its DNA contribution of the total fermenting population). In an embodiment, after a total 90 fermentation cycles (comprising both the initial fermentation cycle and 89 further fermentation cycles) according to the present process, the persistent yeast cells of the present disclosure are present in a proportion of at least 91% in the fermenting population (when measured with respect to its DNA contribution of the total fermenting population). In an embodiment, after a total 90 fermentation cycles (comprising both the initial fermentation cycle and 89 further fermentation cycles) according to the present process, the persistent yeast cells of the present disclosure are present in a proportion of at least 92% in the fermenting population (when measured with respect to its DNA contribution of the total fermenting population). In an embodiment, after a total 90 fermentation cycles (comprising both the initial fermentation cycle and 89 further fermentation cycles of the total fermenting population) according to the present process, the persistent yeast cells of the present disclosure are present in a proportion of at least 93% in the fermenting population (when measured with respect to its DNA contribution of the total fermenting population). In an embodiment, after a total 90 fermentation cycles (comprising both the initial fermentation cycle and 89 further fermentation cycles of the total fermenting population) according to the present process, the persistent yeast cells of the present disclosure are present in a proportion of at least 94% in the fermenting population (when measured with respect to its DNA contribution of the total fermenting population). In an embodiment, after a total 90 fermentation cycles (comprising both the initial fermentation cycle and 89 further fermentation cycles of the total fermenting population) according to the present process, the persistent yeast cells of the present disclosure are present in a proportion of at least 95% in the fermenting population (when measured with respect to its DNA contribution of the total fermenting population). In an embodiment, after a total 90 fermentation cycles (comprising both the initial fermentation cycle and 89 further fermentation cycles of the total fermenting population) according to the present process, the persistent yeast cells of the present disclosure are present in a proportion of at least 96% in the fermenting population (when measured with respect to its DNA contribution of the total fermenting population). In an embodiment, after a total 90 fermentation cycles (comprising both the initial fermentation cycle and 89 further fermentation cycles) according to the present process, the persistent yeast cells of the present disclosure are present in a proportion of at least 97% in the fermenting population (when measured with respect to its DNA contribution of the total fermenting population). In an embodiment, after a total 90 fermentation cycles (comprising both the initial fermentation cycle and 89 further fermentation cycles of the total fermenting population) according to the present process, the persistent yeast cells of the present disclosure are present in a proportion of at least 98% in the fermenting population (when measured with respect to its DNA contribution of the total fermenting population). In an embodiment, after a total 90 fermentation cycles (comprising both the initial fermentation cycle and 89 further fermentation cycles) according to the present process, the persistent yeast cells of the present disclosure are present in a proportion of at least 99% in the fermenting population (when measured with respect to its DNA contribution of the total fermenting population). In an embodiment, after a total 90 fermentation cycles (comprising both the initial fermentation cycle and 89 further fermentation cycles) according to the present process, the persistent yeast cells of the present disclosure are present in a proportion of at least 99.1% in the fermenting population (when measured with respect to its DNA contribution of the total fermenting population). In an embodiment, after a total 90 fermentation cycles (comprising both the initial fermentation cycle and 89 further fermentation cycles) according to the present process, the persistent yeast cells of the present disclosure are present in a proportion of at least 99.2% in the fermenting population (when measured with respect to its DNA contribution of the total fermenting population). In an embodiment, after a total 90 fermentation cycles (comprising both the initial fermentation cycle and 89 further fermentation cycles) according to the present process, the persistent yeast cells of the present disclosure are present in a proportion of at least 99.3% in the fermenting population (when measured with respect to its DNA contribution of the total fermenting population). In an embodiment, after a total 90 fermentation cycles (comprising both the initial fermentation cycle and 89 further fermentation cycles) according to the present process, the persistent yeast cells of the present disclosure are present in a proportion of at least 99.4% in the fermenting population (when measured with respect to its DNA contribution of the total fermenting population). In an embodiment, after a total 90 fermentation cycles (comprising both the initial fermentation cycle and 89 further fermentation cycles) according to the present process, the persistent yeast cells of the present disclosure are present in a proportion of at least 99.5% in the fermenting population (when measured with respect to its DNA contribution of the total fermenting population). In an embodiment, after a total 90 fermentation cycles (comprising both the initial fermentation cycle and 89 further fermentation cycles) according to the present process, the persistent yeast cells of the present disclosure are present in a proportion of at least 99.6% in the fermenting population (when measured with respect to its DNA contribution of the total fermenting population). In an embodiment, after a total 90 fermentation cycles (comprising both the initial fermentation cycle and 89 further fermentation cycles) according to the present process, the persistent yeast cells of the present disclosure are present in a proportion of at least 99.7% in the fermenting population (when measured with respect to its DNA contribution of the total fermenting population). In an embodiment, after a total 90 fermentation cycles (comprising both the initial fermentation cycle and 89 further fermentation cycles) according to the present process, the persistent yeast cells of the present disclosure are present in a proportion of at least 99.8% in the fermenting population (when measured with respect to its DNA contribution of the total fermenting population). In an embodiment, after a total 90 fermentation cycles (comprising both the initial fermentation cycle and 89 further fermentation cycles) according to the present process, the persistent yeast cells of the present disclosure are present in a proportion of at least 99.9% in the fermenting population (when measured with respect to its DNA contribution of the total fermenting population).

In an embodiment, after a total 100 fermentation cycles (comprising both the initial fermentation cycle and 99 further fermentation cycles) according to the present process, the persistent yeast cells of the present disclosure are present in a proportion of at least 90% in the fermenting population (when measured with respect to its DNA contribution of the total fermenting population). In an embodiment, after a total 100 fermentation cycles (comprising both the initial fermentation cycle and 99 further fermentation cycles) according to the present process, the persistent yeast cells of the present disclosure are present in a proportion of at least 91% in the fermenting population (when measured with respect to its DNA contribution of the total fermenting population). In an embodiment, after a total 100 fermentation cycles (comprising both the initial fermentation cycle and 99 further fermentation cycles) according to the present process, the persistent yeast cells of the present disclosure are present in a proportion of at least 92% in the fermenting population (when measured with respect to its DNA contribution of the total fermenting population). In an embodiment, after a total 100 fermentation cycles (comprising both the initial fermentation cycle and 99 further fermentation cycles of the total fermenting population) according to the present process, the persistent yeast cells of the present disclosure are present in a proportion of at least 93% in the fermenting population (when measured with respect to its DNA contribution of the total fermenting population). In an embodiment, after a total 100 fermentation cycles (comprising both the initial fermentation cycle and 99 further fermentation cycles of the total fermenting population) according to the present process, the persistent yeast cells of the present disclosure are present in a proportion of at least 94% in the fermenting population (when measured with respect to its DNA contribution of the total fermenting population). In an embodiment, after a total 100 fermentation cycles (comprising both the initial fermentation cycle and 99 further fermentation cycles of the total fermenting population) according to the present process, the persistent yeast cells of the present disclosure are present in a proportion of at least 95% in the fermenting population (when measured with respect to its DNA contribution of the total fermenting population). In an embodiment, after a total 100 fermentation cycles (comprising both the initial fermentation cycle and 99 further fermentation cycles of the total fermenting population) according to the present process, the persistent yeast cells of the present disclosure are present in a proportion of at least 96% in the fermenting population (when measured with respect to its DNA contribution of the total fermenting population). In an embodiment, after a total 100 fermentation cycles (comprising both the initial fermentation cycle and 99 further fermentation cycles) according to the present process, the persistent yeast cells of the present disclosure are present in a proportion of at least 97% in the fermenting population (when measured with respect to its DNA contribution of the total fermenting population). In an embodiment, after a total 100 fermentation cycles (comprising both the initial fermentation cycle and 99 further fermentation cycles of the total fermenting population) according to the present process, the persistent yeast cells of the present disclosure are present in a proportion of at least 98% in the fermenting population (when measured with respect to its DNA contribution of the total fermenting population). In an embodiment, after a total 100 fermentation cycles (comprising both the initial fermentation cycle and 99 further fermentation cycles) according to the present process, the persistent yeast cells of the present disclosure are present in a proportion of at least 99% in the fermenting population (when measured with respect to its DNA contribution of the total fermenting population). In an embodiment, after a total 100 fermentation cycles (comprising both the initial fermentation cycle and 99 further fermentation cycles) according to the present process, the persistent yeast cells of the present disclosure are present in a proportion of at least 99.1% in the fermenting population (when measured with respect to its DNA contribution of the total fermenting population). In an embodiment, after a total 100 fermentation cycles (comprising both the initial fermentation cycle and 99 further fermentation cycles) according to the present process, the persistent yeast cells of the present disclosure are present in a proportion of at least 99.2% in the fermenting population (when measured with respect to its DNA contribution of the total fermenting population). In an embodiment, after a total 100 fermentation cycles (comprising both the initial fermentation cycle and 99 further fermentation cycles) according to the present process, the persistent yeast cells of the present disclosure are present in a proportion of at least 99.3% in the fermenting population (when measured with respect to its DNA contribution of the total fermenting population). In an embodiment, after a total 100 fermentation cycles (comprising both the initial fermentation cycle and 99 further fermentation cycles) according to the present process, the persistent yeast cells of the present disclosure are present in a proportion of at least 99.4% in the fermenting population (when measured with respect to its DNA contribution of the total fermenting population). In an embodiment, after a total 100 fermentation cycles (comprising both the initial fermentation cycle and 99 further fermentation cycles) according to the present process, the persistent yeast cells of the present disclosure are present in a proportion of at least 99.5% in the fermenting population (when measured with respect to its DNA contribution of the total fermenting population). In an embodiment, after a total 100 fermentation cycles (comprising both the initial fermentation cycle and 99 further fermentation cycles) according to the present process, the persistent yeast cells of the present disclosure are present in a proportion of at least 99.6% in the fermenting population (when measured with respect to its DNA contribution of the total fermenting population). In an embodiment, after a total 100 fermentation cycles (comprising both the initial fermentation cycle and 99 further fermentation cycles) according to the present process, the persistent yeast cells of the present disclosure are present in a proportion of at least 99.7% in the fermenting population (when measured with respect to its DNA contribution of the total fermenting population). In an embodiment, after a total 100 fermentation cycles (comprising both the initial fermentation cycle and 99 further fermentation cycles) according to the present process, the persistent yeast cells of the present disclosure are present in a proportion of at least 99.8% in the fermenting population (when measured with respect to its DNA contribution of the total fermenting population). In an embodiment, after a total 100 fermentation cycles (comprising both the initial fermentation cycle and 99 further fermentation cycles) according to the present process, the persistent yeast cells of the present disclosure are present in a proportion of at least 99.9% in the fermenting population (when measured with respect to its DNA contribution of the total fermenting population).

In an embodiment, after a total 110 fermentation cycles (comprising both the initial fermentation cycle and 109 further fermentation cycles) according to the present process, the persistent yeast cells of the present disclosure are present in a proportion of at least 90% in the fermenting population (when measured with respect to its DNA contribution of the total fermenting population). In an embodiment, after a total 110 fermentation cycles (comprising both the initial fermentation cycle and 109 further fermentation cycles) according to the present process, the persistent yeast cells of the present disclosure are present in a proportion of at least 91% in the fermenting population (when measured with respect to its DNA contribution of the total fermenting population). In an embodiment, after a total 110 fermentation cycles (comprising both the initial fermentation cycle and 109 further fermentation cycles) according to the present process, the persistent yeast cells of the present disclosure are present in a proportion of at least 92% in the fermenting population (when measured with respect to its DNA contribution of the total fermenting population). In an embodiment, after a total 110 fermentation cycles (comprising both the initial fermentation cycle and 109 further fermentation cycles of the total fermenting population) according to the present process, the persistent yeast cells of the present disclosure are present in a proportion of at least 93% in the fermenting population (when measured with respect to its DNA contribution of the total fermenting population). In an embodiment, after a total 110 fermentation cycles (comprising both the initial fermentation cycle and 109 further fermentation cycles of the total fermenting population) according to the present process, the persistent yeast cells of the present disclosure are present in a proportion of at least 94% in the fermenting population (when measured with respect to its DNA contribution of the total fermenting population). In an embodiment, after a total 110 fermentation cycles (comprising both the initial fermentation cycle and 109 further fermentation cycles of the total fermenting population) according to the present process, the persistent yeast cells of the present disclosure are present in a proportion of at least 95% in the fermenting population (when measured with respect to its DNA contribution of the total fermenting population). In an embodiment, after a total 110 fermentation cycles (comprising both the initial fermentation cycle and 109 further fermentation cycles of the total fermenting population) according to the present process, the persistent yeast cells of the present disclosure are present in a proportion of at least 96% in the fermenting population (when measured with respect to its DNA contribution of the total fermenting population). In an embodiment, after a total 110 fermentation cycles (comprising both the initial fermentation cycle and 109 further fermentation cycles) according to the present process, the persistent yeast cells of the present disclosure are present in a proportion of at least 97% in the fermenting population (when measured with respect to its DNA contribution of the total fermenting population). In an embodiment, after a total 110 fermentation cycles (comprising both the initial fermentation cycle and 109 further fermentation cycles of the total fermenting population) according to the present process, the persistent yeast cells of the present disclosure are present in a proportion of at least 98% in the fermenting population (when measured with respect to its DNA contribution of the total fermenting population). In an embodiment, after a total 110 fermentation cycles (comprising both the initial fermentation cycle and 109 further fermentation cycles) according to the present process, the persistent yeast cells of the present disclosure are present in a proportion of at least 99% in the fermenting population (when measured with respect to its DNA contribution of the total fermenting population). In an embodiment, after a total 110 fermentation cycles (comprising both the initial fermentation cycle and 109 further fermentation cycles) according to the present process, the persistent yeast cells of the present disclosure are present in a proportion of at least 99.1% in the fermenting population (when measured with respect to its DNA contribution of the total fermenting population). In an embodiment, after a total 110 fermentation cycles (comprising both the initial fermentation cycle and 109 further fermentation cycles) according to the present process, the persistent yeast cells of the present disclosure are present in a proportion of at least 99.2% in the fermenting population (when measured with respect to its DNA contribution of the total fermenting population). In an embodiment, after a total 110 fermentation cycles (comprising both the initial fermentation cycle and 109 further fermentation cycles) according to the present process, the persistent yeast cells of the present disclosure are present in a proportion of at least 99.3% in the fermenting population (when measured with respect to its DNA contribution of the total fermenting population). In an embodiment, after a total 110 fermentation cycles (comprising both the initial fermentation cycle and 109 further fermentation cycles) according to the present process, the persistent yeast cells of the present disclosure are present in a proportion of at least 99.4% in the fermenting population (when measured with respect to its DNA contribution of the total fermenting population). In an embodiment, after a total 110 fermentation cycles (comprising both the initial fermentation cycle and 109 further fermentation cycles) according to the present process, the persistent yeast cells of the present disclosure are present in a proportion of at least 99.5% in the fermenting population (when measured with respect to its DNA contribution of the total fermenting population). In an embodiment, after a total 110 fermentation cycles (comprising both the initial fermentation cycle and 109 further fermentation cycles) according to the present process, the persistent yeast cells of the present disclosure are present in a proportion of at least 99.6% in the fermenting population (when measured with respect to its DNA contribution of the total fermenting population). In an embodiment, after a total 110 fermentation cycles (comprising both the initial fermentation cycle and 109 further fermentation cycles) according to the present process, the persistent yeast cells of the present disclosure are present in a proportion of at least 99.7% in the fermenting population (when measured with respect to its DNA contribution of the total fermenting population). In an embodiment, after a total 110 fermentation cycles (comprising both the initial fermentation cycle and 109 further fermentation cycles) according to the present process, the persistent yeast cells of the present disclosure are present in a proportion of at least 99.8% in the fermenting population (when measured with respect to its DNA contribution of the total fermenting population). In an embodiment, after a total 110 fermentation cycles (comprising both the initial fermentation cycle and 109 further fermentation cycles) according to the present process, the persistent yeast cells of the present disclosure are present in a proportion of at least 99.9% in the fermenting population (when measured with respect to its DNA contribution of the total fermenting population).

In an embodiment, after a total 120 fermentation cycles (comprising both the initial fermentation cycle and 119 further fermentation cycles) according to the present process, the persistent yeast cells of the present disclosure are present in a proportion of at least 90% in the fermenting population (when measured with respect to its DNA contribution of the total fermenting population). In an embodiment, after a total 120 fermentation cycles (comprising both the initial fermentation cycle and 119 further fermentation cycles) according to the present process, the persistent yeast cells of the present disclosure are present in a proportion of at least 91% in the fermenting population (when measured with respect to its DNA contribution of the total fermenting population). In an embodiment, after a total 120 fermentation cycles (comprising both the initial fermentation cycle and 119 further fermentation cycles) according to the present process, the persistent yeast cells of the present disclosure are present in a proportion of at least 92% in the fermenting population (when measured with respect to its DNA contribution of the total fermenting population). In an embodiment, after a total 120 fermentation cycles (comprising both the initial fermentation cycle and 119 further fermentation cycles of the total fermenting population) according to the present process, the persistent yeast cells of the present disclosure are present in a proportion of at least 93% in the fermenting population (when measured with respect to its DNA contribution of the total fermenting population). In an embodiment, after a total 120 fermentation cycles (comprising both the initial fermentation cycle and 119 further fermentation cycles of the total fermenting population) according to the present process, the persistent yeast cells of the present disclosure are present in a proportion of at least 94% in the fermenting population (when measured with respect to its DNA contribution of the total fermenting population). In an embodiment, after a total 120 fermentation cycles (comprising both the initial fermentation cycle and 119 further fermentation cycles of the total fermenting population) according to the present process, the persistent yeast cells of the present disclosure are present in a proportion of at least 95% in the fermenting population (when measured with respect to its DNA contribution of the total fermenting population). In an embodiment, after a total 120 fermentation cycles (comprising both the initial fermentation cycle and 119 further fermentation cycles of the total fermenting population) according to the present process, the persistent yeast cells of the present disclosure are present in a proportion of at least 96% in the fermenting population (when measured with respect to its DNA contribution of the total fermenting population). In an embodiment, after a total 120 fermentation cycles (comprising both the initial fermentation cycle and 119 further fermentation cycles) according to the present process, the persistent yeast cells of the present disclosure are present in a proportion of at least 97% in the fermenting population (when measured with respect to its DNA contribution of the total fermenting population). In an embodiment, after a total 120 fermentation cycles (comprising both the initial fermentation cycle and 119 further fermentation cycles of the total fermenting population) according to the present process, the persistent yeast cells of the present disclosure are present in a proportion of at least 98% in the fermenting population (when measured with respect to its DNA contribution of the total fermenting population). In an embodiment, after a total 120 fermentation cycles (comprising both the initial fermentation cycle and 119 further fermentation cycles) according to the present process, the persistent yeast cells of the present disclosure are present in a proportion of at least 99% in the fermenting population (when measured with respect to its DNA contribution of the total fermenting population). In an embodiment, after a total 120 fermentation cycles (comprising both the initial fermentation cycle and 119 further fermentation cycles) according to the present process, the persistent yeast cells of the present disclosure are present in a proportion of at least 99.1% in the fermenting population (when measured with respect to its DNA contribution of the total fermenting population). In an embodiment, after a total 120 fermentation cycles (comprising both the initial fermentation cycle and 119 further fermentation cycles) according to the present process, the persistent yeast cells of the present disclosure are present in a proportion of at least 99.2% in the fermenting population (when measured with respect to its DNA contribution of the total fermenting population). In an embodiment, after a total 120 fermentation cycles (comprising both the initial fermentation cycle and 119 further fermentation cycles) according to the present process, the persistent yeast cells of the present disclosure are present in a proportion of at least 99.3% in the fermenting population (when measured with respect to its DNA contribution of the total fermenting population). In an embodiment, after a total 120 fermentation cycles (comprising both the initial fermentation cycle and 119 further fermentation cycles) according to the present process, the persistent yeast cells of the present disclosure are present in a proportion of at least 99.4% in the fermenting population (when measured with respect to its DNA contribution of the total fermenting population). In an embodiment, after a total 120 fermentation cycles (comprising both the initial fermentation cycle and 119 further fermentation cycles) according to the present process, the persistent yeast cells of the present disclosure are present in a proportion of at least 99.5% in the fermenting population (when measured with respect to its DNA contribution of the total fermenting population). In an embodiment, after a total 120 fermentation cycles (comprising both the initial fermentation cycle and 119 further fermentation cycles) according to the present process, the persistent yeast cells of the present disclosure are present in a proportion of at least 99.6% in the fermenting population (when measured with respect to its DNA contribution of the total fermenting population). In an embodiment, after a total 120 fermentation cycles (comprising both the initial fermentation cycle and 119 further fermentation cycles) according to the present process, the persistent yeast cells of the present disclosure are present in a proportion of at least 99.7% in the fermenting population (when measured with respect to its DNA contribution of the total fermenting population). In an embodiment, after a total 120 fermentation cycles (comprising both the initial fermentation cycle and 119 further fermentation cycles) according to the present process, the persistent yeast cells of the present disclosure are present in a proportion of at least 99.8% in the fermenting population (when measured with respect to its DNA contribution of the total fermenting population). In an embodiment, after a total 120 fermentation cycles (comprising both the initial fermentation cycle and 119 further fermentation cycles) according to the present process, the persistent yeast cells of the present disclosure are present in a proportion of at least 99.9% in the fermenting population (when measured with respect to its DNA contribution of the total fermenting population).

In an embodiment, after a total 130 fermentation cycles (comprising both the initial fermentation cycle and 129 further fermentation cycles) according to the present process, the persistent yeast cells of the present disclosure are present in a proportion of at least 90% in the fermenting population (when measured with respect to its DNA contribution of the total fermenting population). In an embodiment, after a total 130 fermentation cycles (comprising both the initial fermentation cycle and 129 further fermentation cycles) according to the present process, the persistent yeast cells of the present disclosure are present in a proportion of at least 91% in the fermenting population (when measured with respect to its DNA contribution of the total fermenting population). In an embodiment, after a total 130 fermentation cycles (comprising both the initial fermentation cycle and 129 further fermentation cycles) according to the present process, the persistent yeast cells of the present disclosure are present in a proportion of at least 92% in the fermenting population (when measured with respect to its DNA contribution of the total fermenting population). In an embodiment, after a total 130 fermentation cycles (comprising both the initial fermentation cycle and 129 further fermentation cycles of the total fermenting population) according to the present process, the persistent yeast cells of the present disclosure are present in a proportion of at least 93% in the fermenting population (when measured with respect to its DNA contribution of the total fermenting population). In an embodiment, after a total 130 fermentation cycles (comprising both the initial fermentation cycle and 129 further fermentation cycles of the total fermenting population) according to the present process, the persistent yeast cells of the present disclosure are present in a proportion of at least 94% in the fermenting population (when measured with respect to its DNA contribution of the total fermenting population). In an embodiment, after a total 130 fermentation cycles (comprising both the initial fermentation cycle and 129 further fermentation cycles of the total fermenting population) according to the present process, the persistent yeast cells of the present disclosure are present in a proportion of at least 95% in the fermenting population (when measured with respect to its DNA contribution of the total fermenting population). In an embodiment, after a total 130 fermentation cycles (comprising both the initial fermentation cycle and 129 further fermentation cycles of the total fermenting population) according to the present process, the persistent yeast cells of the present disclosure are present in a proportion of at least 96% in the fermenting population (when measured with respect to its DNA contribution of the total fermenting population). In an embodiment, after a total 130 fermentation cycles (comprising both the initial fermentation cycle and 129 further fermentation cycles) according to the present process, the persistent yeast cells of the present disclosure are present in a proportion of at least 97% in the fermenting population (when measured with respect to its DNA contribution of the total fermenting population). In an embodiment, after a total 130 fermentation cycles (comprising both the initial fermentation cycle and 129 further fermentation cycles of the total fermenting population) according to the present process, the persistent yeast cells of the present disclosure are present in a proportion of at least 98% in the fermenting population (when measured with respect to its DNA contribution of the total fermenting population). In an embodiment, after a total 130 fermentation cycles (comprising both the initial fermentation cycle and 129 further fermentation cycles) according to the present process, the persistent yeast cells of the present disclosure are present in a proportion of at least 99% in the fermenting population (when measured with respect to its DNA contribution of the total fermenting population). In an embodiment, after a total 130 fermentation cycles (comprising both the initial fermentation cycle and 129 further fermentation cycles) according to the present process, the persistent yeast cells of the present disclosure are present in a proportion of at least 99.1% in the fermenting population (when measured with respect to its DNA contribution of the total fermenting population). In an embodiment, after a total 130 fermentation cycles (comprising both the initial fermentation cycle and 129 further fermentation cycles) according to the present process, the persistent yeast cells of the present disclosure are present in a proportion of at least 99.2% in the fermenting population (when measured with respect to its DNA contribution of the total fermenting population). In an embodiment, after a total 130 fermentation cycles (comprising both the initial fermentation cycle and 129 further fermentation cycles) according to the present process, the persistent yeast cells of the present disclosure are present in a proportion of at least 99.3% in the fermenting population (when measured with respect to its DNA contribution of the total fermenting population). In an embodiment, after a total 130 fermentation cycles (comprising both the initial fermentation cycle and 129 further fermentation cycles) according to the present process, the persistent yeast cells of the present disclosure are present in a proportion of at least 99.4% in the fermenting population (when measured with respect to its DNA contribution of the total fermenting population). In an embodiment, after a total 130 fermentation cycles (comprising both the initial fermentation cycle and 129 further fermentation cycles) according to the present process, the persistent yeast cells of the present disclosure are present in a proportion of at least 99.5% in the fermenting population (when measured with respect to its DNA contribution of the total fermenting population). In an embodiment, after a total 130 fermentation cycles (comprising both the initial fermentation cycle and 129 further fermentation cycles) according to the present process, the persistent yeast cells of the present disclosure are present in a proportion of at least 99.6% in the fermenting population (when measured with respect to its DNA contribution of the total fermenting population). In an embodiment, after a total 130 fermentation cycles (comprising both the initial fermentation cycle and 129 further fermentation cycles) according to the present process, the persistent yeast cells of the present disclosure are present in a proportion of at least 99.7% in the fermenting population (when measured with respect to its DNA contribution of the total fermenting population). In an embodiment, after a total 130 fermentation cycles (comprising both the initial fermentation cycle and 129 further fermentation cycles) according to the present process, the persistent yeast cells of the present disclosure are present in a proportion of at least 99.8% in the fermenting population (when measured with respect to its DNA contribution of the total fermenting population). In an embodiment, after a total 130 fermentation cycles (comprising both the initial fermentation cycle and 129 further fermentation cycles) according to the present process, the persistent yeast cells of the present disclosure are present in a proportion of at least 99.9% in the fermenting population (when measured with respect to its DNA contribution of the total fermenting population).

In an embodiment, after a total 140 fermentation cycles (comprising both the initial fermentation cycle and 139 further fermentation cycles) according to the present process, the persistent yeast cells of the present disclosure are present in a proportion of at least 90% in the fermenting population (when measured with respect to its DNA contribution of the total fermenting population). In an embodiment, after a total 140 fermentation cycles (comprising both the initial fermentation cycle and 139 further fermentation cycles) according to the present process, the persistent yeast cells of the present disclosure are present in a proportion of at least 91% in the fermenting population (when measured with respect to its DNA contribution of the total fermenting population). In an embodiment, after a total 140 fermentation cycles (comprising both the initial fermentation cycle and 139 further fermentation cycles) according to the present process, the persistent yeast cells of the present disclosure are present in a proportion of at least 92% in the fermenting population (when measured with respect to its DNA contribution of the total fermenting population). In an embodiment, after a total 140 fermentation cycles (comprising both the initial fermentation cycle and 139 further fermentation cycles of the total fermenting population) according to the present process, the persistent yeast cells of the present disclosure are present in a proportion of at least 93% in the fermenting population (when measured with respect to its DNA contribution of the total fermenting population). In an embodiment, after a total 140 fermentation cycles (comprising both the initial fermentation cycle and 139 further fermentation cycles of the total fermenting population) according to the present process, the persistent yeast cells of the present disclosure are present in a proportion of at least 94% in the fermenting population (when measured with respect to its DNA contribution of the total fermenting population). In an embodiment, after a total 140 fermentation cycles (comprising both the initial fermentation cycle and 139 further fermentation cycles of the total fermenting population) according to the present process, the persistent yeast cells of the present disclosure are present in a proportion of at least 95% in the fermenting population (when measured with respect to its DNA contribution of the total fermenting population). In an embodiment, after a total 140 fermentation cycles (comprising both the initial fermentation cycle and 139 further fermentation cycles of the total fermenting population) according to the present process, the persistent yeast cells of the present disclosure are present in a proportion of at least 96% in the fermenting population (when measured with respect to its DNA contribution of the total fermenting population). In an embodiment, after a total 140 fermentation cycles (comprising both the initial fermentation cycle and 139 further fermentation cycles) according to the present process, the persistent yeast cells of the present disclosure are present in a proportion of at least 97% in the fermenting population (when measured with respect to its DNA contribution of the total fermenting population). In an embodiment, after a total 140 fermentation cycles (comprising both the initial fermentation cycle and 139 further fermentation cycles of the total fermenting population) according to the present process, the persistent yeast cells of the present disclosure are present in a proportion of at least 98% in the fermenting population (when measured with respect to its DNA contribution of the total fermenting population). In an embodiment, after a total 140 fermentation cycles (comprising both the initial fermentation cycle and 139 further fermentation cycles) according to the present process, the persistent yeast cells of the present disclosure are present in a proportion of at least 99% in the fermenting population (when measured with respect to its DNA contribution of the total fermenting population). In an embodiment, after a total 140 fermentation cycles (comprising both the initial fermentation cycle and 139 further fermentation cycles) according to the present process, the persistent yeast cells of the present disclosure are present in a proportion of at least 99.1% in the fermenting population (when measured with respect to its DNA contribution of the total fermenting population). In an embodiment, after a total 140 fermentation cycles (comprising both the initial fermentation cycle and 139 further fermentation cycles) according to the present process, the persistent yeast cells of the present disclosure are present in a proportion of at least 99.2% in the fermenting population (when measured with respect to its DNA contribution of the total fermenting population). In an embodiment, after a total 140 fermentation cycles (comprising both the initial fermentation cycle and 139 further fermentation cycles) according to the present process, the persistent yeast cells of the present disclosure are present in a proportion of at least 99.3% in the fermenting population (when measured with respect to its DNA contribution of the total fermenting population). In an embodiment, after a total 140 fermentation cycles (comprising both the initial fermentation cycle and 139 further fermentation cycles) according to the present process, the persistent yeast cells of the present disclosure are present in a proportion of at least 99.4% in the fermenting population (when measured with respect to its DNA contribution of the total fermenting population). In an embodiment, after a total 140 fermentation cycles (comprising both the initial fermentation cycle and 139 further fermentation cycles) according to the present process, the persistent yeast cells of the present disclosure are present in a proportion of at least 99.5% in the fermenting population (when measured with respect to its DNA contribution of the total fermenting population). In an embodiment, after a total 140 fermentation cycles (comprising both the initial fermentation cycle and 139 further fermentation cycles) according to the present process, the persistent yeast cells of the present disclosure are present in a proportion of at least 99.6% in the fermenting population (when measured with respect to its DNA contribution of the total fermenting population). In an embodiment, after a total 140 fermentation cycles (comprising both the initial fermentation cycle and 139 further fermentation cycles) according to the present process, the persistent yeast cells of the present disclosure are present in a proportion of at least 99.7% in the fermenting population (when measured with respect to its DNA contribution of the total fermenting population). In an embodiment, after a total 140 fermentation cycles (comprising both the initial fermentation cycle and 139 further fermentation cycles) according to the present process, the persistent yeast cells of the present disclosure are present in a proportion of at least 99.8% in the fermenting population (when measured with respect to its DNA contribution of the total fermenting population). In an embodiment, after a total 140 fermentation cycles (comprising both the initial fermentation cycle and 139 further fermentation cycles) according to the present process, the persistent yeast cells of the present disclosure are present in a proportion of at least 99.9% in the fermenting population (when measured with respect to its DNA contribution of the total fermenting population).

In an embodiment, after a total 150 fermentation cycles (comprising both the initial fermentation cycle and 149 further fermentation cycles) according to the present process, the persistent yeast cells of the present disclosure are present in a proportion of at least 90% in the fermenting population (when measured with respect to its DNA contribution of the total fermenting population). In an embodiment, after a total 150 fermentation cycles (comprising both the initial fermentation cycle and 149 further fermentation cycles) according to the present process, the persistent yeast cells of the present disclosure are present in a proportion of at least 91% in the fermenting population (when measured with respect to its DNA contribution of the total fermenting population). In an embodiment, after a total 150 fermentation cycles (comprising both the initial fermentation cycle and 149 further fermentation cycles) according to the present process, the persistent yeast cells of the present disclosure are present in a proportion of at least 92% in the fermenting population (when measured with respect to its DNA contribution of the total fermenting population). In an embodiment, after a total 150 fermentation cycles (comprising both the initial fermentation cycle and 149 further fermentation cycles of the total fermenting population) according to the present process, the persistent yeast cells of the present disclosure are present in a proportion of at least 93% in the fermenting population (when measured with respect to its DNA contribution of the total fermenting population). In an embodiment, after a total 150 fermentation cycles (comprising both the initial fermentation cycle and 149 further fermentation cycles of the total fermenting population) according to the present process, the persistent yeast cells of the present disclosure are present in a proportion of at least 94% in the fermenting population (when measured with respect to its DNA contribution of the total fermenting population). In an embodiment, after a total 150 fermentation cycles (comprising both the initial fermentation cycle and 149 further fermentation cycles of the total fermenting population) according to the present process, the persistent yeast cells of the present disclosure are present in a proportion of at least 95% in the fermenting population (when measured with respect to its DNA contribution of the total fermenting population). In an embodiment, after a total 150 fermentation cycles (comprising both the initial fermentation cycle and 149 further fermentation cycles of the total fermenting population) according to the present process, the persistent yeast cells of the present disclosure are present in a proportion of at least 96% in the fermenting population (when measured with respect to its DNA contribution of the total fermenting population). In an embodiment, after a total 150 fermentation cycles (comprising both the initial fermentation cycle and 149 further fermentation cycles) according to the present process, the persistent yeast cells of the present disclosure are present in a proportion of at least 97% in the fermenting population (when measured with respect to its DNA contribution of the total fermenting population). In an embodiment, after a total 150 fermentation cycles (comprising both the initial fermentation cycle and 149 further fermentation cycles of the total fermenting population) according to the present process, the persistent yeast cells of the present disclosure are present in a proportion of at least 98% in the fermenting population (when measured with respect to its DNA contribution of the total fermenting population). In an embodiment, after a total 150 fermentation cycles (comprising both the initial fermentation cycle and 149 further fermentation cycles) according to the present process, the persistent yeast cells of the present disclosure are present in a proportion of at least 99% in the fermenting population (when measured with respect to its DNA contribution of the total fermenting population). In an embodiment, after a total 150 fermentation cycles (comprising both the initial fermentation cycle and 149 further fermentation cycles) according to the present process, the persistent yeast cells of the present disclosure are present in a proportion of at least 99.1% in the fermenting population (when measured with respect to its DNA contribution of the total fermenting population). In an embodiment, after a total 150 fermentation cycles (comprising both the initial fermentation cycle and 149 further fermentation cycles) according to the present process, the persistent yeast cells of the present disclosure are present in a proportion of at least 99.2% in the fermenting population (when measured with respect to its DNA contribution of the total fermenting population). In an embodiment, after a total 150 fermentation cycles (comprising both the initial fermentation cycle and 149 further fermentation cycles) according to the present process, the persistent yeast cells of the present disclosure are present in a proportion of at least 99.3% in the fermenting population (when measured with respect to its DNA contribution of the total fermenting population). In an embodiment, after a total 150 fermentation cycles (comprising both the initial fermentation cycle and 149 further fermentation cycles) according to the present process, the persistent yeast cells of the present disclosure are present in a proportion of at least 99.4% in the fermenting population (when measured with respect to its DNA contribution of the total fermenting population). In an embodiment, after a total 150 fermentation cycles (comprising both the initial fermentation cycle and 149 further fermentation cycles) according to the present process, the persistent yeast cells of the present disclosure are present in a proportion of at least 99.5% in the fermenting population (when measured with respect to its DNA contribution of the total fermenting population). In an embodiment, after a total 150 fermentation cycles (comprising both the initial fermentation cycle and 149 further fermentation cycles) according to the present process, the persistent yeast cells of the present disclosure are present in a proportion of at least 99.6% in the fermenting population (when measured with respect to its DNA contribution of the total fermenting population). In an embodiment, after a total 150 fermentation cycles (comprising both the initial fermentation cycle and 149 further fermentation cycles) according to the present process, the persistent yeast cells of the present disclosure are present in a proportion of at least 99.7% in the fermenting population (when measured with respect to its DNA contribution of the total fermenting population). In an embodiment, after a total 150 fermentation cycles (comprising both the initial fermentation cycle and 149 further fermentation cycles) according to the present process, the persistent yeast cells of the present disclosure are present in a proportion of at least 99.8% in the fermenting population (when measured with respect to its DNA contribution of the total fermenting population). In an embodiment, after a total 150 fermentation cycles (comprising both the initial fermentation cycle and 149 further fermentation cycles) according to the present process, the persistent yeast cells of the present disclosure are present in a proportion of at least 99.9% in the fermenting population (when measured with respect to its DNA contribution of the total fermenting population).

In an embodiment, after a total 160 fermentation cycles (comprising both the initial fermentation cycle and 159 further fermentation cycles) according to the present process, the persistent yeast cells of the present disclosure are present in a proportion of at least 90% in the fermenting population (when measured with respect to its DNA contribution of the total fermenting population). In an embodiment, after a total 160 fermentation cycles (comprising both the initial fermentation cycle and 159 further fermentation cycles) according to the present process, the persistent yeast cells of the present disclosure are present in a proportion of at least 91% in the fermenting population (when measured with respect to its DNA contribution of the total fermenting population). In an embodiment, after a total 160 fermentation cycles (comprising both the initial fermentation cycle and 159 further fermentation cycles) according to the present process, the persistent yeast cells of the present disclosure are present in a proportion of at least 92% in the fermenting population (when measured with respect to its DNA contribution of the total fermenting population). In an embodiment, after a total 160 fermentation cycles (comprising both the initial fermentation cycle and 159 further fermentation cycles of the total fermenting population) according to the present process, the persistent yeast cells of the present disclosure are present in a proportion of at least 93% in the fermenting population (when measured with respect to its DNA contribution of the total fermenting population). In an embodiment, after a total 160 fermentation cycles (comprising both the initial fermentation cycle and 159 further fermentation cycles of the total fermenting population) according to the present process, the persistent yeast cells of the present disclosure are present in a proportion of at least 94% in the fermenting population (when measured with respect to its DNA contribution of the total fermenting population). In an embodiment, after a total 160 fermentation cycles (comprising both the initial fermentation cycle and 159 further fermentation cycles of the total fermenting population) according to the present process, the persistent yeast cells of the present disclosure are present in a proportion of at least 95% in the fermenting population (when measured with respect to its DNA contribution of the total fermenting population). In an embodiment, after a total 160 fermentation cycles (comprising both the initial fermentation cycle and 159 further fermentation cycles of the total fermenting population) according to the present process, the persistent yeast cells of the present disclosure are present in a proportion of at least 96% in the fermenting population (when measured with respect to its DNA contribution of the total fermenting population). In an embodiment, after a total 160 fermentation cycles (comprising both the initial fermentation cycle and 159 further fermentation cycles) according to the present process, the persistent yeast cells of the present disclosure are present in a proportion of at least 97% in the fermenting population (when measured with respect to its DNA contribution of the total fermenting population). In an embodiment, after a total 160 fermentation cycles (comprising both the initial fermentation cycle and 159 further fermentation cycles of the total fermenting population) according to the present process, the persistent yeast cells of the present disclosure are present in a proportion of at least 98% in the fermenting population (when measured with respect to its DNA contribution of the total fermenting population). In an embodiment, after a total 160 fermentation cycles (comprising both the initial fermentation cycle and 159 further fermentation cycles) according to the present process, the persistent yeast cells of the present disclosure are present in a proportion of at least 99% in the fermenting population (when measured with respect to its DNA contribution of the total fermenting population). In an embodiment, after a total 160 fermentation cycles (comprising both the initial fermentation cycle and 159 further fermentation cycles) according to the present process, the persistent yeast cells of the present disclosure are present in a proportion of at least 99.1% in the fermenting population (when measured with respect to its DNA contribution of the total fermenting population). In an embodiment, after a total 160 fermentation cycles (comprising both the initial fermentation cycle and 159 further fermentation cycles) according to the present process, the persistent yeast cells of the present disclosure are present in a proportion of at least 99.2% in the fermenting population (when measured with respect to its DNA contribution of the total fermenting population). In an embodiment, after a total 160 fermentation cycles (comprising both the initial fermentation cycle and 159 further fermentation cycles) according to the present process, the persistent yeast cells of the present disclosure are present in a proportion of at least 99.3% in the fermenting population (when measured with respect to its DNA contribution of the total fermenting population). In an embodiment, after a total 160 fermentation cycles (comprising both the initial fermentation cycle and 159 further fermentation cycles) according to the present process, the persistent yeast cells of the present disclosure are present in a proportion of at least 99.4% in the fermenting population (when measured with respect to its DNA contribution of the total fermenting population). In an embodiment, after a total 160 fermentation cycles (comprising both the initial fermentation cycle and 159 further fermentation cycles) according to the present process, the persistent yeast cells of the present disclosure are present in a proportion of at least 99.5% in the fermenting population (when measured with respect to its DNA contribution of the total fermenting population). In an embodiment, after a total 160 fermentation cycles (comprising both the initial fermentation cycle and 159 further fermentation cycles) according to the present process, the persistent yeast cells of the present disclosure are present in a proportion of at least 99.6% in the fermenting population (when measured with respect to its DNA contribution of the total fermenting population). In an embodiment, after a total 160 fermentation cycles (comprising both the initial fermentation cycle and 159 further fermentation cycles) according to the present process, the persistent yeast cells of the present disclosure are present in a proportion of at least 99.7% in the fermenting population (when measured with respect to its DNA contribution of the total fermenting population). In an embodiment, after a total 160 fermentation cycles (comprising both the initial fermentation cycle and 159 further fermentation cycles) according to the present process, the persistent yeast cells of the present disclosure are present in a proportion of at least 99.8% in the fermenting population (when measured with respect to its DNA contribution of the total fermenting population). In an embodiment, after a total 160 fermentation cycles (comprising both the initial fermentation cycle and 159 further fermentation cycles) according to the present process, the persistent yeast cells of the present disclosure are present in a proportion of at least 99.9% in the fermenting population (when measured with respect to its DNA contribution of the total fermenting population).

In an embodiment, after a total 170 fermentation cycles (comprising both the initial fermentation cycle and 169 further fermentation cycles) according to the present process, the persistent yeast cells of the present disclosure are present in a proportion of at least 90% in the fermenting population (when measured with respect to its DNA contribution of the total fermenting population). In an embodiment, after a total 170 fermentation cycles (comprising both the initial fermentation cycle and 169 further fermentation cycles) according to the present process, the persistent yeast cells of the present disclosure are present in a proportion of at least 91% in the fermenting population (when measured with respect to its DNA contribution of the total fermenting population). In an embodiment, after a total 170 fermentation cycles (comprising both the initial fermentation cycle and 169 further fermentation cycles) according to the present process, the persistent yeast cells of the present disclosure are present in a proportion of at least 92% in the fermenting population (when measured with respect to its DNA contribution of the total fermenting population). In an embodiment, after a total 170 fermentation cycles (comprising both the initial fermentation cycle and 169 further fermentation cycles of the total fermenting population) according to the present process, the persistent yeast cells of the present disclosure are present in a proportion of at least 93% in the fermenting population (when measured with respect to its DNA contribution of the total fermenting population). In an embodiment, after a total 170 fermentation cycles (comprising both the initial fermentation cycle and 169 further fermentation cycles of the total fermenting population) according to the present process, the persistent yeast cells of the present disclosure are present in a proportion of at least 94% in the fermenting population (when measured with respect to its DNA contribution of the total fermenting population). In an embodiment, after a total 170 fermentation cycles (comprising both the initial fermentation cycle and 169 further fermentation cycles of the total fermenting population) according to the present process, the persistent yeast cells of the present disclosure are present in a proportion of at least 95% in the fermenting population (when measured with respect to its DNA contribution of the total fermenting population). In an embodiment, after a total 170 fermentation cycles (comprising both the initial fermentation cycle and 169 further fermentation cycles of the total fermenting population) according to the present process, the persistent yeast cells of the present disclosure are present in a proportion of at least 96% in the fermenting population (when measured with respect to its DNA contribution of the total fermenting population). In an embodiment, after a total 170 fermentation cycles (comprising both the initial fermentation cycle and 169 further fermentation cycles) according to the present process, the persistent yeast cells of the present disclosure are present in a proportion of at least 97% in the fermenting population (when measured with respect to its DNA contribution of the total fermenting population). In an embodiment, after a total 170 fermentation cycles (comprising both the initial fermentation cycle and 169 further fermentation cycles of the total fermenting population) according to the present process, the persistent yeast cells of the present disclosure are present in a proportion of at least 98% in the fermenting population (when measured with respect to its DNA contribution of the total fermenting population). In an embodiment, after a total 170 fermentation cycles (comprising both the initial fermentation cycle and 169 further fermentation cycles) according to the present process, the persistent yeast cells of the present disclosure are present in a proportion of at least 99% in the fermenting population (when measured with respect to its DNA contribution of the total fermenting population). In an embodiment, after a total 170 fermentation cycles (comprising both the initial fermentation cycle and 169 further fermentation cycles) according to the present process, the persistent yeast cells of the present disclosure are present in a proportion of at least 99.1% in the fermenting population (when measured with respect to its DNA contribution of the total fermenting population). In an embodiment, after a total 170 fermentation cycles (comprising both the initial fermentation cycle and 169 further fermentation cycles) according to the present process, the persistent yeast cells of the present disclosure are present in a proportion of at least 99.2% in the fermenting population (when measured with respect to its DNA contribution of the total fermenting population). In an embodiment, after a total 170 fermentation cycles (comprising both the initial fermentation cycle and 169 further fermentation cycles) according to the present process, the persistent yeast cells of the present disclosure are present in a proportion of at least 99.3% in the fermenting population (when measured with respect to its DNA contribution of the total fermenting population). In an embodiment, after a total 170 fermentation cycles (comprising both the initial fermentation cycle and 169 further fermentation cycles) according to the present process, the persistent yeast cells of the present disclosure are present in a proportion of at least 99.4% in the fermenting population (when measured with respect to its DNA contribution of the total fermenting population). In an embodiment, after a total 170 fermentation cycles (comprising both the initial fermentation cycle and 169 further fermentation cycles) according to the present process, the persistent yeast cells of the present disclosure are present in a proportion of at least 99.5% in the fermenting population (when measured with respect to its DNA contribution of the total fermenting population). In an embodiment, after a total 170 fermentation cycles (comprising both the initial fermentation cycle and 169 further fermentation cycles) according to the present process, the persistent yeast cells of the present disclosure are present in a proportion of at least 99.6% in the fermenting population (when measured with respect to its DNA contribution of the total fermenting population). In an embodiment, after a total 170 fermentation cycles (comprising both the initial fermentation cycle and 169 further fermentation cycles) according to the present process, the persistent yeast cells of the present disclosure are present in a proportion of at least 99.7% in the fermenting population (when measured with respect to its DNA contribution of the total fermenting population). In an embodiment, after a total 170 fermentation cycles (comprising both the initial fermentation cycle and 169 further fermentation cycles) according to the present process, the persistent yeast cells of the present disclosure are present in a proportion of at least 99.8% in the fermenting population (when measured with respect to its DNA contribution of the total fermenting population). In an embodiment, after a total 170 fermentation cycles (comprising both the initial fermentation cycle and 169 further fermentation cycles) according to the present process, the persistent yeast cells of the present disclosure are present in a proportion of at least 99.9% in the fermenting population (when measured with respect to its DNA contribution of the total fermenting population).

In an embodiment, after a total 180 fermentation cycles (comprising both the initial fermentation cycle and 179 further fermentation cycles) according to the present process, the persistent yeast cells of the present disclosure are present in a proportion of at least 90% in the fermenting population (when measured with respect to its DNA contribution of the total fermenting population). In an embodiment, after a total 180 fermentation cycles (comprising both the initial fermentation cycle and 179 further fermentation cycles) according to the present process, the persistent yeast cells of the present disclosure are present in a proportion of at least 91% in the fermenting population (when measured with respect to its DNA contribution of the total fermenting population). In an embodiment, after a total 180 fermentation cycles (comprising both the initial fermentation cycle and 179 further fermentation cycles) according to the present process, the persistent yeast cells of the present disclosure are present in a proportion of at least 92% in the fermenting population (when measured with respect to its DNA contribution of the total fermenting population). In an embodiment, after a total 180 fermentation cycles (comprising both the initial fermentation cycle and 179 further fermentation cycles of the total fermenting population) according to the present process, the persistent yeast cells of the present disclosure are present in a proportion of at least 93% in the fermenting population (when measured with respect to its DNA contribution of the total fermenting population). In an embodiment, after a total 180 fermentation cycles (comprising both the initial fermentation cycle and 179 further fermentation cycles of the total fermenting population) according to the present process, the persistent yeast cells of the present disclosure are present in a proportion of at least 94% in the fermenting population (when measured with respect to its DNA contribution of the total fermenting population). In an embodiment, after a total 180 fermentation cycles (comprising both the initial fermentation cycle and 179 further fermentation cycles of the total fermenting population) according to the present process, the persistent yeast cells of the present disclosure are present in a proportion of at least 95% in the fermenting population (when measured with respect to its DNA contribution of the total fermenting population). In an embodiment, after a total 180 fermentation cycles (comprising both the initial fermentation cycle and 179 further fermentation cycles of the total fermenting population) according to the present process, the persistent yeast cells of the present disclosure are present in a proportion of at least 96% in the fermenting population (when measured with respect to its DNA contribution of the total fermenting population). In an embodiment, after a total 180 fermentation cycles (comprising both the initial fermentation cycle and 179 further fermentation cycles) according to the present process, the persistent yeast cells of the present disclosure are present in a proportion of at least 97% in the fermenting population (when measured with respect to its DNA contribution of the total fermenting population). In an embodiment, after a total 180 fermentation cycles (comprising both the initial fermentation cycle and 179 further fermentation cycles of the total fermenting population) according to the present process, the persistent yeast cells of the present disclosure are present in a proportion of at least 98% in the fermenting population (when measured with respect to its DNA contribution of the total fermenting population). In an embodiment, after a total 180 fermentation cycles (comprising both the initial fermentation cycle and 179 further fermentation cycles) according to the present process, the persistent yeast cells of the present disclosure are present in a proportion of at least 99% in the fermenting population (when measured with respect to its DNA contribution of the total fermenting population). In an embodiment, after a total 180 fermentation cycles (comprising both the initial fermentation cycle and 179 further fermentation cycles) according to the present process, the persistent yeast cells of the present disclosure are present in a proportion of at least 99.1% in the fermenting population (when measured with respect to its DNA contribution of the total fermenting population). In an embodiment, after a total 180 fermentation cycles (comprising both the initial fermentation cycle and 179 further fermentation cycles) according to the present process, the persistent yeast cells of the present disclosure are present in a proportion of at least 99.2% in the fermenting population (when measured with respect to its DNA contribution of the total fermenting population). In an embodiment, after a total 180 fermentation cycles (comprising both the initial fermentation cycle and 179 further fermentation cycles) according to the present process, the persistent yeast cells of the present disclosure are present in a proportion of at least 99.3% in the fermenting population (when measured with respect to its DNA contribution of the total fermenting population). In an embodiment, after a total 180 fermentation cycles (comprising both the initial fermentation cycle and 179 further fermentation cycles) according to the present process, the persistent yeast cells of the present disclosure are present in a proportion of at least 99.4% in the fermenting population (when measured with respect to its DNA contribution of the total fermenting population). In an embodiment, after a total 180 fermentation cycles (comprising both the initial fermentation cycle and 179 further fermentation cycles) according to the present process, the persistent yeast cells of the present disclosure are present in a proportion of at least 99.5% in the fermenting population (when measured with respect to its DNA contribution of the total fermenting population). In an embodiment, after a total 180 fermentation cycles (comprising both the initial fermentation cycle and 179 further fermentation cycles) according to the present process, the persistent yeast cells of the present disclosure are present in a proportion of at least 99.6% in the fermenting population (when measured with respect to its DNA contribution of the total fermenting population). In an embodiment, after a total 180 fermentation cycles (comprising both the initial fermentation cycle and 179 further fermentation cycles) according to the present process, the persistent yeast cells of the present disclosure are present in a proportion of at least 99.7% in the fermenting population (when measured with respect to its DNA contribution of the total fermenting population). In an embodiment, after a total 180 fermentation cycles (comprising both the initial fermentation cycle and 179 further fermentation cycles) according to the present process, the persistent yeast cells of the present disclosure are present in a proportion of at least 99.8% in the fermenting population (when measured with respect to its DNA contribution of the total fermenting population). In an embodiment, after a total 180 fermentation cycles (comprising both the initial fermentation cycle and 179 further fermentation cycles) according to the present process, the persistent yeast cells of the present disclosure are present in a proportion of at least 99.9% in the fermenting population (when measured with respect to its DNA contribution of the total fermenting population).

In an embodiment, after a total 190 fermentation cycles (comprising both the initial fermentation cycle and 189 further fermentation cycles) according to the present process, the persistent yeast cells of the present disclosure are present in a proportion of at least 90% in the fermenting population (when measured with respect to its DNA contribution of the total fermenting population). In an embodiment, after a total 190 fermentation cycles (comprising both the initial fermentation cycle and 189 further fermentation cycles) according to the present process, the persistent yeast cells of the present disclosure are present in a proportion of at least 91% in the fermenting population (when measured with respect to its DNA contribution of the total fermenting population). In an embodiment, after a total 190 fermentation cycles (comprising both the initial fermentation cycle and 189 further fermentation cycles) according to the present process, the persistent yeast cells of the present disclosure are present in a proportion of at least 92% in the fermenting population (when measured with respect to its DNA contribution of the total fermenting population). In an embodiment, after a total 190 fermentation cycles (comprising both the initial fermentation cycle and 189 further fermentation cycles of the total fermenting population) according to the present process, the persistent yeast cells of the present disclosure are present in a proportion of at least 93% in the fermenting population (when measured with respect to its DNA contribution of the total fermenting population). In an embodiment, after a total 190 fermentation cycles (comprising both the initial fermentation cycle and 189 further fermentation cycles of the total fermenting population) according to the present process, the persistent yeast cells of the present disclosure are present in a proportion of at least 94% in the fermenting population (when measured with respect to its DNA contribution of the total fermenting population). In an embodiment, after a total 190 fermentation cycles (comprising both the initial fermentation cycle and 189 further fermentation cycles of the total fermenting population) according to the present process, the persistent yeast cells of the present disclosure are present in a proportion of at least 95% in the fermenting population (when measured with respect to its DNA contribution of the total fermenting population). In an embodiment, after a total 190 fermentation cycles (comprising both the initial fermentation cycle and 189 further fermentation cycles of the total fermenting population) according to the present process, the persistent yeast cells of the present disclosure are present in a proportion of at least 96% in the fermenting population (when measured with respect to its DNA contribution of the total fermenting population). In an embodiment, after a total 190 fermentation cycles (comprising both the initial fermentation cycle and 189 further fermentation cycles) according to the present process, the persistent yeast cells of the present disclosure are present in a proportion of at least 97% in the fermenting population (when measured with respect to its DNA contribution of the total fermenting population). In an embodiment, after a total 190 fermentation cycles (comprising both the initial fermentation cycle and 189 further fermentation cycles of the total fermenting population) according to the present process, the persistent yeast cells of the present disclosure are present in a proportion of at least 98% in the fermenting population (when measured with respect to its DNA contribution of the total fermenting population). In an embodiment, after a total 190 fermentation cycles (comprising both the initial fermentation cycle and 189 further fermentation cycles) according to the present process, the persistent yeast cells of the present disclosure are present in a proportion of at least 99% in the fermenting population (when measured with respect to its DNA contribution of the total fermenting population). In an embodiment, after a total 190 fermentation cycles (comprising both the initial fermentation cycle and 189 further fermentation cycles) according to the present process, the persistent yeast cells of the present disclosure are present in a proportion of at least 99.1% in the fermenting population (when measured with respect to its DNA contribution of the total fermenting population). In an embodiment, after a total 190 fermentation cycles (comprising both the initial fermentation cycle and 189 further fermentation cycles) according to the present process, the persistent yeast cells of the present disclosure are present in a proportion of at least 99.2% in the fermenting population (when measured with respect to its DNA contribution of the total fermenting population). In an embodiment, after a total 190 fermentation cycles (comprising both the initial fermentation cycle and 189 further fermentation cycles) according to the present process, the persistent yeast cells of the present disclosure are present in a proportion of at least 99.3% in the fermenting population (when measured with respect to its DNA contribution of the total fermenting population). In an embodiment, after a total 190 fermentation cycles (comprising both the initial fermentation cycle and 189 further fermentation cycles) according to the present process, the persistent yeast cells of the present disclosure are present in a proportion of at least 99.4% in the fermenting population (when measured with respect to its DNA contribution of the total fermenting population). In an embodiment, after a total 190 fermentation cycles (comprising both the initial fermentation cycle and 189 further fermentation cycles) according to the present process, the persistent yeast cells of the present disclosure are present in a proportion of at least 99.5% in the fermenting population (when measured with respect to its DNA contribution of the total fermenting population). In an embodiment, after a total 190 fermentation cycles (comprising both the initial fermentation cycle and 189 further fermentation cycles) according to the present process, the persistent yeast cells of the present disclosure are present in a proportion of at least 99.6% in the fermenting population (when measured with respect to its DNA contribution of the total fermenting population). In an embodiment, after a total 190 fermentation cycles (comprising both the initial fermentation cycle and 189 further fermentation cycles) according to the present process, the persistent yeast cells of the present disclosure are present in a proportion of at least 99.7% in the fermenting population (when measured with respect to its DNA contribution of the total fermenting population). In an embodiment, after a total 190 fermentation cycles (comprising both the initial fermentation cycle and 189 further fermentation cycles) according to the present process, the persistent yeast cells of the present disclosure are present in a proportion of at least 99.8% in the fermenting population (when measured with respect to its DNA contribution of the total fermenting population). In an embodiment, after a total 190 fermentation cycles (comprising both the initial fermentation cycle and 189 further fermentation cycles) according to the present process, the persistent yeast cells of the present disclosure are present in a proportion of at least 99.9% in the fermenting population (when measured with respect to its DNA contribution of the total fermenting population).

In an embodiment, after a total 200 fermentation cycles (comprising both the initial fermentation cycle and 199 further fermentation cycles) according to the present process, the persistent yeast cells of the present disclosure are present in a proportion of at least 90% in the fermenting population (when measured with respect to its DNA contribution of the total fermenting population). In an embodiment, after a total 200 fermentation cycles (comprising both the initial fermentation cycle and 199 further fermentation cycles) according to the present process, the persistent yeast cells of the present disclosure are present in a proportion of at least 91% in the fermenting population (when measured with respect to its DNA contribution of the total fermenting population). In an embodiment, after a total 200 fermentation cycles (comprising both the initial fermentation cycle and 199 further fermentation cycles) according to the present process, the persistent yeast cells of the present disclosure are present in a proportion of at least 92% in the fermenting population (when measured with respect to its DNA contribution of the total fermenting population). In an embodiment, after a total 200 fermentation cycles (comprising both the initial fermentation cycle and 199 further fermentation cycles of the total fermenting population) according to the present process, the persistent yeast cells of the present disclosure are present in a proportion of at least 93% in the fermenting population (when measured with respect to its DNA contribution of the total fermenting population). In an embodiment, after a total 200 fermentation cycles (comprising both the initial fermentation cycle and 199 further fermentation cycles of the total fermenting population) according to the present process, the persistent yeast cells of the present disclosure are present in a proportion of at least 94% in the fermenting population (when measured with respect to its DNA contribution of the total fermenting population). In an embodiment, after a total 200 fermentation cycles (comprising both the initial fermentation cycle and 199 further fermentation cycles of the total fermenting population) according to the present process, the persistent yeast cells of the present disclosure are present in a proportion of at least 95% in the fermenting population (when measured with respect to its DNA contribution of the total fermenting population). In an embodiment, after a total 200 fermentation cycles (comprising both the initial fermentation cycle and 199 further fermentation cycles of the total fermenting population) according to the present process, the persistent yeast cells of the present disclosure are present in a proportion of at least 96% in the fermenting population (when measured with respect to its DNA contribution of the total fermenting population). In an embodiment, after a total 200 fermentation cycles (comprising both the initial fermentation cycle and 199 further fermentation cycles) according to the present process, the persistent yeast cells of the present disclosure are present in a proportion of at least 97% in the fermenting population (when measured with respect to its DNA contribution of the total fermenting population). In an embodiment, after a total 200 fermentation cycles (comprising both the initial fermentation cycle and 199 further fermentation cycles of the total fermenting population) according to the present process, the persistent yeast cells of the present disclosure are present in a proportion of at least 98% in the fermenting population (when measured with respect to its DNA contribution of the total fermenting population). In an embodiment, after a total 200 fermentation cycles (comprising both the initial fermentation cycle and 199 further fermentation cycles) according to the present process, the persistent yeast cells of the present disclosure are present in a proportion of at least 99% in the fermenting population (when measured with respect to its DNA contribution of the total fermenting population). In an embodiment, after a total 200 fermentation cycles (comprising both the initial fermentation cycle and 199 further fermentation cycles) according to the present process, the persistent yeast cells of the present disclosure are present in a proportion of at least 99.1% in the fermenting population (when measured with respect to its DNA contribution of the total fermenting population). In an embodiment, after a total 200 fermentation cycles (comprising both the initial fermentation cycle and 199 further fermentation cycles) according to the present process, the persistent yeast cells of the present disclosure are present in a proportion of at least 99.2% in the fermenting population (when measured with respect to its DNA contribution of the total fermenting population). In an embodiment, after a total 200 fermentation cycles (comprising both the initial fermentation cycle and 199 further fermentation cycles) according to the present process, the persistent yeast cells of the present disclosure are present in a proportion of at least 99.3% in the fermenting population (when measured with respect to its DNA contribution of the total fermenting population). In an embodiment, after a total 200 fermentation cycles (comprising both the initial fermentation cycle and 199 further fermentation cycles) according to the present process, the persistent yeast cells of the present disclosure are present in a proportion of at least 99.4% in the fermenting population (when measured with respect to its DNA contribution of the total fermenting population). In an embodiment, after a total 200 fermentation cycles (comprising both the initial fermentation cycle and 199 further fermentation cycles) according to the present process, the persistent yeast cells of the present disclosure are present in a proportion of at least 99.5% in the fermenting population (when measured with respect to its DNA contribution of the total fermenting population). In an embodiment, after a total 200 fermentation cycles (comprising both the initial fermentation cycle and 199 further fermentation cycles) according to the present process, the persistent yeast cells of the present disclosure are present in a proportion of at least 99.6% in the fermenting population (when measured with respect to its DNA contribution of the total fermenting population). In an embodiment, after a total 200 fermentation cycles (comprising both the initial fermentation cycle and 199 further fermentation cycles) according to the present process, the persistent yeast cells of the present disclosure are present in a proportion of at least 99.7% in the fermenting population (when measured with respect to its DNA contribution of the total fermenting population). In an embodiment, after a total 200 fermentation cycles (comprising both the initial fermentation cycle and 199 further fermentation cycles) according to the present process, the persistent yeast cells of the present disclosure are present in a proportion of at least 99.8% in the fermenting population (when measured with respect to its DNA contribution of the total fermenting population). In an embodiment, after a total 200 fermentation cycles (comprising both the initial fermentation cycle and 199 further fermentation cycles) according to the present process, the persistent yeast cells of the present disclosure are present in a proportion of at least 99.9% in the fermenting population (when measured with respect to its DNA contribution of the total fermenting population).

The initial and further fermentation medium comprises or is derived from a biomass. The biomass that can be fermented with the recombinant host cell described herein includes any type of biomass known in the art and described herein. For example, the biomass can include, but is not limited to, starch, sugar and lignocellulosic materials. Starch materials can include, but are not limited to, mashes such as corn, wheat, rye, barley, rice, or milo. Sugar materials can include, but are not limited to, sugar beets, artichoke tubers, sweet sorghum, molasses or sugarcane. The terms “lignocellulosic material”, “lignocellulosic substrate” and “cellulosic biomass” mean any type of biomass comprising cellulose, hemicellulose, lignin, or combinations thereof, such as but not limited to woody biomass, forage grasses, herbaceous energy crops, non-woody-plant biomass, agricultural wastes and/or agricultural residues, forestry residues and/or forestry wastes, paper-production sludge and/or waste paper sludge, waste-water-treatment sludge, municipal solid waste, corn fiber from wet and dry mill corn ethanol plants and sugar-processing residues. The terms “hemicellulosics”, “hemicellulosic portions” and “hemicellulosic fractions” mean the non-lignin, non-cellulose elements of lignocellulosic material, such as but not limited to hemicellulose (i.e., comprising xyloglucan, xylan, glucuronoxylan, arabinoxylan, mannan, glucomannan and galactoglucomannan), pectins (e.g., homogalacturonans, rhamnogalacturonan I and II, and xylogalacturonan) and proteoglycans (e.g., arabinogalactan-protein, extensin, and pro line-rich proteins). In a further embodiment, the initial and/or the further fermentation medium comprises sucrose as the main fermentable carbohydrate. In one embodiment, the initial and/or the further fermentation medium comprises or is derived from sugarcane, molasses, derivatives thereof as well as mixtures thereof.

In a non-limiting example, the lignocellulosic material can include, but is not limited to, woody biomass, such as recycled wood pulp fiber, sawdust, hardwood, softwood, and combinations thereof; grasses, such as switch grass, cord grass, rye grass, reed canary grass, miscanthus, or a combination thereof; sugar-processing residues, such as but not limited to sugar cane bagasse; agricultural wastes, such as but not limited to rice straw, rice hulls, barley straw, corn cobs, cereal straw, wheat straw, canola straw, oat straw, oat hulls, and corn fiber; stover, such as but not limited to soybean stover, corn stover; succulents, such as but not limited to, agave; and forestry wastes, such as but not limited to, recycled wood pulp fiber, sawdust, hardwood (e.g., poplar, oak, maple, birch, willow), softwood, or any combination thereof. Lignocellulosic material may comprise one species of fiber; alternatively, lignocellulosic material may comprise a mixture of fibers that originate from different lignocellulosic materials. Other lignocellulosic materials are agricultural wastes, such as cereal straws, including wheat straw, barley straw, canola straw and oat straw; corn fiber; stovers, such as corn stover and soybean stover; grasses, such as switch grass, reed canary grass, cord grass, and miscanthus; or combinations thereof.

Substrates for cellulose activity assays can be divided into two categories, soluble and insoluble, based on their solubility in water. Soluble substrates include cellodextrins or derivatives, carboxymethyl cellulose (CMC), or hydroxyethyl cellulose (HEC). Insoluble substrates include crystalline cellulose, microcrystalline cellulose (Avicel), amorphous cellulose, such as phosphoric acid swollen cellulose (PASC), dyed or fluorescent cellulose, and pretreated lignocellulosic biomass. These substrates are generally highly ordered cellulosic material and thus only sparingly soluble.

It will be appreciated that suitable lignocellulosic material may be any feedstock that contains soluble and/or insoluble cellulose, where the insoluble cellulose may be in a crystalline or non-crystalline form. In various embodiments, the lignocellulosic biomass comprises, for example, wood, corn, corn stover, sawdust, bark, molasses, sugarcane, leaves, agricultural and forestry residues, grasses such as switchgrass, ruminant digestion products, municipal wastes, paper mill effluent, newspaper, cardboard or combinations thereof.

Paper sludge is also a viable feedstock for lactate or acetate production. Paper sludge is solid residue arising from pulping and paper-making, and is typically removed from process wastewater in a primary clarifier. The cost of disposing of wet sludge is a significant incentive to convert the material for other uses, such as conversion to ethanol. Processes provided by the present invention are widely applicable. Moreover, the saccharification and/or fermentation products may be used to produce ethanol or higher value added chemicals, such as organic acids, aromatics, esters, acetone and polymer intermediates.

In the process described herein, it is possible to add an exogenous source (e.g., to dose) of an enzyme to facilitate saccharification or improve fermentation yield. As such, the process can comprise including one or more dose(s) of one or more enzyme(s). The exogenous enzyme that can be used can include, without limitation, an alpha-amylase, a glucoamylase, a protease, a phytase, a pullulanase, a cellulase, a hemi-cellulase such as a xylanase, a trehalase, or any combination thereof. The exogenous enzyme can be provided, in some embodiments, in a purified form and/or provided as part of a cocktail.

The production of ethanol can be performed, for example, at temperatures of at least about 30° C., about 31° C., about 32° C., about 33°, about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C. In some embodiments, the production of ethanol from cellulose can be performed, for example, at temperatures above about 30° C., about 31° C., about 32° C., about 33° C., about 34° C., about 35° C., about 36° C., about 37° C., about 38° C., about 39° C., about 40° C., about 41° C., about 42° C., or about 43° C., or about 44° C., or about 45° C., or about 50° C. In some embodiments, the persistent yeast can produce ethanol from cellulose at temperatures from about 30° C. to 60° C., about 30° C. to 55° C., about 30° C. to 50° C., about 40° C. to 60° C., about 40° C. to 55° C. or about 40° C. to 50° C.

In the processes described herein, at the end of the fermentation, the fermenting population is substantially isolated from the fermented fermentation medium. As used in the context of the present disclosure, the expression “substantially isolating” refers to the removal of the majority of the components of the fermented fermentation medium from the fermenting population. In some embodiments, “substantially isolating” refers to concentrating the fermenting population to at least 5, 10, 15, 20, 25, 30, 35, 45% or more when compared to the concentration of the fermenting population prior to the substantially isolation. In order to substantially isolate to fermenting population, the fermented fermentation medium can be centrifuged. Cell separation and recovery in the fuel ethanol process is carried out using stacked-disk, nozzle discharge type centrifuges (see Brociner et al.). In these machines, the feed-broth from the end of fermentation, often referred to in the process as “vinho bruto” or “beer” is introduced into the top of the machine, circulates to the bottom, and is then forced upward through a set of rotating disks. The rotation of these disks imparts a centrifugal force on the total feed, and particles.

Yeast cells and other solids are forced downward and to the side of the machine. The cells then exit through nozzles at the outer edge of the machine creating a concentrated yeast cream. Clarified liquid, often called “vinho,”, “vinho delevurado” or “wine” exits the machine out the top.

Optionally the substantially isolated fermenting population can be washed. In a specific embodiment, the substantially isolated fermenting population can be submitted to an acid washing step. In the acid washing step, an acid or an acidic solution is put into contact with the fermenting population. In some embodiments, the acid or the acidic solution has a pH of between 2.0 and 2.2. In some embodiments, the contact between the substantially isolated fermenting population and the acid/acidic solution is maintained so as to reduce the contaminating bacterial population that may be present. For example, the contact between the substantially isolated fermenting population and the acid or the acidic solution can last at least 30, 40, 50, 60, 70, 80, 90, 100, 110, 120 minutes or more. In certain embodiments, the acid is sulphuric acid and/or the acidic solution comprises sulphuric acid. After the acid washing step, the pH of the acid washed fermenting population can be adjusted prior to the further fermentation cycle.

In some embodiments, methods of producing ethanol can comprise contacting the fermentation substrate with a persistent yeast described herein and additionally contacting the substrate with externally produced enzymes which can be provided in a purified form. Exemplary externally produced enzymes include, but are not limited to starch degrading enzymes, dextran degrading enzymes, phytase, protease, cellulases and/or xylose isomerase. Specific externally produced (and optionally purified) enzymes include, but are not limited to, trehalases, glucoamylases, alpha-amylases, alpha-glucosidases, glucanases (endo/exo), pullulanases, phytases and/or proteases.

In some embodiments, the methods comprise producing ethanol at a particular rate. For example, in some embodiments, ethanol is produced at a rate of at least about 0.1 mg per hour per liter, at least about 0.25 mg per hour per liter, at least about 0.5 mg per hour per liter, at least about 0.75 mg per hour per liter, at least about 1.0 mg per hour per liter, at least about 2.0 mg per hour per liter, at least about 5.0 mg per hour per liter, at least about 10 mg per hour per liter, at least about 15 mg per hour per liter, at least about 20.0 mg per hour per liter, at least about 25 mg per hour per liter, at least about 30 mg per hour per liter, at least about 50 mg per hour per liter, at least about 100 mg per hour per liter, at least about 200 mg per hour per liter, at least about 300 mg per hour per liter, at least about 400 mg per hour per liter, at least about 500 mg per hour per liter, at least about 600 mg per hour per liter, at least about 700 mg per hour per liter, at least about 800 mg per hour per liter, at least about 900 mg per hour per liter, at least about 1 g per hour per liter, at least about 1.5 g per hour per liter, at least about 2 g per hour per liter, at least about 2.5 g per hour per liter, at least about 3 g per hour per liter, at least about 3.5 g per hour per liter, at least about 4 g per hour per liter, at least about 4.5 g per hour per liter, at least about 5 g per hour per liter, at least about 5.5 g per hour per liter, at least about 6 g per hour per liter, at least about 6.5 g per hour per liter, at least about 7 g per hour per liter, at least about 7.5 g per hour per liter, at least about 8 g per hour per liter, at least about 8.5 g per hour per liter, at least about 9 g per hour per liter, at least about 9.5 g per hour per liter, at least about 10 g per hour per liter, at least about 10.5 g per hour per liter, at least about 11 g per hour per liter, at least about 11.5 g per hour per liter, at least about 12 g per hour per liter, at least about 12.5 g per hour per liter, at least about 13 g per hour per liter, at least about 13.5 g per hour per liter, at least about 14 g per hour per liter, at least about 14.5 g per hour per liter or at least about 15 g per hour per liter.

In some embodiments, the persistent yeast cells can produce ethanol at a rate of at least about 0.1 mg per hour per liter, at least about 0.25 mg per hour per liter, at least about 0.5 mg per hour per liter, at least about 0.75 mg per hour per liter, at least about 1.0 mg per hour per liter, at least about 2.0 mg per hour per liter, at least about 5.0 mg per hour per liter, at least about 10 mg per hour per liter, at least about 15 mg per hour per liter, at least about 20.0 mg per hour per liter, at least about 25 mg per hour per liter, at least about 30 mg per hour per liter, at least about 50 mg per hour per liter, at least about 100 mg per hour per liter, at least about 200 mg per hour per liter, at least about 300 mg per hour per liter, at least about 400 mg per hour per liter, at least about 500 mg per hour per liter, at least about 600 mg per hour per liter, at least about 700 mg per hour per liter, at least about 800 mg per hour per liter, at least about 900 mg per hour per liter, at least about 1 g per hour per liter, at least about 1.5 g per hour per liter, at least about 2 g per hour per liter, at least about 2.5 g per hour per liter, at least about 3 g per hour per liter, at least about 3.5 g per hour per liter, at least about 4 g per hour per liter, at least about 4.5 g per hour per liter, at least about 5 g per hour per liter, at least about 5.5 g per hour per liter, at least about 6 g per hour per liter, at least about 6.5 g per hour per liter, at least about 7 g per hour per liter, at least about 7.5 g per hour per liter, at least about 8 g per hour per liter, at least about 8.5 g per hour per liter, at least about 9 g per hour per liter, at least about 9.5 g per hour per liter, at least about 10 g per hour per liter, at least about 10.5 g per hour per liter, at least about 11 g per hour per liter, at least about 11.5 g per hour per liter, at least about 12 g per hour per liter, at least about 12.5 g per hour per liter, at least about 13 g per hour per liter, at least about 13.5 g per hour per liter, at least about 14 g per hour per liter, at least about 14.5 g per hour per liter, at least about 15 g per hour per liter or more than a control strain (e.g., a wild-type strain, such as, for example, strain PE-2) and grown under the same conditions. In some embodiments, the ethanol can be produced in the absence of any externally added cellulases.

Ethanol production can be measured using any method known in the art. For example, the quantity of ethanol in fermentation samples can be assessed using HPLC analysis. Many ethanol assay kits are commercially available that use, for example, alcohol oxidase enzyme based assays.

### Processes for Screening/Generating Persistent Yeast Cells

The present disclosure also provides for a process for determining if a test yeast is considered persistent or not. The process comprises conducting a plurality of fermentation cycles using, in the initial fermenting population a test yeast and determining, after a specific number of fermentation cycles, if the test yeast is present in the fermented medium and if so, the proportion of the test yeast in the fermentation medium. The test yeast has a detectable feature which is absent from other wild yeasts which may contaminate the fermentation medium. If the test yeast is present, after a total of 40, 50, 60, 70, 80, 90, 100, 110, 120, 130, 140, 150, 160, 170, 180, 190, 200 fermentation cycles or more, in a proportion at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more in the fermenting population, the test yeast is considered to be persistent. If the test yeast is present, after a total of 40, 50, 60, 70, 80, 90, 100, 110, 120, 130, 140, 150, 160, 170, 180, 190, 200 fermentation cycles or more, in a proportion lower than 90% in the fermenting population, the test yeast is not considered to be persistent. The process can include, prior to performing the plurality of fermentation cycles, providing a test yeast as a recombinant yeast host cell capable of modulating the activity or the expression of a first polypeptide and/or a second polypeptide for increasing, when compared to a parental cell, the conversion of a biomass into a fermentation product and/or for reducing the conversion of the biomass into a fermentation by-product as described herein. In some embodiments, the process can include introducing one or more genetic modification, for example those presented above, to modulate the activity or the expression of the first polypeptide and/or the second polypeptide in the test yeast which is considered to be persistent or intended to be screened for persistence phenotypes. The process can include, in some embodiments, determining if the test yeast is persistent by determining the presence or the absence of at least one or any combination of phenotypic traits. A test yeast comprising at least one or any combination of the phenotypic traits described herein is considered to be persistent.

The present disclosure also provides for a process for generating a persistent yeast. The process comprises conducting a plurality of fermentation cycles using, in the initial fermenting population comprising an initial yeast or a combination of initial yeasts and conducting a specific number of fermentation cycles. The initial yeast(s) may or may not be considered to be persistent. In some embodiments, it is not known if the initial yeast(s) is persistent or not. The fermenting population obtained after a total of 40, 50, 60, 70, 80, 90, 100, 110, 120, 130, 140, 150, 160, 170, 180, 190, 200 fermentation cycles or more is considered to include persistent yeast cells. The persistent yeast cells may or may not corresponding to the initial yeast(s) initially inoculated in the fermentation medium. The process can include, in some embodiments, determining if the fermenting population obtained after a total of at least 40 fermentation cycles is persistent by determining the presence or the absence of the at least one phenotypic traits in one or more cells of the fermenting population. A yeast cell obtained (and in some embodiments substantially isolated) from the fermenting population after at least 40 fermentation cycles exhibiting at least one or any combination of the phenotypic traits described herein is considered to be persistent. The persistent yeast cells obtained by this process can, in some embodiments, be genetically modified to have the ability of modulating the activity or the expression of a first polypeptide and/or a second polypeptide for increasing, when compared to a parental cell, the conversion of a biomass into a fermentation product and/or for reducing the conversion of the biomass into a fermentation by-product as described herein.

The present invention will be more readily understood by referring to the following examples which are given to illustrate the invention rather than to limit its scope.

### Example

### Material and Methods

Gene sequencing. DNA was extracted from particular fermentation cycles throughout the implementation test and Illumina sequenced (2×126 bp read data; short reads of all DNA present).

Quantitative PCR (qPCR) assay. The amount of the recombinant yeast strain was measured by a quantitative polymerase chain reaction (qPCR) technique that quantifies the amount of the IME1 gene present in a sample compared to the amount of the ALG9 gene. Since the recombinant yeast strain does not have the IME1 gene (because it includes at least one genetic modification disrupting the IME1 gene), the amount of quantified IME1 DNA relative to the amount of ALG9 DNA increases as the amount of contaminating DNA (from wild yeasts) increases. The absolute quantification method was used to analyze the samples by comparison to a standard curve for each gene of interest. The qPCR reactions for the unknowns contains the following components, genomic DNA (loaded at 0.05 ng/μl), SsoAdvanced™ Universal SYBR® Green Supermix (Biorad #172-5271), and primer pairs specific to either the IME1 or the ALG9 gene at 0.5 uM final concentration. The IME1 gene was amplified with primer X33463 (cacgctgccttagaagatgg, SEQ ID NO: 1) and primer X33464 (gttctgcagctgagatgagg, SEQ ID NO: 2). The ALG9 gene was amplified with primer X34382 (gtttaatccgggctggttccat, SEQ ID NO: 3) and primer X34383 (TAGACCCAGTGGACAGATAGCG, SEQ ID NO: 4). The amplification was performed in a BioRad CFX96 Touch Real-Time PCR Detection System using 2-step PCR cycling conditions with a melting temperature of 60° C. for 30 seconds for a total of 40 cycles. Finally, the starting quantity (SQ) of the sample DNA was determined by comparing the cycle of quantitation (Cq) for the unknown to the Cq of the known DNA quantities in the standard curve for each primer set. The calculation (IME1 SQ/ALG9 SQ*100) determined the percentage of wild type DNA in the population and was used to estimate the presence of the recombinant yeast strain.

Scanning election microscopy. Scanning electron microscopy (SEM) was used to study the morphological shape of yeast cells. The cells were fixed in glutaraldehyde, post-fixed in OSO₄, dried and sputter-coated with gold/palladium (Au/Pd) for viewing. The specimen were fixed by immersing them in 2.5%-4% glutaraldehyde in 0.1 M sodium cacodylate or phosphate buffer solution (pH 6.8-7.4 as appropriate) and incubating for at least two hours. The specimen were washed by immersing them 3 times with the 0.1 M cacodylate or phosphate buffer solution for 10 minutes each to remove excess fixative. The specimen were then submitted to a post-fixation treatment by immersing them in a 1% OsO₄ in buffer at 4° C. and further washed in distilled or de-ionized water to remove all traces of fixative and buffer solutions. The specimens were dehydrated by immersing them for 10 minutes each in 25%, 50%, 70%, 85%, 95%, and three 100% ethanol solutions to remove all traces of water. The specimens were submitted to a critical point dry or three washes in HMDS for 10 minutes each followed by air drying. The specimens were mounted and a sputter coat a thin layer (˜5 nm) of AuPd was applied.

Commercial fed batch fermentation process. A detailed description of the fermentation process with yeast recycle with its various forms can be found in Basso et al. (2001). Briefly, non-sterilized sugarcane juice and/or molasses with a typical concentration of between 18% and 22% total sugars was fermented at a high yeast inoculum (10 to 14% wet weight per volume) to achieve fast (8 to 12 hours) fermentations. The sugar concentration can vary over the course of the sugarcane harvest season, and is typically lowest at the start and end of the season. The yeast was repeatedly recycled over the crop season (˜200 days). After each cycle of fermentation, yeast cells were separated from the whole fermentation broth by centrifugation, with some of the yeast being lost with the wine from the centrifuge and proceeding to distillation, and the retained yeast remaining as a concentrated yeast cream. The yeast cream was then subjected to a treatment with sulphuric acid where water and acid are added until the yeast cream reaches a pH of between 3.5 to 1.8 (although in some cases no acid treatment is used). This treatment lasted 1 to 2 hours and was used to decrease bacterial contamination and break up flocculation of the yeast cells. In some cases antimicrobials were added to the yeast cream prior to fermentation as well. After the treatment was complete, the yeast cell cream was repitched to a fermentation tank and fresh substrate (molasses, sugarcane juice, or a mixture) was then added to the cells and fermentation is started again. Fermentation temperatures varied based on the configuration of the industrial facility, but were generally targeted to between 32 and 35° C. In many cases, the temperature cannot be controlled in this range and can reach as high as 40° C. or higher. Fermentation vessels were typically only agitated by the CO₂ generated during fermentation escaping the broth and by the action of a pump around loop with a heat exchanger to remove excess heat.

Laboratory scale fed batch cell recycle fermentation process. Lab scale fermentations were set up to mimic the industrial scale process. They were carried out using 50 mL vessels filled with yeast cream either from propagation, or from a previous fermentation, at a level to reproduce standard yeast concentrations in fermentations recycling yeasts (˜10% wet cell mass). This yeast cream was subjected to acid treatment under the conditions provided above in the section “Commercial fed batch fermentation process”. A feeding system was used to provide a feed of substrate or “must” (sugar cane must sourced from operating facilities), again at rates and concentrations dictated by average conditions occurring in commercial facilities. This feed stream was provided via a syringe pump to each reactor. Fermentations were held under temperature controlled conditions (generally 32 to 35° C.) and gently agitated, and were allowed to proceed until the evolution of CO₂ falls below a minimum threshold. Once complete, samples were taken for analysis by HPLC to compare the production of ethanol, glycerol, organic acids, and other compounds. Yeast cultures were run as either pure cultures or as mixtures of yeast strains as indicated in the experimental description or figure legends. The amounts of different strains present after a particular number of cycles was determined using a qPCR technique specific to the strain of interest.

Batch laboraty scale fermentation. Anaerobic batch fermentations were run in 60 mL pressure bottles with 20 mL of media (commercially sourced must or defined laboratory media). Fermentations were held under temperature controlled conditions and gently agitated, and were allowed to proceed until the evolution of CO₂ falls below a minimum threshold. Once complete, samples were taken for analysis by HPLC to compare the production of ethanol, glycerol, organic acids, and other compounds. Aerobic cultures were similarly set-up but with a permeable cap. Cultures of yeast strains Y2, Y4 and Y8 were pitched in monoculture or in co-culture with ˜15% of the wild type strain Y7. The fermentations were monitored by HPLC and yeast populations were monitored by qPCR.

Suspension assay. Cells were suspended evenly at time zero, taking a sample from the top of the suspension and measuring the sample's optical density at 600 nm. The sample was then incubated for a designated amount of time (3.5 or 5 min). After the incubation, another sample was obtained from the top of the suspension and the optical density is again measured at 600 nm. The percentage of sedimentation corresponds to the percentage change in optical density between the two samples. Samples in this assay included a control, which was a non-clustered yeast strain freshly grown up on media, as well as samples taken directly from the centrifugation process.

Wash out rate determination. Washout rates were determined by measuring the amount of non-engineered strain present in the fermentation system of the facility by carrying out qPCR of composite samples (as described above). Then, overall exponential washout rates (K value) were calculated using the formula

$$K = ln ( \frac{{C}_{FINAL}}{{C}_{INITIAL}} ) / ( {CYCLE}_{FINAL} - {CYCLE}_{INITIAL} )$$

where C_{FINAL}=the fraction of contaminant yeast DNA at the final cycle measured; C_{INITIAL}=the fraction of contaminant yeast DNA at the first cycle measured; CYCLE_{FINAL}=the number of the final cycle measured; CYCLE_{INITIAL}=the number of the first cycle measured.

Genome-wide association tests. The software Plink (Purcell et al. 2007) can conduct a genome-wide scan for alleles that significantly associate with a phenotype of interest. Following ploidy inference, the filtered variant callset of the appropriate ploidy were converted to Plink format using BCFtools (Li et al. 2009; Li, 2011) to create a chromosome-map file, and VCFtools (Danecek et al., 2011) to execute the conversion. Each strain was coded as being either smooth (0) or rugose (1) and conducted the association test for SNPs and indels independently, log-transforming the resulting p-values to plot the relative strength of the genotype-phenotype association across each position. In conventional genome-wide association studies (GWAS) involving hundreds to thousands of samples, p-values <1×10⁻⁵ are considered suggestive and p-values <5×10⁻⁸ are considered significant. In the present case, comparatively few samples (N=25), even perfect associations may not yield conventionally significant p-values depending on the level of noise in the genome-wide background, but the results can be used to narrow down the field of candidate causal mutations by quantifying the relative strength of the association across a genome-wide panel of variants.

Invertase assay. Cells were grown overnight in aerobic or anaerobic culture, or harvested at the end of one or more fermentation cycles in the fed-batch cell recycle system. Cultures were diluted to 9 mg/mL (wet weight basis, ˜2 g/L dry weight basis) in a citrate-phosphate buffer pH 5 with 40 g/L sucrose and incubated at 35° C. for 12 to 15 minutes. The mixture is then incubated with 3,5-dinitrosalicylic acid (DNS). DNS reacts with reducing sugars and to form 3-amino-5-nitrosalicylic acid, which absorbs light at 540 nm. Glucose and fructose released by the yeast invertase activity is quantified by spectrophotometry at 540 nm. Dry weight measurements of cell samples are used to calculate the amount of dry cells added in each reaction and the sugar release per gram of dry cells loaded per time.

Response to cAMP. Cell were grown in YPD for 48 hours until they were glucose depleted followed by a spike of 100 mM glucose to the cells. Cells were snap frozen before glucose addition and after 5 minutes of incubation with the glucose. Cells were thawed, lysed and the amount of cAMP was then measured by a standard kit.

TABLE 1
Description of the strains that were made and/or characterizes. GPD2 had the amino
acid sequence of SEQ ID NO: 5 was encoded by the nucleic acid sequence of SEQ ID NO: 6.
STL1 had the amino acid sequence of SEQ ID NO: 7 was encoded by the nucleic acid
sequence of SEQ ID NO: 8. SmGAPN had the amino acid sequence of SEQ ID NO: 29 and
was encoded by the nucleic acid sequence of SEQ ID NO: 30. The presence of the a plurality
of acronyms in a strain (STL1-STL1 for example) refers that two copies of a gene encoding for
the polypeptide referred to by the acronym has been inserted at each integration site.

| Genetically | | |
|---|---|---|
| modified strain | Parental strain | Genes overexpresses in the genetically |
| phenotype | phenotype | modified strain |
| Y1: smooth | M710: smooth | gpd1Δ::gpd2 fcy1Δ::STL1-STL1 |
| | | ime1Δ::STL1-STL1 |
| Y2: rugose | Isolate from Y1: | gpd1Δ::gpd2 fcy1Δ::STL1-STL1 |
| | smooth | ime1Δ::STL1-STL1 |
| Y3: rugose, fast | NA-wild-type | NA-wild-type |
| settling, high | | |
| invertase activity, | | |
| triploid, | | |
| hyperactivated | | |
| Ras/cAMP | | |
| Y4: rugose, fast | Y3 | fcy1Δ::STL1-STL1 ime1Δ::STL1-STL1 |
| settling, high | | |
| invertase activity, | | |
| triploid, | | |
| hyperactivated | | |
| Ras/cAMP | | |
| Y5: rugose, fast | NA-wild-type | NA-wild-type |
| settling, high | | |
| invertase activity, | | |
| triploid, | | |
| hyperactivated | | |
| Ras/cAMP | | |
| Y6: rugose, fast | Y5 | fcy1Δ::STL1-STL1 ime1Δ::STL1-STL1 |
| settling, high | | |
| invertase activity, | | |
| triploid, | | |
| hyperactivated | | |
| Ras/cAMP | | |
| Y7: rugose, fast | NA - wild-type | NA-wild-type |
| settling, triploid, | | |
| hyperactivated | | |
| Ras/cAMP | | |
| Y8: rugose, fast | Y7 | gpd1Δ::gpd2 fcy1A::STL1-STL1 |
| settling, triploid, | | |
| hyperactivated | | |
| Ras/cAMP | | |
| Y61 | Y3: rugose, fast | gpd1Δ::gpd2-STL1-STL1 |
| | settling, high | |
| | invertase activity, | |
| | triploid, | |
| | hyperactivated | |
| | Ras/cAMP | |
| Y62 | Y3: rugose, fast | fcy1Δ::SmGAPN-SmGAPN |
| | settling, high | |
| | invertase activity, | |
| | triploid, | |
| | hyperactivated | |
| | Ras/cAMP | |
| Y63 | Y3: rugose, fast | zwf1Δ::SmGAPN-SmGAPN |
| | settling, high | |
| | invertase activity, | |
| | triploid, | |
| | hyperactivated | |
| | Ras/cAMP | |
| Y64 | Y6: rugose, fast | fur1Δ::SmGAPN-SmGAPN |
| | settling, high | fcy1Δ::STL1-STL1 |
| | invertase activity, | ime1Δ::STL1-STL1 |
| | triploid, | |
| | hyperactivated | |
| | Ras/cAMP | |
| Y65 | Y6 | fcy1Δ::STL1-STL1 |
| | | ime1Δ::STL1-STL1 |
| | | fur1Δ::SmGAPN-SmGAPN |
| Y66 | Y6 | zwf1Δ::SmGAPN-SmGAPN |
| | | fcy1Δ::STL1-STL1 |
| | | ime1Δ::STL1-STL1 |

### Fast Settling and Rugose Phenotype

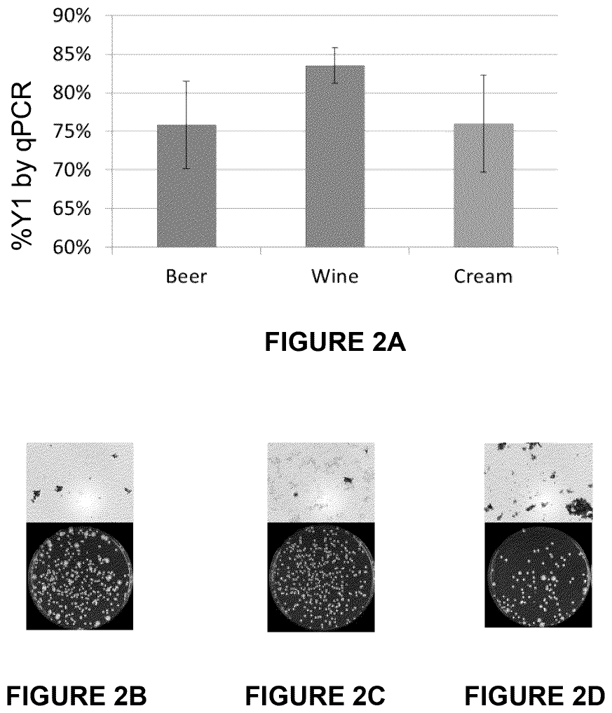

Strain Y1 was submitted to a commercial fermentation. The input and output streams of yeast strain Y1 from a centrifuge obtained at a commercial ethanol mill were examined. FIG. 2A shows that the percentage of Y1 DNA is higher in the wine stream as compared to the beer stream, indicating that the yeasts exiting the fermentation process and heading to the distillation process is enriched for strain Y1 compared to wild yeast strains. In addition, the Y1 strain percentage of total yeast DNA in the yeast cream being recycled to the process is the same or somewhat lower than what is found in the beer. These results suggest that yeast strain Y1 is being selected against during the process (most likely during the centrifuge step).

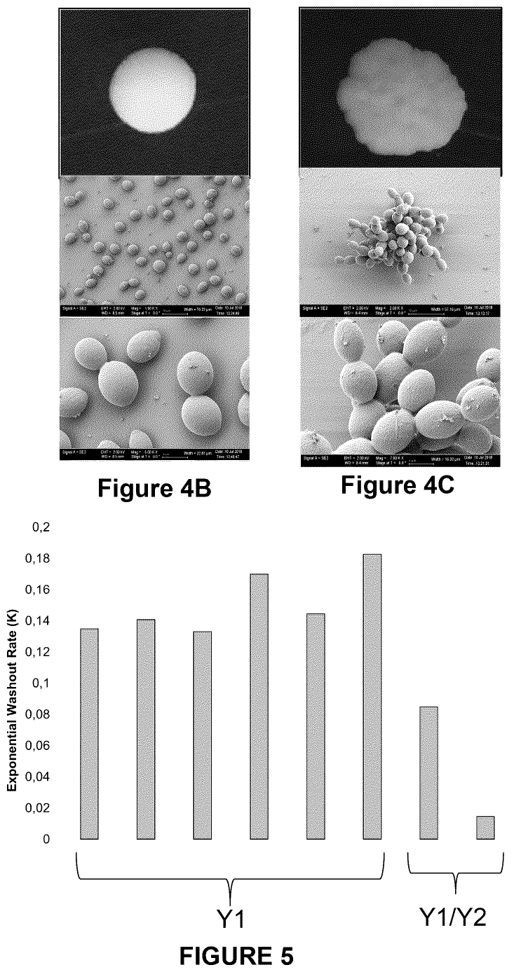

The Y1 strain is a smooth colony on a plate and does not present the rugose morphology. Under the microscope, the Y1 strain forms single or doublet cells similar to what is depicted in FIGS. 4A and 4B.

Microscopic analysis showed that the feed to the centrifuge, the beer, was composed of a mix of particle of different sizes (FIG. 2B), whereas the wine was composed of mostly small particles (FIG. 2C). The cream yeast, which is usually retained and sent back to the fermentation process, was composed of mostly larger particles (FIG. 2D). FIGS. 2B to 2D show the presence of “rugose” type colonies found in these streams. The feed to the centrifuge (beer) was composed of cells that form a mixture of rugose and smooth colonies (FIG. 2B). The wine was composed of cells that form exclusively smooth colonies (FIG. 2C). The yeast cream, which was retained, showed a mixture of cells forming smooth and rugose colonies, but was enriched for those cells that form rugose colonies relative to the beer (FIG. 2D). This data suggets that the centrifuge was preferentially selecting for cells that form rugose colonies to be retained in the process. Conversely, it showed that the centrifuge selected against those cells that form smooth colonies.

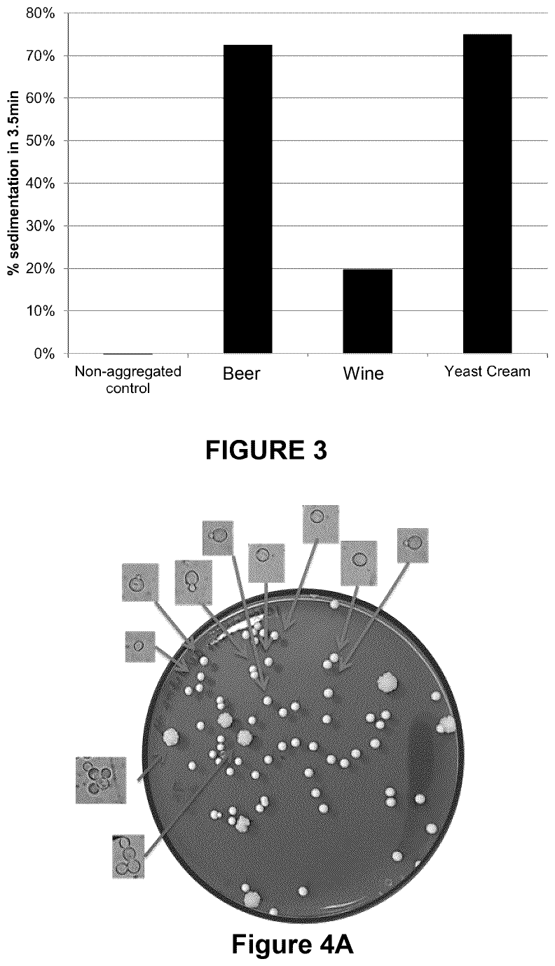

The settling velocity of the various yeast samples was determined. FIG. 3 shows the measurements of the rate of settling by gravity in a suspension after incubation for 3.5 minutes. The control, non-aggregated strain, does not settle at all under the conditions used for the assay. The input to the centrifuge (the beer) settles rapidly, as does the yeast cream output that will be recycled to the process. The wine showed much less/much slower settling of the particles it contained. This supports the principle that the centrifuge was acting to retain those particles that settle more rapidly.

Colonies that appear rugose on a growth plate form clusters of daughter yeast cells still connected to their mother (FIGS. 4A and 4C), while those that are smooth show the typical single cell or the budding morphology typical of S. cerevisiae (FIGS. 4A and 4B). The rugose/smooth phenotype of various commercial wild yeast populations was determined and is provided in Table 2.

TABLE 2
Colony morphology of yeast isolated from
various commercial cane fuel ethanol mills

| Sample | % Rugose |
|---|---|
| Mill 1 | 83% |
| Mill 2 | 91% |
| Mill 3 2017 | 90% |
| Mill 3 2018 | 100% |
| Mill 4 | 100% |
| Mill 4 End of season | 100% |
| Mill 5 | 100% |
| Mill 5 End of season | 100% |
| Mill 6 | 100% |
| Mill 6 End of season | 100% |
| Mill 7 | 80% |
| Mill 8 | 98% |
| All Mills | 95% |

Six commercial scale fermentations of the Y1 strain and two commercial scale fermentation of a 75:25 (weight) mixture of the Y1 and Y2 strains were conducted and the wash-out rates were determined. As shown in FIG. 5, when the rugose strain, Y2, was present, the exponential rate of washout slowed considerably. In one case (facility 7), it decreased by >40%, and in another (facility 8) it decreased by >90%.

Various isolates from the Y1 strain (like the Y2 strain) from the commercial process were found to be rugose after five months in a commercial fed batch fermentation process. Genome sequencing of various rugose isolates was performed to understand how yeasts can change from smooth to rugose during commercial implementations. Out of 53 936 SNPs and 7 273 indels present in the panel of 25 sequenced strains, only 1 SNP and 1 indel (both on a section of Y1 scaffold 6 corresponding to chromosome XII; data not shown) were associated with the rugose phenotype via genome-wide association test (p=1×10⁻⁶; data not shown). Both variants on scaffold 6 were nonsynonymous changes that predicted the rugose phenotype: a T->C variant in Choline Kinase (CK11) converting phenylalanine (TTT) to serine (TCT) at scaffold position 858 413, and a deletion at scaffold position 854 924 causing a translation frameshift in the Activator of CUP1 Expression (ACE2). These two candidate variants may be in linkage disequilibrium due to their close proximity (3 489 bp) and both fall in a distinct region of lost heterozygosity among rugose strains (data not shown). Given the prior functional evidence for ACE2, it is reasonable to assume that the ACE2 deletion was the strongest candidate causal mutation for the rugose phenotype, and the association at the CK11 SNP is a consequence of proximity.

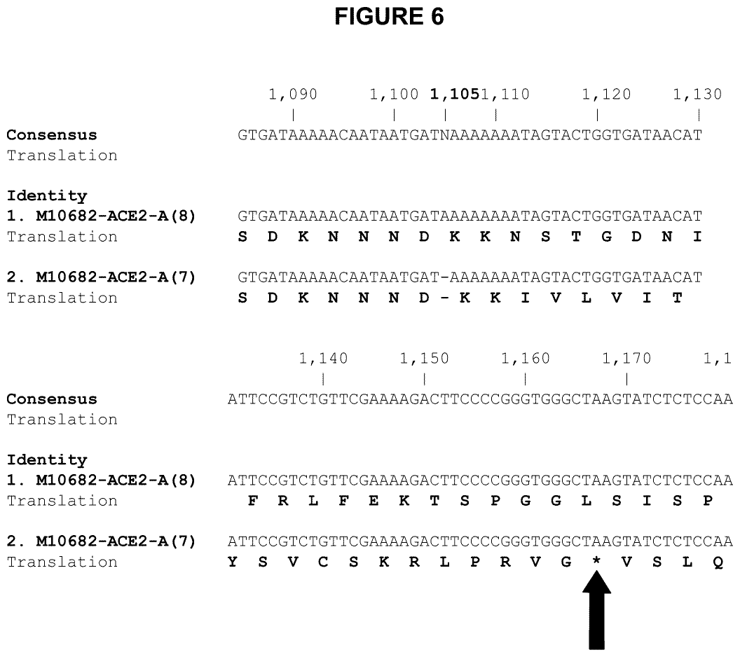

All the characterized rugose strains were homozygous for a single base pair deletion at base pair 1,112 in the poly-A region of ACE2 (genotype A(7):A(7), SEQ ID NO: 11), causing a translation frameshift and introducing an early stop codon at amino acid residue 389, 21 residues downstream of the deletion (FIG. 6). In comparison, as shown in Table 3, all smooth mill inoculants and isolates were heterozygous for the deletion (retaining one functional copy of ACE2, genotype A(7):A(8)), including Y1 itself, and the wild type parental strain PE-2. In contrast to the smooth industrial mill isolates, neither of two smooth isolates from a laboratory experiment possessed the deletion (homozygous genotype A(8):A(8)).

TABLE 3
Source, phenotype, and ACE2 genotype for smooth and rugose strains
sequenced for comparative genomics. A(8) is encoded by the gene
having the nucleic acid sequence of SEQ ID NO: 9 and has the amino
acid sequence of SEQ ID NO: 10. A(7) is encoded by the gene having
the nucleic acid sequence of SEQ ID NO: 11 and has the amino
acid sequence of SEQ ID: 12

| | | Strain | ACE2 |
|---|---|---|---|
| Source | Phenotype | designation | genotype |
| Wild type parental strain | Smooth | Y0 | A(7):A(8) |
| Industrial mill inoculants | Smooth | Y1 | A(7):A(8) |
| | Smooth | Y41 | A(7):A(8) |
| | Smooth | Y42 | A(7):A(8) |
| Lab experiment isolates | Smooth | Y43 | A(8):A(8) |
| | Smooth | Y44 | A(8):A(8) |
| | Rugose | Y45 | A(7):A(7) |
| | Rugose | Y46 | A(7):A(7) |
| Surviving mill isolates | Smooth | Y47 | A(7):A(8) |
| | Smooth | Y48 | A(7):A(8) |
| | Smooth | Y49 | A(7):A(8) |
| | Smooth | Y50 | A(7):A(8) |
| | Rugose | Y2 | A(7):A(7) |
| | Rugose | Y51 | A(7):A(7) |
| | Rugose | Y52 | A(7):A(7) |
| | Rugose | Y53 | A(7):A(7) |
| | Rugose | Y54 | A(7):A(7) |
| | Rugose | Y55 | A(7):A(7) |
| | Rugose | Y56 | A(7):A(7) |
| | Rugose | Y57 | A(7):A(7) |
| | Rugose | Y58 | A(7):A(7) |
| | Rugose | Y59 | A(7):A(7) |
| | Rugose | Y60 | A(7):A(7) |
| Contaminant mill isolates | Rugose | Y3 | A(7):A(7) |
| | Rugose | Y39 | A(7):A(7) |

As indicated above, the evaluation of wild contaminating yeasts in the fed batch fermentation process showed that >90% of colonies analyzed were rugose (Table 3) suggesting that the rugose morphology provides an advantage during the fermentation process.

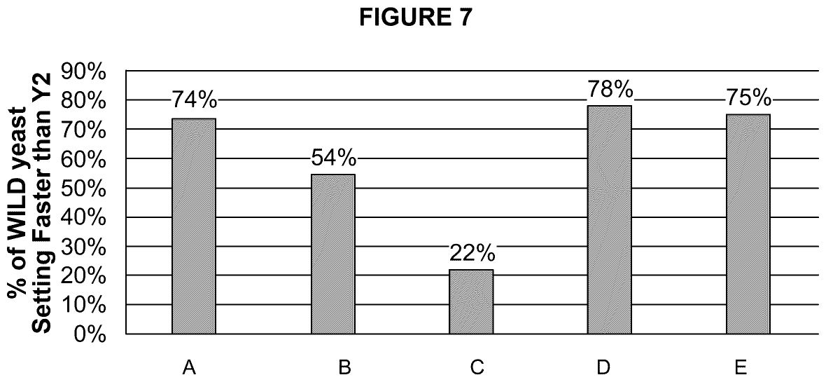

In addition, it was noted that the majority of yeasts in the mills were also extremely fast settling (e.g., fast sedimentation) when isolated and tested in a liquid settling assay (FIG. 7). The settling rate was maintained in the yeast even over 100 generations of passaging in rich glucose media showing that this is a stable feature of these mill yeasts (data not shown). Ninety (90) yeasts from various mills were tested in sedimentation and while all were fast settling, as expected due to their rugose phenotype, the majority at most mills settling even faster than the Y2 rugose natural isolate (data not shown).

Additionally, when the wild population was analyzed for mutations in the ACE2 gene, it was found that there was a high percentage of the truncated Ace2,7 A poly A tract, allele in the mill populations (FIG. 8).

Other mutations in the ACE2 gene were also identified amongst the wild yeasts that, similar to the A8 to A7, cause a premature stop codon. These mutations (presented in Table 4) can be seen in the wild population as shown in Table 5.

TABLE 4
Mutations observed in the ACE2 gene

| | | Effect of | |
|---|---|---|---|
| Mutation | Nucleotide | mutation | |
| location | location | on ACE2 | |
| in genome | in Ace2 | polypeptide | Amino acid sequence |
| 385, | 549 | Stop codon | MDNVVDPWYINPSGFAKDTQDEEYVQHHDNVNPTIPPPDN |
| 423-385, | | position at | YILNNENDDGLDNLLGMDYYNIDDLLTQELRDLDIPLVPS |
| 424: | | 169 | PKTGDGSSDKKNIDRTWNLGDENNKVSHYSKKSMSSHKRG |
| A6 > A7 | | | LSGTAIFGFLGHNKTLSISSLQQSILNMSKDPQPMELINE |
| insertion | | | LGNHNTVKK (SEQ ID NO: 13) |
| 385, 379: | 551 | Stop codon | MDNVVDPWYINPSGFAKDTQDEEYVQHHDNVNPTIPPPDN |
| A4 > A3 | | at position | YILNNENDDGLDNLLGMDYYNIDDLLTQELRDLDIPLVPS |
| deletion | | 186 | PKTGDGSSDKKNIDRTWNLGDENNKVSHYSKKSMSSHKRG |
| | | | LSGTAIFGFLGHNKTLSISSLQQSILNMSKDPQPMELINE |
| | | | LGNHNTVKNNNDDFDHIRENDGEIAI |
| | | | (SEQ ID NO: 14) |
| 385, 358: | 572 | Stop codon | MDNVVDPWYINPSGFAKDTQDEEYVQHHDNVNPTIPPPDN |
| T4 > T3 | | at position | YILNNENDDGLDNLLGMDYYNIDDLLTQELRDLDIPLVPS |
| deletion | | 191 | PKTGDGSSDKKNIDRTWNLGDENNKVSHYSKKSMSSHKRG |
| | | | LSGTAIFGFLGHNKTLSISSLQQSILNMSKDPQPMELINE |
| | | | LGNHNTVKNNNDDFDHIRENDGENSYLSQVC |
| | | | (SEQ ID NO: 15) |
| 384, 818: | 1112 | Stop codon | MDNVVDPWYINPSGFAKDTQDEEYVQHHDNVNPTIPPPDN |
| A8 > A7 | | position 389 | YILNNENDDGLDNLLGMDYYNIDDLLTQELRDLDIPLVPS |
| deletion | | | PKTGDGSSDKKNIDRTWNLGDENNKVSHYSKKSMSSHKRG |
| | | | LSGTAIFGFLGHNKTLSISSLQQSILNMSKDPQPMELINE |
| | | | LGNHNTVKNNNDDFDHIRENDGENSYLSQVLLKQQEELRI |
| | | | ALEKQKEVNEKLEKQLRDNQIQQEKLRKVLEEQEEVAQKL |
| | | | VSGATNSNSKPGSPVILKTPAMQNGRMKDNAIIVTTNSAN |
| | | | GGYQFPPPTLISPRMSNTSINGSPSRKYHRQRYPNKSPES |
| | | | NGLNLFSSNSGYLRDSELLSFSPQNYNLNLDGLTYNDHNN |
| | | | TSDKNNNDKKIVLVITYSVCSKRLPRVG |
| | | | (SEQ ID NO: 16) |

TABLE 5
Percentage of ACE2 mutants observed in wild populations. A = % of contaminating
cells, B = % of products, C = % of Y1, D = % of Y2, E = % ACE2 A7 @384,818,
F = % ACE2 T3 @385,358, G = % ACE2 A3 @385,379, H = % ACE2 A7 @385,423

| Sample | A | B | C | D | E | F | G | H |
|---|---|---|---|---|---|---|---|---|
| 1 | 0 | 100 | 100 | 0 | 49 | | | |
| 2 | 0 | 100 | 100 | 0 | 52 | | | |
| 3 | 0 | 100 | 100 | 0 | 52 | | | |
| 4 | 0 | 100 | 100 | 0 | 30 | | | |
| 5 | 0 | 100 | 100 | 0 | 57 | | | |
| 6 | 0 | 100 | 100 | 0 | 49 | | | |
| 7 | 0 | 100 | 100 | 0 | 47 | | | |
| 8 | 0 | 100 | 100 | 0 | 50 | | | |
| 9 | 0 | 100 | 100 | 0 | 62 | | | |
| 10 | 1 | 99 | 99 | 0 | 46 | | | |
| 11 | 1 | 99 | 99 | 0 | 50 | | | |
| 12 | 43 | 57 | 57 | 0 | 40 | | | |
| 13 | 94 | 6 | 6 | 0 | 64 | | | |
| 14 | 94 | 6 | 6 | 0 | 60 | | | |
| 15 | 19 | 81 | 81 | 0 | 46 | | | |
| 16 | 58 | 42 | 42 | 0 | 29 | | | |
| 17 | 0 | 100 | 74 | 26 | 60 | | | |
| 18 | 0 | 100 | 100 | 0 | 51 | | | |
| 19 | 0 | 100 | 86 | 14 | 62 | | | |
| 20 | 0 | 100 | 96 | 4 | 58 | | | |
| 21 | 0 | 100 | 86 | 14 | 71 | | | |
| 22 | 0 | 100 | 72 | 28 | 65 | | | |
| 23 | 0 | 100 | 76 | 24 | 65 | | | |
| 24 | 0 | 100 | 67 | 32 | 71 | 0 | 0 | 0 |
| 25 | 3 | 97 | 64 | 33 | 71 | 0 | 0 | 0 |
| 26 | 33 | 67 | 40 | 27 | 41 | 0 | 11 | 14 |
| 27 | 85 | 15 | 11 | 5 | 12 | 0 | 21 | 51 |
| 28 | 94 | 6 | 6 | 0 | 3 | 0 | 28 | 70 |
| 29 | 88 | 12 | 12 | 0 | 92 | | | |
| 30 | 99 | 1 | 1 | 0 | 35 | 0 | 10 | 0 |
| 31 | 0 | 100 | 78 | 22 | 64 | 0 | 0 | 0 |
| 32 | 0 | 100 | 64 | 36 | 68 | | | |
| 33 | 0 | 100 | 96 | 4 | 64 | | | |
| 34 | 0 | 100 | 66 | 34 | 76 | | | |
| 35 | 0 | 100 | 100 | 0 | 72 | | | |
| 36 | 0 | 100 | 80 | 20 | 54 | | | |
| 37 | 0 | 100 | 76 | 24 | 66 | | | |
| 38 | 0 | 100 | 80 | 20 | 66 | | | |
| 39 | 0 | 100 | 84 | 16 | 62 | | | |
| 40 | 0 | 100 | 74 | 26 | 70 | | | |
| 41 | 0 | 100 | 74 | 26 | 61 | | | |
| 42 | 0 | 100 | 80 | 20 | 70 | | | |
| 43 | 0 | 100 | 72 | 28 | 72 | | | |
| 44 | 0 | 100 | 92 | 8 | 71 | | | |
| 45 | 0 | 100 | 80 | 20 | 68 | 0 | 0 | 0 |
| 46 | 0 | 100 | 66 | 34 | 64 | 0 | 0 | 0 |
| 47 | 0 | 100 | 74 | 26 | 59 | 0 | 0 | 0 |
| 48 | 0 | 100 | 68 | 32 | 63 | 0 | 0 | 0 |
| 49 | 1 | 99 | 64 | 35 | 68 | 0 | 0 | 0 |
| 50 | 97 | 3 | 3 | 0 | 53 | | | |
| 51 | 99 | 1 | 1 | 0 | 55 | 23 | 28 | 0 |
| 52 | 96 | 4 | 4 | 0 | 18 | | | |
| 53 | 98 | 2 | 2 | 0 | 20 | 0 | 85 | 0 |
| 54 | 89 | 11 | 4 | 7 | 40 | | | |
| 56 | 99 | 1 | 1 | 0 | 16 | 0 | 82 | 0 |
| 57 | 7 | 93 | 90 | 3 | 39 | | | |
| 58 | 97 | 3 | 3 | 0 | 40 | | | |

The Y1 strain was run in co-inoculation with Y2 (at a 75:25 ratio) at commercial scale to determine if selection of the Y2 strain occurred in a commercial process. DNA was extracted from particular fermentation cycles throughout the implementation test and Illumina sequenced (2×126 bp read data; short reads of all DNA present). Read data was processed and aligned against the Y0 (e.g., PE-2) reference genome, computing read coverage across the genome and calling variants from each alignment. The relative abundance was determined for Y1 vs. Y2 in co-pitched fermentations. Tracking of two alleles that are present at higher levels in the Y2 background (cda1-2 A:A and Ace2 A7:A7) allowed to estimate if Y2 was increasing relative to Y1. During the implementation the level of Y2 as reported by the CDA1-2 A and Ace2 A7 alleles increase by ˜2-fold over the 30 cycles of the commercial test (FIG. 9).

### Invertase Activity

The acid treatment in the fed batch fermentation process is typically run at pH 2-3. After acid treatment sugarcane must is fed to the fermentation over 3-8 hours which buffers the pH of the fermentation. A typical fermentation starts at pH 2-3 and ends with a pH of 4-5.5 depending on the buffering capacity of the fed must. Sucrose is the predominate sugar in must and needs to be hydrolyzed by yeast expressed invertase to glucose and fructose. The typical pH optimum for S. cerevisiae invertase ranges over 3.5-5. It was determined if strains with increased invertase activity in the process could be more competitive. Without wishing to be bound to theory, increased invertase activity could provide an advantage during the first few hours of feeding when the pH is low coming out of acid treatment. The improved invertase activity could be derived from expressing an enzyme that has higher activity at lower pH, expressing and/or secreting higher level of an invertase (which may be more acid stable).

Invertase activity of 75 yeast isolates from a commercial mill was measured on YPD and commercial must after 48 hours of anaerobic growth. Invertase activity was measured by mixing cells and supernatant with a 40 g/L sucrose solution at pH 5 and incubated at 32° C. for 12 minutes and reducing sugar was determined using DNS and monitored by OD₅₄₀. In Table 6, the average fold increase in invertase activity vs. Y0 is ˜12 fold higher on either YPD or must.

TABLE 6
Invertase activity of 75 isolates from a commercial
mill compared to commercial strain Y0.

| | fold difference in |
|---|---|
| | invertase activity from Y0 |
| YPD anaerobic | 13.8 |
| Must anaerobic | 12.0 |

The invertase activity was also measured for two mill isolates on a variety of substrates after 48 h of anaerobic growth compared to yeast samples taken from the end of 15 cycles of acid treatment and fermentation. The mill isolated yeasts had invertase activity on all media tested but drastically higher activity when the activity is tested on cells directly out of the fed-batch process (Table 7).

TABLE 7
Fold change in invertase activity of engineered
wild yeast vs. Y0 on various media

| | | | | | | | 15 cycles |
|---|---|---|---|---|---|---|---|
| | | | | | | | in cell |
| | | | | Commercial | Commercial | Commercial | recycle |
| | YPD | YPS | YPF | Must 1 | Must 2 | Must 3 | system |
| Y4 | 2.0 | 2.6 | 2.6 | 1.8 | 2.2 | 1.7 | 11.8 |
| Y6 | 2.3 | 2.5 | 2.9 | 1.9 | 2.5 | 1.7 | 7.0 |

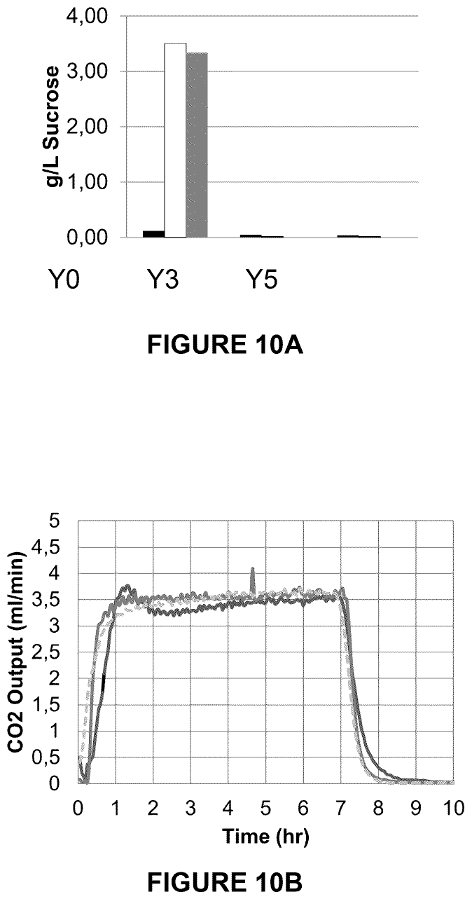

This higher level of invertase was reflected in the faster hydrolysis of sucrose of strains Y3 and Y5 during a fed-batch sucrose fermentation compared to the Y0 strain (FIG. 10A). Faster sucrose hydrolysis allowed strains Y3 and Y5 to ferment faster than strain Y0 as seen by the faster production of ethanol (FIG. 10B).

The rate of invertase activity associated with strains Y0, Y2, Y3, Y4, Y7 and Y8 (as the amount of reducing sugar/dry cell weight per minute) was determined on commercial must and is shown on FIG. 19 as a box and whisker plot obtained using the JMP software.

The rate of invertse activity associated with Y61 was determined to be equal to or higher than its parental strain Y3.

### RAS/cAMP/PKA Activity

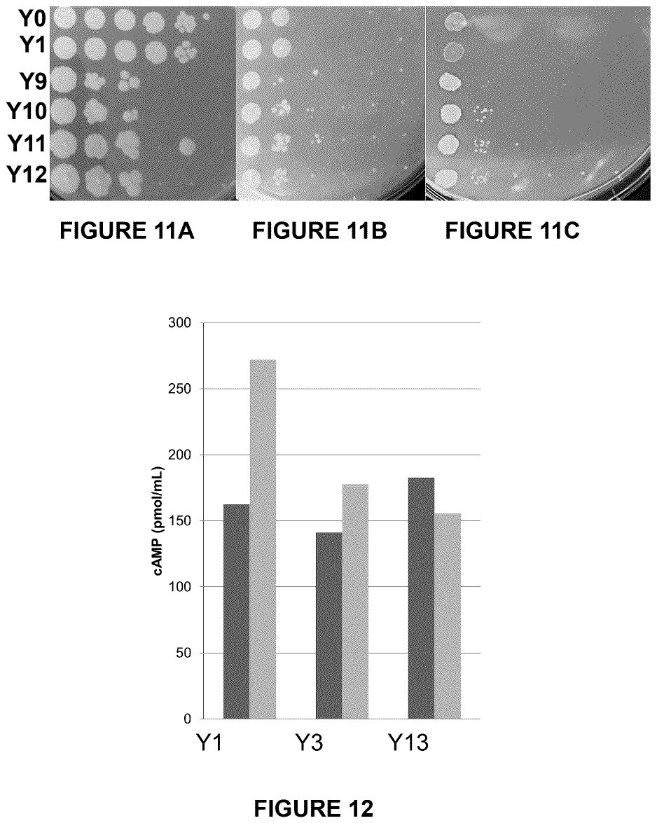

Strains showing normal RAS/cAMP/PKA activity (Y0 and Y1) and strains showing hyperactivated RAS/cAMP activity (Y9, Y10, Y11 and Y12) were tested on media containing 2-deoxyglucose and rapamycin. As shown in FIGS. 11B and 11C, the strains exhibiting hyperactivated Ras/cAMP activity were less sensitive to these compounds (which perturb glucose and nitrogen sensing in yeast). Without wishing to be bound to theory, these results suggests that the strains may have differential regulation of the RAS/cAMP/PKA pathway which provide them with a growth.

The ability of strains Y0, Y3 and Y13 to modulate their cAMP production was measured before and after (5 min) a 100 mM glucose spike. As shown in Table 8, strains Y3 and Y13 were not able to produce as much cAMP as strain Y0 (which does not exhibit an increase in the signaling on the RAS/cAMP/PKA pathway).

TABLE 8
cAMP production following glucose spike

| | | | Fold increase | % change in |
|---|---|---|---|---|
| | | cAMP levels | in cAMP level | fold increase |
| | | 5 min after a | at 5 min | when |
| | cAMP levels in | 100 mM glucose | following | compared to |
| Strain | basal medium | spike | glucose spike | Y0 |
| Y0 | 162.592 | 272.416 | 1.7 | N. A. |
| Y3 | 141.108 | 178.044 | 1.3 | −25% |
| Y13 | 182.816 | 155.808 | 0.9 | −49% |

The RAS/cAMP/PKA activity was specifically measured for recombinant strain Y1 as well as wild yeast strains Y3 and Y13. Strain Y1 showed almost 2 fold increase in cAMP production upon glucose spike showing a typical upregulation of the cAMP/PKA pathway (FIG. 12). Two commercial isolates, Y3 and Y13 showed no cAMP spike after the addition of glucose. This suggests that the cAMP pathway is hyperactive even at a basal state in strains Y3 and Y13.

Yeast cream samples were taken from various commercial mills (from which the Y3 and Y13 isolates were obtained) and Illumina sequencing was performed on the mixed commercial samples. The sequence of genes involved in the cAMP/PKA pathway were analyzed and a homozygous IRA2 Lys:Lys 2440/3079 at position was identified that differed from the beginning of the season where the strains were homozygous Glu:Glu (Table 9) The prevalence of this mutation in various mills suggested an adaptive advantage. Consistent with the IRA2 Lys mutation in these strain leading to derepression cAMP/PKA pathway.

TABLE 9
IRA2 mutations observed in wild populations.
A = % of contaminating cells, B = % of products, C = % of Y1,
D = % of Y2, E = % cells expressing the CDA1/2 A allele and
F = % of cells expressing an homozygous Lys:Lys mutation at
positions 2440 and 3079. The sample presented in this table
correspond to the samples presented in Table 5.

| Sample | A | B | C | D | E | F |
|---|---|---|---|---|---|---|
| 1 | 0 | 100 | 100 | 0 | 53 | 0 |
| 2 | 0 | 100 | 100 | 0 | 42 | 0 |
| 3 | 0 | 100 | 100 | 0 | 57 | 0 |
| 4 | 0 | 100 | 100 | 0 | 45 | 0 |
| 5 | 0 | 100 | 100 | 0 | 47 | 0 |
| 6 | 0 | 100 | 100 | 0 | 56 | 0 |
| 7 | 0 | 100 | 100 | 0 | 51 | 0 |
| 8 | 0 | 100 | 100 | 0 | 51 | 0 |
| 9 | 0 | 100 | 100 | 0 | 56 | 0 |
| 10 | 1 | 99 | 99 | 0 | 47 | 0 |
| 11 | 1 | 99 | 99 | 0 | 65 | 0 |
| 12 | 43 | 57 | 57 | 0 | 23 | 21 |
| 13 | 94 | 6 | 6 | 0 | 9 | 61 |
| 14 | 94 | 6 | 6 | 0 | 0 | 66 |
| 15 | 19 | 81 | 81 | 0 | 0 | 17 |
| 16 | 58 | 42 | 42 | 0 | 0 | 48 |
| 17 | 0 | 100 | 74 | 26 | 63 | 0 |
| 18 | 0 | 100 | 100 | 0 | 43 | 0 |
| 19 | 0 | 100 | 86 | 14 | 57 | 0 |
| 20 | 0 | 100 | 96 | 4 | 52 | 0 |
| 21 | 0 | 100 | 86 | 14 | 57 | 0 |
| 22 | 0 | 100 | 72 | 28 | 64 | 0 |
| 23 | 0 | 100 | 76 | 24 | 62 | 0 |
| 24 | 0 | 100 | 67 | 32 | 66 | 0 |
| 25 | 3 | 97 | 64 | 33 | 65 | 0 |
| 26 | 33 | 67 | 40 | 27 | 47 | 33 |
| 27 | 85 | 15 | 11 | 5 | 10 | 79 |
| 28 | 94 | 6 | 6 | 0 | 0 | 93 |
| 29 | 88 | 12 | 12 | 0 | 0 | 96 |
| 30 | 99 | 1 | 1 | 0 | 0 | 68 |
| 31 | 0 | 100 | 78 | 22 | 61 | 0 |
| 32 | 0 | 100 | 64 | 36 | 68 | 0 |
| 33 | 0 | 100 | 96 | 4 | 52 | 0 |
| 34 | 0 | 100 | 66 | 34 | 67 | 0 |
| 35 | 0 | 100 | 100 | 0 | 47 | 0 |
| 36 | 0 | 100 | 80 | 20 | 60 | 0 |
| 37 | 0 | 100 | 76 | 24 | 62 | 0 |
| 38 | 0 | 100 | 80 | 20 | 60 | 0 |
| 39 | 0 | 100 | 84 | 16 | 58 | 0 |
| 40 | 0 | 100 | 74 | 26 | 63 | 0 |
| 41 | 0 | 100 | 74 | 26 | 63 | 0 |
| 42 | 0 | 100 | 80 | 20 | 60 | 0 |
| 43 | 0 | 100 | 72 | 28 | 64 | 0 |
| 44 | 0 | 100 | 92 | 8 | 54 | 0 |
| 45 | 0 | 100 | 80 | 20 | 60 | 0 |
| 46 | 0 | 100 | 66 | 34 | 67 | 0 |
| 47 | 0 | 100 | 74 | 26 | 63 | 0 |
| 48 | 0 | 100 | 68 | 32 | 66 | 0 |
| 49 | 1 | 99 | 64 | 35 | 67 | 0 |
| 50 | 97 | 3 | 3 | 0 | 0 | 100 |
| 51 | 99 | 1 | 1 | 0 | 0 | 100 |
| 52 | 96 | 4 | 4 | 0 | 0 | 78 |
| 53 | 98 | 2 | 2 | 0 | 0 | 89 |
| 54 | 89 | 11 | 4 | 7 | 9 | 49 |
| 56 | 99 | 1 | 1 | 0 | 0 | 65 |
| 57 | 7 | 93 | 90 | 3 | 48 | 0 |
| 58 | 97 | 3 | 3 | 0 | 0 | |

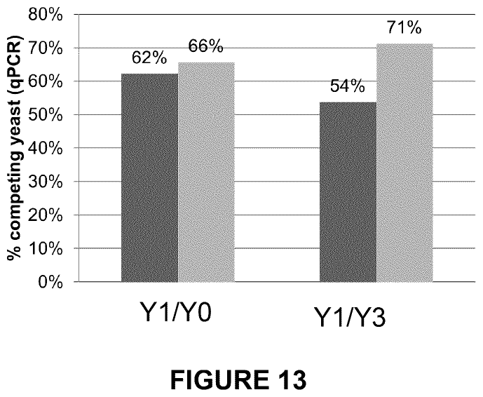

Strain Y1 was admixed with strain Y0 or a commercial isolate bearing a IRA Lys mutation, Y3 (ratio is provided on FIG. 13). As shown on FIG. 13, isolate Y3 rapidly outcompeted Y1 in three cycles of the fed-batch lab scale system rising from 54% to 71% of the population demonstrating the competitive advantage of the wild yeast.

### Triploid Phenotype

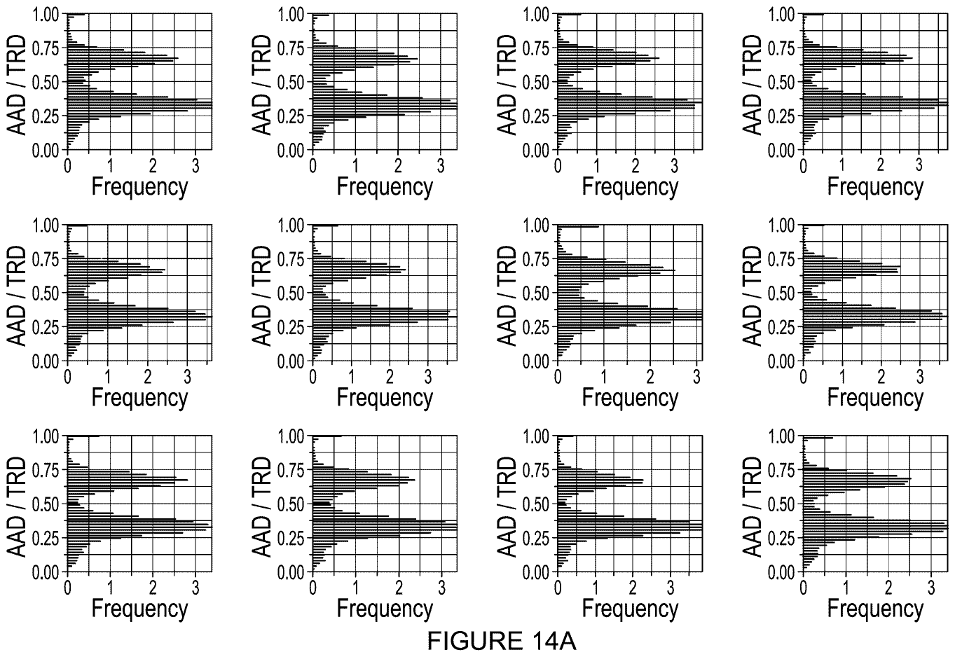

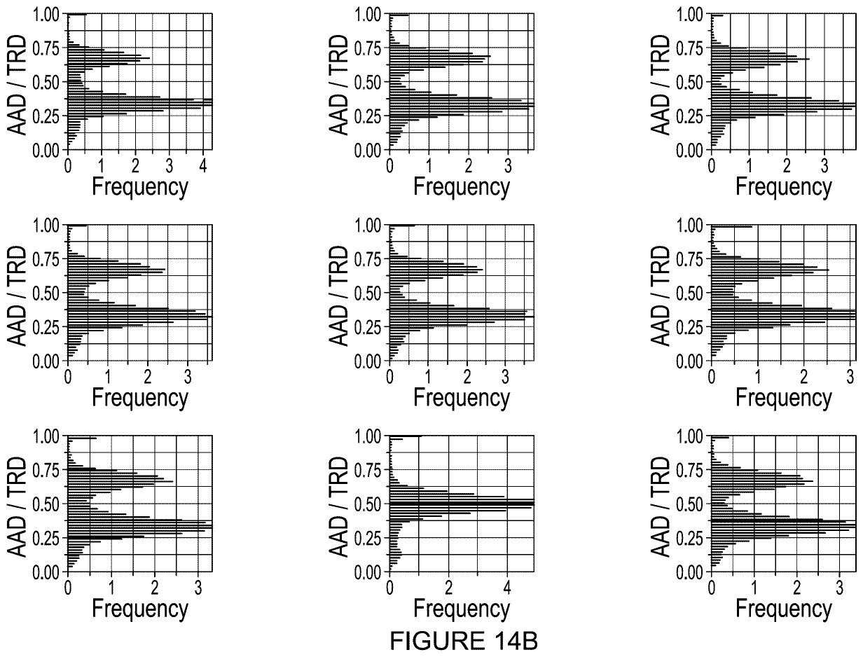

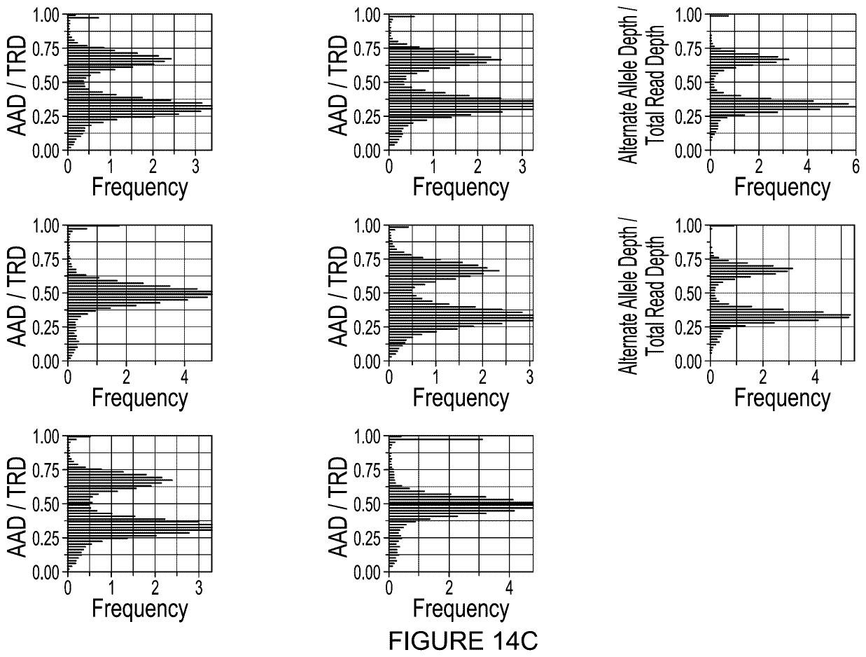

Various commercial and contaminating yeasts from mills were isolated and sequenced by Illumina sequencing. While each of the available commercial strains were found to be highly heterozygous diploid (data not shown), the majority of the strains isolated from the fermentation process in the end of the season were found to be highly heterozygous triploids (3n) (FIG. 14).

### Recombinant Yeast Expressing Heterologous STL1

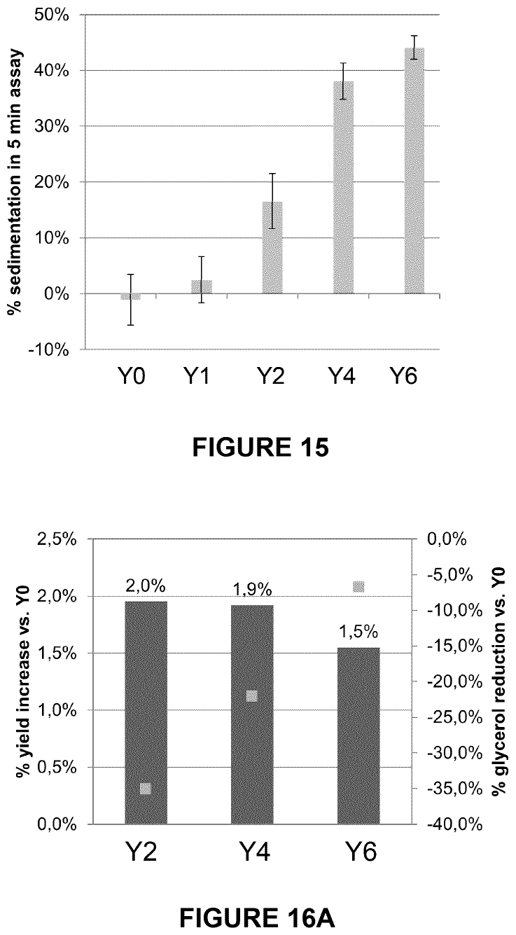

Strains Y4, Y6 and Y61 were genetically engineer to express the STL1 polypeptide. Briefly, two parental yeast strains (Y3 and Y5) isolated from the fed batch process with the dominant features of rugose colony formation, fast settling, high invertase activity post-acid treatment, triploid and features of cAMP/PKA hyperactivation were selected. These parental strains were then engineered with additional copies of STL1 to reduce glycerol and increase ethanol yields (see Table 1). After engineering, the strains were tested and demonstrated that they maintained the characteristics that they were chosen for fast settling (FIG. 15 for Y4 and Y6), hyperactivation of the RAS/cAMP pathway (data not shown) and high invertase activity (data not shown).

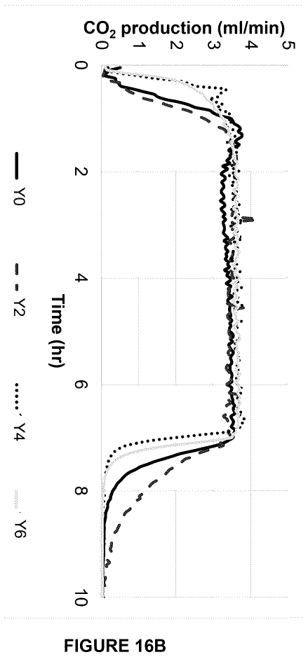

The performance of strains Y4 and Y6 was then monitored in a fed-batch high cell density fermentation with acid recycle for 13 rounds of fermentation. Over the 13 cycles, the yield of ethanol and glycerol were compared to the conventional strain Y0 and achieved a 1.9% yield increase for Y4 and a 1.5% yield increase for Y6 (FIG. 16A). These yield were close to the corresponding Y0-based strain (Y2) that is engineered and achieved a 1.9% yield increase over Y0. In addition, faster kinetics was maintained for strains Y4 and Y6 after engineering (FIG. 16B).

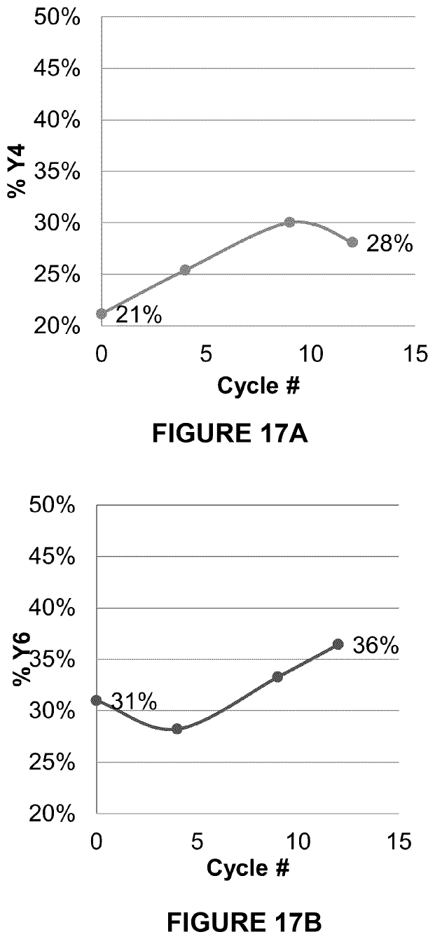

The strains Y4 and Y6 were then mixed in co-culture with Y2 to determine if they had growth advantage in co-culture and could displace the Y2 strain. A mixture of 20% Y4 to 80% Y2 and 30% Y6 to 70% Y2 were co-fermented in the fed-batch system with acid recycle. Populations were monitored using the qPCR method described above. Over the course of the 13 cycles of fermentation, both Y4 and Y6 increased within the population at an average of about 0.5% per cycle demonstrating their competitive fitness and growth advantage in this type of fermentation process (FIGS. 17A and 17B).

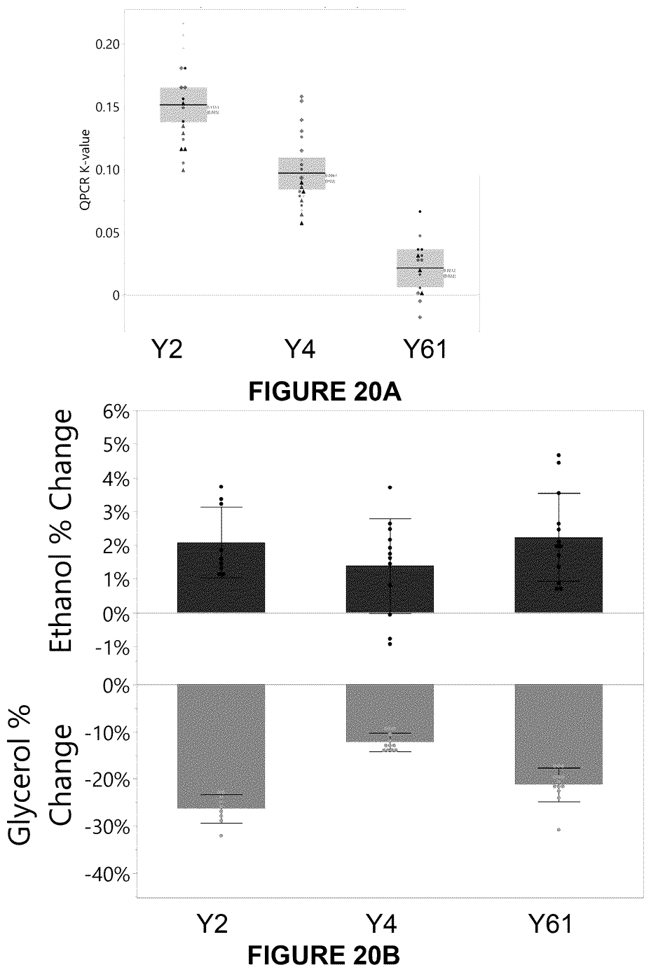

The performance of strain Y61 was monitored in co-culture fermentation experiments with strain Y3 on seven different commercial substrates and compared to the performances of strains Y2 or Y4 also in co-culture fermentation experiments with Y3. Populations were monitored using the qPCR method described above. Ethanol and glycerol levels were determined using HPLC. Over the various fermentations, the washout rate of strains Y4 and Y61 was lower than the washout rate of strain Y2 (FIG. 20A). These results indicated that the strains built in the Y3 background have improved competitive fitness during fermentation compared to the Y2 strain which was built in the Y0 background. In addition, strains Y2, Y4 and Y61 exhibited an increase in the percent in ethanol change as well as a decrease in the percent in glycerol changed when compared to strain Y0 (FIG. 20B). These results indicated that the strains with the persistent phenotypic traits maintained fermentation performances during multiple rounds of fermentation even though they included genetic modifications and expressed the heterologous STL1 gene.

### Recombinant Yeast Having Inactivated GPD1

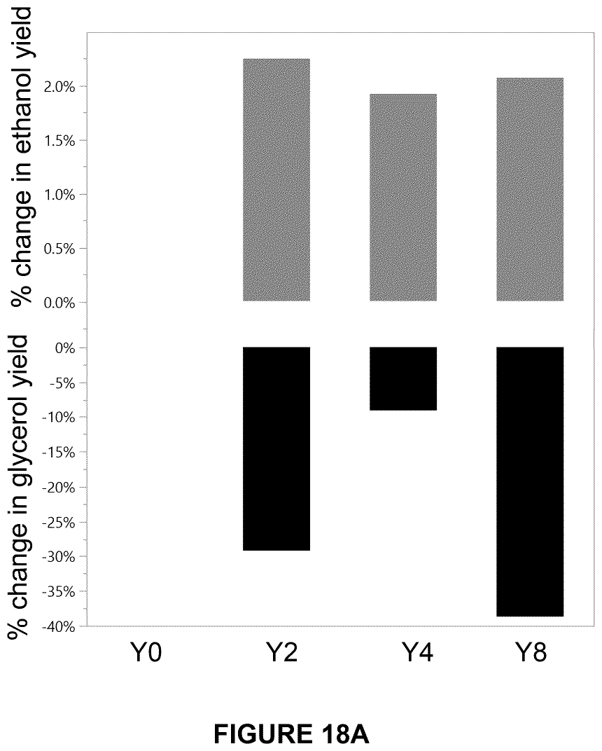

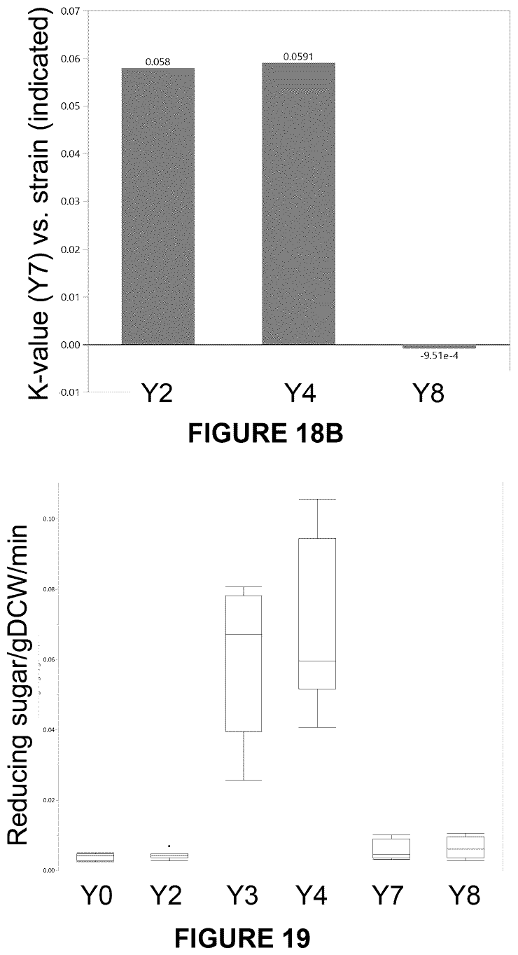

Strains Y0, Y2, Y4 and Y8 were pitched in monoculture or in co-culture with ˜15% of the wild type strain Y7. As described in Table 1 above, strain Y8 contained an additional modification that is not present in Y4, a replacement of the native GPD1 gene with GPD2 (Δgpd1::gpd2) in addition to the overexpression of STL1 (Δfcy1::st|1). This additional modification lead to a 2% yield increase over Y0 as well as higher levels of glycerol reduction (>−35% vs. Y0, FIG. 18A). In addition, strain Y8 showed a high levels of persistence over the recycle testing when in co-culture with Y7 (FIG. 18B).

### Improved Persistence

Several strains were submitted to commercial fermentations on cane must in which they were recycled and submitted to several consecutive cycles of fermentations. It was then determined, using quantitative PCR, the number of cycles at which the strain (or the combination of strains) represented 99%, 90% or 50% of the fermenting population. The results are presented at Table 10.

TABLE 10
Number of cycles at which the strain (or the combination of strains)
represented 99% (“number of cycles to 99%), 90% (“number of cycles
to 90%”) or 50% (“number of cycles to 90%”) of total the fermenting
population in function of each mill.

| | | Number | Number | Number |
|---|---|---|---|---|
| Mill | | of cycles | of cycles | of cycles |
| Code | Strain | to 99% | to 90% | to 50% |
| Mill #1 | Y1 | 50 | 53 | 72 |
| Mill #2 | Y1 | 29 | 45 | 53 |
| Mill #3 | Y1 | 41 | 48 | 53 |
| Mill #4 | Y1 | 26 | 30 | 38 |
| Mill #5 | Y1 | 29 | 38 | 42 |
| Mill #6 | Y2 | 100 | 120 | NA |
| Mill #7 | Y2 | 23 | 35 | 39 |
| Mill #8 | Y2 | 42 | 58 | 80 |
| Mill #9 | Y2 | 47 | 59 | 69 |
| Mill #10 | Y2 | 64 | 73 | 90 |
| Mill #11 | Y2 | 48 | 57 | 75 |
| Mill #12 | Y2 | 37 | 47 | 58 |
| Mill #13 | Y2 | 96 | 115 | 140 |
| Mill #14 | Y2 | 60 | 65 | 82 |
| Mill #15 | Y2 | 53 | 55 | 65 |
| Mill #16 | Y2 | 56 | 60 | 70 |
| Mill #17 | Y2 | 30 | 40 | 46 |
| Mill #18 | Y2 | 60 | 72 | 83 |
| Mill #19 | Y2 | 63 | 75 | 94 |
| Mill #20 | Y4 | 124 | 160 | 170 |
| Mill #21 | Y4 | 120 | 133 | 163 |
| Mill #22 | Y4 | 160 | 190 | 200 |
| Mill #23 | Y4 | 225 | 250 | 270 |
| Mill #24 | Y2 & Y4 | 75 | 100 | 116 |
| Mill #25 | Y2 & Y4 | 110 | 135 | 160 |
| Mill #26 | Y2 & Y4 | 88 | 100 | 120 |
| Mill #27 | Y2 & Y4 | 42 | 70 | 97 |

While the invention has been described in connection with specific embodiments thereof, it will be understood that the scope of the claims should not be limited by the preferred embodiments set forth in the examples, but should be given the broadest interpretation consistent with the description as a whole.

### REFERENCES

Luiz Carlos Basso, Thiago Olitta Basso and Saul Nitsche Rocha (Sep. 15, 2011). Ethanol Production in Brazil: The Industrial Process and Its Impact on Yeast Fermentation, Biofuel Production—Recent Developments and Prospects, Marco Aurelio dos Santos Bernardes, IntechOpen, DOI: 10.5772/17047.
Brociner R. E., Vollans E. C. (1973) Thickening flocculated kaolinite slurries in the nozzle discharge, multi disc, bowl centrifuge. Clay Minerals 10 (99).
Conrad M, Schothorst J, Kankipati H N, Van Zeebroeck G, Rubio-Texeira M, Thevelein J M. Nutrient sensing and signaling in the yeast Saccharomyces cerevisiae. FEMS Microbiol Rev. 2014; 38(2):254-299.
Danecek, Petr, Adam Auton, Goncalo Abecasis, Cornelis A. Albers, Eric Banks, Mark A. DePristo, Robert E. Handsaker, et al. “The Variant Call Format and VCFtools.” Bioinformatics (Oxford, England) 27, no. 15 (Aug. 1, 2011): 2156-58.
Fisher K J, Buskirk S W, Vignogna R C, Marad D A, Lang G I. Adaptive genome duplication affects patterns of molecular evolution in Saccharomyces cerevisiae. PLoS Genet. 2018; 14(5):e1007396. Published 2018 May 25.
Li, Heng. “A Statistical Framework for SNP Calling, Mutation Discovery, Association Mapping and Population Genetical Parameter Estimation from Sequencing Data.” Bioinformatics 27, no. 21 (Nov. 1, 2011): 2987-93.

Li, Heng, Bob Handsaker, Alec Wysoker, Tim Fennell, Jue Ruan, Nils Homer, Gabor Marth, Goncalo Abecasis, Richard Durbin, and 1000 Genome Project Data Processing Subgroup. “The Sequence Alignment/Map Format and SAMtools.” Bioinformatics (Oxford, England) 25, no. 16 (Aug. 15, 2009): 2078-79.
Oud B, Guadalupe-Medina V, Nijkamp J F, de Ridder D, Pronk J T, van Maris A J, Daran J M. Genome duplication and mutations in ACE2 cause multicellular, fast-sedimenting phenotypes in evolved Saccharomyces cerevisiae. Proc Natl Acad Sci USA. 2013 Nov. 5; 110(45):E4223-31. doi: 10.1073/pnas.1305949110. Epub 2013 Oct. 21. PMID: 24145419; PMCID: PMC3831460.
Purcell, Shaun, Benjamin Neale, Kathe Todd-Brown, Lori Thomas, Manuel A. R. Ferreira, David Bender, Julian Maller, “PLINK: A Tool Set for Whole-Genome Association and Population-Based Linkage Analyses.” American Journal of Human Genetics 81, no. 3 (September 2007): 559-75.
Scott A L, Richmond P A, Dowell R D, Selmecki A M. The Influence of Polyploidy on the Evolution of Yeast Grown in a Sub-Optimal Carbon Source. Mol Biol Evol. 2017; 34(10):2690-2703.
Selmecki A M, Maruvka Y E, Richmond P A, et al. Polyploidy can drive rapid adaptation in yeast. Nature. 2015; 519(7543):349-352.
Zadrag-Tecza R, Kwolek-Mirek M, Alabrudzihska M, Skoneczna A. Cell Size Influences the Reproductive Potential and Total Lifespan of the Saccharomyces cerevisiae Yeast as Revealed by the Analysis of Polyploid Strains. Oxid Med Cell Longev. 2018; 2018:1898421. Published 2018 Mar. 20.
Zhang, K., Fang, Y., Gao, K. et al. Effects of genome duplication on phenotypes and industrial applications of Saccharomyces cerevisiae strains. Appl Microbiol Biotechnol 101, 5405-5414 (2017).

1. A recombinant yeast host cell is capable of modulating the activity or the expression of a first polypeptide and/or a second polypeptide for increasing, when compared to a parental cell, the conversion of a biomass into a fermentation product and/or for reducing the conversion of the biomass into a fermentation by-product, wherein the recombinant yeast host cell has at least one of phenotypic trait providing persistence of the recombinant yeast host cell in a plurality of fermentation cycles, wherein the at least one phenotypic trait is:
a fast settling phenotype;
a rugose phenotype;
an improved invertase activity;
triploidy; or
increased signaling in a RAS/cAMP/PKA pathway.

2. The recombinant yeast host cell of claim 1 being capable of:
increasing the expression of the first polypeptide; and/or
decreasing or inactivating the expression of the second polypeptide, wherein the second polypeptide is a native polypeptide.

3. The recombinant yeast host cell of claim 2, wherein the first polypeptide comprises a sugar transporter-like protein (STL1).

4. The recombinant yeast host cell of claim 2, wherein the second polypeptide has NAD-dependent glycerol-3-phosphate dehydrogenase activity.

5. The recombinant yeast host cell of claim 4, wherein the second polypeptide is NAD-dependent glycerol-3-phosphate dehydrogenase comprises glycerol-3-phosphate 1 (GPD1) and/or NAD-dependent glycerol-3-phosphate is glycerol-3-phosphate 2 (GPD2).

6. The recombinant yeast host cell of claim 1 exhibiting the fast settling phenotype.

7. The recombinant yeast host cell of claim 6, wherein at least 5% of a population consisting essentially of the recombinant yeast host cells is able to sediment by gravity after 5 minutes and/or wherein the population consisting essentially of the recombinant yeast host cells is able to sediment by gravity in 5 minutes in a proportion equal to or higher than 15% than a control population consisting essentially of control yeast cells lacking the fast settling phenotype, and wherein the control yeast cells are from a Saccharomyces cerevisiae PE-2 strain.

8. The recombinant yeast host cell of claim 1 exhibiting the rugose phenotype.

9. The recombinant yeast host cell of claim 8, wherein at least 90% of a population consisting essentially of the recombinant yeast host cells, after exponential growth in a medium inoculated at low recombinant yeast host cell density, has at least two daughter cells attached.

10. The recombinant yeast host cell of claim 8 being capable of:
reducing the transcription factor activity of a Activator of CUP1 Expression (ACE2) polypeptide; and/or
expressing a mutated ACE2 polypeptide, wherein the mutated ACE2 polypeptide has decreased activity when compared to a wild type ACE2 polypeptide.

11. The recombinant yeast host cell of claim 1 exhibiting the improved invertase activity.

12. The recombinant yeast host cell of claim 11, wherein a population consisting essentially of the recombinant yeast host cells is able to hydrolyze more than 0.05 gram of sucrose per gram of dry cell weight per minute and/or exhibits more than 1.0 time invertase activity than a control population consisting essentially of control yeast cells lacking the improved invertase activity phenotypic trait, wherein the control yeast cells are from a Saccharomyces cerevisiae PE-2 strain, and wherein invertase activity is measured after exponential growth of the population diluted to a concentration of 9 mg/mL on a wet cell weight in a buffer and wherein the buffer comprises 40 g/L of sucrose, is at of pH 5 and at a temperature of 35° C.

13. The recombinant yeast host cell of claim 1 being capable of increasing the enzymatic activity of at least one polypeptide having invertase activity.

14. The recombinant yeast host cell of claim 13, wherein the at least one polypeptide having invertase activity comprises SUC1, SUC2, SUC3, SUC4, SUC5, SUC6, SUC7, SUC8 or SUC9.

15. The recombinant yeast host cell of claim 1 being a triploid cell.

16. The recombinant yeast host cell of claim 1 exhibiting increased signaling in the RAS/cAMP/PKA pathway.

17. The recombinant yeast host cell of claim 16, wherein a population consisting essentially of the recombinant yeast host cells is able to exhibit a fold increase in the production of cAMP of equal to or less than 1.7 and/or a fold increase in the production of cAMP production of less than 70% when compared a control population consisting essentially of control yeast cells lacking the increased signaling in the RAS/cAMP/PKA pathway phenotypic trait, wherein the control yeast cells are from a Saccharomyces cerevisiae PE-2 strain, and wherein the production of cAMP is measured in the population having been glucose depleted and 5 minutes after a glucose spike.

18. The recombinant yeast host cell of claim 16 being capable of expressing a mutated polypeptide involved in the RAS/cAMP/PKA pathway.

19. The recombinant yeast host cell of claim 18, wherein the mutated polypeptide involved in the RAS/cAMP/PKA pathway comprises a mutated RAS2 polypeptide having increased activity when compared to a wild-type RAS2 polypeptide, a mutated IRA2 polypeptide having a reduced inhibitory activity towards a wild-type RAS1 and/or a wild-type RAS2 polypeptide when compared to a wild-type IRA2 polypeptide.

20. The recombinant yeast host cell of claim 1 being from the genus Saccharomyces sp. or from the species Saccharomyces cerevisiae.

21. The recombinant yeast host cell of claim 2, wherein the first polypeptide comprises a glyceraldehyde-3-phosphate dehydrogenase.

22. The recombinant yeast host cell of claim 2, wherein the first polypeptide comprises a saccharolytic enzyme.
